(19) United States
(12) Patent Application Publication
(10) Pub. No.: US 2025/0324908 A1
(43) Pub. Date: Oct. 16, 2025

(54) LIGHT-EMITTING ELEMENT, AMINE COMPOUND FOR THE SAME, AND ELECTRONIC APPARATUS INCLUDING THE SAME

(71) Applicant: Samsung Display Co., Ltd., Yongin-si (KR)

(72) Inventor: Ichinori TAKADA, Tsurumi-ku (JP)

(21) Appl. No.: 19/060,348

(22) Filed: Feb. 21, 2025

(30) Foreign Application Priority Data
Apr. 15, 2024 (KR) 10-2024-0050306

(51) Int. Cl. H10K 85/60; C07C 211/54; H10K 50/12; H10K 50/15; H10K 50/17; H10K 59/38; H10K 101/25

(52) U.S. Cl. CPC H10K 85/633; C07C 211/54; H10K 85/636; C07C 2603/26; H10K 50/121; H10K 50/156; H10K 50/17; H10K 59/38; H10K 85/626; H10K 85/6572; H10K 85/6574; H10K 85/6576; H10K 2101/25

(57) ABSTRACT

A light-emitting element includes a first electrode, a second electrode on the first electrode, and at least one functional layer between the first electrode and the second electrode and including an amine compound represented by Formula 1.

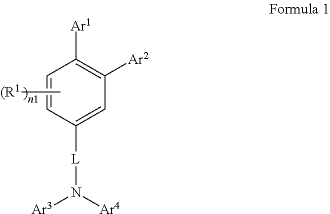

### CROSS-REFERENCE TO RELATED APPLICATION

The present application claims priority to and the benefit of Korean Patent Application No. 10-2024-0050306, filed on Apr. 15, 2024, in the Korean Intellectual Property Office, the entire disclosure of which is incorporated herein by reference.

### BACKGROUND

### 1. Field

One or more embodiments of the present disclosure relate to a light-emitting element, an amine compound for the light-emitting element, and an electronic apparatus including the light-emitting element.

### 2. Description of the Related Art

An electronic apparatus includes a display device that displays an image. Recently, the research and development of organic electroluminescence display devices and/or the like as image display devices have been actively conducted. The organic electroluminescence display devices and/or the like include self-luminous light-emitting elements where holes and electrons injected separately from a first electrode and a second electrode recombine in an emission layer (i.e., light-emitting layer). This recombination causes a light-emitting material in the emission layer to emit light, thereby achieving image display (e.g., display of images).

In the application of light-emitting elements in display devices, high luminous efficiency and long lifespan are desired or required. Therefore, the development of materials for light-emitting elements that can stably achieve such desired characteristics is being pursued actively.

Additionally, to implement light-emitting elements with high efficiency and long lifespan, there is active development of materials for hole transport regions that have improved stability and charge transportability.

### SUMMARY

One or more aspects of embodiments of the present disclosure are directed toward a light-emitting element having improved luminous efficiency and element lifespan.

One or more aspects of embodiments of the present disclosure are directed toward an amine compound capable of improving luminous efficiency and element lifespan of the light-emitting element including the amine compound.

One or more aspects of embodiments of the present disclosure are directed toward an electronic apparatus including the light-emitting element having improved luminous efficiency and lifespan and thus exhibiting excellent or suitable display quality.

Additional aspects will be set forth in part in the description which follows and, in part, will be apparent from the description, or may be learned by practice of the presented embodiments.

According to one or more embodiments of the present disclosure, a light-emitting element includes a first electrode, a second electrode on the first electrode, and at least one functional layer between the first electrode and the second electrode and containing an amine compound represented by Formula 1.

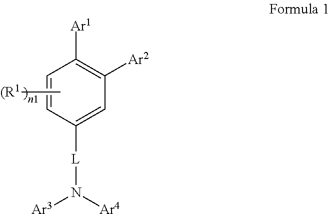

In Formula, any one selected from among Ar¹ and Ar² may be represented by Formula 2 or Formula 3, and the other may be an unsubstituted phenyl group, Ar³ and Ar⁴ may each independently be a substituted or unsubstituted aryl group having 6 to 30 ring-forming carbons or a substituted or unsubstituted heteroaryl group having 2 to 30 ring-forming carbons, L may be a direct linkage, a substituted or unsubstituted arylene group having 6 to 30 ring-forming carbons, a substituted or unsubstituted heteroarylene group having 2 to 30 ring-forming carbons, R¹ may be hydrogen, deuterium, or a substituted or unsubstituted alkyl group having 1 to 10 carbons, and n1 may be an integer of 0 to 3.

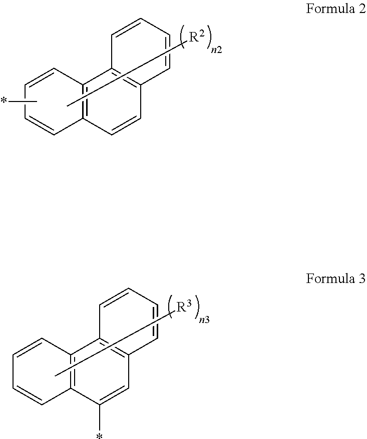

In Formula 2 and Formula 3, R² and R³ may each independently be hydrogen, deuterium, a substituted or unsubstituted alkyl group having 1 to 10 carbons, a substituted or unsubstituted aryl group having 6 to 30 ring-forming carbons, or a substituted or unsubstituted heteroaryl group having 2 to 30 ring-forming carbons, n2 and n3 may each independently be an integer of 0 to 9, -* is a position at which the rest portion of Formula 1 that is not represented by Formula 2 or Formula 3 is connected, in Formula 1, if (e.g., when) any one selected from among Ar¹ and Ar² is represented by Formula 3, at least one of Ar³ or Ar⁴ includes a substituted or unsubstituted naphthylphenyl group, a substituted or unsubstituted dibenzofuranyl group, a substituted or unsubstituted dibenzothiophenyl group, a substituted or unsubstituted 9-phenylcarbazolyl group, or a substituted or unsubstituted 9,9-diphenylfluorenyl group, in Formula 1, Ar³ and Ar⁴ do not include (e.g., exclude) a substituted or unsubstituted nitrogen-containing six-membered hetero ring, a substituted or unsubstituted 9,9-dimethylfluorenyl group, and a boron-containing derivative (e.g., boron-containing moiety), if (e.g., when) the amine compound represented by Formula 1 includes a four-membered fused hetero ring, the four-membered fused hetero ring is directly bonded to the nitrogen atom in Formula 1, in Formula 1, a case where at least one selected from among Ar³ and Ar⁴ is a substituted or unsubstituted phenanthrenyl group is excluded, and the amine compound may include a structure in which at least one hydrogen is substituted with deuterium, e.g., at least one hydrogen of the amine compound may be optionally substituted with deuterium.

In one or more embodiments, the at least one functional layer may include an light-emitting layer, a hole transport region between the first electrode and the light-emitting layer and an electron transport region between the light-emitting layer and the second electrode, wherein the hole transport region may include the amine compound represented by formula 1.

In one or more embodiments, the hole transport region may include a hole injection layer on the first electrode and a hole transport layer on the hole injection layer, wherein the hole transport layer includes the amine compound represented by Formula 1.

In one or more embodiments, the amine compound represented by Formula 1 may be represented by Formula 2-1 or Formula 2-2.

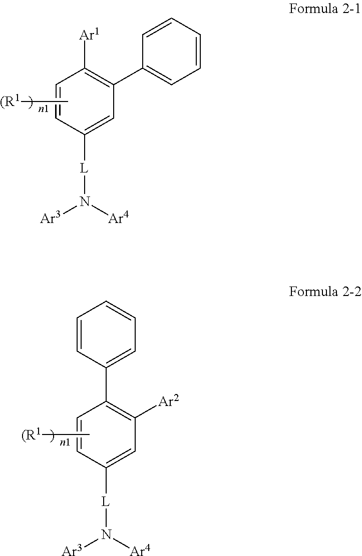

The descriptions as defined in Formula 1 may be similarly applied to R¹, n1, L, Ar¹, Ar², Ar³, and Ar⁴ in Formula 2-1 and Formula 2-2. In other words, R¹, n1, L, Ar¹, Ar², Ar³, and Ar⁴ in Formula 2-1 and Formula 2-2 may each independently be the same as defined in Formula 1.

In one or more embodiments, Ar³ and Ar⁴ may each independently be a substituted or unsubstituted phenyl group, a substituted or unsubstituted biphenylyl group, a substituted or unsubstituted naphthyl group, a substituted or unsubstituted naphthylphenyl group, a substituted or unsubstituted dibenzofuranyl group, a substituted or unsubstituted dibenzothiophenyl group, a substituted or unsubstituted carbazolyl group, or a substituted or unsubstituted fluorenyl group.

In one or more embodiments, Ar³ and Ar⁴ may each independently be substituted with deuterium, a substituted or unsubstituted phenyl group, a substituted or unsubstituted naphthyl group, a substituted or unsubstituted naphthylphenyl group, a substituted or unsubstituted fluorenyl group, a substituted or unsubstituted carbazolyl group, a substituted or unsubstituted dibenzofuranyl group, a substituted or unsubstituted dibenzothiophenyl group, or a substituted or unsubstituted phenanthrenyl group.

In one or more embodiments, the amine compound represented by Formula 1 is represented by Formula 3.

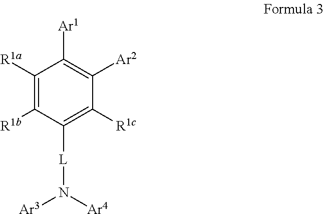

In Formula 3, R^{1a}, R^{1b}, and R^{1c} may each independently be hydrogen or deuterium.

The descriptions defined in Formula 1 may be similarly applied to L, Ar¹, Ar², Ar³, and Ar⁴ in Formula 3. In other words, L, Ar¹, Ar², Ar³, and Ar⁴ in Formula 3 may each independently be the same as defined in Formula 1.

In one or more embodiments, the amine compound represented by Formula 1 may be represented by any one selected from among Formula 4-1 to Formula 4-4.

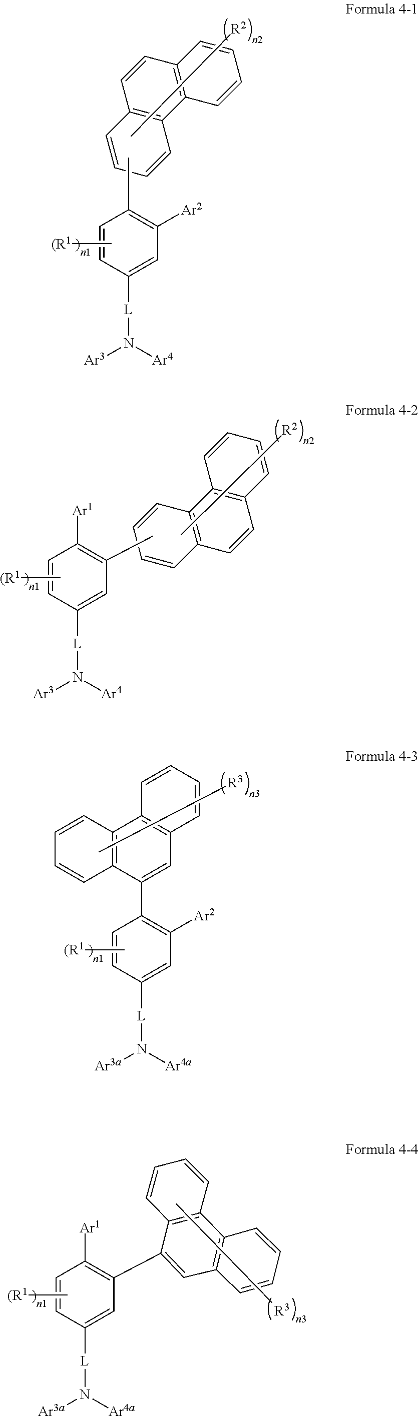

In Formula 4-3 and Formula 4-4, Ar^{3a} and Ar^{4a} may each independently be a substituted or unsubstituted aryl group having 6 to 30 ring-forming carbons or a substituted or unsubstituted heteroaryl group having 2 to 30 ring-forming carbons, and at least one selected from among Ar^{3a} and Ar^{4a} may include a substituted or unsubstituted naphthylphenyl group, a substituted or unsubstituted dibenzofuranyl group, a substituted or unsubstituted dibenzothiophenyl group, a substituted or unsubstituted 9-phenylcarbazolyl group, or a substituted or unsubstituted 9,9-diphenylfluorenyl group.

In Formula 4-1 to Formula 4-4, the descriptions defined in Formula 1 may be similarly applied to R¹, n1, L, Ar¹, Ar², Ar³, and Ar⁴. In other words, R¹, n1, L, Ar¹, Ar², Ar³, and Ar⁴ in Formula 4-1 to Formula 4-4 may each independently be the same as defined in Formula 1.

In Formula 4-1 to Formula 4-4, the descriptions defined in Formula 2 and Formula 3 may be similarly applied to R², R³, n2, and n3. In other words, R², R³, n2, and n3 in Formula 4-1 to Formula 4-4 may each independently be the same as defined in Formula 2 and Formula 3.

In one or more embodiments, the amine compound represented by Formula 1 may be represented by any one selected from among Formula 5-1 to Formula 5-3.

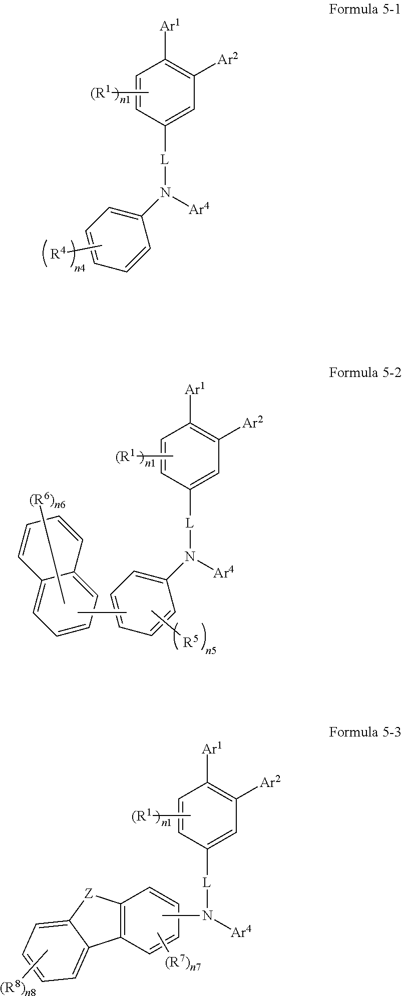

In Formula 5-1 to Formula 5-3, R⁴ to R⁸ may each independently be hydrogen, deuterium, a substituted or unsubstituted alkyl group having 1 to 20 carbons, a substituted or unsubstituted aryl group having 6 to 30 ring-forming carbons, 1 or a substituted or unsubstituted heteroaryl group having 2 to 30 ring-forming carbons, Z may be O, S, NR⁹, or CR¹⁰R¹¹, R⁹ to R¹¹ may each independently be a substituted or unsubstituted aryl group having 6 to 30 ring-forming carbons, n4 may be an integer of 0 to 5, n5 and n8 may each independently be an integer of 0 to 4, n6 may be an integer of 0 to 7, n7 may be an integer of 0 to 3, and in Formula 5-1, if (e.g., when) any one selected from among Ar¹ and Ar² is represented by Formula 3, R⁴ may be a substituted or unsubstituted dibenzofuranyl group, a substituted or unsubstituted dibenzothiophenyl group, a substituted or unsubstituted 9-phenylcarbazolyl group, or a substituted or unsubstituted 9,9-diphenylfluorenyl group.

In Formula 5-1 to Formula 5-3, the descriptions defined in Formula 1 above may be similarly applied to R¹, n1, L, Ar¹, Ar², and Ar⁴. In other words, R¹, n1, L, Ar¹, Ar², and Ar⁴ in Formula 5-1 to Formula 5-3 may each independently be the same as defined in Formula 1.

In one or more embodiments, the amine compound represented by Formula 1 may be represented by Formula 6, and the amine compound may satisfy any one selected from among combinations present in Compound Combination Table 1.

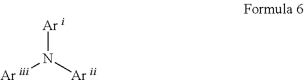

In Formula 6, Arⁱ may be any one selected from Substituent Group A, Arⁱⁱ may be any one selected from Substituent Group B, and Arⁱⁱⁱ may be any one selected from Substituent Group C.

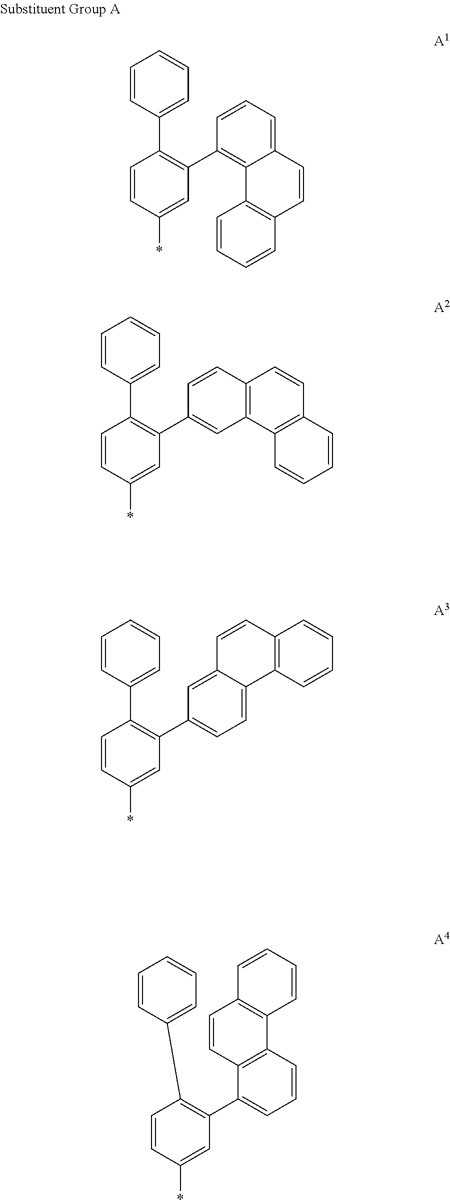 [SMILES: *c1ccc(-c2ccccc2)c(-c2cccc3ccc4ccccc4c23)c1 ; *c1ccc(-c2ccccc2)c(-c2ccc3ccc4ccccc4c3c2)c1 ; *c1ccc(-c2ccccc2)c(-c2ccc3c(ccc4ccccc43)c2)c1 ; *c1ccc(-c2ccccc2)c(-c2cccc3c2ccc2ccccc23)c1]

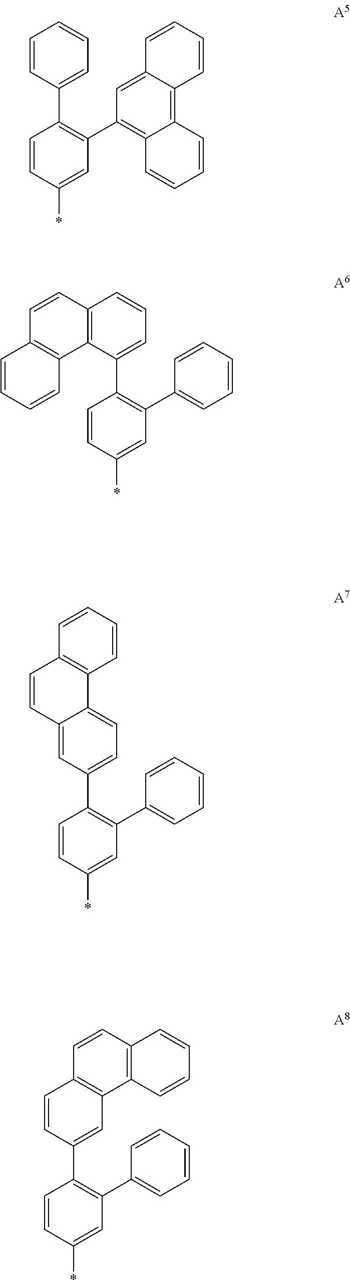 [SMILES: *c1ccc(-c2ccccc2)c(-c2cc3ccccc3c3ccccc23)c1 ; *c1ccc(-c2cccc3ccc4ccccc4c23)c(-c2ccccc2)c1 ; *c1ccc(-c2ccc3c(ccc4ccccc43)c2)c(-c2ccccc2)c1 ; *c1ccc(-c2ccc3ccc4ccccc4c3c2)c(-c2ccccc2)c1]

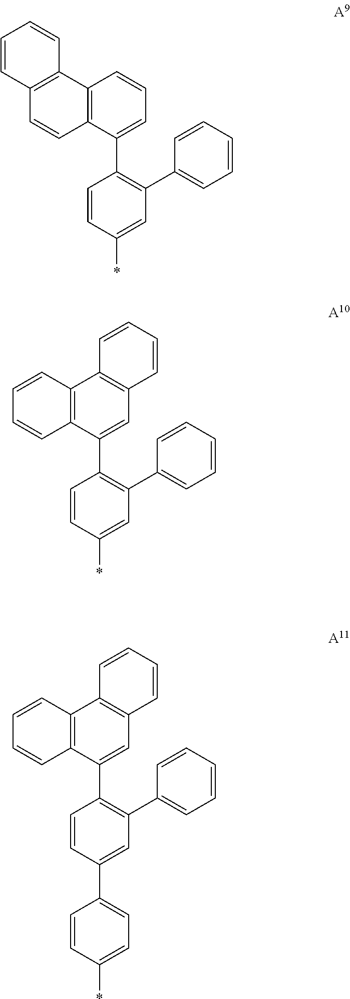 [SMILES: *c1ccc(-c2cccc3c2ccc2ccccc23)c(-c2ccccc2)c1 ; *c1ccc(-c2cc3ccccc3c3ccccc23)c(-c2ccccc2)c1 ; *c1ccc(-c2ccc(-c3cc4ccccc4c4ccccc34)c(-c3ccccc3)c2)cc1]

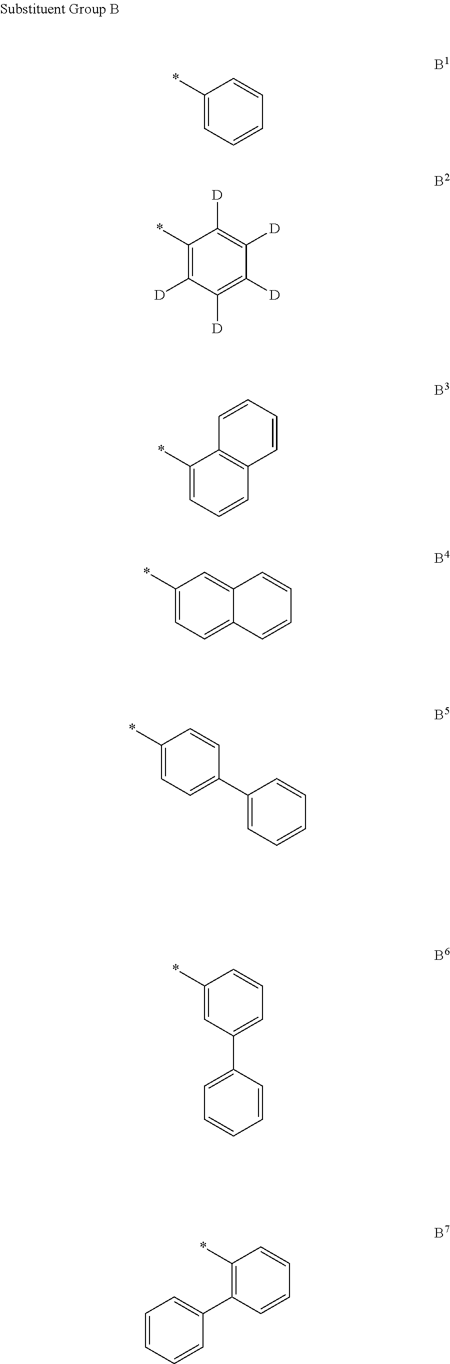

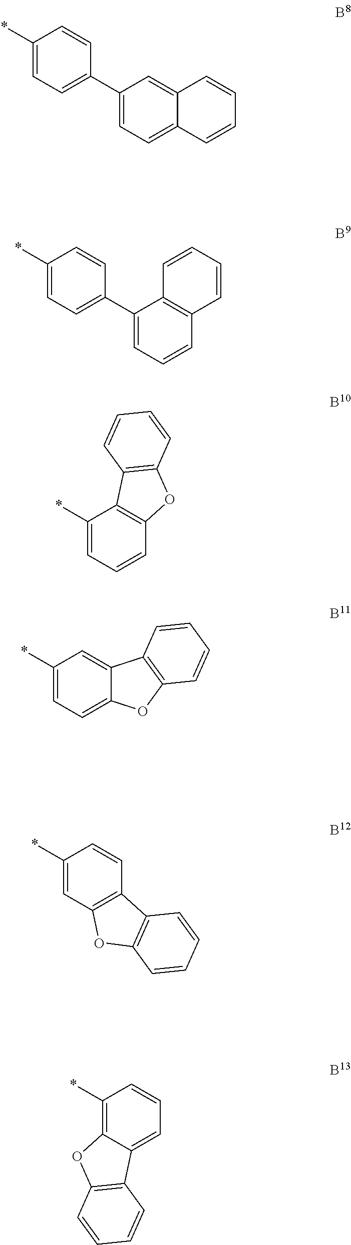 [SMILES: *c1ccc(-c2ccc3ccccc3c2)cc1 ; *c1ccc(-c2cccc3ccccc23)cc1 ; *c1cccc2oc3ccccc3c12 ; *c1ccc2oc3ccccc3c2c1 ; *c1ccc2c(c1)oc1ccccc12 ; *c1cccc2c1oc1ccccc12]

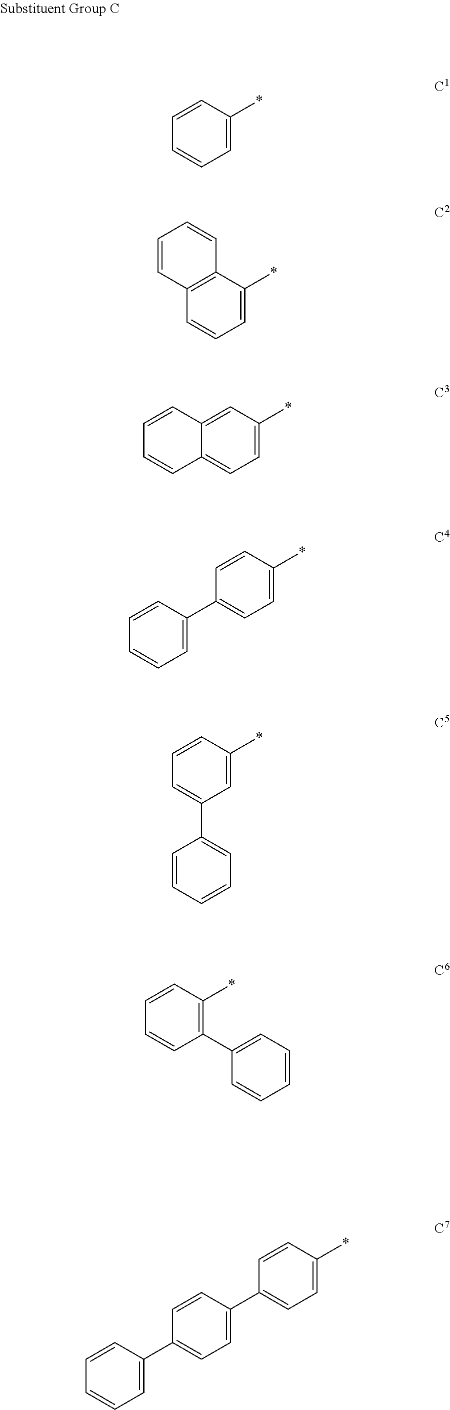

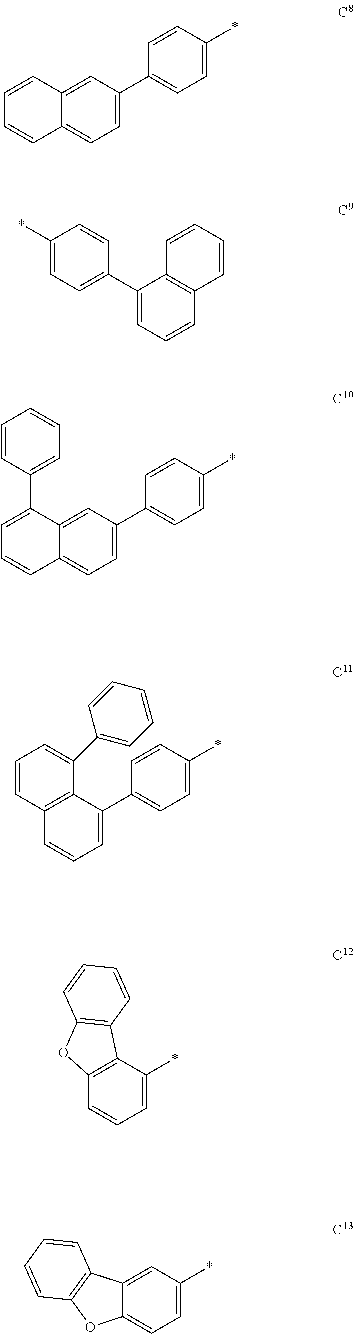 [SMILES: *c1ccc(-c2ccc3ccccc3c2)cc1 ; *c1ccc(-c2cccc3ccccc23)cc1 ; *c1ccc(-c2ccc3cccc(-c4ccccc4)c3c2)cc1 ; *c1ccc(-c2cccc3cccc(-c4ccccc4)c23)cc1 ; *c1cccc2oc3ccccc3c12 ; *c1ccc2oc3ccccc3c2c1]

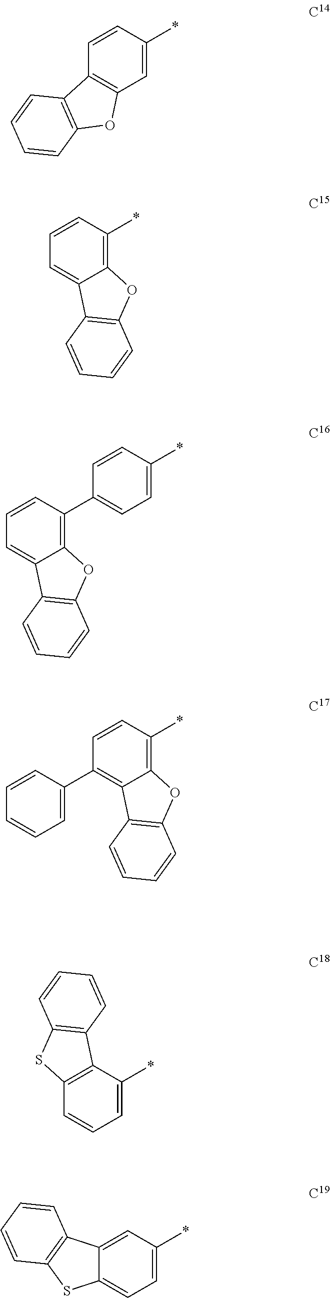 [SMILES: *c1ccc2c(c1)oc1ccccc12 ; *c1cccc2c1oc1ccccc12 ; *c1ccc(-c2cccc3c2oc2ccccc23)cc1 ; *c1ccc(-c2ccccc2)c2c1oc1ccccc12 ; *c1cccc2sc3ccccc3c12 ; *c1ccc2sc3ccccc3c2c1]

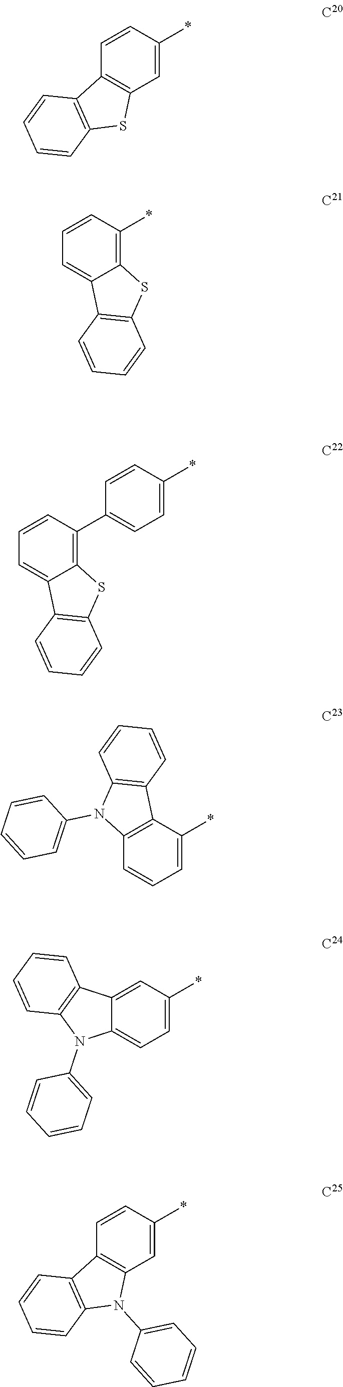 [SMILES: *c1ccc2c(c1)sc1ccccc12 ; *c1cccc2c1sc1ccccc12 ; *c1ccc(-c2cccc3c2sc2ccccc23)cc1 ; *c1cccc2c1c1ccccc1n2-c1ccccc1 ; *c1ccc2c(c1)c1ccccc1n2-c1ccccc1 ; *c1ccc2c3ccccc3n(-c3ccccc3)c2c1]

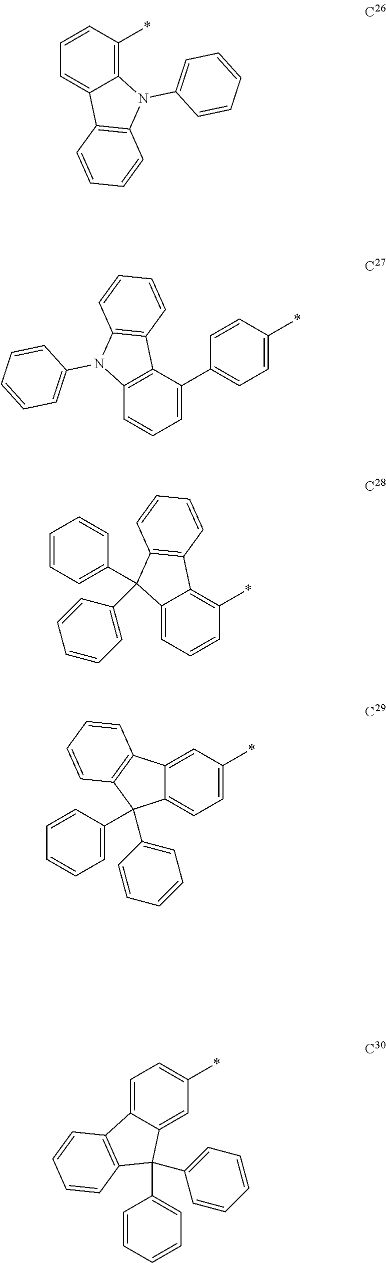 [SMILES: *c1cccc2c3ccccc3n(-c3ccccc3)c12 ; *c1ccc(-c2cccc3c2c2ccccc2n3-c2ccccc2)cc1 ; *c1cccc2c1-c1ccccc1C2(c1ccccc1)c1ccccc1 ; *c1ccc2c(c1)-c1ccccc1C2(c1ccccc1)c1ccccc1 ; *c1ccc2c(c1)C(c1ccccc1)(c1ccccc1)c1ccccc1-2]

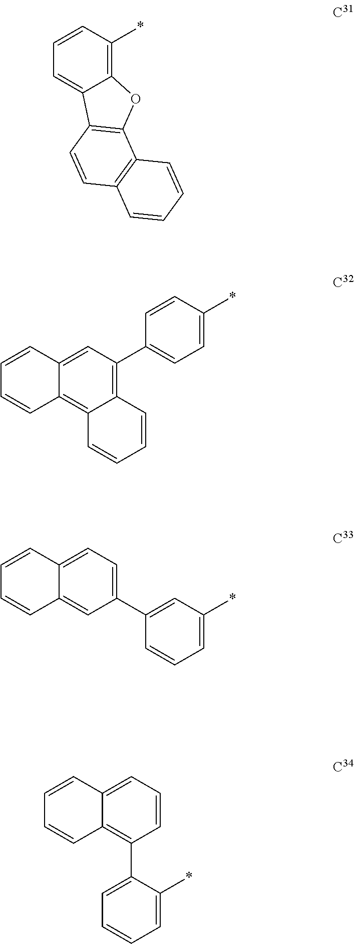 [SMILES: *c1cccc2c1oc1c3ccccc3ccc21 ; *c1ccc(-c2cc3ccccc3c3ccccc23)cc1 ; *c1cccc(-c2ccc3ccccc3c2)c1 ; *c1ccccc1-c1cccc2ccccc12]

In one or more embodiments of the present disclosure, a display device includes a base layer, a circuit layer on (e.g., arranged on) the base layer, and a display element layer on (e.g., arranged on) the circuit layer and including a light-emitting layer, wherein, the light-emitting element includes a first electrode, a second electrode on (e.g., arranged on) the first electrode, and at least one functional layer between the first and the second electrode and including an amine compound represented by Formula 1.

In one or more embodiments, the light-emitting element further may include a capping layer on (e.g., arranged on) the second electrode, and the capping layer may have a refractive index of at least about 1.6 for light in a wavelength range of about 550 nanometers (nm) to about 660 nm.

In one or more embodiments a light control layer on (e.g., arranged on) the display element layer and including a quantum dot may be further included, wherein the light-emitting element may be to emit first color light, and the light control layer may include a first light control part containing a first quantum dot that converts the first color light to second color light in a longer wavelength region than the first color light, a second light control part containing a second quantum dot that converts the first color light to third color light in a longer wavelength region than both the first color light and the second color light, and a third light control part configured to transmit the first color light.

In one or more embodiments, a color filter layer may be further included on the light control layer, the color filter layer may include a first filter configured to transmit the second color light, a second filter configured to transmit the third color light, and a third filter configured to transmit the first color light.

In one or more embodiments of the present disclosure, provided is an amine compound represented by Formula 1.

### BRIEF DESCRIPTION OF DRAWINGS

The accompanying drawings are included to provide a further understanding of the present disclosure, and are incorporated in and constitute a part of this disclosure. The drawings illustrate example embodiments of the disclosure and, together with the description, serve to explain principles of the present disclosure. Above and/or other aspects of the disclosure should become apparent and appreciated from the following description of embodiments taken in conjunction with the accompanying drawings. In the drawings:

FIG. 1 is a plan view illustrating a display apparatus according to one or more embodiments of the present disclosure;

FIG. 2 is a cross-sectional view of a display apparatus according to one or more embodiments of the present disclosure;

FIG. 3 is a cross-sectional view schematically illustrating a light-emitting element according to one or more embodiments of the present disclosure;

FIG. 4 is a cross-sectional view schematically illustrating a light-emitting element according to one or more embodiments of the present disclosure;

FIG. 5 is a cross-sectional view schematically illustrating a light-emitting element according to one or more embodiments of the present disclosure;

FIG. 6 is a cross-sectional view schematically illustrating a light-emitting element according to one or more embodiments of present disclosure;

FIG. 7 is a cross-sectional view of a display apparatus according to one or more embodiments of the present disclosure;

FIG. 8 is a cross-sectional view of a display apparatus according to one or more embodiments of the present disclosure;

FIG. 9 is a cross-sectional view illustrating a display apparatus according to one or more embodiments of the present disclosure;

FIG. 10 is a cross-sectional view illustrating a display apparatus according to one or more embodiments of the present disclosure;

FIG. 11 is a view illustrating a vehicle in which display apparatuses according to one or more embodiments of the present disclosure are arranged;

FIG. 12 is a perspective view showing an electronic apparatus according to one or more embodiments of present disclosure;

FIG. 13 is an exploded perspective view showing an electronic apparatus according to one or more embodiments of the present disclosure;

FIG. 14 is a block diagram of an electronic apparatus according to one or more embodiments of the present disclosure;

FIG. 15 is a diagram showing an electronic apparatus according to one or more embodiments of the present disclosure; and

FIG. 16 is a diagram showing an electronic apparatus according to one or more embodiments of the present disclosure.

### DETAILED DESCRIPTION

The present disclosure may be modified and implemented in one or more suitable manners and have many forms, and thus specific/example embodiments will be exemplified in the drawings and described in more detail in the detailed description of present disclosure. It should be understood, however, that it is not intended to limit the disclosure to the particular forms disclosed, but rather, is intended to cover all modifications, equivalents, and alternatives falling within the spirit and scope of the disclosure.

When explaining each of drawings, like reference numbers are used for referring to like elements. In the accompanying drawings, the dimensions of each structure may be exaggeratingly illustrated for clarity of the present disclosure. It will be understood that, although the terms “first,” “second,” and/or the like, may be used herein to describe one or more suitable components, these components should not be limited by these terms. These terms are only used to distinguish one component from another. For example, a first component could be termed a second component, and, similarly, a second component could be termed a first component, without departing from the scope of example embodiments of the disclosure. As used herein, the singular forms, “a,” “an,” “one,” and “the” are intended to include the plural forms as well, unless the context clearly indicates otherwise. Further, the utilization of “may” when describing embodiments of the present disclosure refers to “one or more embodiments of the present disclosure”.

In the present disclosure, it will be understood that the terms “comprise(s)/comprising,” “include(s)/including,” “have (has)/having,” and/or the like specify the presence of features, numbers, steps, operations, component, parts, and/or one or more (e.g., any suitable) combinations thereof disclosed in the disclosure, but do not exclude the possibility of presence or addition of one or more other features, numbers, steps, operations, component, parts, and/or one or more (e.g., any suitable) combinations thereof. As used herein, the terms “and,” “or,” and “and/or” may include any and all combinations of one or more of the associated listed items. Expressions such as “at least one of,” “one of,” and “selected from,” when preceding a list of elements, modify the entire list of elements and do not modify the individual elements of the list. For example, “at least one of a, b, or c”, “at least one selected from a, b, and c”, “at least one selected from among a to c”, etc., may indicate only a, only b, only c, both (e.g., simultaneously) a and b, both (e.g., simultaneously) a and c, both (e.g., simultaneously) b and c, all of a, b, and c, or variations thereof. The “/” utilized herein may be interpreted as “and” or as “or” depending on the situation.

In the present disclosure, if (e.g., when) a layer, a film, a region, or a plate is referred to as being “on” or “in an upper portion of” another layer, film, region, or plate, it may be not only “directly on” the layer, film, region, or plate, but one or more intervening layers, films, regions, or plates may also be present therebetween. Opposite this, if (e.g., when) a layer, a film, a region, or a plate is referred to as being “below”, “in a lower portion of” another layer, film, region, or plate, it may be not only directly under the layer, film, region, or plate, but one or more intervening layers, films, regions, or plates may also be present therebetween. In addition, it will be understood that if (e.g., when) a part is referred to as being “on” another part, it may be arranged above the other part, or arranged under the other part as well. In the present disclosure, “directly on” may refer to that there are no additional layers, films, regions, plates, and/or like, between a layer, a film, a region, a plate, etc. and the other part. For example, “directly on” may refer to two layers or two members are arranged without utilizing an additional member such as an adhesive member therebetween.

In the present disclosure, the term “substituted or unsubstituted” may refer to substituted or unsubstituted with at least one substituent selected from the group consisting of deuterium, a halogen, a cyano group, a nitro group, an amine group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In addition, each of the substituents exemplified above may be substituted or unsubstituted. For example, a biphenylyl group may be interpreted as an aryl group or a phenyl group substituted with a phenyl group.

In the present disclosure, the phrase “bonded to an adjacent group to form a ring” may refer to that a group is bonded to an adjacent group to form a substituted or unsubstituted hydrocarbon ring or a substituted or unsubstituted heterocycle. The hydrocarbon ring may include an aliphatic hydrocarbon ring and/or an aromatic hydrocarbon ring. The heterocycle may include an aliphatic heterocycle and/or an aromatic heterocycle. The hydrocarbon ring and the heterocycle may each be monocyclic or polycyclic. In addition, the rings formed by adjacent groups being bonded to each other may be connected to another ring to form a spiro structure.

In the present disclosure, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In one or more embodiments, two methyl groups in 4,5-dimethylphenanthrene may be interpreted as “adjacent groups” to each other.

In the present disclosure, examples of a halogen may include fluorine, chlorine, bromine, or iodine.

In the present disclosure, an alkyl group may be linear or branched. The number of carbons in the alkyl group may be 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl 1 group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, and/or the like, but embodiments of the disclosure are not limited thereto.

In the present disclosure, a cycloalkyl group may refer to a cyclic alkyl group. The number of carbons in the cycloalkyl group may be 3 to 50, 3 to 30, 3 to 20, or 3 to 10. Examples of the cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, and/or the like, but embodiments of the present disclosure are not limited thereto.

In the present disclosure, an alkenyl group refers to a hydrocarbon group including at least one carbon-carbon double bond in the middle or terminal of an alkyl group having 2 or more carbon atoms. The alkenyl group may be linear or branched. The number of carbon atoms in the alkenyl group is not specifically limited, for example, may be 2 to 30, 2 to 20, or 2 to 10. Examples of the alkenyl group may include a vinyl group, a 1-butenyl group, a 1-pentenyl group, a 1,3-butadienyl aryl group, a styrenyl group, a styryl vinyl group, and/or the like, but embodiments of the present disclosure are not limited thereto.

In the present disclosure, an aryl group refers to any functional group or substituent derived from an aromatic hydrocarbon ring. The aryl group may be a monocyclic aryl group or a polycyclic aryl group. The number of ring-forming carbon atoms in the aryl group may be 6 to 60, 6 to 50, 6 to 40, 6 to 30, 6 to 20, or 6 to 15. Examples of the aryl group may include a phenyl group, a naphthyl group, a fluorenyl group, an anthracenyl group, a phenanthrenyl group, a biphenylyl group, a terphenylyl group, a quaterphenylyl group, a quinquephenylyl group, a sexiphenylyl group, a triphenylenyl group, a pyrenyl group, a benzofluoranthenyl group, a chrysenyl group, and/or the like, but embodiments of the present disclosure are not limited thereto.

In the present disclosure, a fluorenyl group may be substituted, and two substituents may be bonded to each other to form a spiro structure. Examples of the substituted fluorenyl group are as follows. However, embodiments of the present disclosure are not limited thereto.

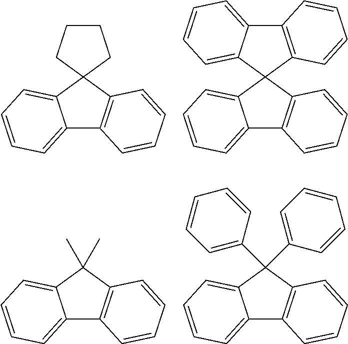

A heterocyclic group as used herein refers to any functional group or substituent derived from a ring containing at least one of B, O, N, P, Si, or Se as a heteroatom. The heterocyclic group includes an aliphatic heterocyclic group and/or an aromatic heterocyclic group. The aromatic heterocyclic group may be a heteroaryl group. The aliphatic heterocycle and the aromatic heterocycle may each be monocyclic or polycyclic.

In the present disclosure, the heteroaryl group may contain at least one of B, O, N, P, Si, or S as a heteroatom. If (e.g., when) the heteroaryl group contains two or more heteroatoms, the two or more heteroatoms may be the same as or different from each other. The heteroaryl group may be a monocyclic heterocyclic group or a polycyclic heterocyclic group. The number of ring-forming carbon atoms in the heteroaryl group may be 2 to 60, 2 to 50, 2 to 40, 2 to 30, 2 to 20, or 2 to 10. Examples of the heteroaryl group may include a thienyl group, a furyl group, a pyrrolyl group, an imidazolyl group, a pyridyl group, a bipyridinyl group, a triazolyl group, an acridinyl group, a pyridazinyl group, a pyazinyl group, a quinolyl group, a quinazolinyl group, a quinoxalinyl group, a phenoxazinyl group, a phthalazinyl group, a pyridopyrimidinyl group, a pyridopyrazinyl group, a pyrazinopyrazinyl group, an isoquinolinyl group, an indolyl group, a carbazolyl group, an N-arylcarbazolyl group, an N-heteroarylcarbazolyl group, an N-alkylcarbazolyl group, a benzoxazolyl group, a benzimidazolyl group, a benzothiazolyl group, a benzocarbazolyl group, a benzothiophenyl group, a dibenzothiophenyl group, a thienothiophenyl group, a benzofuranyl group, a phenanthrolinyl group, a thiazolyl group, an isoxazolyl group, an oxazolyl group, an oxadiazolyl group, a thiadiazolyl group, a phenothiazinyl group, a dibenzosilolyl group, a dibenzofuranyl group, and/or the like, but embodiments of the present disclosure are not limited thereto.

In the present disclosure, the above description of the aryl group may be applied to an arylene group except that the arylene group is a divalent group. The above description of the heteroaryl group may be applied to a heteroarylene group except that the heteroarylene group is a divalent group.

In the present disclosure, a silyl group may include an alkylsilyl group and/or an arylsilyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and/or the like, but embodiments of the present disclosure are not limited thereto.

In the present disclosure, a thio group may include an alkylthio group and/or an arylthio group. The thio group may refer to that a sulfur atom is bonded to the alkyl group or the aryl group defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but embodiments of the present disclosure are not limited thereto.

In the present disclosure, an oxy group may refer to that an oxygen atom is bonded to the alkyl group or the aryl group defined above. The oxy group may include an alkoxy group and/or an aryl oxy group. The alkoxy group may be a linear chain, a branched chain, or a ring. The number of carbon atoms in the alkoxy group is not specifically limited, but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, and/or the like, but embodiments of the present disclosure are not limited thereto.

A boron group as used herein may refer to that a boron atom is bonded to the alkyl group or the aryl group defined above. The boron group may include an alkyl boron group and/or an aryl boron group. Examples of the boron group may include a dimethylboron group, a trimethylboron group, a t-butyldimethylboron group, a diphenylboron group, a phenylboron group, and/or the like, but embodiments of the present disclosure are not limited thereto.

In the present disclosure, the number of carbon atoms in an amine group is not specifically limited, for example, may be 1 to 30. The amine group may include an alkyl amine group and/or an aryl amine group. Examples of the amine group may include a methylamine group, a dimethylamine group, a phenylamine group, a diphenylamine group, a naphthylamine group, a 9-methyl-anthracenylamine group, and/or the like, but embodiments of the present disclosure are not limited thereto.

In the present disclosure, the alkyl group among an alkylthio group, an alkylsulfoxy group, an alkylaryl group, an alkylamine group, an alkyl boron group, an alkyl silyl group, and an alkyl amine group may be the same as the examples of the alkyl group described above.

In the present disclosure, the aryl group among an aryloxy group, an arylthio group, an arylsulfoxy group, an arylamine group, an arylboron group, an arylsilyl group, an arylamine group may be the same as the examples of the aryl group described above.

In the present disclosure, a direct linkage may refer to a single bond.

In the present disclosure, and

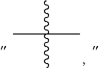

and

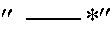

each refer to a position to be connected.

Hereinafter, embodiments of the disclosure will be described in more detail with reference to the accompanying drawings. In the present disclosure, the terms “display device” and “display apparatus” may be used interchangeably.

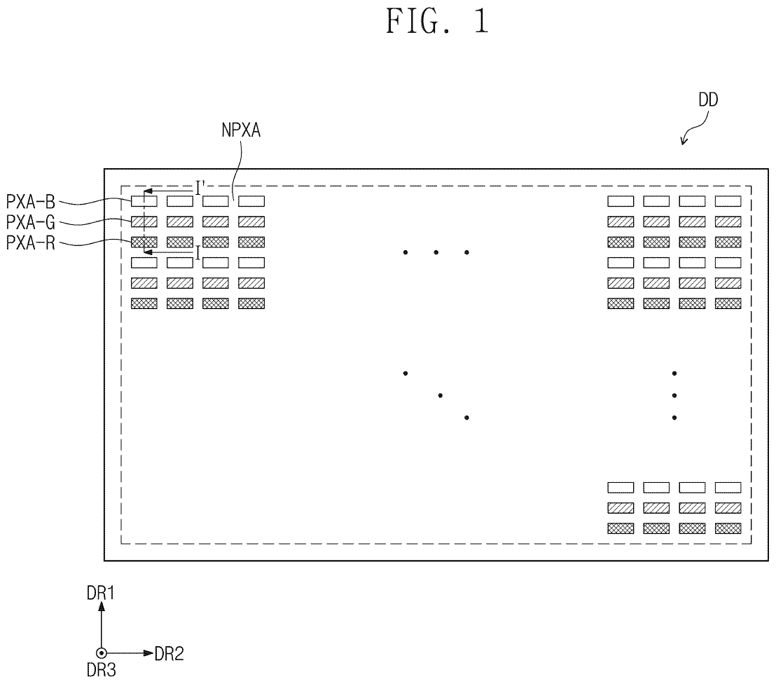

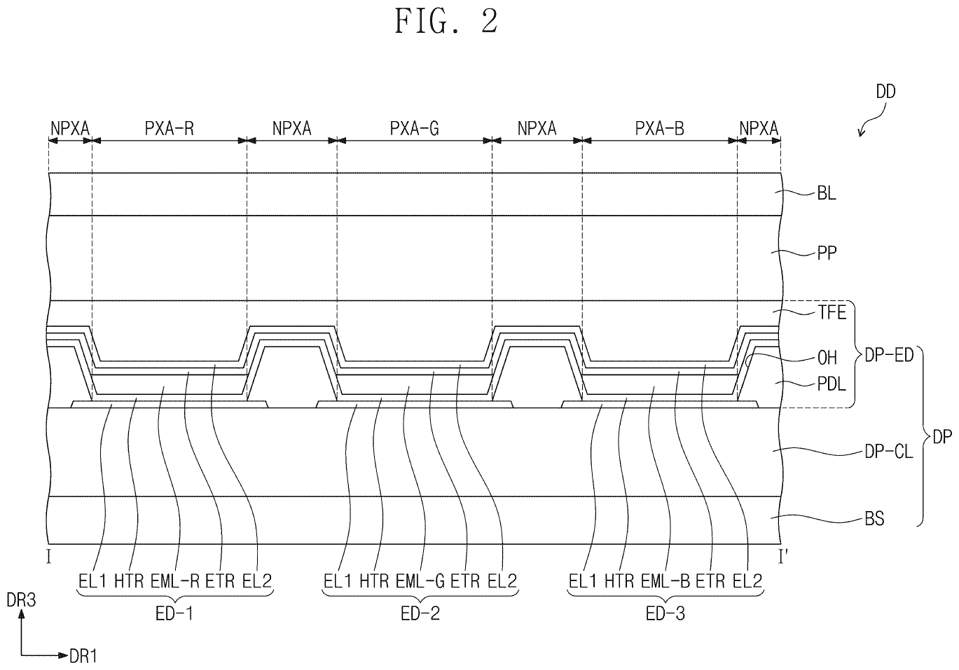

FIG. 1 is a plan view illustrating a display apparatus DD according to one or more embodiments of the present disclosure. FIG. 2 is a cross-sectional view of the display apparatus DD. FIG. 2 is a cross-sectional view illustrating a part taken along the line I-I′ of the display apparatus DD of FIG. 1.

The display apparatus DD may include a display panel DP and an optical layer PP arranged on the display panel DP. The display panel DP may include light-emitting elements ED-1, ED-2, and ED-3. The display apparatus DD may include a plurality of light-emitting elements ED-1, ED-2, and ED-3. The optical layer PP may be arranged on the display panel DP to control reflected light in the display panel DP due to external light. The optical layer PP may include, for example, a polarization layer and/or a color filter layer. In one or more embodiments, the optical layer PP may not be provided in the display apparatus DD.

A base substrate BL may be arranged on the optical layer PP. The base substrate BL may be a member which provides a base surface on which the optical layer PP arranged. The base substrate BL may be a glass substrate, a metal substrate, a plastic substrate, and/or the like. However, embodiments of the present disclosure are not limited thereto, and the base substrate BL may be an inorganic layer, an organic layer, or a composite material layer. In one or more embodiments, the base substrate BL may not be provided.

The display apparatus DD according to one or more embodiments may further include a filling layer. The filling layer may be arranged between a display device layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and a display device layer DP-ED. The display device layer DP-ED may include a pixel defining film PDL, the light-emitting elements ED-1, ED-2, and ED-3 arranged between respective portions of the pixel defining film PDL, and an encapsulation layer TFE arranged on the light-emitting elements ED-1, ED-2, and ED-3.

The base layer BS may be a member which provides a base surface on which the display device layer DP-ED is arranged. The base layer BS may be a glass substrate, a metal substrate, a plastic substrate, and/or the like. However, embodiments of the present disclosure are not limited thereto, and the base layer BS may be an inorganic layer, an organic layer, or a composite material layer.

In one or more embodiments, the circuit layer DP-CL may be arranged on the base layer BS, and the circuit layer DP-CL may include a plurality of transistors. Each of the transistors may include a control electrode, an input electrode, and an output electrode. For example, in one or more embodiments, the circuit layer DP-CL may include switching transistor(s) and driving transistor(s) for driving the light-emitting elements ED-1, ED-2, and ED-3 of the display device layer DP-ED.

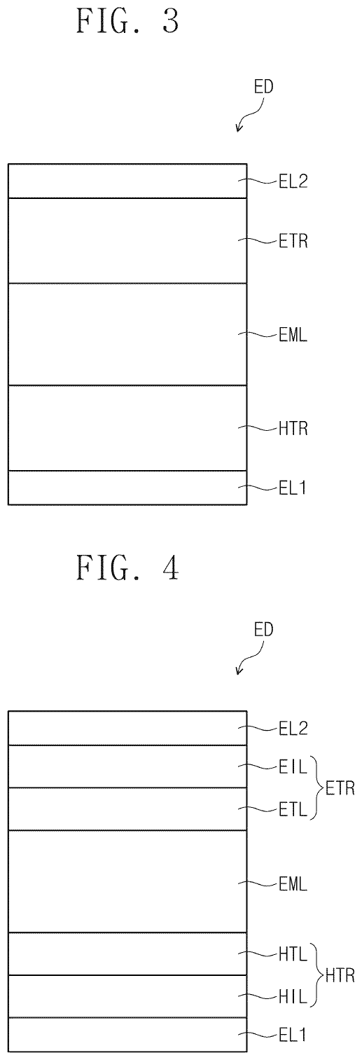

Each of the light-emitting elements ED-1, ED-2, and ED-3 may have a structure of one of light-emitting elements ED of embodiments according to FIGS. 3 to 6, which will be described in more detail later. Each of the light-emitting elements ED-1, ED-2, and ED-3 may include a first electrode EL1, a hole transport region HTR, respective light-emitting layers EML-R, EML-G, and EML-B, an electron transport region ETR, and a second electrode EL2.

FIG. 2 illustrates one or more embodiments in which the respective light-emitting layers EML-R, EML-G, and EML-B of the light-emitting elements ED-1, ED-2, and ED-3 are arranged in openings OH defined in the pixel defining film PDL, and the hole transport region HTR, the electron transport region ETR, and the second electrode EL2 are each provided as a common layer in the entire light-emitting elements ED-1, ED-2, and ED-3. However, embodiments of the present disclosure are not limited thereto, for example, in one or more embodiments, the hole transport region HTR and the electron transport region ETR may each be provided by being patterned inside the openings OH defined in the pixel defining film PDL. For example, the hole transport region HTR, the respective light-emitting layers EML-R, EML-G, and EML-B, and the electron transport region ETR of the light-emitting elements ED-1, ED-2, and ED-3 in one or more embodiments may be provided by being patterned in an inkjet printing method.

The encapsulation layer TFE may cover the light-emitting elements ED-1, ED-2, and ED-3. The encapsulation layer TFE may seal the display device layer DP-ED. The encapsulation layer TFE may be a thin film encapsulation layer. The encapsulation layer TFE may be formed by laminating one layer or a plurality of layers. The encapsulation layer TFE may include at least one insulation layer. The encapsulation layer TFE according to one or more embodiments may include at least one inorganic film (hereinafter, an encapsulation-inorganic film). The encapsulation layer TFE according to one or more embodiments may include at least one organic film (hereinafter, an encapsulation-organic film) and at least one encapsulation-inorganic film.

The encapsulation-inorganic film protects the display device layer DP-ED from moisture/oxygen, and the encapsulation-organic film protects the display device layer DP-ED from foreign substances such as dust particles. The encapsulation-inorganic film may include silicon nitride, silicon oxynitride, silicon oxide, titanium oxide, aluminum oxide, and/or the like, but embodiments of the present disclosure are not particularly limited thereto. In one or more embodiments, the encapsulation-organic film may include an acrylic-based compound, an epoxy-based compound, and/or the like. In one or more embodiments, the encapsulation-organic film may include a photopolymerizable organic material, but embodiments of the present disclosure are not particularly limited thereto.

The encapsulation layer TFE may be arranged on the second electrode EL2 and may be arranged filling the opening OH.

Referring to FIG. 1 and FIG. 2, the display apparatus DD may include a non-light emitting region NPXA and light emitting regions PXA-R, PXA-G, and PXA-B. The light emitting regions PXA-R, PXA-G, and PXA-B may be regions in which light generated by the respective light-emitting elements ED-1, ED-2, and ED-3 is emitted. The light emitting regions PXA-R, PXA-G, and PXA-B may be spaced and/or apart (e.g., spaced apart or separated) from each other on a plane (e.g., in a plan view).

Each of the light emitting regions PXA-R, PXA-G, and PXA-B may be a region divided by the pixel defining film PDL. The non-light emitting areas NPXA may be areas between adjacent light emitting regions PXA-R, PXA-G, and PXA-B, which correspond to the pixel defining film PDL. In one or more embodiments, the light emitting regions PXA-R, PXA-G, and PXA-B may respectively correspond to pixels. The pixel defining film PDL may divide the light-emitting elements ED-1, ED-2, and ED-3. The respective light-emitting layers EML-R, EML-G, and EML-B of the light-emitting elements ED-1, ED-2, and ED-3 may be arranged in openings OH defined in the pixel defining film PDL and separated from each other.

The light emitting regions PXA-R, PXA-G, and PXA-B may be divided into a plurality of groups according to the color of light generated from the light-emitting elements ED-1, ED-2, and ED-3. In the display apparatus DD of one or more embodiments illustrated in FIG. 1 and FIG. 2, three light emitting regions PXA-R, PXA-G, and PXA-B, which emit red light, green light, and blue light, respectively, are exemplarily illustrated. For example, the display device DD of one or more embodiments may include a red light emitting region PXA-R, a green light emitting region PXA-G, and a blue light emitting region PXA-B that are separated from each other.

In the display apparatus DD according to one or more embodiments, the plurality of light-emitting elements ED-1, ED-2, and ED-3 may be to emit light beams having wavelengths different from each other. For example, in one or more embodiments, the display apparatus DD may include a first light-emitting element ED-1 that emits red light, a second light-emitting element ED-2 that emits green light, and a third light-emitting element ED-3 that emits blue light. For example, the red light emitting region PXA-R, the green light emitting region PXA-G, and the blue light emitting region PXA-B of the display apparatus DD may correspond to the first light-emitting element ED-1, the second light-emitting element ED-2, and the third light-emitting element ED-3, respectively.

However, embodiments of the present disclosure are not limited thereto, and the first to third light-emitting elements ED-1, ED-2, and ED-3 may be to emit light beams in substantially the same wavelength range, or at least one light-emitting element may be to emit a light beam in a wavelength range different from the others. For example, in one or mor embodiments, the first to third light-emitting elements ED-1, ED-2, and ED-3 may all emit blue light.

The light emitting regions PXA-R, PXA-G, and PXA-B in the display apparatus DD according to one or more embodiments may be arranged in a stripe form. Referring to FIG. 1, a plurality of red light emitting regions PXA-R may be arranged with each other along a second direction axis DR2, a plurality of green light emitting regions PXA-G may be arranged with each other along the second direction axis DR2, and a plurality of blue light emitting regions PXA-B may be arranged with each other along the second direction axis DR2. In addition, the red light emitting region PXA-R, the green light emitting region PXA-G, and the blue light emitting region PXA-B may be alternately arranged in this order along a first direction axis DR1.

FIG. 1 and FIG. 2 illustrate that all the light emitting regions PXA-R, PXA-G, and PXA-B have similar area, but embodiments of the present disclosure are not limited thereto. Thus, the light emitting regions PXA-R, PXA-G, and PXA-B may have different areas from each other according to the wavelength range of the emitted light. The areas of the light emitting regions PXA-R, PXA-G, and PXA-B may refer to areas if (e.g., when) viewed on a plane defined by the first direction axis DR1 and the second direction axis DR2 (e.g., the areas in a plan view of the light emitting regions PXA-R, PXA-G, and PXA-B).

In one or more embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality desired or required in the display apparatus DD. For example, in one or more embodiments, the arrangement form of the light emitting regions PXA-R, PXA-G, and PXA-B may be a pentile (PENTILE®) arrangement form (for example, an RGBG matrix, an RGBG structure, or an RGBG matrix structure) or a diamond (Diamond Pixel™) arrangement form (e.g., a display (e.g., an OLED display) containing red, blue, and green (RGB) light-emitting regions arranged in the shape of diamonds). PENTILE® is a duly registered trademark of Samsung Display Co., Ltd. Diamond Pixel™ is a trademark of Samsung Display Co., Ltd.

In one or more embodiments, the areas of the light emitting regions PXA-R, PXA-G, and PXA-B may be different from each other. For example, in one or more embodiments, the area of the green light emitting region PXA-G may be smaller than that of the blue light emitting region PXA-B, but embodiments of the present disclosure are not limited thereto.

Hereinafter, FIG. 3 to FIG. 6 are each a cross-sectional view schematically showing a light-emitting element according to one or more embodiments of the present disclosure. The light-emitting element ED according to one or more embodiments may include a first electrode EL1, a second electrode EL2 oppositely on (e.g., arranged on) the first electrode EL1, and at least one functional layer between the first electrode EL1 and the second electrode EL2. The light-emitting element ED of one or more embodiments may include an amine compound of one or more embodiments of the present disclosure, which will be explained later, in the at least one functional layer.

In one or more embodiments, the light-emitting element ED may include a hole transport region HTR, a light-emitting layer EML, an electron transport region ETR, and/or the like, stacked in order (e.g., in the stated order), as the at least one functional layer. Referring to FIG. 3, the light-emitting element ED of one or more embodiments may include a first electrode EL1, a hole transport region HTR, a light-emitting layer EML, an electron transport region ETR, and a second electrode EL2, stacked in the stated order.

The light-emitting element ED according to one or more embodiments may include an amine compound of one or more embodiments of the present disclosure, which will be explained later, in the hole transport region HTR between the first electrode EL1 and the second electrode EL2. However, embodiments of the present disclosure are not limited thereto. In addition to the hole transport region HTR, the light-emitting element ED according to one or more embodiments may include an amine compound of one or more embodiments of the present disclosure, which will be explained later, in the light-emitting layer EML or the electron transport region ETR, each of which is a functional layer between the first electrode EL1 and the second electrode EL2, or may include an amine compound of one or more embodiments of the present disclosure, which will be explained later, in a capping layer CPL on the second electrode EL2.

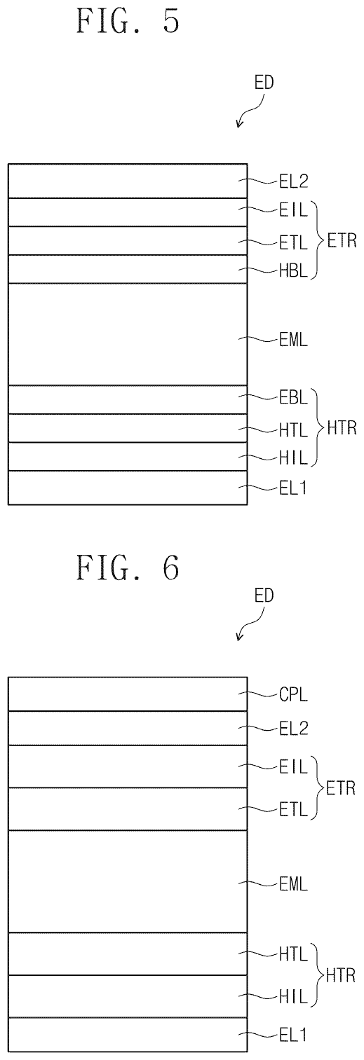

Compared with FIG. 3, FIG. 4 illustrates a cross-sectional view of a light-emitting element ED of one or more embodiments, in which a hole transport region HTR includes a hole injection layer HIL and a hole transport layer HTL, and an electron transport region ETR includes an electron injection layer EIL and an electron transport layer ETL. In addition, compared with FIG. 3, FIG. 5 illustrates a cross-sectional view of a light-emitting element ED of one or more embodiments, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared with FIG. 4, FIG. 6 illustrates a cross-sectional view of a light-emitting element ED of one or more embodiments including a capping layer CPL arranged on a second electrode EL2.

The light-emitting element ED according to one or more embodiments may include an amine compound of one or more embodiments of the present disclosure, which will be explained later, in the hole transport region HTR. In the light-emitting element ED according to one or more embodiments, at least one selected from among the hole injection layer HIL, the hole transport layer HTL, and the electron blocking layer EBL may include an amine compound of one or more embodiments of the present disclosure. In one or more embodiments, a layer adjacent to the light-emitting layer selected from among the plurality of layers included in the hole transport region HTR may contain an amine compound of one or more embodiments represented by Formula 1. For example, In the light-emitting element ED according to one or more embodiments, the hole transport layer HTL may include an amine compound of one or more embodiments represented by Formula 1. That is, in one or more embodiments, the light-emitting element ED may include the disclosed amine compound in the hole transport region HTR. This amine compound can be present in at least one of the hole injection layer HIL, the hole transport layer HTL, or the electron blocking layer EBL. Specifically, for example, the layer adjacent to the emission layer within the hole transport region HTR may contain an amine compound represented by Formula 1. For instance, the hole transport layer HTL in the light-emitting element ED may include this amine compound.

The first electrode EL1 has conductivity (e.g., is a conductor). The first electrode EL1 may be formed of a metal material, a metal alloy, and/or a conductive compound. The first electrode EL1 may be an anode or a cathode. However, embodiments of the present disclosure are not limited thereto. In one or more embodiments, the first electrode EL1 may be a pixel electrode. The first electrode EL1 may be a transmissive electrode, a transflective electrode, or a reflective electrode. The first electrode EL1 may include at least one selected from among silver (Ag), magnesium (Mg), copper (Cu), aluminum (Al), platinum (Pt), palladium (Pd), gold (Au), nickel (Ni), neodymium (Nd), iridium (Ir), chromium (Cr), lithium (Li), calcium (Ca), lithium fluoride (LiF), molybdenum (Mo), titanium (Ti), tungsten (W,) indium (In), tin (Sn), and zinc (Zn), a compound of two or more selected therefrom, a mixture of two or more selected therefrom, and/or an oxide thereof.

If (e.g., when) the first electrode EL1 is a transmissive electrode, the first electrode EL1 may include a transparent metal oxide such as indium tin oxide (ITO), indium zinc oxide (IZO), zinc oxide (ZnO), and/or indium tin zinc oxide (ITZO). If (e.g., when) the first electrode EL1 is a transflective electrode or a reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound thereof, or a mixture thereof (e.g., a mixture of Ag and Mg). In one or 1 more embodiments, the first electrode EL1 may have a multilayer structure including a reflective film or a transflective film formed of one or more of the above-described materials, and a transparent conductive film formed of ITO, IZO, ZnO, ITZO, and/or the like. For example, in one or more embodiments, the first electrode EL1 may have a three-layer structure of ITO/Ag/ITO, but embodiments of the present disclosure are not limited thereto. In one or more embodiments, the first electrode EL1 may include one of the above-described metal materials, one or more combinations of at least two metal materials of the above-described metal materials, any oxide of the above-described metal materials, and/or the like. A thickness of the first electrode EL1 may be from about 700 ångströms (Å) to about 10,000 Å. For example, in one or more embodiments, the thickness of the first electrode EL1 may be from about 1,000 Å to about 3,000 Å.

The hole transport region HTR may be provided on the first electrode EL1. The hole transport region HTR may include at least one of a hole injection layer HIL, a hole transport layer HTL, a buffer layer or an emission-auxiliary layer, or an electron blocking layer EBL. A thickness of the hole transport region HTR may be, for example, from about 50 Å to about 15,000 Å.

The hole transport region HTR may have a single layer formed of a single material, a single layer formed of a plurality of different materials, or a multilayer structure including a plurality of layers formed of a plurality of different materials.

For example, in one or more embodiments, the hole transport region HTR may have a single layer structure of a hole injection layer HIL or a hole transport layer HTL, or may have a single layer structure formed of a hole injection material and a hole transport material. In one or more embodiments, the hole transport region HTR may have a single layer structure formed of a plurality of different materials, or a structure in which a hole injection layer HIL/hole transport layer HTL, a hole injection layer HIL/hole transport layer HTL/buffer layer, a hole injection layer HIL/buffer layer, a hole transport layer HTL/buffer layer, or a hole injection layer HIL/hole transport layer HTL/electron blocking layer EBL are stacked in order (e.g., in the stated order) from the first electrode EL1, but embodiments of the present disclosure are not limited thereto.

A thickness of the hole transport region HTR may be, for example, about 50 Å to about 15,000 Å. The hole transport region HTR may be formed using one or more suitable methods such as a vacuum deposition method, a spin coating method, a casting method, a Langmuir-Blodgett (LB) method, an inkjet printing method, a laser printing method, and/or a laser induced thermal imaging (LITI) method.

The light-emitting element ED according to one or more embodiments may include an amine compound according to one or more embodiments of the present disclosure in the hole transport region HTR. In the light-emitting element ED according to one or more embodiments, the hole transport region HTR may include a hole injection layer HIL and a hole transport layer HTL, and the hole transport layer HTL may include the amine compound according to one or more embodiments of the present disclosure. The amine compound according to one or more embodiments may be contained in an adjacent layer to the light-emitting layer EML among layers included in the hole transport region HTR.

The amine compound according to one or more embodiments may include an amine group, and may further include a first substituent, a second substituent, and a third substituent, each connected to the amine group. For example, the amine compound according to one or more embodiments may include the amine group, which includes a core nitrogen atom, and may include a structure in which the first substituent, the second substituent, and the third substituent are all bonded to the core nitrogen atom.

The first substituent may include a benzene moiety, and a substituted or unsubstituted phenanthrenyl group and an unsubstituted phenyl group, each connected to the benzene moiety. In the first substituent, the substituted or unsubstituted phenanthrenyl group and the unsubstituted phenyl group may be connected to the benzene moiety to be in an ortho relationship with each other. In addition, in the first substituent, any one selected from among the substituted or unsubstituted phenanthrenyl group and the unsubstituted phenyl group may be connected to the benzene moiety at a para position with respect to the nitrogen atom, and the other may be connected to the benzene moiety at an ortho position with respect to the above-described any one. The first substituent may be connected to the core nitrogen atom of the amine group via a linker, or may be directly linked to the core nitrogen atom without additional linker.

The first substituent may be represented by Formula S1. In Formula S1, any one selected from among Ar¹ and Ar² may be a substituted or unsubstituted phenanthrenyl group and the other may be an unsubstituted phenyl group. For example, in one or more embodiments, in Formula S1, Ar¹ may be a substituted or unsubstituted phenanthrenyl group, and Ar² may be an unsubstituted phenyl group. In one or more embodiments, in Formula S1, Ar¹ may be an unsubstituted phenyl group, and Ar² may be a substituted or unsubstituted phenanthrenyl group. As illustrated in Formula S1, the first substituent in the amine compound according to one or more embodiments may be directly bonded to the nitrogen atom of the amine group at the “a1” position, or may be bonded to the nitrogen atom of the amine group at the “a1” position via a linker. Here, for convenience of description, a substituent that may be substituted on the benzene moiety is omitted in Formula S1. Unlike Formula S1, in one or more embodiments, the benzene moiety in the first substituent may have at least one substituent in addition to hydrogens.

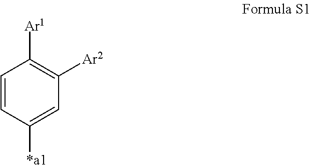

In the amine compound according to one or more embodiments, the second substituent and the third substituent may each be directly bonded to the nitrogen atom of the amine group. For example, each of the second substituent and the third substituent may be directly bonded to the nitrogen atom of the amine group without an additional linker. In one or more embodiments, the second and third substituents may each independently be a substituted or unsubstituted aryl group having 6 to 30 ring-forming carbons or a substituted or unsubstituted heteroaryl group having 2 to 30 ring-forming carbons In the present disclosure, the second substituent may refer to a substituent represented by Ar³ in Formula 1 which will be described in more detail later, and the third substituent may refer to a substituent represented by Ar⁴ in Formula 1 which will be described in more detail later.

Because the amine compound according to one or more embodiments includes the first substituent, a charge balance may be beneficially adjusted due to a steric effect, and thus excellent or suitable charge transportability may be exhibited, and high thermal stability may be exhibited and obtained. For example, the amine compound according to one or more embodiments includes the first substituent including a substituted or unsubstituted phenanthrenyl group and an unsubstituted phenyl group which are bonded to a benzene moiety in a specific position relationship, and thus have excellent or suitable charge transportability and material stability. Consequently, this structural configuration may contribute to improvements in high efficiency and long lifespan of the light-emitting element.

The amine compound according to one or more embodiments may be a monoamine compound including one (e.g., exactly one) amine group. The amine compound according to one or more embodiments may be a monoamine compound which contains only one amine group present in its molecular structure without forming a ring.

In one or more embodiments, the amine compound may be represented by Formula 1.

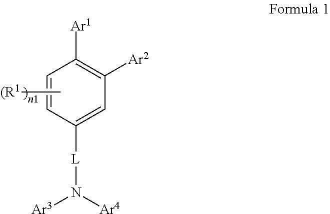

In Formula 1, any one selected from among Ar¹ and Ar² may be represented by Formula 2 or Formula 3, and the other may be an unsubstituted phenyl group. For example, in one or more embodiments, one selected from among Ar¹ and Ar² is represented by Formula 2, and the other is an unsubstituted phenyl group. In one or more embodiments, one selected from among Ar¹ and Ar² is represented by Formula 3, and the other is an unsubstituted phenyl group.

In Formula 1, Ar³ and Ar⁴ may each independently be a substituted or unsubstituted aryl group having 6 to 30 ring-forming carbons or a substituted or unsubstituted heteroaryl group having 2 to 30 ring-forming carbons. In one or more embodiments, Ar³ and Ar⁴ may each independently be a substituted or unsubstituted phenyl group, a substituted or unsubstituted biphenylyl group, a substituted or unsubstituted naphthyl group, a substituted or unsubstituted naphthylphenyl group, a substituted or unsubstituted fluorenyl group, a substituted or unsubstituted carbazolyl group, a substituted or unsubstituted dibenzofuranyl group, or a substituted or unsubstituted dibenzothiophenyl group.

In one or more embodiments, if (e.g., when) each of Ar³ and Ar⁴ is substituted, a substituent thereof may independently be deuterium, a substituted or unsubstituted phenyl group, a substituted or unsubstituted naphthyl group, a substituted or unsubstituted naphthylphenyl group, a substituted or unsubstituted fluorenyl group, a substituted or unsubstituted carbazolyl group, a substituted or unsubstituted dibenzofuranyl group, a substituted or unsubstituted dibenzothiophenyl group, or a substituted or unsubstituted phenanthrenyl group.

In Formula 1, L may be a direct linkage, a substituted or unsubstituted arylene group having 6 to 30 ring-forming carbons, or a substituted or unsubstituted heteroarylene group having 2 to 30 ring-forming carbons. For example, in one or more embodiments, L may be a direct linkage or a substituted or unsubstituted phenylene group.

In Formula 1, R¹ may be hydrogen, deuterium, or a substituted or unsubstituted alkyl group having 1 to 10 carbons. For example, in one or more embodiments, R¹ may be hydrogen.

In Formula 1, n1 may be an integer of 0 to 3. If (e.g., when) n1 is 0, the amine compound according to one or more embodiments may be unsubstituted with R¹. An embodiment in which n1 is 3 and R¹ are all hydrogens in Formula 1 may be the same as the embodiment in which n1 is 0 in Formula 1. If (e.g., when) n1 is an integer of 2 or greater, R¹ provided in plural may be all the same, or at least one selected from among the plurality of R¹ (a) may be different.

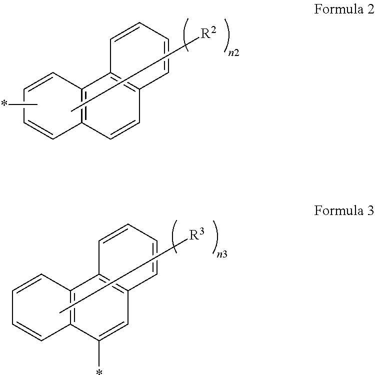

In Formula 2 and Formula 3, R² and R³ may each independently be hydrogen, deuterium, a substituted or unsubstituted alkyl group having 1 to 10 carbons, a substituted or unsubstituted aryl group having 6 to 30 ring-forming carbons, or a substituted or unsubstituted heteroaryl group having 2 to 30 ring-forming carbons. For example, in one or more embodiments, R² and R³ may each be hydrogen.

In Formula 2 and Formula 3, n2 and n3 may each independently be an integer of 0 to 9. If (e.g., when) n2 and n3 are each 0, the amine compound according to one or more embodiments may be unsubstituted with each of R² and R³.

Embodiments in which n2 and n3 are each 9 and R²(a) and R³(a) are each hydrogen in Formula 2 and Formula 3 may be the same as the embodiments in which n2 and n3 are each 0 in Formula 2 and Formula 3. If (e.g., when) n2 and n3 are each an integer of 2 or greater, R² and R³ each provided in plural may be all the same or at least one selected from among a plurality of R²(a) and R³(a) may be different.

In Formula 2 and Formula 3, -* may be a position at which the rest portion of Formula 1 that is not represented by Formula 2 or Formula 3 is connected.

In the amine compound according to one or more embodiments, represented by Formula 1, if (e.g., when) any one selected from among Ar¹ and Ar² is represented by Formula 3, at least one selected from among Ar³ and Ar⁴ may include a substituted or unsubstituted naphthylphenyl group, a substituted or unsubstituted dibenzofuranyl group, a substituted or unsubstituted dibenzothiophenyl group, a substituted or unsubstituted 9-phenylcarbazolyl group, or a substituted or unsubstituted 9,9-diphenylfluorenyl group. For example, if (e.g., when) a phenanthrene moiety in the amine compound according to one or more embodiments is, as represented below, connected to a phenylene linker via a carbon atom at “a2” position, at least one selected from among Ar³ and Ar⁴ may include a substituted or unsubstituted naphthylphenyl group, a substituted or unsubstituted dibenzofuranyl group, a substituted or unsubstituted dibenzothiophenyl group, a substituted or unsubstituted 9-phenylcarbazolyl group, or a substituted or unsubstituted 9,9-diphenylfluorenyl group. In the present disclosure, the phenanthrene moiety represented by Formula 3 may be referred to a 9-phenanthrenyl group.

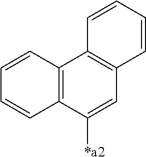

In one or more embodiments, as used herein, “Substituent a contains Substituent b” refers to that Substituent a is Substituent b or Substituent a contains Substituent b as a substituent. For example, in Formula 1, if (e.g., when) any one selected from among Ar¹ and Ar² is represented by Formula 3, at least one selected from among Ar³ and Ar⁴ may be a substituted or unsubstituted naphthylphenyl group, a substituted or unsubstituted dibenzofuranyl group, a substituted or unsubstituted dibenzothiophenyl group, a substituted or unsubstituted 9-phenylcarbazolyl group, or a substituted or unsubstituted 9,9-diphenylfluorenyl group, or at least one selected from among Ar³ and Ar⁴ may be substituted with a substituted or unsubstituted naphthylphenyl group, a substituted or unsubstituted dibenzofuranyl group, a substituted or unsubstituted dibenzothiophenyl group, a substituted or unsubstituted 9-phenylcarbazolyl group, or a substituted or unsubstituted 9,9-diphenylfluorenyl group.

In the amine compound represented by Formula 1, if (e.g., when) any one selected from among Ar¹ and Ar² is a 9-phenanthrenyl group, distortion of molecules is caused, and thus thermal stability of the amine compound may deteriorate. However, according to the present disclosure, if (e.g., when) any one selected from among Ar¹ and Ar² is represented by Formula 3, at least one substituent selected from among Ar³ and Ar⁴ may contain a substituted or unsubstituted naphthylphenyl group, a substituted or unsubstituted dibenzofuranyl group, a substituted or unsubstituted dibenzothiophenyl group, a substituted or unsubstituted 9-phenylcarbazolyl group, or a substituted or unsubstituted 9,9-diphenylfluorenyl group. As such, thermal stability of the amine compound may be secured. Therefore, if (e.g., when) the amine compound according to one or more embodiments is applied to the light-emitting element, characteristics of high efficiency and long lifespan may be achieved.

In the amine compound according to one or more embodiments, represented by Formula 1, Ar³ and Ar⁴ each does not contain (e.g., each exclude) a substituted or unsubstituted nitrogen-containing six-membered hetero ring, a substituted or unsubstituted 9,9-dimethylfluorenyl group, and a boron-containing derivative (e.g., boron-containing moiety). In the amine compound according to one or more embodiments, represented by Formula 1, cases where at least one (e.g., any one) selected from among Ar³ and Ar⁴ includes a substituted or unsubstituted nitrogen-containing six-membered hetero ring, a substituted or unsubstituted 9,9-dimethylfluorenyl group, and/or a boron-containing derivative (e.g., boron-containing moiety) are excluded. For example, in Formula 1, cases where at least one (e.g., any one) selected from among Ar³ and Ar⁴ is a substituted or unsubstituted nitrogen-containing six-membered hetero ring, a substituted or unsubstituted 9,9-dimethylfluorenyl group, and/or a boron-containing derivative (e.g., boron-containing moiety) as a substituent, or at least one (e.g., any one) selected from among Ar³ and Ar⁴ includes a substituted or unsubstituted nitrogen-containing six-membered hetero ring, a substituted or unsubstituted 9,9-dimethylfluorenyl group, and/or a boron-containing derivative (e.g., boron-containing moiety) as a substituent, may be excluded. For example, in Formula 1, cases where Ar³ and Ar⁴ include a pyridyl group such as

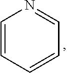

a 9,9-dimethylfluorenyl group such as

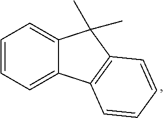

and/or a boronic acid group such as *—B(OH)₂ may be excluded. When each of Ar³ and Ar⁴ includes a substituent as described above, hole transport characteristics and thermal stability of molecules deteriorate, and thus such amine compounds may be unsuitable for hole transport materials of the light-emitting element ED. According to the present disclosure, because the cases where Ar³ and Ar⁴ include a substituted or unsubstituted six-membered hetero ring, a substituted or unsubstituted 9,9-dimethylfluorenyl group, and/or a boron-containing derivative (e.g., boron-containing moiety) are excluded, hole transport characteristics of molecules and thermal stability of the amine compound may be improved. Therefore, if (e.g., when) the amine compound according to one or more embodiments is applied to the light-emitting element, characteristics of high efficiency and long lifespan may be achieved.

When the amine compound according to one or more embodiments, represented by Formula 1, includes a four-membered fused hetero ring, the four-membered fused hetero ring is directly bonded to the nitrogen atom in Formula 1. For example, if (e.g., when) the amine compound according to one or more embodiments, represented by Formula 1, includes a four-membered fused hetero ring, the four-membered fused hetero ring may be directly bonded to the nitrogen atom in Formula 1 without additional linker. In the present disclosure, the term “four-membered fused hetero ring” may refer to a fused hetero ring including 4 rings fused together.

In one or more embodiments, if (e.g., when) the amine compound represented by Formula 1 includes a four-membered fused hetero ring, the four-membered fused hetero ring may correspond to any one selected from among Ar³ and Ar⁴, which is directly bonded to the nitrogen atom in Formula 1. For example, if (e.g., when) the amine compound according to one or more embodiments, represented by Formula 1, includes a four-membered fused hetero ring such as

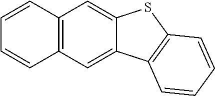 [SMILES: c1ccc2cc3c(cc2c1)sc1ccccc13]

(benzonaphthothiophene), the four-membered fused hetero ring may correspond to any one selected from among Ar³ and Ar⁴ directly bonded to the nitrogen atom in Formula 1. When, a heterocycle with high planarity, such as the four-membered fused hetero ring, is connected to the amine group via a linker, intermolecular interaction excessively (or substantially) increases, and thus deposition temperature may increase, which may cause the decomposition of molecules to increase during deposition. However, in the present disclosure, if (e.g., when) the amine compound according to one or more embodiments includes a four-membered fused hetero ring, the four-membered fused hetero ring is directly connected the nitrogen atom of the amine group; as a result, the intermolecular interaction may be suppressed or significantly reduced. Therefore, if (e.g., when) the amine compound according to one or more embodiments is applied to the light-emitting element, characteristics of high efficiency and long lifespan maybe achieved.

In the amine compound according to one or more embodiments, represented by Formula 1, a case where at least one selected from among Ar³ and Ar⁴ is a substituted or unsubstituted phenanthrenyl group is excluded. For example, in the amine compound according to one or more embodiments, a case where at least one selected from among Ar³ and Ar⁴ is represented by Formula P-1 is excluded.

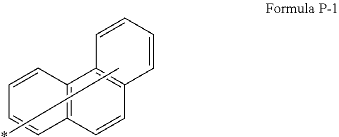

When the amine compound according to one or more embodiments, represented by Formula 1, includes an additional phenanthrenyl group in addition to Ar¹ or Ar², a case where the additional phenanthrenyl group is directly bonded to the nitrogen atom of the amine group is excluded. When the additional phenanthrenyl group in addition to Ar¹ or Ar² is directly bonded to the nitrogen atom of the amine group, it may cause that intermolecular interaction increases and thus deposition temperature excessively (or substantially) increases too. In the present disclosure, the case where at least one selected from among Ar³ and Ar⁴ is a substituted or unsubstituted phenanthrenyl group is excluded, and thus an excessive increase in deposition temperature due to the intermolecular interaction may be suppressed or reduced. Therefore, characteristics of element lifespan and efficiency of the light-emitting element may be improved.

In one or more embodiments, the amine compound represented by Formula 1 may be represented by Formula 2-1 or Formula 2-2.

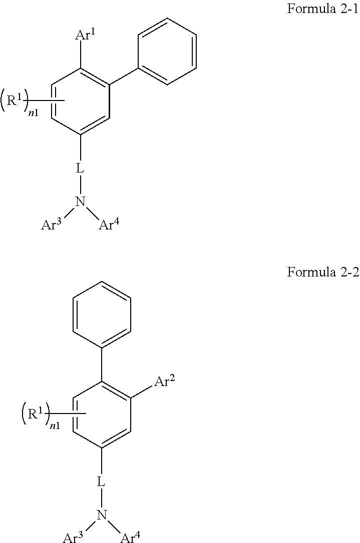

Formula 2-1 and Formula 2-2 embody cases where the types (kinds) of Ar¹ and Ar² in Formula 1 are specified. Formula 2-1 corresponds to a case where Ar¹ is represented by Formula 2 or Formula 3 and Ar² is an unsubstituted phenyl group. Formula 2-2 corresponds to a case where Ar¹ is an unsubstituted phenyl group and Ar² is represented by Formula 2 or Formula 3.

The descriptions of R¹, n1, L, Ar¹, Ar², Ar³, and Ar⁴ in Formula 1 may be similarly applied to Formula 2-1 and Formula 2-2. In other words, R¹, n1, L, Ar¹, Ar², Ar³, and Ar⁴ in Formula 2-1 and Formula 2-2 may each independently be the same as defined in Formula 1.

In Formula 2-1 and Formula 2-2, Ar³ and Ar⁴ may each independently be a substituted or unsubstituted aryl group having 6 to 30 ring-forming carbons or a substituted or unsubstituted heteroaryl group having 2 to 30 ring-forming carbons. In one or more embodiments, Ar³ and Ar⁴ may each independently be a substituted or unsubstituted phenyl group, a substituted or unsubstituted biphenylyl group, a substituted or unsubstituted naphthyl group, a substituted or unsubstituted naphthylphenyl group, a substituted or unsubstituted fluorenyl group, a substituted or unsubstituted carbazolyl group, a substituted or unsubstituted dibenzofuranyl group, or a substituted or unsubstituted dibenzothiophenyl group.

In one or more embodiments, in Formula 2-1 and Formula 2-2, if (e.g., when) each of Ar³ and Ar⁴ is substituted, the substituent thereof may be deuterium, a substituted or unsubstituted phenyl group, a substituted or unsubstituted naphthyl group, a substituted or unsubstituted naphthylphenyl group, a substituted or unsubstituted fluorenyl group, a substituted or unsubstituted carbazolyl group, a substituted or unsubstituted dibenzofuranyl group, a substituted or unsubstituted dibenzothiophenyl group, or a substituted or unsubstituted phenanthrenyl group.

In one or more embodiments, the amine compound represented by Formula 1 may be represented by Formula 3.

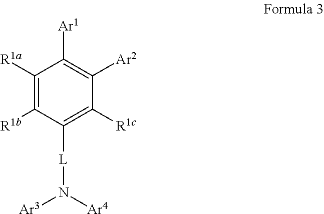

Formula 3 embodies a case where the types (kinds) of R¹ in Formula 1 is specified.

In Formula 3, R^{1a}, R^{1b}, and R^{1c} may each independently be hydrogen or deuterium. For example, in one or more embodiments, R^{1a}, R^{1b}, and R^{1c} may be each hydrogen.

The descriptions of L, Ar¹, Ar², Ar³, and Ar⁴ in Formula 1 may be similarly applied to Formula 3. In other words, L, Ar¹, Ar², Ar³, and Ar⁴ in Formula 3 may each independently be the same as defined in Formula 1.

In one or more embodiments, the amine compound represented by Formula 1 may be represented by any one selected from among Formula 4-1 to Formula 4-4.

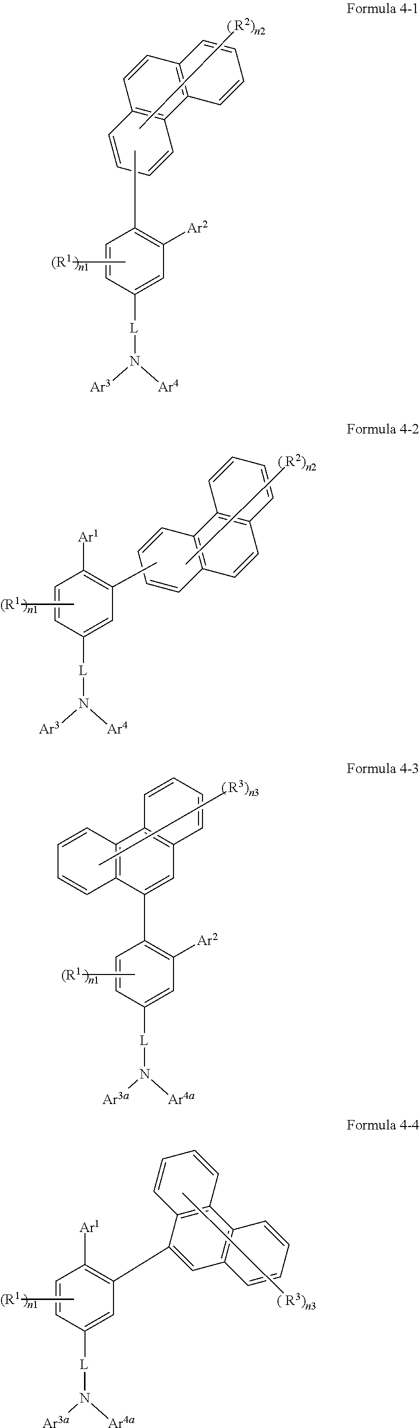

In Formula 4-3 and Formula 4-4, Ar^{3a} and Ar^{4a} may each independently be a substituted or unsubstituted aryl group having 6 to 30 ring-forming carbons or a substituted or unsubstituted heteroaryl group having 2 to 30 ring-forming carbons.

In one or more embodiments, in Formula 4-3 and Formula 4-4, at least one selected from among Ar^{3a} and Ar^{4a} contains a substituted or unsubstituted naphthylphenyl group, a substituted or unsubstituted dibenzofuranyl group, a substituted or unsubstituted dibenzothiophenyl group, a substituted or unsubstituted 9-phenylcarbazolyl group, or a substituted or unsubstituted 9,9-diphenylfluorenyl group. For example, in one or more embodiments, at least one selected from among Ar^{3a} and Ar^{4a} may be a substituted or unsubstituted naphthylphenyl group, a substituted or unsubstituted dibenzofuranyl group, a substituted or unsubstituted dibenzothiophenyl group, a substituted or unsubstituted 9-phenylcarbazolyl group, or a substituted or unsubstituted 9,9-diphenylfluorenyl group, or at least one selected from among Ar^{3a} and Ar^{4a} may be substituted with a substituted or unsubstituted naphthylphenyl group, a substituted or unsubstituted dibenzofuranyl group, a substituted or unsubstituted dibenzothiophenyl group, a substituted or unsubstituted 9-phenylcarbazolyl group, or a substituted or unsubstituted 9,9-diphenylfluorenyl group.

The descriptions of R¹, n1, L, Ar¹, Ar², Ar³, and Ar⁴ in Formula 1 above may be similarly applied to Formula 4-1 to Formula 4-4. In other words, R¹, n1, L, Ar¹, Ar², Ar³, and Ar⁴ in Formula 4-1 to Formula 4-4 may each independently be the same as defined in Formula 1.

The descriptions of R², R³, n2, and n3 in Formula 2 and Formula 3 above may be similarly applied to Formula 4-1 to Formula 4-4. In other words, R², R³, n2, and n3 in Formula 4-1 to Formula 4-4 may each independently be the same as defined in Formula 2 and Formula 3.

In one or more embodiments, the amine compound represented by Formula 1 may be represented by any one selected from among Formula 5-1 to Formula 5-3.

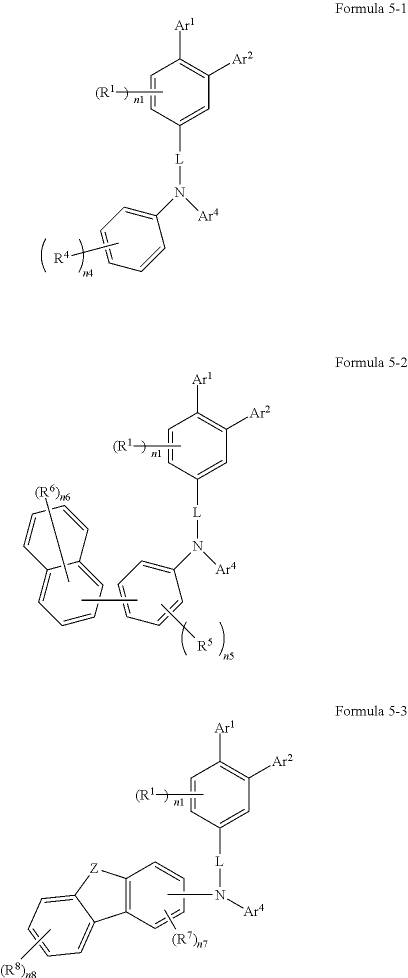

Formula 5-1 to Formula 5-3 embody cases where the types (kinds) of Ar³ in Formula 1 are specified.

In Formula 5-1 to Formula 5-3, R⁴ to R⁸ may each independently be hydrogen, deuterium, a substituted or unsubstituted alkyl group having 1 to 20 carbons, a substituted or unsubstituted aryl group having 6 to 30 ring-forming carbons, or a substituted or unsubstituted heteroaryl group having 2 to 30 ring-forming carbons. For example, in one or more embodiments, R⁴ may be hydrogen, deuterium, a substituted or unsubstituted phenyl group, a substituted or unsubstituted naphthylphenyl group, a substituted or unsubstituted dibenzofuranyl group, a substituted or unsubstituted dibenzothiophenyl group, a substituted or unsubstituted 9-phenylcarbozolyl group, or a substituted or unsubstituted 9,9-diphenylfluorenyl group, and R⁵ to R⁸ may each independently be hydrogen, deuterium, or a substituted or unsubstituted phenyl group.

In Formula 5-3, Z may be O, S, NR⁹, or CR¹⁰R¹¹.

In Formula 5-3, R⁹ to R¹¹ may each independently be a substituted or unsubstituted aryl group having 6 to 30 ring-forming carbons. For example, in one or more embodiments, R⁹ to R¹¹ may each independently be a substituted or unsubstituted phenyl group.

In Formula 5-1, n4 is an integer of 0 to 5. If (e.g., when) n4 is 0, the amine compound according to one or more embodiments may be unsubstituted with R⁴. An embodiment in which n4 is 5 and R⁴(s) are all hydrogens in Formula 5-1, may be the same as the embodiment in which n4 is 0 in Formula 5-1. If (e.g., when) n4 is an integer of 2 or greater, R⁴ provided in plural may be all the same, or at least one selected from among a plurality of R⁴(s) may be different.

In Formula 5-2 and Formula 5-3, n5 and n8 may each independently be an integer of 0 to 4. If (e.g., when) n5 and n8 are each 0, the amine compound according to one or more embodiments may be unsubstituted with each of R⁵ and R⁸. Embodiments in which n5 and n8 are each 4 and each of R⁵ and R⁸ is hydrogen in Formula 5-2 and Formula 5-3 may be the same as the embodiments in which n5 and n8 are each 0 in Formula 5-2 and Formula 5-3. If (e.g., when) n5 and n8 are each an integer of 2 or greater, R⁵ and R⁸ each provided in plural may be all the same, or at least one selected from among the plurality of R⁵(s) and R⁸(s) may be different.

In Formula 5-2, n6 is an integer of 0 to 7. If (e.g., when) n6 is 0, the amine compound according to one or more embodiments may be unsubstituted with R⁶. An embodiment in which n6 is 7 and R⁶(s) are all hydrogens in Formula 5-2 may be the same as the embodiment in which n6 is 0 in Formula 5-2. If (e.g., when) n6 is an integer of 2 or greater, R⁶ provided in plural may be all the same, or at least one selected from among the plurality of R⁶(s) may be different.

In Formula 5-3, n7 is an integer of 0 to 3. If (e.g., when) n7 is 0, the amine compound according to one or more embodiments may be unsubstituted with R⁷. An embodiment in which n7 is 3 and R⁷(s) are all hydrogens in Formula 5-3 may be the same as the embodiment in which n7 is 0 in Formula 5-3. If (e.g., when) n7 is an integer of 2 or greater, R⁷ provided in plural may be all the same, or at least one selected from among the plurality of R⁷(s) may be different.

In Formula 5-1, if (e.g., when) any one selected from among Ar¹ and Ar² is represented by Formula 3, R⁴ may be a substituted or unsubstituted dibenzofuranyl group, a substituted or unsubstituted dibenzothiophenyl group, a substituted or unsubstituted 9-phenylcarbazolyl group, or a substituted or unsubstituted 9,9-diphenylfluorenyl group.

The descriptions of R¹, n1, L, Ar¹, Ar², and Ar⁴ in Formula 1 may be similarly applied to Formula 5-1 to Formula 5-3. In other words, R¹, n1, L, Ar¹, Ar², Ar³, and Ar⁴ in Formula 5-1 to Formula 5-3 may each independently be the same as defined in Formula 1.

In one or more embodiments, the amine compound represented by Formula 1 may include at least one deuterium as a substituent. The amine compound according to one or more embodiments, represented by Formula 1, may contain a structure in which at least one hydrogen is substituted with deuterium.

In one or more embodiments, the amine compound represented by Formula 1 may be represented by Formula 6, and the amine compound may satisfy any one selected from among combinations present in Compound Combination Table.

The hole transport region HTR of the light-emitting element ED may include at least one selected from among amine compounds satisfying the combination present in Compound Combination Table 1. For example, in one or more embodiments, at least one selected from among the amine compounds satisfying the combination present in Compound Combination Table 1 may be contained in the hole transport layer HRL of the light-emitting element ED.

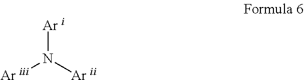

In Formula 6, Arⁱ may be any one selected from Substituent Group A, Arⁱⁱ may be any one selected from Substituent Group B, and Arⁱⁱⁱ may be any one selected from Substituent Group C. The superscript utilized in Substituent Group A, Substituent Group B, and Substituent Group C corresponds to the reference numeral used in Compound Combination Table 1 for each corresponding substituent moiety.

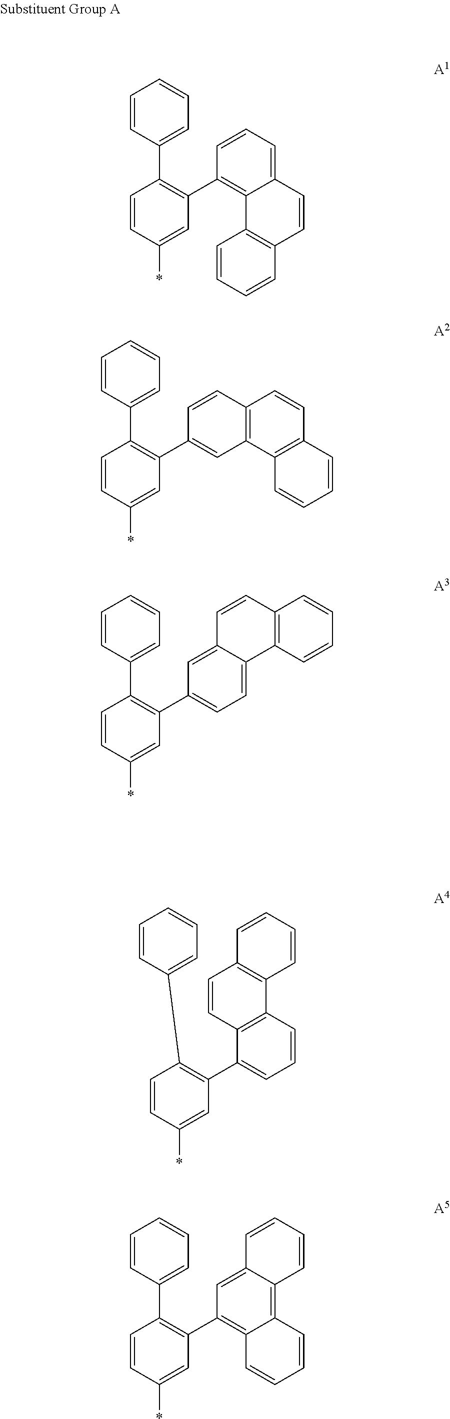 [SMILES: *c1ccc(-c2ccccc2)c(-c2cccc3ccc4ccccc4c23)c1 ; *c1ccc(-c2ccccc2)c(-c2ccc3ccc4ccccc4c3c2)c1 ; *c1ccc(-c2ccccc2)c(-c2ccc3c(ccc4ccccc43)c2)c1 ; *c1ccc(-c2ccccc2)c(-c2cccc3c2ccc2ccccc23)c1 ; *c1ccc(-c2ccccc2)c(-c2cc3ccccc3c3ccccc23)c1]

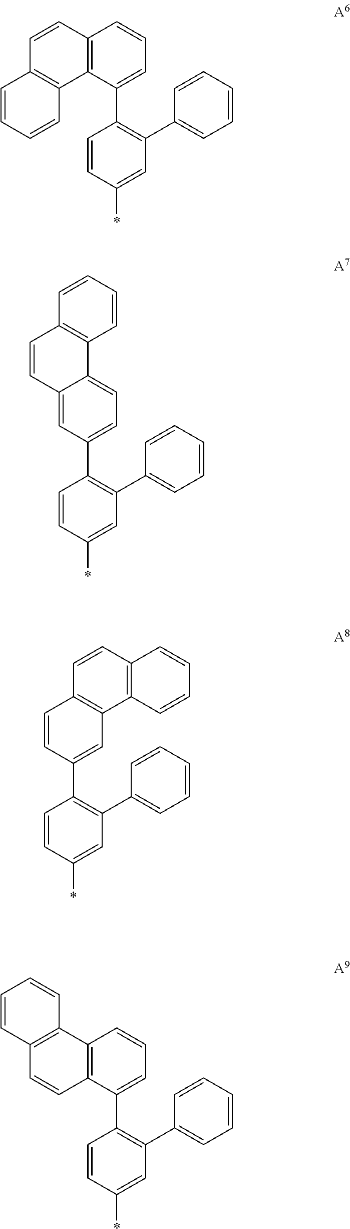 [SMILES: *c1ccc(-c2cccc3ccc4ccccc4c23)c(-c2ccccc2)c1 ; *c1ccc(-c2ccc3c(ccc4ccccc43)c2)c(-c2ccccc2)c1 ; *c1ccc(-c2ccc3ccc4ccccc4c3c2)c(-c2ccccc2)c1 ; *c1ccc(-c2cccc3c2ccc2ccccc23)c(-c2ccccc2)c1]

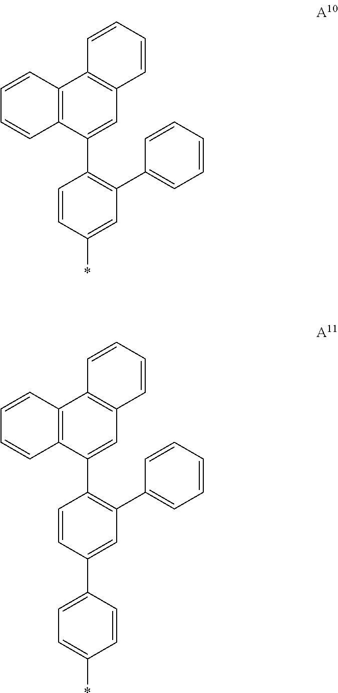 [SMILES: *c1ccc(-c2cc3ccccc3c3ccccc23)c(-c2ccccc2)c1 ; *c1ccc(-c2ccc(-c3cc4ccccc4c4ccccc34)c(-c3ccccc3)c2)cc1]

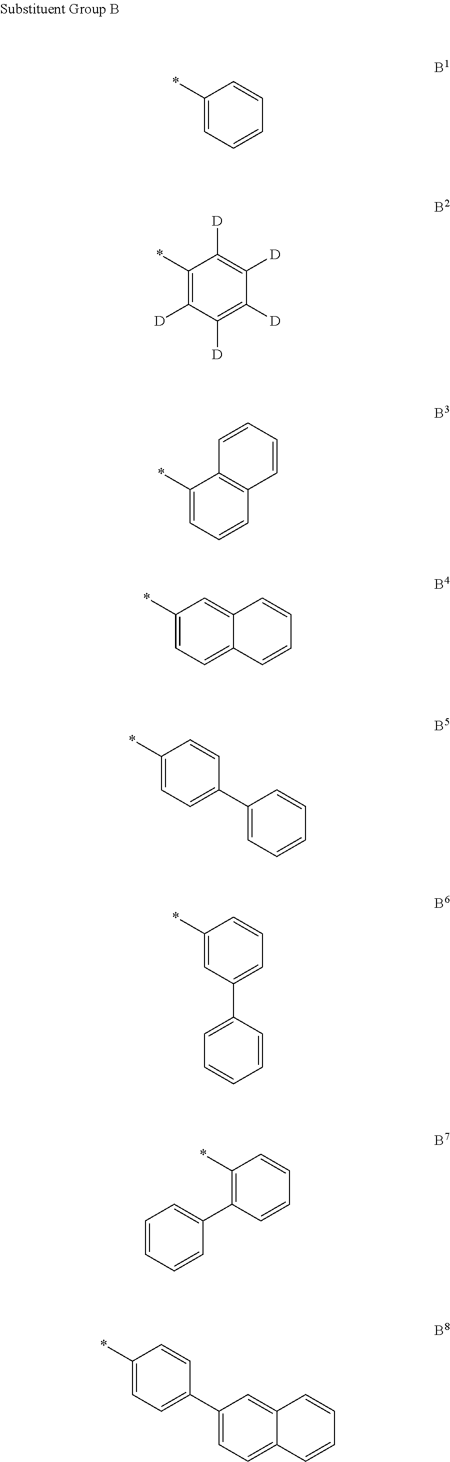

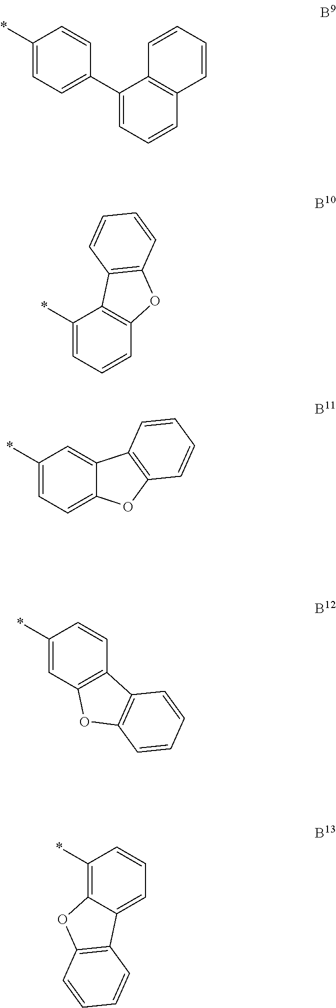 [SMILES: *c1ccc(-c2cccc3ccccc23)cc1 ; *c1cccc2oc3ccccc3c12 ; *c1ccc2oc3ccccc3c2c1 ; *c1ccc2c(c1)oc1ccccc12 ; *c1cccc2c1oc1ccccc12]

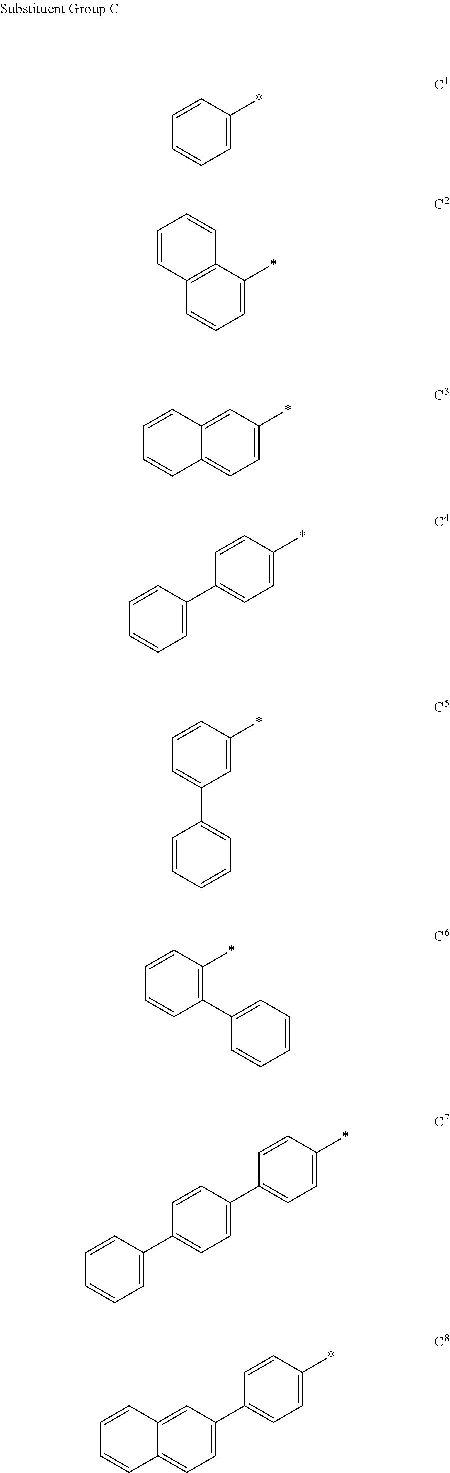

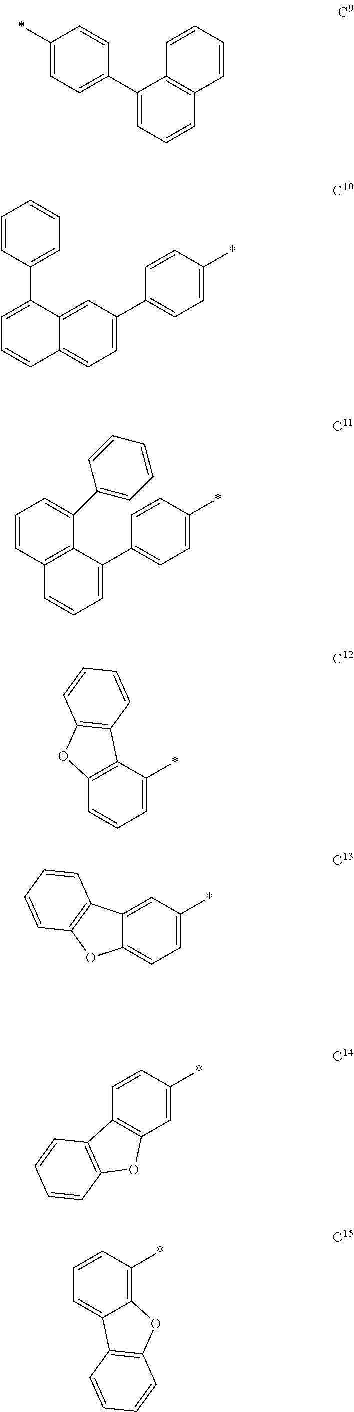

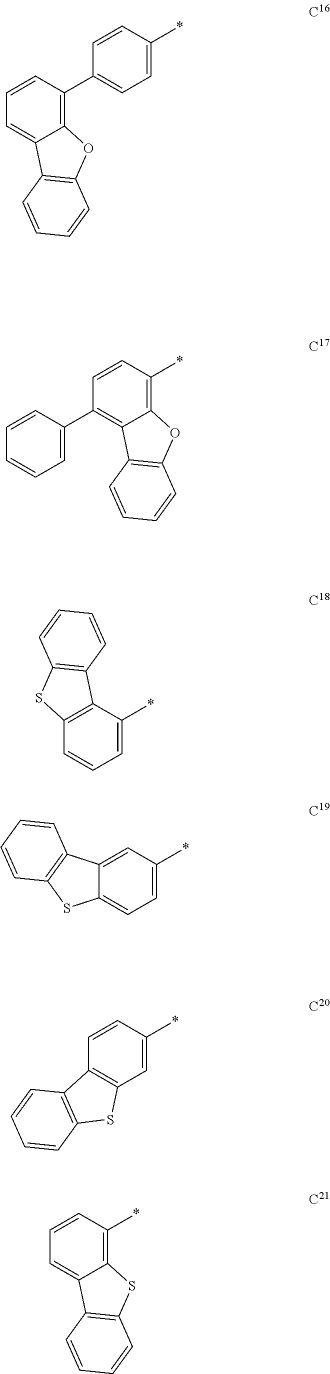 [SMILES: *c1ccc(-c2cccc3c2oc2ccccc23)cc1 ; *c1ccc(-c2ccccc2)c2c1oc1ccccc12 ; *c1cccc2sc3ccccc3c12 ; *c1ccc2sc3ccccc3c2c1 ; *c1ccc2c(c1)sc1ccccc12 ; *c1cccc2c1sc1ccccc12]

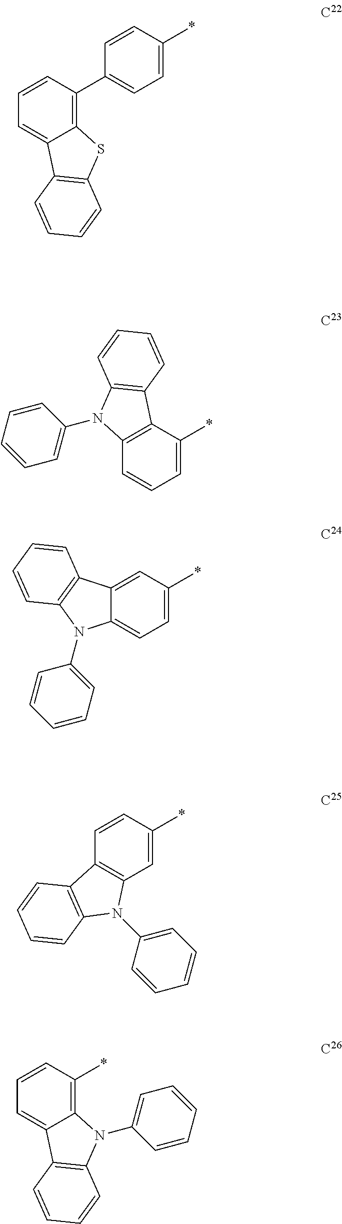 [SMILES: *c1ccc(-c2cccc3c2sc2ccccc23)cc1 ; *c1cccc2c1c1ccccc1n2-c1ccccc1 ; *c1ccc2c(c1)c1ccccc1n2-c1ccccc1 ; *c1ccc2c3ccccc3n(-c3ccccc3)c2c1 ; *c1cccc2c3ccccc3n(-c3ccccc3)c12]

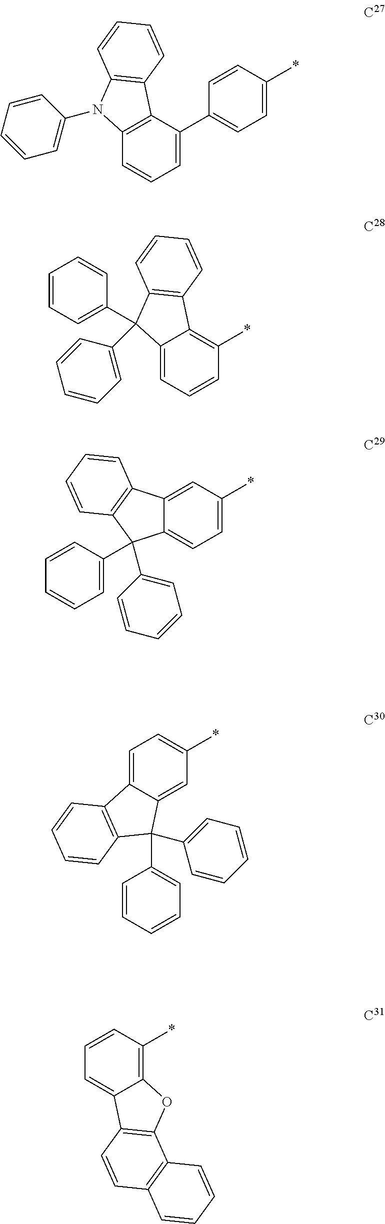 [SMILES: *c1ccc(-c2cccc3c2c2ccccc2n3-c2ccccc2)cc1 ; *c1cccc2c1-c1ccccc1C2(c1ccccc1)c1ccccc1 ; *c1ccc2c(c1)-c1ccccc1C2(c1ccccc1)c1ccccc1 ; *c1ccc2c(c1)C(c1ccccc1)(c1ccccc1)c1ccccc1-2 ; *c1cccc2c1oc1c3ccccc3ccc21]

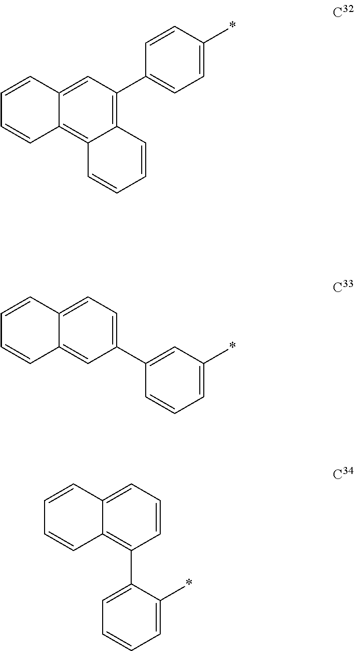 [SMILES: *c1ccc(-c2cc3ccccc3c3ccccc23)cc1 ; *c1cccc(-c2ccc3ccccc3c2)c1 ; *c1ccccc1-c1cccc2ccccc12]

TABLE 1
Compound Combination

| No. | A | B | C |
|---|---|---|---|
| 1 | 1 | 1 | 1 |
| 2 | 2 | 1 | 1 |
| 3 | 3 | 1 | 1 |
| 4 | 4 | 1 | 1 |
| 5 | 6 | 1 | 1 |
| 6 | 7 | 1 | 1 |
| 7 | 8 | 1 | 1 |
| 8 | 9 | 1 | 1 |
| 9 | 1 | 1 | 2 |
| 10 | 2 | 1 | 2 |
| 11 | 3 | 1 | 2 |
| 12 | 4 | 1 | 2 |
| 13 | 5 | 1 | 33 |
| 14 | 6 | 1 | 2 |
| 15 | 7 | 1 | 2 |
| 16 | 8 | 1 | 2 |
| 17 | 9 | 1 | 2 |
| 18 | 10 | 1 | 33 |
| 19 | 11 | 1 | 33 |
| 20 | 1 | 1 | 3 |
| 21 | 2 | 1 | 3 |
| 22 | 3 | 1 | 3 |
| 23 | 4 | 1 | 3 |
| 24 | 5 | 1 | 34 |
| 25 | 6 | 1 | 3 |
| 26 | 7 | 1 | 3 |
| 27 | 8 | 1 | 3 |
| 28 | 9 | 1 | 3 |
| 29 | 10 | 1 | 34 |
| 30 | 11 | 1 | 34 |
| 31 | 1 | 1 | 8 |
| 32 | 2 | 1 | 8 |
| 33 | 3 | 1 | 8 |
| 34 | 4 | 1 | 8 |
| 35 | 5 | 1 | 8 |
| 36 | 6 | 1 | 8 |
| 37 | 7 | 1 | 8 |
| 38 | 8 | 1 | 8 |
| 39 | 9 | 1 | 8 |
| 40 | 10 | 1 | 8 |
| 41 | 11 | 1 | 8 |
| 42 | 1 | 2 | 1 |
| 43 | 2 | 2 | 1 |
| 44 | 3 | 2 | 1 |
| 45 | 4 | 2 | 1 |
| 46 | 6 | 2 | 1 |
| 47 | 7 | 2 | 1 |
| 48 | 8 | 2 | 1 |
| 49 | 9 | 2 | 1 |
| 50 | 1 | 2 | 2 |
| 51 | 2 | 2 | 2 |
| 52 | 3 | 2 | 2 |
| 53 | 4 | 2 | 2 |
| 54 | 5 | 2 | 33 |
| 55 | 6 | 2 | 2 |
| 56 | 7 | 2 | 2 |
| 57 | 8 | 2 | 2 |
| 58 | 9 | 2 | 2 |
| 59 | 10 | 2 | 33 |
| 60 | 11 | 2 | 33 |
| 61 | 1 | 2 | 3 |
| 62 | 2 | 2 | 3 |
| 63 | 3 | 2 | 3 |
| 64 | 4 | 2 | 3 |
| 65 | 5 | 2 | 34 |
| 66 | 6 | 2 | 3 |
| 67 | 7 | 2 | 3 |
| 68 | 8 | 2 | 3 |
| 69 | 9 | 2 | 3 |
| 70 | 10 | 2 | 34 |
| 71 | 11 | 2 | 34 |
| 72 | 1 | 2 | 8 |
| 73 | 2 | 2 | 8 |
| 74 | 3 | 2 | 8 |
| 75 | 4 | 2 | 8 |
| 76 | 5 | 2 | 8 |
| 77 | 6 | 2 | 8 |
| 78 | 7 | 2 | 8 |
| 79 | 8 | 2 | 8 |
| 80 | 9 | 2 | 8 |
| 81 | 10 | 2 | 8 |
| 82 | 11 | 2 | 8 |
| 83 | 1 | 5 | 1 |
| 84 | 2 | 5 | 1 |
| 85 | 3 | 5 | 1 |
| 86 | 4 | 5 | 1 |
| 87 | 6 | 5 | 1 |
| 88 | 7 | 5 | 1 |
| 89 | 8 | 5 | 1 |
| 90 | 9 | 5 | 1 |
| 91 | 1 | 5 | 2 |
| 92 | 2 | 5 | 2 |
| 93 | 3 | 5 | 2 |
| 94 | 4 | 5 | 2 |
| 95 | 5 | 5 | 33 |
| 96 | 6 | 5 | 2 |
| 97 | 7 | 5 | 2 |
| 98 | 8 | 5 | 2 |
| 99 | 9 | 5 | 2 |
| 100 | 10 | 5 | 33 |
| 101 | 11 | 5 | 33 |
| 102 | 1 | 5 | 3 |
| 103 | 2 | 5 | 3 |
| 104 | 3 | 5 | 3 |
| 105 | 4 | 5 | 3 |
| 106 | 5 | 5 | 34 |
| 107 | 6 | 5 | 3 |
| 108 | 7 | 5 | 3 |
| 109 | 8 | 5 | 3 |
| 110 | 9 | 5 | 3 |
| 111 | 10 | 5 | 34 |
| 112 | 11 | 5 | 34 |
| 113 | 1 | 5 | 8 |
| 114 | 2 | 5 | 8 |
| 115 | 3 | 5 | 8 |
| 116 | 4 | 5 | 8 |
| 117 | 5 | 5 | 8 |
| 118 | 6 | 5 | 8 |
| 119 | 7 | 5 | 8 |
| 120 | 8 | 5 | 8 |
| 121 | 9 | 5 | 8 |
| 122 | 10 | 5 | 8 |
| 123 | 11 | 5 | 8 |
| 124 | 2 | 6 | 1 |
| 125 | 2 | 7 | 1 |
| 126 | 2 | 9 | 1 |
| 127 | 2 | 10 | 1 |
| 128 | 2 | 11 | 1 |
| 129 | 2 | 12 | 1 |
| 130 | 2 | 13 | 1 |
| 131 | 2 | 3 | 2 |
| 132 | 2 | 4 | 2 |
| 133 | 2 | 6 | 2 |
| 134 | 2 | 7 | 2 |
| 135 | 2 | 8 | 2 |
| 136 | 2 | 9 | 2 |
| 137 | 2 | 10 | 2 |
| 138 | 2 | 11 | 2 |
| 139 | 2 | 12 | 2 |
| 140 | 2 | 13 | 2 |
| 141 | 2 | 2 | 4 |
| 142 | 2 | 5 | 4 |
| 143 | 2 | 6 | 4 |
| 144 | 2 | 7 | 4 |
| 145 | 2 | 9 | 4 |
| 146 | 2 | 10 | 4 |
| 147 | 2 | 11 | 4 |
| 148 | 2 | 12 | 4 |
| 149 | 2 | 13 | 4 |
| 150 | 2 | 4 | 8 |
| 151 | 2 | 6 | 8 |
| 152 | 2 | 7 | 8 |
| 153 | 2 | 8 | 8 |
| 154 | 2 | 9 | 8 |
| 155 | 2 | 10 | 8 |
| 156 | 2 | 11 | 8 |
| 157 | 2 | 12 | 8 |
| 158 | 2 | 13 | 8 |
| 159 | 2 | 1 | 16 |
| 160 | 2 | 2 | 16 |
| 161 | 2 | 3 | 16 |
| 162 | 2 | 4 | 16 |
| 163 | 2 | 5 | 16 |
| 164 | 2 | 6 | 16 |
| 165 | 2 | 7 | 16 |
| 166 | 2 | 8 | 16 |
| 167 | 2 | 9 | 16 |
| 168 | 2 | 10 | 16 |
| 169 | 2 | 11 | 16 |
| 170 | 2 | 12 | 16 |
| 171 | 2 | 13 | 16 |
| 172 | 2 | 1 | 22 |
| 173 | 2 | 2 | 22 |
| 174 | 2 | 3 | 22 |
| 175 | 2 | 4 | 22 |
| 176 | 2 | 5 | 22 |
| 177 | 2 | 6 | 22 |
| 178 | 2 | 7 | 22 |
| 179 | 2 | 8 | 22 |
| 180 | 2 | 9 | 22 |
| 181 | 2 | 10 | 22 |
| 182 | 2 | 11 | 22 |
| 183 | 2 | 12 | 22 |
| 184 | 2 | 13 | 22 |
| 185 | 2 | 1 | 27 |
| 186 | 2 | 2 | 27 |
| 187 | 2 | 3 | 27 |
| 188 | 2 | 4 | 27 |
| 189 | 2 | 5 | 27 |
| 190 | 2 | 6 | 27 |
| 191 | 2 | 7 | 27 |
| 192 | 2 | 8 | 27 |
| 193 | 2 | 9 | 27 |
| 194 | 2 | 10 | 27 |
| 195 | 2 | 11 | 27 |
| 196 | 2 | 12 | 27 |
| 197 | 2 | 13 | 27 |
| 198 | 5 | 9 | 1 |
| 199 | 5 | 10 | 1 |
| 200 | 5 | 11 | 1 |
| 201 | 5 | 12 | 1 |
| 202 | 5 | 13 | 1 |
| 203 | 5 | 3 | 33 |
| 204 | 5 | 4 | 33 |
| 205 | 5 | 6 | 33 |
| 206 | 5 | 7 | 33 |
| 207 | 5 | 8 | 2 |
| 208 | 5 | 9 | 2 |
| 209 | 5 | 10 | 2 |
| 210 | 5 | 11 | 2 |
| 211 | 5 | 12 | 2 |
| 212 | 5 | 13 | 2 |
| 213 | 5 | 9 | 4 |
| 214 | 5 | 10 | 4 |
| 215 | 5 | 11 | 4 |
| 216 | 5 | 12 | 4 |
| 217 | 5 | 13 | 4 |
| 218 | 5 | 4 | 8 |
| 219 | 5 | 6 | 8 |
| 220 | 5 | 7 | 8 |
| 221 | 5 | 8 | 8 |
| 222 | 5 | 9 | 8 |
| 223 | 5 | 10 | 8 |
| 224 | 5 | 11 | 8 |
| 225 | 5 | 12 | 8 |
| 226 | 5 | 13 | 8 |
| 227 | 5 | 1 | 16 |
| 228 | 5 | 2 | 16 |
| 229 | 5 | 3 | 16 |
| 230 | 5 | 4 | 16 |
| 231 | 5 | 5 | 16 |
| 232 | 5 | 6 | 16 |
| 233 | 5 | 7 | 16 |
| 234 | 5 | 8 | 16 |
| 235 | 5 | 9 | 16 |
| 236 | 5 | 10 | 16 |
| 237 | 5 | 11 | 16 |
| 238 | 5 | 12 | 16 |
| 239 | 5 | 13 | 16 |
| 240 | 5 | 1 | 22 |
| 241 | 5 | 2 | 22 |
| 242 | 5 | 3 | 22 |
| 243 | 5 | 4 | 22 |
| 244 | 5 | 5 | 22 |
| 245 | 5 | 6 | 22 |
| 246 | 5 | 7 | 22 |
| 247 | 5 | 8 | 22 |
| 248 | 5 | 9 | 22 |
| 249 | 5 | 10 | 22 |
| 250 | 5 | 11 | 22 |
| 251 | 5 | 12 | 22 |
| 252 | 5 | 13 | 22 |
| 253 | 5 | 1 | 27 |
| 254 | 5 | 2 | 27 |
| 255 | 5 | 3 | 27 |
| 256 | 5 | 4 | 27 |
| 257 | 5 | 5 | 27 |
| 258 | 5 | 6 | 27 |
| 259 | 5 | 7 | 27 |
| 260 | 5 | 8 | 27 |
| 261 | 5 | 9 | 27 |
| 262 | 5 | 10 | 27 |
| 263 | 5 | 11 | 27 |
| 264 | 5 | 12 | 27 |
| 265 | 5 | 13 | 27 |
| 266 | 8 | 6 | 1 |
| 267 | 8 | 7 | 1 |
| 268 | 8 | 9 | 1 |
| 269 | 8 | 10 | 1 |
| 270 | 8 | 11 | 1 |
| 271 | 8 | 12 | 1 |
| 272 | 8 | 13 | 1 |
| 273 | 8 | 3 | 2 |
| 274 | 8 | 4 | 2 |
| 275 | 8 | 6 | 2 |
| 276 | 8 | 7 | 2 |
| 277 | 8 | 8 | 2 |
| 278 | 8 | 9 | 2 |
| 279 | 8 | 10 | 2 |
| 280 | 8 | 11 | 2 |
| 281 | 8 | 12 | 2 |
| 282 | 8 | 13 | 2 |
| 283 | 8 | 2 | 4 |
| 284 | 8 | 5 | 4 |
| 285 | 8 | 6 | 4 |
| 286 | 8 | 7 | 4 |
| 287 | 8 | 9 | 4 |
| 288 | 8 | 10 | 4 |
| 289 | 8 | 11 | 4 |
| 290 | 8 | 12 | 4 |
| 291 | 8 | 13 | 4 |
| 292 | 8 | 4 | 8 |
| 293 | 8 | 6 | 8 |
| 294 | 8 | 7 | 8 |
| 295 | 8 | 8 | 8 |
| 296 | 8 | 9 | 8 |
| 297 | 8 | 10 | 8 |
| 298 | 8 | 11 | 8 |
| 299 | 8 | 12 | 8 |
| 300 | 8 | 13 | 8 |
| 301 | 8 | 1 | 16 |
| 302 | 8 | 2 | 16 |
| 303 | 8 | 3 | 16 |
| 304 | 8 | 4 | 16 |
| 305 | 8 | 5 | 16 |
| 306 | 8 | 6 | 16 |
| 307 | 8 | 7 | 16 |
| 308 | 8 | 8 | 16 |
| 309 | 8 | 9 | 16 |
| 310 | 8 | 10 | 16 |
| 311 | 8 | 11 | 16 |
| 312 | 8 | 12 | 16 |
| 313 | 8 | 13 | 16 |
| 314 | 8 | 1 | 22 |
| 315 | 8 | 2 | 22 |
| 316 | 8 | 3 | 22 |
| 317 | 8 | 4 | 22 |
| 318 | 8 | 5 | 22 |
| 319 | 8 | 6 | 22 |
| 320 | 8 | 7 | 22 |
| 321 | 8 | 8 | 22 |
| 322 | 8 | 9 | 22 |
| 323 | 8 | 10 | 22 |
| 324 | 8 | 11 | 22 |
| 325 | 8 | 12 | 22 |
| 326 | 8 | 13 | 22 |
| 327 | 8 | 1 | 27 |
| 328 | 8 | 2 | 27 |
| 329 | 8 | 3 | 27 |
| 330 | 8 | 4 | 27 |
| 331 | 8 | 5 | 27 |
| 332 | 8 | 6 | 27 |
| 333 | 8 | 7 | 27 |
| 334 | 8 | 8 | 27 |
| 335 | 8 | 9 | 27 |
| 336 | 8 | 10 | 27 |
| 337 | 8 | 11 | 27 |
| 338 | 8 | 12 | 27 |
| 339 | 8 | 13 | 27 |
| 340 | 10 | 9 | 1 |
| 341 | 10 | 10 | 1 |
| 342 | 10 | 11 | 1 |
| 343 | 10 | 12 | 1 |
| 344 | 10 | 13 | 1 |
| 345 | 10 | 3 | 33 |
| 346 | 10 | 4 | 33 |
| 347 | 10 | 6 | 33 |
| 348 | 10 | 7 | 33 |
| 349 | 10 | 8 | 2 |
| 350 | 10 | 9 | 2 |
| 351 | 10 | 10 | 2 |
| 352 | 10 | 11 | 2 |
| 353 | 10 | 12 | 2 |
| 354 | 10 | 13 | 2 |
| 355 | 10 | 9 | 4 |
| 356 | 10 | 10 | 4 |
| 357 | 10 | 11 | 4 |
| 358 | 10 | 12 | 4 |
| 359 | 10 | 13 | 4 |
| 360 | 10 | 4 | 8 |
| 361 | 10 | 6 | 8 |
| 362 | 10 | 7 | 8 |
| 363 | 10 | 8 | 8 |
| 364 | 10 | 9 | 8 |
| 365 | 10 | 10 | 8 |
| 366 | 10 | 11 | 8 |
| 367 | 10 | 12 | 8 |
| 368 | 10 | 13 | 8 |
| 369 | 10 | 1 | 16 |
| 370 | 10 | 2 | 16 |
| 371 | 10 | 3 | 16 |
| 372 | 10 | 4 | 16 |
| 373 | 10 | 5 | 16 |
| 374 | 10 | 6 | 16 |
| 375 | 10 | 7 | 16 |
| 376 | 10 | 8 | 16 |
| 377 | 10 | 9 | 16 |
| 378 | 10 | 10 | 16 |
| 379 | 10 | 11 | 16 |
| 380 | 10 | 12 | 16 |
| 381 | 10 | 13 | 16 |
| 382 | 10 | 1 | 22 |
| 383 | 10 | 2 | 22 |
| 384 | 10 | 3 | 22 |
| 385 | 10 | 4 | 22 |
| 386 | 10 | 5 | 22 |
| 387 | 10 | 6 | 22 |
| 388 | 10 | 7 | 22 |
| 389 | 10 | 8 | 22 |
| 390 | 10 | 9 | 22 |
| 391 | 10 | 10 | 22 |
| 392 | 10 | 11 | 22 |
| 393 | 10 | 12 | 22 |
| 394 | 10 | 13 | 22 |
| 395 | 10 | 1 | 27 |
| 396 | 10 | 2 | 27 |
| 397 | 10 | 3 | 27 |
| 398 | 10 | 4 | 27 |
| 399 | 10 | 5 | 27 |
| 400 | 10 | 6 | 27 |
| 401 | 10 | 7 | 27 |
| 402 | 10 | 8 | 27 |
| 403 | 10 | 9 | 27 |
| 404 | 10 | 10 | 27 |
| 405 | 10 | 11 | 27 |
| 406 | 10 | 12 | 27 |
| 407 | 10 | 13 | 27 |
| 408 | 2 | 1 | 7 |
| 409 | 2 | 1 | 10 |
| 410 | 2 | 1 | 11 |
| 411 | 2 | 1 | 17 |
| 412 | 2 | 1 | 18 |
| 413 | 2 | 1 | 19 |
| 414 | 2 | 1 | 20 |
| 415 | 2 | 1 | 21 |
| 416 | 2 | 1 | 23 |
| 417 | 2 | 1 | 24 |
| 418 | 2 | 1 | 25 |
| 419 | 2 | 1 | 26 |
| 420 | 2 | 1 | 28 |
| 421 | 2 | 1 | 29 |
| 422 | 2 | 1 | 30 |
| 423 | 2 | 1 | 31 |
| 424 | 2 | 1 | 32 |
| 425 | 2 | 2 | 5 |
| 426 | 2 | 2 | 6 |
| 427 | 2 | 2 | 7 |
| 428 | 2 | 2 | 9 |
| 429 | 2 | 2 | 10 |
| 430 | 2 | 2 | 11 |
| 431 | 2 | 2 | 12 |
| 432 | 2 | 2 | 13 |
| 433 | 2 | 2 | 14 |
| 434 | 2 | 2 | 15 |
| 435 | 2 | 2 | 17 |
| 436 | 2 | 2 | 18 |
| 437 | 2 | 2 | 19 |
| 438 | 2 | 2 | 20 |
| 439 | 2 | 2 | 21 |
| 440 | 2 | 2 | 23 |
| 441 | 2 | 2 | 24 |
| 442 | 2 | 2 | 25 |
| 443 | 2 | 2 | 26 |
| 444 | 2 | 2 | 28 |
| 445 | 2 | 2 | 29 |
| 446 | 2 | 2 | 30 |
| 447 | 2 | 2 | 31 |
| 448 | 2 | 2 | 32 |
| 449 | 2 | 12 | 3 |
| 450 | 2 | 12 | 5 |
| 451 | 2 | 12 | 6 |
| 452 | 2 | 12 | 7 |
| 453 | 2 | 12 | 9 |
| 454 | 2 | 12 | 10 |
| 455 | 2 | 12 | 11 |
| 456 | 2 | 12 | 12 |
| 457 | 2 | 12 | 13 |
| 458 | 2 | 12 | 14 |
| 459 | 2 | 12 | 15 |
| 460 | 2 | 12 | 17 |
| 461 | 2 | 12 | 18 |
| 462 | 2 | 12 | 19 |
| 463 | 2 | 12 | 20 |
| 464 | 2 | 12 | 21 |
| 465 | 2 | 12 | 23 |
| 466 | 2 | 12 | 24 |
| 467 | 2 | 12 | 25 |
| 468 | 2 | 12 | 26 |
| 469 | 2 | 12 | 28 |
| 470 | 2 | 12 | 29 |
| 471 | 2 | 12 | 30 |
| 472 | 2 | 3 | 31 |
| 473 | 2 | 3 | 32 |
| 474 | 5 | 1 | 10 |
| 475 | 5 | 1 | 11 |
| 476 | 5 | 1 | 17 |
| 477 | 5 | 1 | 18 |
| 478 | 5 | 1 | 19 |
| 479 | 5 | 1 | 20 |
| 480 | 5 | 1 | 21 |
| 481 | 5 | 1 | 23 |
| 482 | 5 | 1 | 24 |
| 483 | 5 | 1 | 25 |
| 484 | 5 | 1 | 26 |
| 485 | 5 | 1 | 28 |
| 486 | 5 | 1 | 29 |
| 487 | 5 | 1 | 30 |
| 488 | 5 | 1 | 31 |
| 489 | 5 | 1 | 32 |
| 490 | 5 | 2 | 9 |
| 491 | 5 | 2 | 10 |
| 492 | 5 | 2 | 11 |
| 493 | 5 | 2 | 12 |
| 494 | 5 | 2 | 13 |
| 495 | 5 | 2 | 14 |
| 496 | 5 | 2 | 15 |
| 497 | 5 | 2 | 17 |
| 498 | 5 | 2 | 18 |
| 499 | 5 | 2 | 19 |
| 500 | 5 | 2 | 20 |
| 501 | 5 | 2 | 21 |
| 502 | 5 | 2 | 23 |
| 503 | 5 | 2 | 24 |
| 504 | 5 | 2 | 25 |
| 505 | 5 | 2 | 26 |
| 506 | 5 | 2 | 28 |
| 507 | 5 | 2 | 29 |
| 508 | 5 | 2 | 30 |
| 509 | 5 | 2 | 31 |
| 510 | 5 | 2 | 32 |
| 511 | 5 | 12 | 3 |
| 512 | 5 | 12 | 5 |
| 513 | 5 | 12 | 6 |
| 514 | 5 | 12 | 7 |
| 515 | 5 | 12 | 9 |
| 516 | 5 | 12 | 10 |
| 517 | 5 | 12 | 11 |
| 518 | 5 | 12 | 12 |
| 519 | 5 | 12 | 13 |
| 520 | 5 | 12 | 14 |
| 521 | 5 | 12 | 15 |
| 522 | 5 | 12 | 17 |
| 523 | 5 | 12 | 18 |
| 524 | 5 | 12 | 19 |
| 525 | 5 | 12 | 20 |
| 526 | 5 | 12 | 21 |
| 527 | 5 | 12 | 23 |
| 528 | 5 | 12 | 24 |
| 529 | 5 | 12 | 25 |
| 530 | 5 | 12 | 26 |
| 531 | 5 | 12 | 28 |
| 532 | 5 | 12 | 29 |
| 533 | 5 | 12 | 30 |
| 534 | 5 | 1 | 31 |
| 535 | 5 | 1 | 32 |
| 536 | 8 | 1 | 7 |
| 537 | 8 | 1 | 10 |
| 538 | 8 | 1 | 11 |
| 539 | 8 | 1 | 17 |
| 540 | 8 | 1 | 18 |
| 541 | 8 | 1 | 19 |
| 542 | 8 | 1 | 20 |
| 543 | 8 | 1 | 21 |
| 544 | 8 | 1 | 23 |
| 545 | 8 | 1 | 24 |
| 546 | 8 | 1 | 25 |
| 547 | 8 | 1 | 26 |
| 548 | 8 | 1 | 28 |
| 549 | 8 | 1 | 29 |
| 550 | 8 | 1 | 30 |
| 551 | 8 | 1 | 31 |
| 552 | 8 | 1 | 32 |
| 553 | 8 | 2 | 5 |
| 554 | 8 | 2 | 6 |
| 555 | 8 | 2 | 7 |
| 556 | 8 | 2 | 9 |
| 557 | 8 | 2 | 10 |
| 558 | 8 | 2 | 11 |
| 559 | 8 | 2 | 12 |
| 560 | 8 | 2 | 13 |
| 561 | 8 | 2 | 14 |
| 562 | 8 | 2 | 15 |
| 563 | 8 | 2 | 17 |
| 564 | 8 | 2 | 18 |
| 565 | 8 | 2 | 19 |
| 566 | 8 | 2 | 20 |
| 567 | 8 | 2 | 21 |
| 568 | 8 | 2 | 23 |
| 569 | 8 | 2 | 24 |
| 570 | 8 | 2 | 25 |
| 571 | 8 | 2 | 26 |
| 572 | 8 | 2 | 28 |
| 573 | 8 | 2 | 29 |
| 574 | 8 | 2 | 30 |
| 575 | 8 | 2 | 31 |
| 576 | 8 | 2 | 32 |
| 577 | 8 | 12 | 3 |
| 578 | 8 | 12 | 5 |
| 579 | 8 | 12 | 6 |
| 580 | 8 | 12 | 7 |
| 581 | 8 | 12 | 9 |
| 582 | 8 | 12 | 10 |
| 583 | 8 | 12 | 11 |
| 584 | 8 | 12 | 12 |
| 585 | 8 | 12 | 13 |
| 586 | 8 | 12 | 14 |
| 587 | 8 | 12 | 15 |
| 588 | 8 | 12 | 17 |
| 589 | 8 | 12 | 18 |
| 590 | 8 | 12 | 19 |
| 591 | 8 | 12 | 20 |
| 592 | 8 | 12 | 21 |
| 593 | 8 | 12 | 23 |
| 594 | 8 | 12 | 24 |
| 595 | 8 | 12 | 25 |
| 596 | 8 | 12 | 26 |
| 597 | 8 | 12 | 28 |
| 598 | 8 | 12 | 29 |
| 599 | 8 | 12 | 30 |
| 600 | 8 | 12 | 31 |
| 601 | 8 | 12 | 32 |
| 602 | 10 | 1 | 10 |
| 603 | 10 | 1 | 11 |
| 604 | 10 | 1 | 17 |
| 605 | 10 | 1 | 18 |
| 606 | 10 | 1 | 19 |
| 607 | 10 | 1 | 20 |
| 608 | 10 | 1 | 21 |
| 609 | 10 | 1 | 23 |
| 610 | 10 | 1 | 24 |
| 611 | 10 | 1 | 25 |
| 612 | 10 | 1 | 26 |
| 613 | 10 | 1 | 28 |
| 614 | 10 | 1 | 29 |
| 615 | 10 | 1 | 30 |
| 616 | 10 | 1 | 31 |
| 617 | 10 | 1 | 32 |
| 618 | 10 | 2 | 9 |
| 619 | 10 | 2 | 10 |
| 620 | 10 | 2 | 11 |
| 621 | 10 | 2 | 12 |
| 622 | 10 | 2 | 13 |
| 623 | 10 | 2 | 14 |
| 624 | 10 | 2 | 15 |
| 625 | 10 | 2 | 17 |
| 626 | 10 | 2 | 18 |
| 627 | 10 | 2 | 19 |
| 628 | 10 | 2 | 20 |
| 629 | 10 | 2 | 21 |
| 630 | 10 | 2 | 23 |
| 631 | 10 | 2 | 24 |
| 632 | 10 | 2 | 25 |
| 633 | 10 | 2 | 26 |
| 634 | 10 | 2 | 28 |
| 635 | 10 | 2 | 29 |
| 636 | 10 | 2 | 30 |
| 637 | 10 | 2 | 31 |
| 638 | 10 | 2 | 32 |
| 639 | 10 | 12 | 3 |
| 640 | 10 | 12 | 5 |
| 641 | 10 | 12 | 6 |
| 642 | 10 | 12 | 7 |
| 643 | 10 | 12 | 9 |
| 644 | 10 | 12 | 10 |
| 645 | 10 | 12 | 11 |
| 646 | 10 | 12 | 12 |
| 647 | 10 | 12 | 13 |
| 648 | 10 | 12 | 14 |
| 649 | 10 | 12 | 15 |
| 650 | 10 | 12 | 17 |
| 651 | 10 | 12 | 18 |
| 652 | 10 | 12 | 19 |
| 653 | 10 | 12 | 20 |
| 654 | 10 | 12 | 21 |
| 655 | 10 | 12 | 23 |
| 656 | 10 | 12 | 24 |
| 657 | 10 | 12 | 25 |
| 658 | 10 | 12 | 26 |
| 659 | 10 | 12 | 28 |
| 660 | 10 | 12 | 29 |
| 661 | 10 | 12 | 30 |
| 662 | 10 | 3 | 31 |
| 663 | 10 | 3 | 32 |

The amine compound according to one or more embodiments includes the first substituent, the second substituent, and the third substituent, each connected to the core nitrogen atom of the amine compound, and thus may achieve improvements in high efficiency and long lifespan of the light-emitting element.

The amine compound according to one or more embodiments includes an amine group, and has a structure in which the first substituent, the second substituent, and the third substituent are bonded to the amine group. In the amine compound according to one or more embodiments, represented by Formula 1, the first substituent may include a benzene moiety, and a substituted or unsubstituted phenanthrenyl group and an unsubstituted phenyl group, each connected to the benzene moiety. In this regard, the substituted or unsubstituted phenanthrenyl group and the unsubstituted phenyl group may be connected to the benzene moiety to be in an ortho relationship with each other. In addition, in the first substituent, one selected from among the substituted or unsubstituted phenanthrenyl group and the unsubstituted phenyl group may be connected to the benzene moiety at a para position with respect to the nitrogen atom, and the other may be connected to the benzene moiety at an ortho position with respect to the above-described one.

The amine compound according to one or more embodiments may have excellent or suitable thermal stability and high charge transportability due to introduction of such substituents and their specified substitution positions. Therefore, the amine compound according to one or more embodiments may have improved efficiency and lifespan. In addition, the light-emitting element according to one or more embodiments including the amine compound according to one or more embodiments may have improved luminous efficiency and lifespan.

Referring back to FIG. 3 to FIG. 6, the light-emitting element according to one or more embodiments of the disclosure will be further described.

As described above, the hole transport region HTR contains the above-described amine compound according to one or more embodiments of the present disclosure. For example, the hole transport region HTR contains the amine compound represented by Formula 1.

When the hole transport region HTR has a multi-layered structure having a plurality of layers, any one layer selected from among the plurality of layers may contain the amine compound represented by Formula 1. For example, in one or more embodiments, the hole transport region HTR includes a hole injection layer HIL arranged on the first electrode EL1, and a hole transport layer HTL arranged on the hole injection layer HIL, and the hole transport layer HTL may include the amine compound represented by Formula 1. However, embodiments of the present disclosure are not limited thereto, and for example, in one or more embodiments, the hole injection layer HIL may include the amine compound represented by Formula 1.

In one or more embodiments, the hole transport region HTR may include one or more amine compounds represented by Formula 1. For example, in one or more embodiments, the hole transport region HTR may include at least one selected from among the compounds present in the above-described Compound Combination Table 1.

In one or more embodiments, the hole transport region HTR may further include a compound represented by Formula H-1.

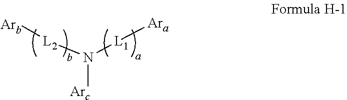

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. a and b may each independently be an integer of 0 to 10. In one or more embodiments, if (e.g., when) a and/or b are each an integer of 2 or greater, a plurality of L₁'s and/or a plurality of L₂'s may each independently be a substituted or unsubstituted arylene group having 6 to 30 ring-forming carbon atoms or a substituted or unsubstituted heteroarylene group having 2 to 30 ring-forming carbon atoms.

In Formula H-1, Arₐ and Ar_{b} may each independently be a substituted or unsubstituted aryl group having 6 to 30 ring-forming carbon atoms or a substituted or unsubstituted heteroaryl group having 2 to 30 ring-forming carbon atoms. In addition, in Formula H-1, Arc may be a substituted or unsubstituted aryl group having 6 to 30 ring-forming carbon atoms or a substituted or unsubstituted heteroaryl group having 2 to 30 ring-forming carbon atoms.

In one or more embodiments, the compound represented by Formula H-1 may be a monoamine compound. In one or more embodiments, the compound represented by Formula H-1 may be a diamine compound in which at least one selected from among Arₐ to Ar_{c} includes an amine group as a substituent. In one or more embodiments, the compound represented by Formula H-1 may be a carbazole-based compound including a substituted or unsubstituted carbazole group in at least one of Arₐ or Ar_{b}, or a fluorene-based compound including a substituted or unsubstituted fluorene group in at least one of Arₐ or Ar_{b}.

The compound represented by Formula H-1 may be any one selected from among compounds in Compound Group H. However, the compounds listed in Compound Group H are mere examples, and the compounds represented by Formula H-1 are not limited to those represented in Compound Group H:

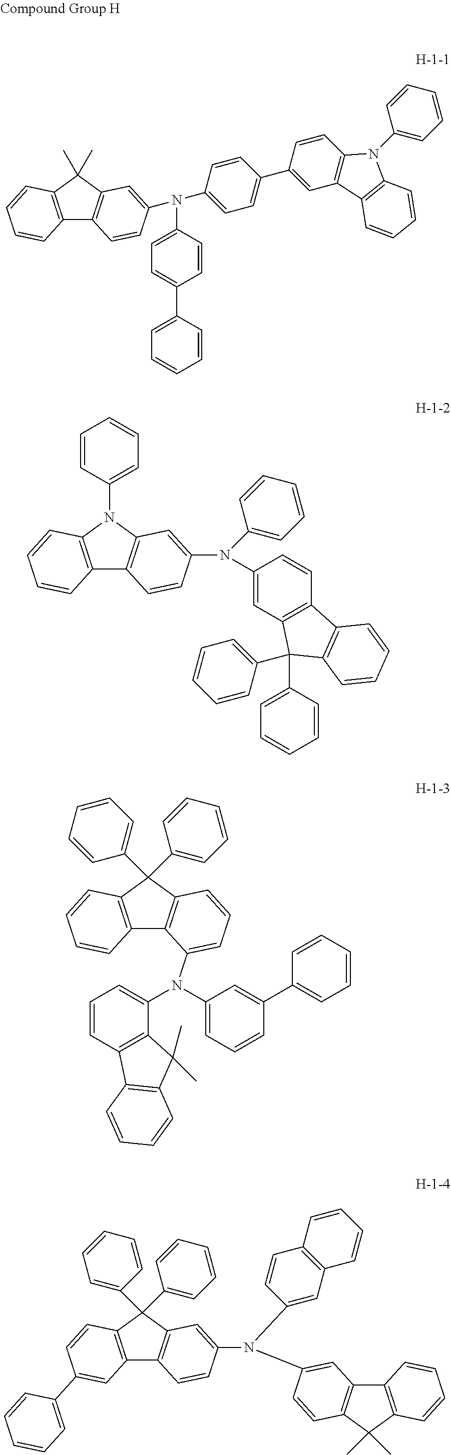 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

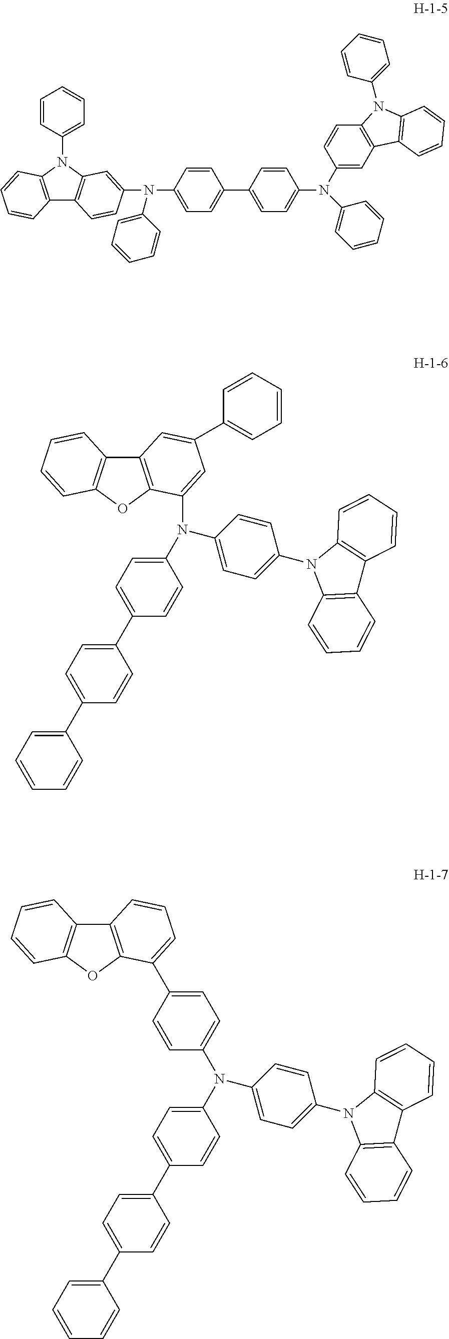 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

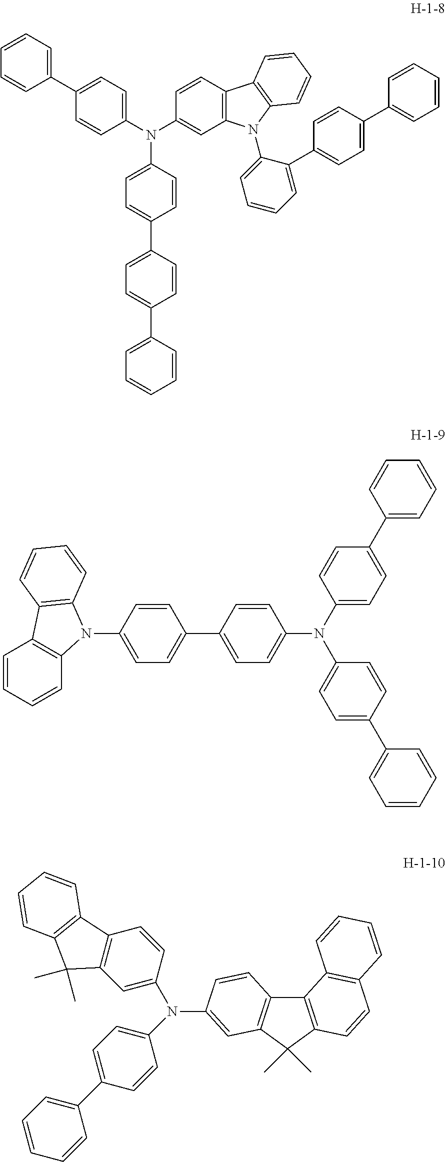 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

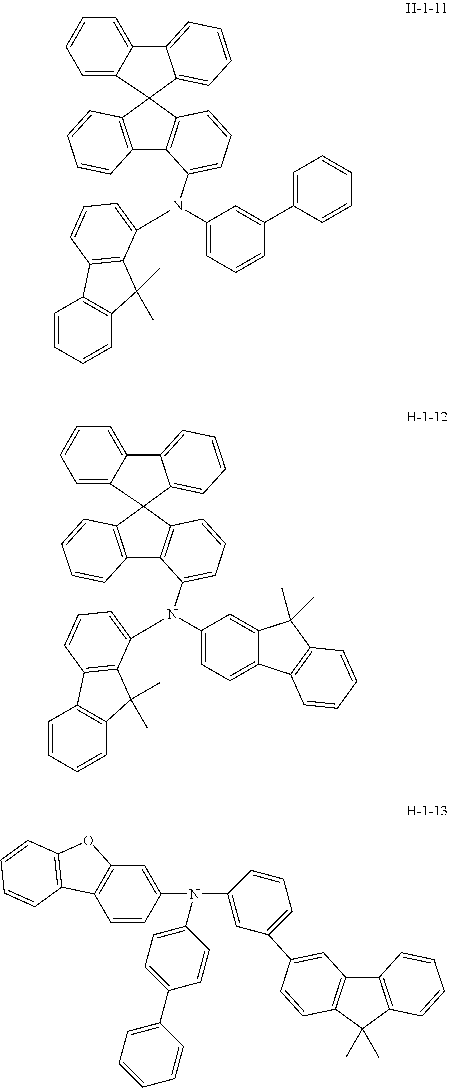 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

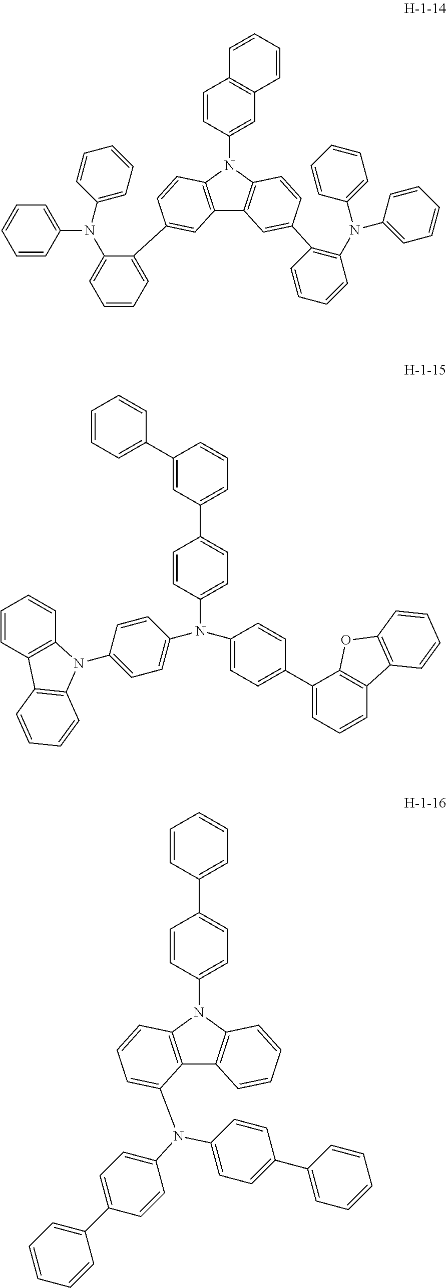 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

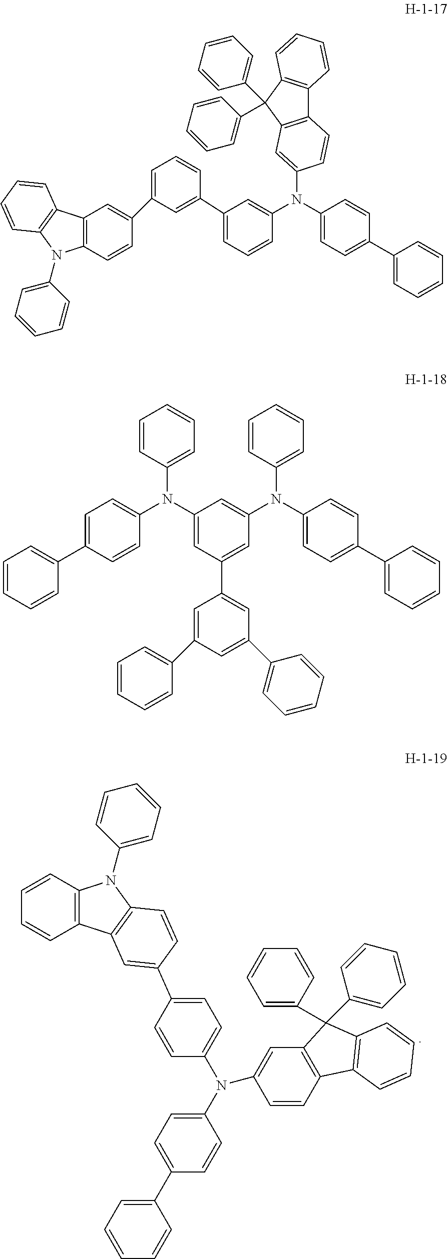 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

In one or more embodiments, the hole transport region HTR may further include a suitable hole transport material.

For example, in one or more embodiments, the hole transport region HTR may include one or more selected from among a phthalocyanine compound such as copper phthalocyanine; N1, N1′-([1,1′-biphenyl]-4,4′-diyl)bis(N1-phenyl-N4, N4-di-m-tolylbenzene-1,4-diamine) (DNTPD), 4,4′,4″-[tris(3-methylphenyl)phenylamino]triphenylamine (m-MTDATA), 4,4′,4″-tris(N,N-diphenylamino) triphenylamine (TDATA), 4,4′,4″-tris[N (2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalen-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HATCN), and/or the like.

In one or more embodiments, the hole transport region HTR may include one or more selected from among a carbazole-based derivative such as N-phenyl carbazole and/or polyvinyl carbazole, a fluorene-based derivative, a triphenylamine-based derivative such as N,N′-bis(3-methylphenyl)-N,N′-diphenyl-[1,1-biphenyl]-4,4′-diamine (TPD) and/or 4,4′,4″-tris(N-carbazolyl)triphenylamine (TCTA), N,N′-di(naphthalen-1-yl)-N,N′-diphenyl-benzidine (NPB), 4,4′-cyclohexylidene bis[N,N-bis(4-methylphenyl]benzenamine] (TAPC), 4,4′-bis[N,N′-(3-tolyl)amino]-3,3′-dimethylbiphenyl (HMTPD), 1,3-bis(N-carbazolyl)benzene (mCP), and/or the like.

In one or more embodiments, the hole transport region HTR may include one or more selected from among 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), and/or the like.

The hole transport region HTR may include one or more of the above-described compounds of the hole transport region in at least one of a hole injection layer HIL, a hole transport layer HTL, or an electron blocking layer EBL.

A thickness of the hole transport region HTR may be from about 100 Å to about 10,000 Å, for example, from about 100 Å to about 5,000 Å. When the hole transport region HTR includes a hole injection layer HIL, the hole injection layer HIL may have, for example, a thickness of about 30 Å to about 1,000 Å. When the hole transport region HTR includes a hole transport layer HTL, the hole transport layer HTL may have a thickness of about 250 Å to about 1,000 Å. For example, if (e.g., when) the hole transport region HTR includes an electron blocking layer EBL, the electron blocking layer EBL may have a thickness of about 10 Å to about 1,000 Å. If (e.g., when) the thicknesses of the hole transport region HTR, the hole injection layer HIL, the hole transport layer HTL, and the electron blocking layer EBL satisfy the above-described ranges, satisfactory hole transport properties may be achieved without a substantial increase in driving voltage.

In one or more embodiments, the hole transport region HTR may further include a charge generating material to increase conductivity (e.g., electric conductivity) in addition to the above-described materials. The charge generating material may be dispersed uniformly (e.g., substantially uniformly) or non-uniformly in the hole transport region HTR. The charge generating material may be, for example, a p-dopant. The p-dopant may include at least one of a halogenated metal compound, a quinone derivative, a metal oxide, or a cyano group-containing compound, but embodiments of the present disclosure are not limited thereto. For example, in one or more embodiments, the p-dopant may include a metal halide compound such as CuI and/or RbI, a quinone derivative such as tetracyanoquinodimethane (TCNQ) and/or 2,3,5,6-tetrafluoro-7,7,8,8-tetracyanoquinodimethane (F4-TCNQ), a metal oxide such as tungsten oxide and/or molybdenum oxide, a cyano group-containing compound such as dipyrazino[2,3-f: 2′,3′-h] quinoxaline-2,3,6,7,10, 11-hexacarbonitrile (HATCN) and/or 4-[2,3-bis [cyano-(4-cyano-2,3,5,6-tetrafluorophenyl)methylidene]cyclopropylidene]-cyanomethyl]-2,3,5,6-tetrafluorobenzonitrile (NDP9), and/or the like, but embodiments of the present disclosure are not limited thereto.

As described above, the hole transport region HTR may further include at least one of the buffer layer or the electron blocking layer EBL in addition to the hole injection layer HIL and the hole transport layer HTL. The buffer layer may compensate for a resonance distance according to the wavelength of light emitted from the light-emitting layer EML and may thus increase light emission efficiency. A material that may be included in the hole transport region HTR may be used as a material to be included in the buffer layer. The electron blocking layer EBL is a layer that serves to prevent or reduce the electron injection from the electron transport region ETR to the hole transport region HTR.

The light-emitting layer EML may be provided on the hole transport region HTR. The light-emitting layer EML may have a thickness of, for example, about 100 Å 1 to about 1,000 Å or about 100 Å to about 300 Å. The light-emitting layer EML may have a single layer formed of a single material, a single layer formed of multiple different materials, or a multi-layered structure having a plurality of layers formed of multiple different materials.

In the light-emitting element ED of one or more embodiments, the light-emitting layer EML may be to emit blue light. The light-emitting element ED of one or more embodiments may include the amine compound of one or more embodiments in the hole transport region HTR and may thus show high efficiency and long-life characteristics in a blue emission region. However, embodiments of the present disclosure are not limited thereto.

In the light-emitting element ED of one or more embodiments, the light-emitting layer EML may include at least one of anthracene derivatives, pyrene derivatives, fluoranthene derivatives, chrysene derivatives, dihydrobenzanthracene derivatives, or triphenylene derivatives. For example, in one or more embodiments, the light-emitting layer EML may include one or more anthracene derivatives or one or more pyrene derivatives.

In the light-emitting elements ED of embodiments, shown in FIG. 3 to FIG. 6, the light-emitting layer EML may include a host and a dopant, and the light-emitting layer EML may include a compound represented by Formula E-1. The compound represented by Formula E-1 may be used as a fluorescence host material.

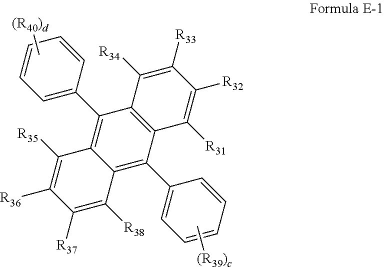

In Formula E-1, R₃₁ to R₄₀ may each independently be hydrogen, deuterium, a halogen, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, and/or may be independently combined with an adjacent group to form a ring. In one or more embodiments, one or more selected from among R₃₁ to R₄₀ may be each independently combined with an adjacent group to form a saturated hydrocarbon ring, an unsaturated hydrocarbon ring, a saturated heterocycle, or an unsaturated heterocycle.

In Formula E-1, “c” and “d” may each independently be an integer of 0 to 5.

The compound represented by Formula E-1 may be any one selected from among Compound E1 to Compound E19.

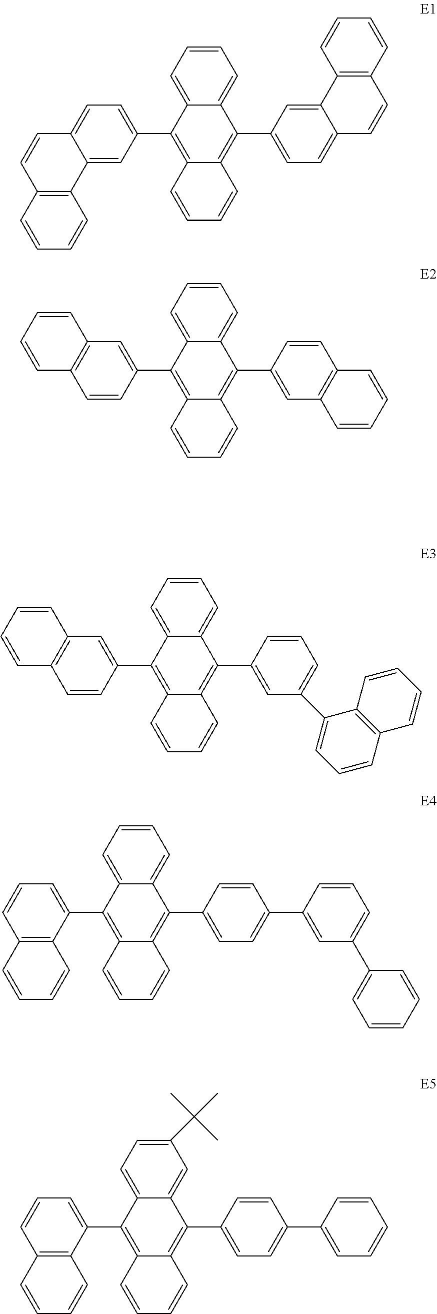

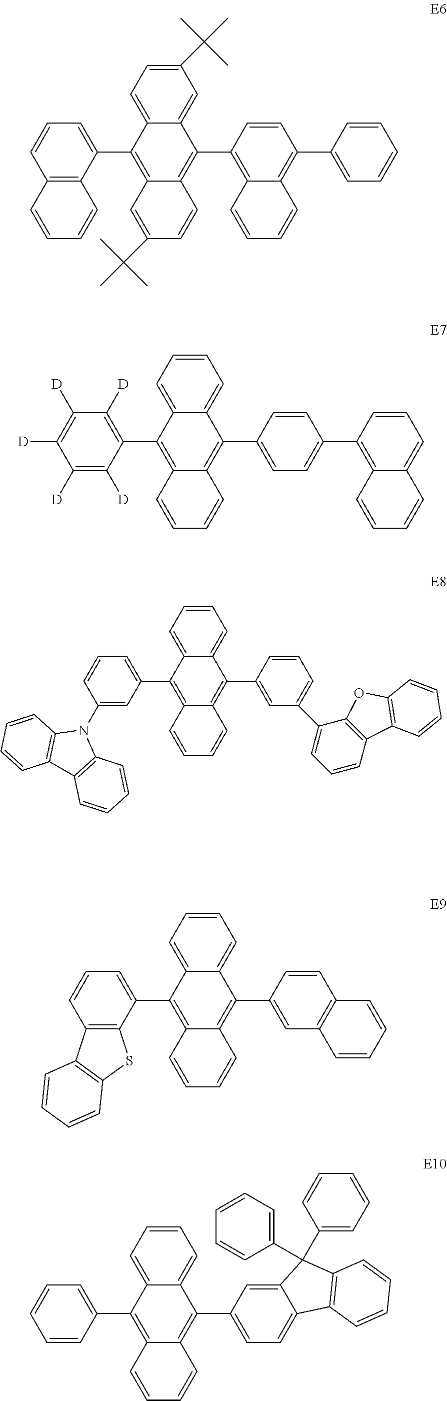

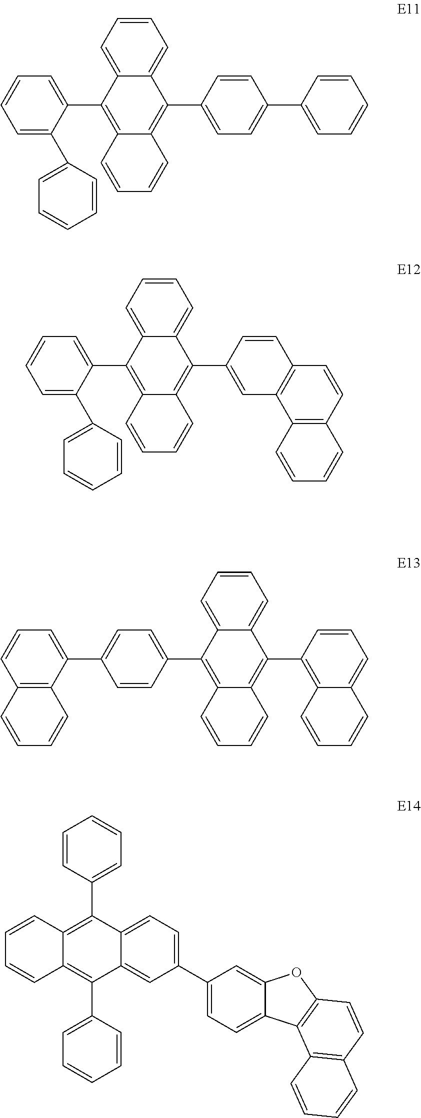

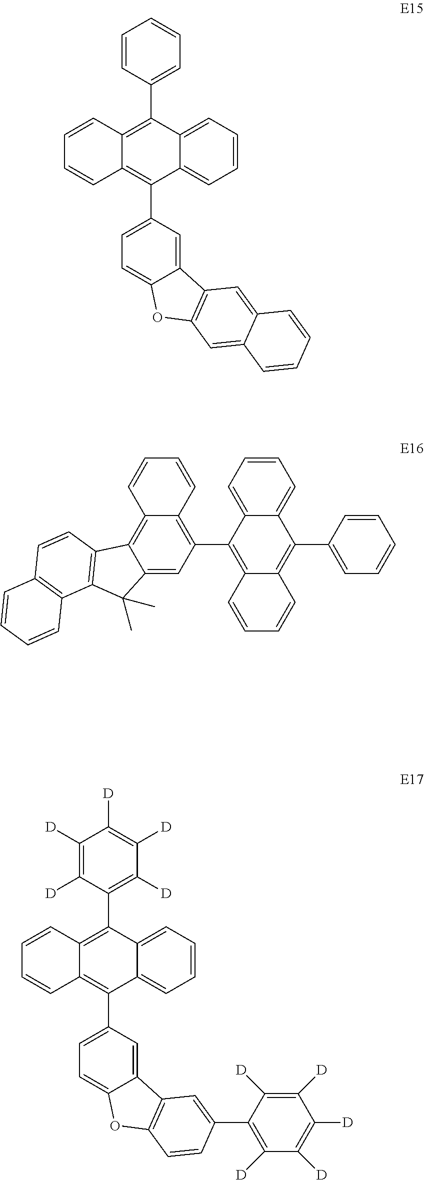

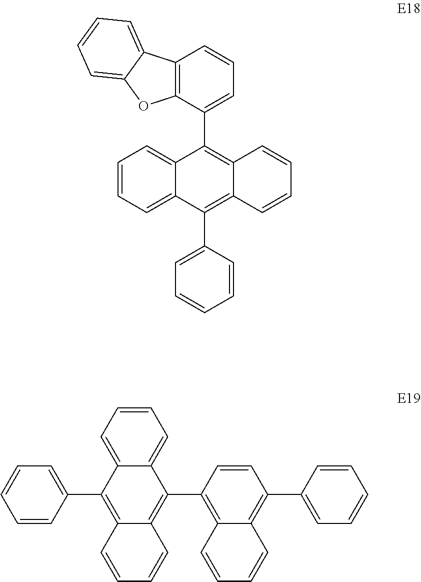

In one or more embodiments, the light-emitting layer EML may include a compound represented by Formula E-2a or Formula E-2b. The compound represented by Formula E-2a or Formula E-2b may be used as a phosphorescence host material.

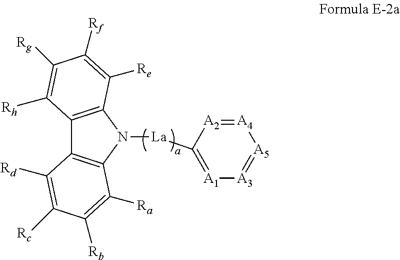

In Formula E-2a, “a” may be an integer of 0 to 10, Lₐ may be a direct linkage, a substituted or unsubstituted arylene group of 6 to 30 ring-forming carbon atoms, or a substituted or unsubstituted heteroarylene group of 2 to 30 ring-forming carbon atoms. In one or more embodiments, if “a” is an integer of 2 or greater, multiple Lₐ's may each independently be a substituted or unsubstituted arylene group of 6 to 30 ring-forming carbon atoms or a substituted or unsubstituted heteroarylene group of 2 to 30 ring-forming carbon atoms.

In addition, in Formula E-2a, A₁ to A₅ may each independently be N or CRi. Rₐ to Rᵢ may each independently be hydrogen, deuterium, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be independently combined with an adjacent group to form a ring. In one or more embodiments, one or more selected from among Rₐ to Rᵢ may be independently combined with an adjacent group to form a hydrocarbon ring or a heterocycle including N, O, S, and/or the like, as a ring-forming atom.

In one or more embodiments, in Formula E-2a, two or three selected from among A₁ to A₅ may be N, and the remainder may be CRᵢ.

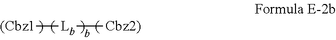

In Formula E-2b, Cbz1 and Cbz2 may each independently be an unsubstituted carbazole group or a carbazole group substituted with an aryl group of 6 to 30 ring-forming carbon atoms. L_{b} may be a direct linkage, a substituted or unsubstituted arylene group of 6 to 30 ring-forming carbon atoms, or a substituted or unsubstituted heteroarylene group of 2 to 30 ring-forming carbon atoms. “b” may be an integer of 0 to 10, and if (e.g., when) “b” is an integer of 2 or greater, multiple L_{b}'s may each independently be a substituted or unsubstituted arylene group of 6 to 30 ring-forming carbon atoms or a substituted or unsubstituted heteroarylene group of 2 to 30 ring-forming carbon atoms.

The compound represented by Formula E-2a or Formula E-2b may be any one selected from among compounds in Compound Group E-2. However, the compounds listed in Compound Group E-2 are mere examples, and the compound represented by Formula E-2a or Formula E-2b is not limited to the compounds represented in Compound Group E-2.

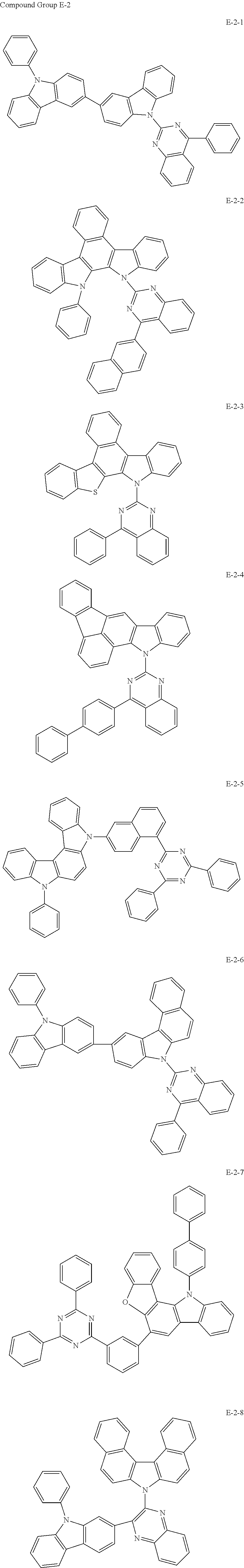

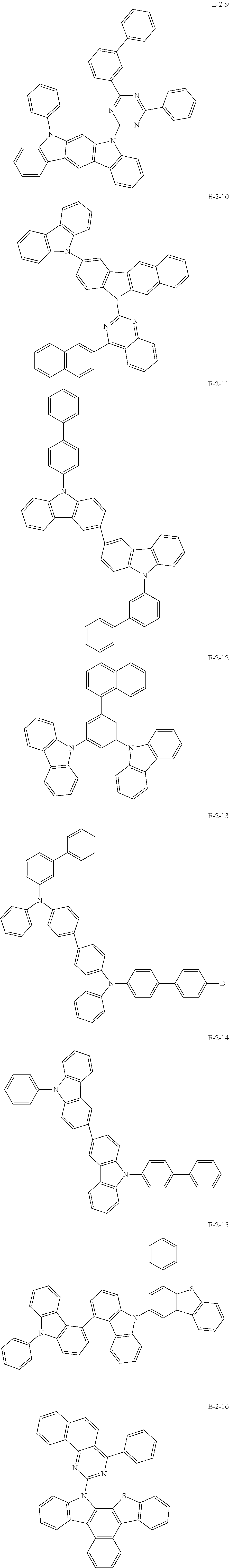

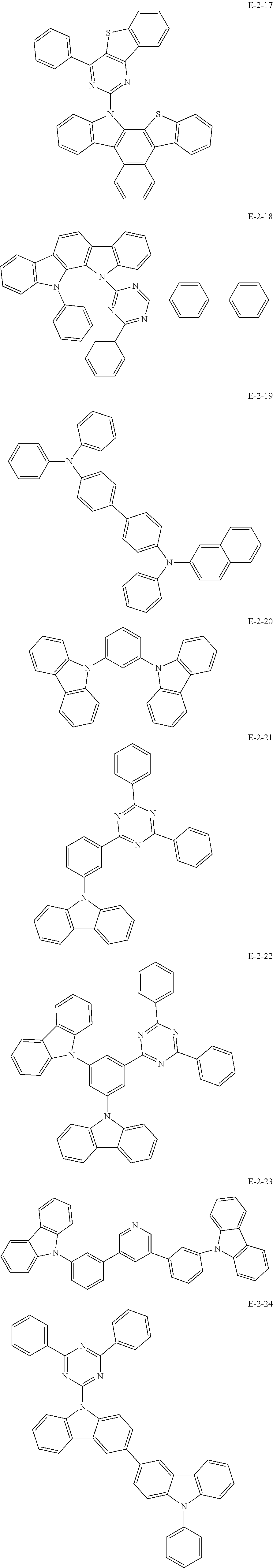

In one or more embodiments, the light-emitting layer EML may further include a material well-suitable in the art as a host material. For example, the light-emitting layer EML may include as a host material, at least one of bis(4-(9H-carbazol-9-yl)phenyl) diphenylsilane (BCPDS), (4-(1-(4-(diphenylamino)phenyl) cyclohexyl)phenyl) diphenyl-phosphine oxide (POPCPA), bis[2-(diphenylphosphino)phenyl] ether oxide (DPEPO), 4,4′-bis(carbazol-9-yl)biphenyl (CBP), 1,3-bis(carbazol-9-yl)benzene (mCP), 2,8-bis (diphenylphosphoryl)dibenzo[b,d]furan (PPF), 4,4′,4″-tris(carbazol-9-yl)-triphenylamine (TCTA), or 1,3,5-tris (1-phenyl-1H-benzo[d]imidazol-2-yl)benzene (TPBi). However, embodiments of the present disclosure are not limited thereto. For example, tris(8-hydroxyquinolinato)aluminum (Alq₃), 9,10-di(naphthalen-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9, 10-bis(naphthalen-2-yl) anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), and/or the like may be used as the host material.

In one or more embodiments, the light-emitting layer EML may include a compound represented by Formula M-a or Formula M-b. The compound represented by Formula M-a or Formula M-b may be used as a phosphorescence dopant material. In addition, in one or more embodiments, the compound represented by Formula M-a or Formula M-b may be used as an auxiliary dopant material.

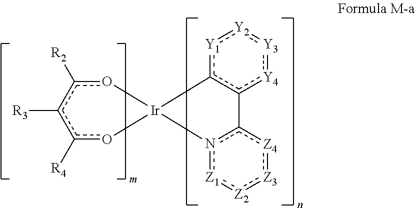

In Formula M-a, Y₁ to Y₄, and Z₁ to Z₄ may each independently be CR₁ or N, and R₁ to R₄ may each independently be hydrogen, deuterium, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be independently combined with an adjacent group to form a ring. In Formula M-a, “m” is 0 or 1, and “n” is 2 or 3. In Formula M-a, if (e.g., when) “m” is 0, “n” is 3, and if (e.g., when) “m” is 1, “n” is 2.

In one or more embodiments, the compound represented by Formula M-a may be used as a phosphorescence dopant.

The compound represented by Formula M-a may be any one selected from among Compounds M-a1 to M-a25. However, Compounds M-a1 to M-a25 are mere examples, and the compound represented by Formula M-a is not limited to the compounds represented by Compounds M-a1 to M-a25.

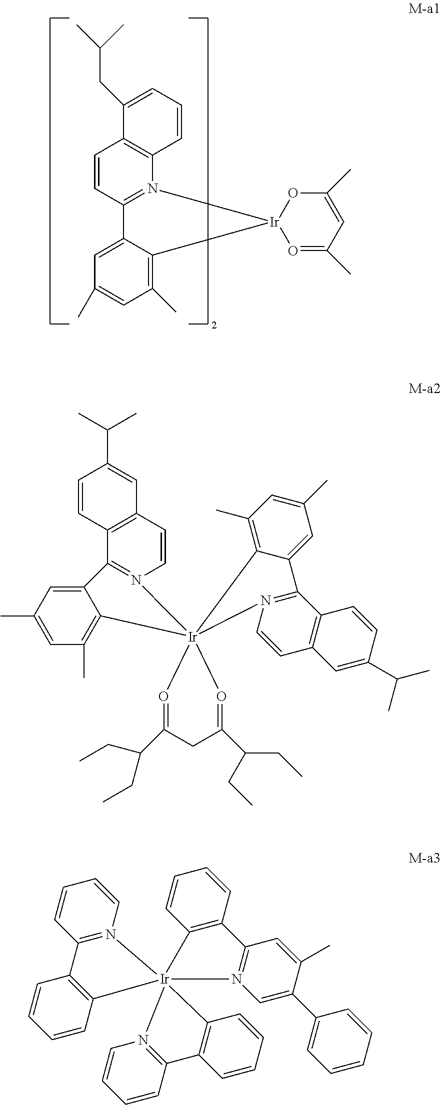

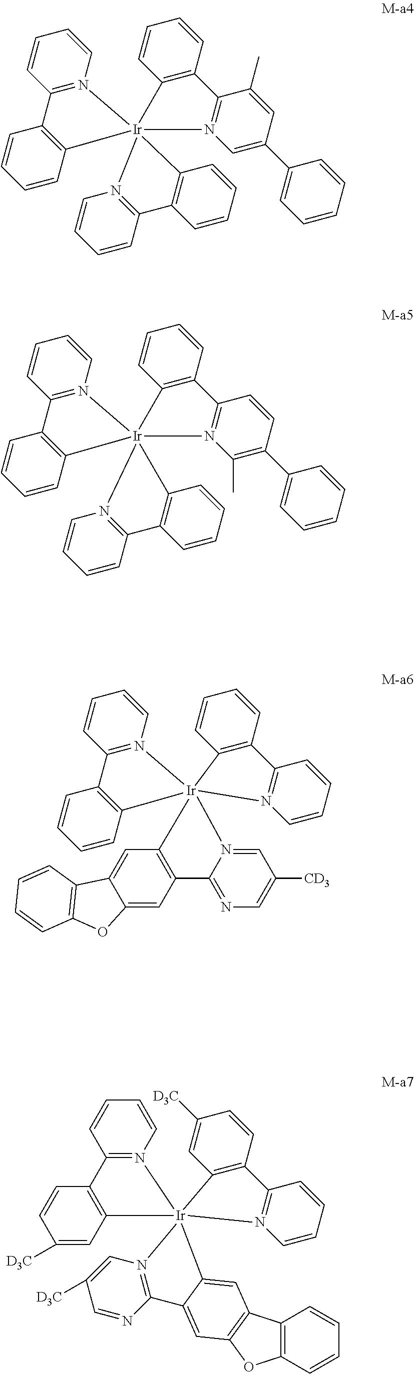

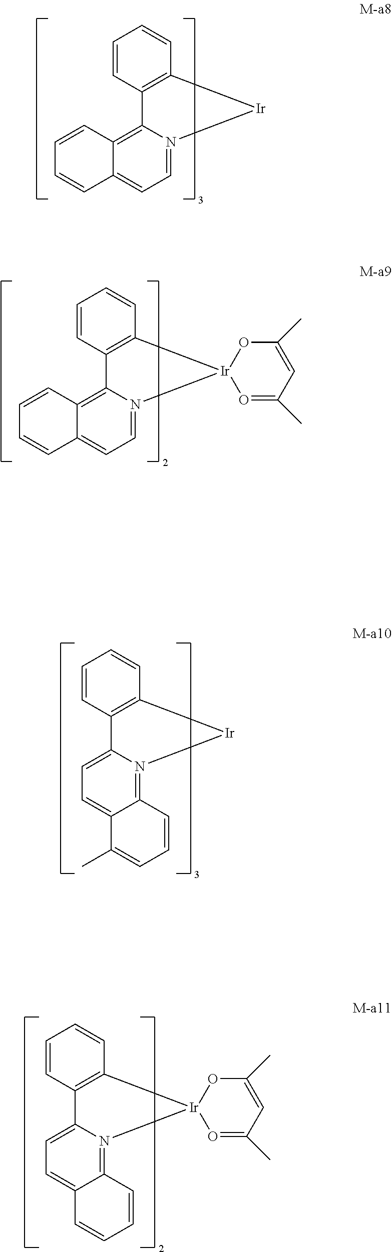

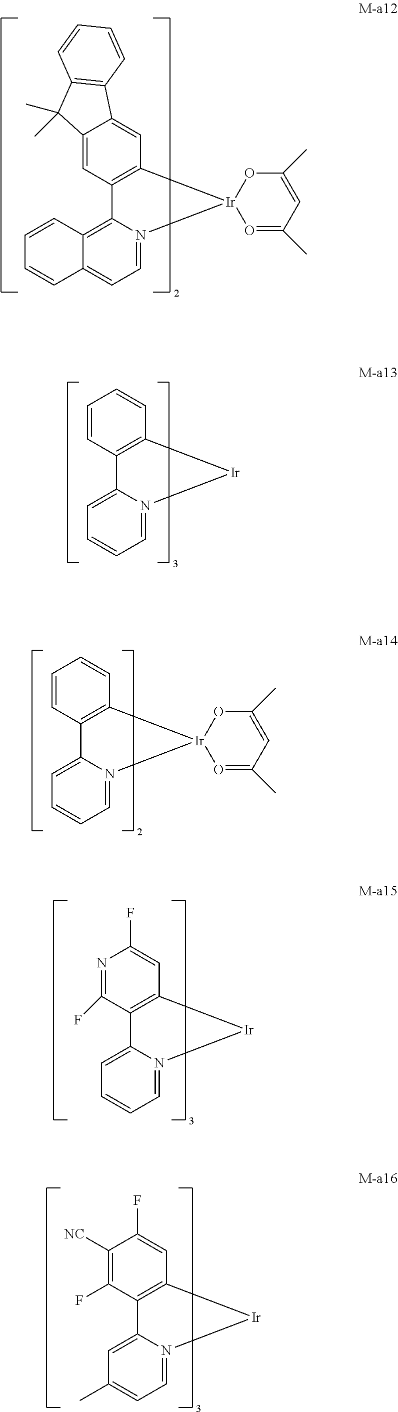

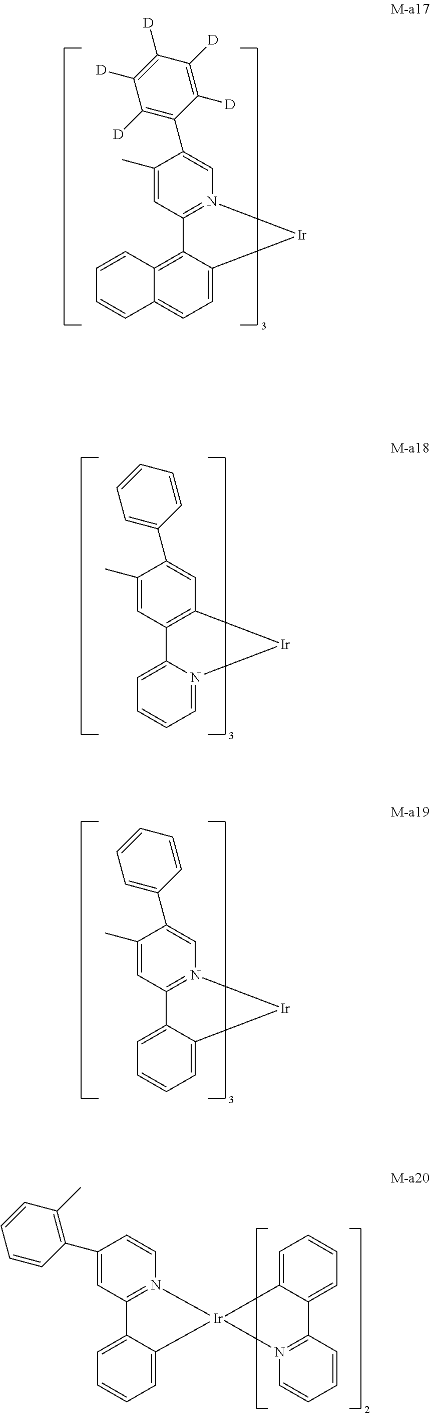

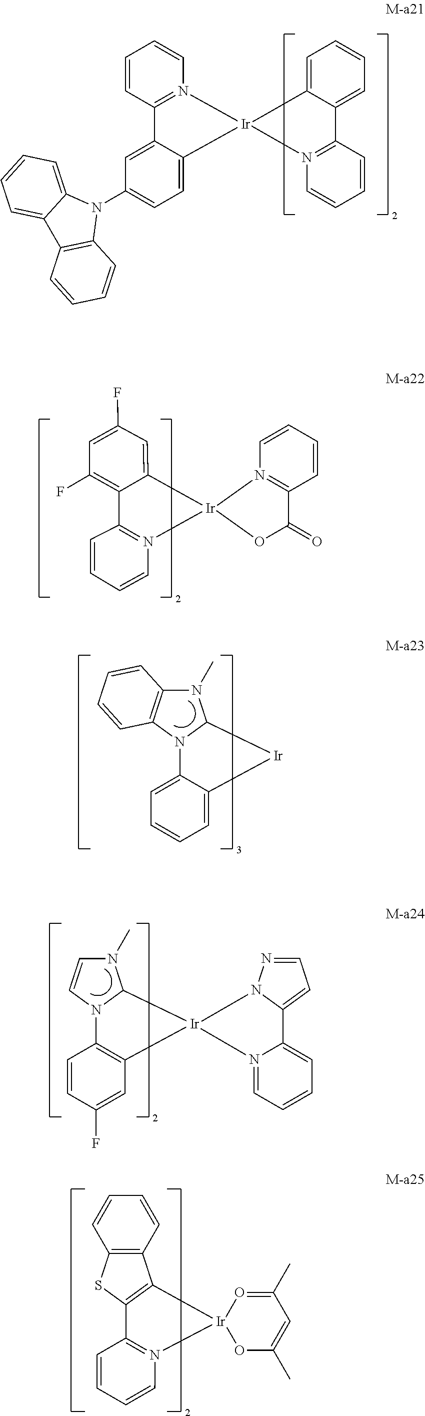

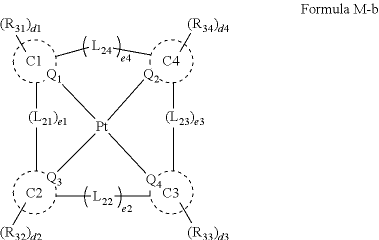

In Formula M-b, Q₁ to Q₄ may each independently be C or N, C1 to C4 may each independently be a substituted or unsubstituted hydrocarbon ring of 5 to 30 ring-forming carbon atoms or a substituted or unsubstituted heterocycle of 2 to 30 ring-forming carbon atoms. L₂₁ to L₂₄ may each independently be a direct linkage,

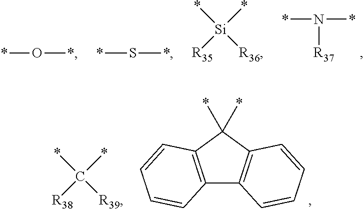

a substituted or unsubstituted alkylene group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and e1 to e4 may each independently be 0 or 1. R³¹ to R³⁹ may each independently be hydrogen, deuterium, a halogen, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, and d1 to d4 may each independently be an integer of 0 to 4.

The compound represented by Formula M-b may be used as a blue phosphorescence dopant or a green phosphorescence dopant. In one or more embodiments, the compound represented by Formula M-b may be an auxiliary dopant and may be further included in the light-emitting layer EML.

The compound represented by Formula M-b may be any one selected from among Compound M-b-1 to Compound M-b-11. However, Compound M-b-1 to Compound M-b-11 are mere examples, and the compound represented by Formula M-b is not limited to Compound M-b-1 to Compound M-b-11.

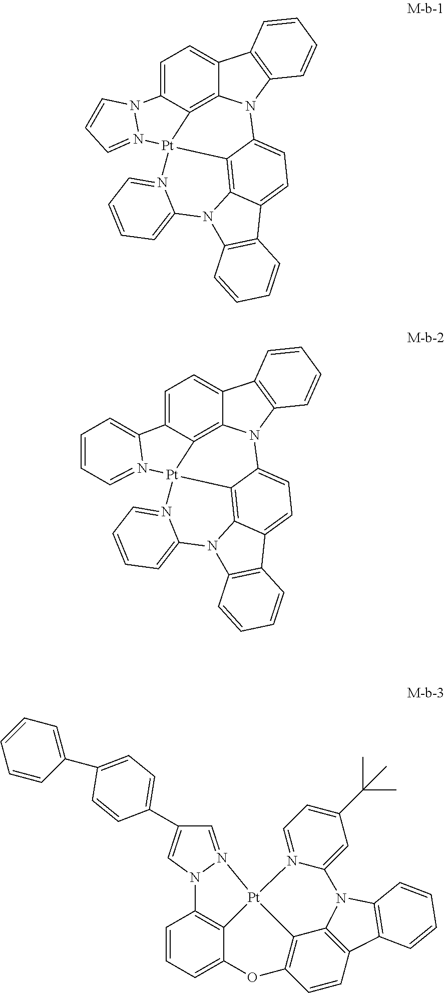

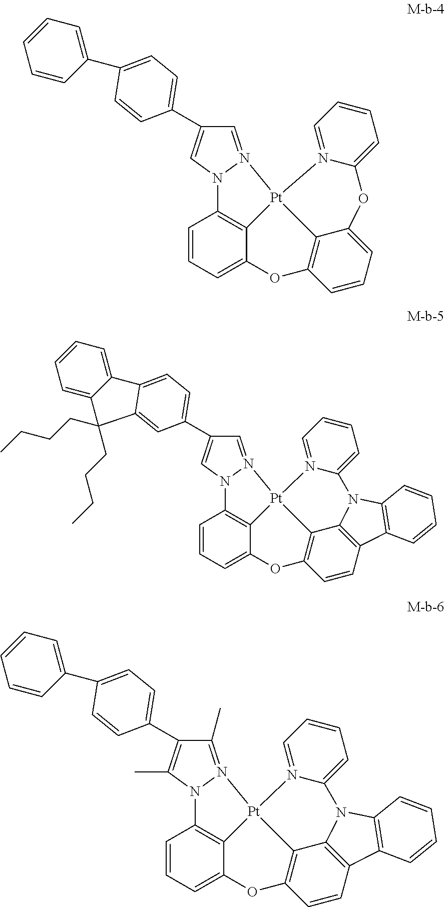

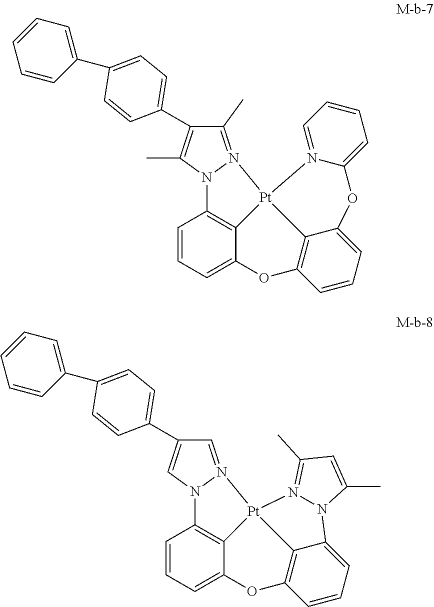

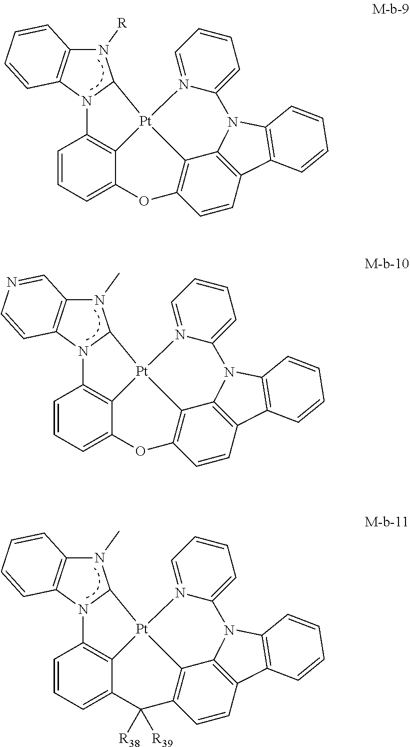

In the compounds above, R, R₃₈, and R₃₉ may each independently be hydrogen, deuterium, a halogen, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In one or more embodiments, the light-emitting layer EML may contain a first compound represented by any one selected from among Formulas F-a to F-c, a second compound represented by Formula HT-1, a third compound represented by Formula ET-1, and a fourth compound represented by Formula D-1.

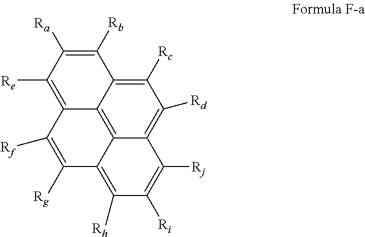

In Formula F-a, two selected from among Rₐ to Rⱼ may each independently be substituted with *—NAr₁Ar₂. The remainder not substituted with *—NAr₁Ar₂ among Rₐ to Rⱼ may each independently be hydrogen, deuterium, a halogen, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In *—NAr₁Ar₂, Ar₁ and Ar₂ may each independently be a substituted or unsubstituted aryl group of 6 to 30 ring-forming carbon atoms or a substituted or unsubstituted heteroaryl group of 2 to 30 ring-forming carbon atoms. For example, in one or more embodiments, at least one selected from among Ar₁ and Ar₂ may be a heteroaryl group including O or S as a ring-forming atom.

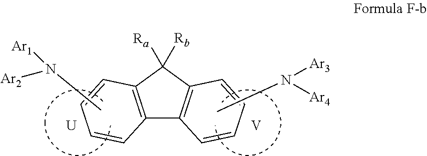

In Formula F-b, Ra and Rb may each independently be hydrogen, deuterium, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be independently combined with an adjacent group to form a ring.

In Formula F-b, Ar₁ to Ar₄ may each independently be a substituted or unsubstituted aryl group of 6 to 30 ring-forming carbon atoms or a substituted or unsubstituted heteroaryl group of 2 to 30 ring-forming carbon atoms.

In Formula F-b, U and V may each independently be a substituted or unsubstituted hydrocarbon ring of 5 to 30 ring-forming carbon atoms or a substituted or unsubstituted heterocycle of 2 to 30 ring-forming carbon atoms.

In Formula F-b, the number of rings represented by U and V may each independently be 0 or 1. For example, in Formula F-b, if (e.g., when) the number of U or V is 1, one ring forms a part of a fused ring at the designated part by U or V, and if (e.g., when) the number of U or V is 0, a ring is not present at the designated part by U or V. For example, if (e.g., when) the number of U is 0, and the number of V is 1, or if (e.g., when) the number of U is 1, and the number of V is 0, a fused ring having the fluorene core of Formula F-b may be a ring compound with four rings. In one or more embodiments, if (e.g., when) the number of both (e.g., simultaneously) U and V is 0, the fused ring of Formula F-b may be a ring compound with three rings. In one or more embodiments, if (e.g., when) the number of both (e.g., simultaneously) U and V is 1, a fused ring having the fluorene core of Formula F-b may be a ring compound with five rings.

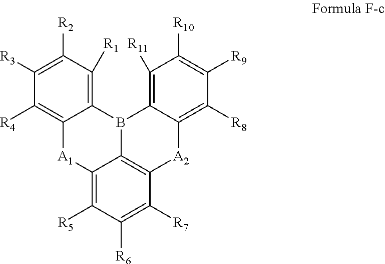

In Formula F-c, A₁ and A₂ may each independently be O, S, Se, or NRₘ, and Rₘ may be hydrogen, deuterium, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ may each independently be hydrogen, deuterium, a halogen, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or independently combined with an adjacent group to form a ring.

In Formula F-c, A₁ and A₂ may each independently be combined with the substituents of an adjacent ring to form a fused ring. For example, if (e.g., when) A₁ and A₂ may each independently be NRₘ, A₁ may be combined with R₄ or R₅ to form a ring. In addition, A₂ may be combined with R₇ or R₈ to form a ring.

In one or more embodiments, the second compound may be used as a hole transporting host material of the light-emitting layer EML.

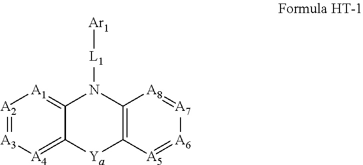

In Formula HT-1, A₁ to A₈ may each independently be N or CR₅₁. For example, in one or more embodiments, all A₁ to A₈ may be CR₅₁. In one or more embodiments, any one selected from among A₁ to A₈ may be N, and the remainder may be CR₅₁.

In Formula HT-1, L₁ may be a direct linkage, a substituted or unsubstituted arylene group of 6 to 30 ring-forming carbon atoms, or a substituted or unsubstituted heteroarylene group of 2 to 30 ring-forming carbon atoms. For example, in one or more embodiments, L₁ may be a direct linkage, a substituted or unsubstituted phenylene group, a substituted or unsubstituted divalent biphenyl group, a substituted or unsubstituted divalent carbazole group, and/or the like, but embodiments of the present disclosure are not limited thereto.

In Formula HT-1, Yₐ may be a direct linkage, CR₅₂R₅₃, or SiR₅₄R₅₅. For example, it may refer to that two six-membered rings (e.g., two benzene rings) connected with the nitrogen atom of Formula HT-1 may be connected via a direct linkage,

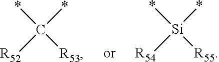

In Formula HT-1, if (e.g., when) Yₐ is a direct linkage, the compound represented by Formula HT-1 may include a carbazole moiety.

In Formula HT-1, Ar₁ may be a substituted or unsubstituted aryl group of 6 to 30 ring-forming carbon atoms or a substituted or unsubstituted heteroaryl group of 2 to 30 ring-forming carbon atoms. For example, in one or more embodiments, Ar₁ may be a substituted or unsubstituted carbazole group, a substituted or unsubstituted dibenzofuran group, a substituted or unsubstituted dibenzothiophene group, a substituted or unsubstituted biphenyl group, or the like, but embodiments of the present disclosure are not limited thereto.

In Formula HT-1, R₅₁ to R₅₅ may each independently be hydrogen, deuterium, a halogen, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbon atoms, a substituted or unsubstituted alkenyl group of 2 to 20 carbon atoms, a substituted or unsubstituted aryl group of 6 to 60 ring-forming carbon atoms, or a substituted or unsubstituted heteroaryl group of 2 to 60 ring-forming carbon atoms. In one or more embodiments, one or more selected from among R₅₁ to R₅₅ may be each independently combined with an adjacent group to form a ring. For example, in one or more embodiments, R₅₁ to R₅₅ may each independently be hydrogen or deuterium. In one or more embodiments, R₅₁ to R₅₅ may each independently be an unsubstituted methyl group or an unsubstituted phenyl group.

In one or more embodiments, the second compound represented by Formula HT-1 may be any one selected from among compounds represented in Compound Group 2. The light-emitting layer EML may include at least one selected from among the compounds represented in Compound Group 2 as a hole transporting host material.

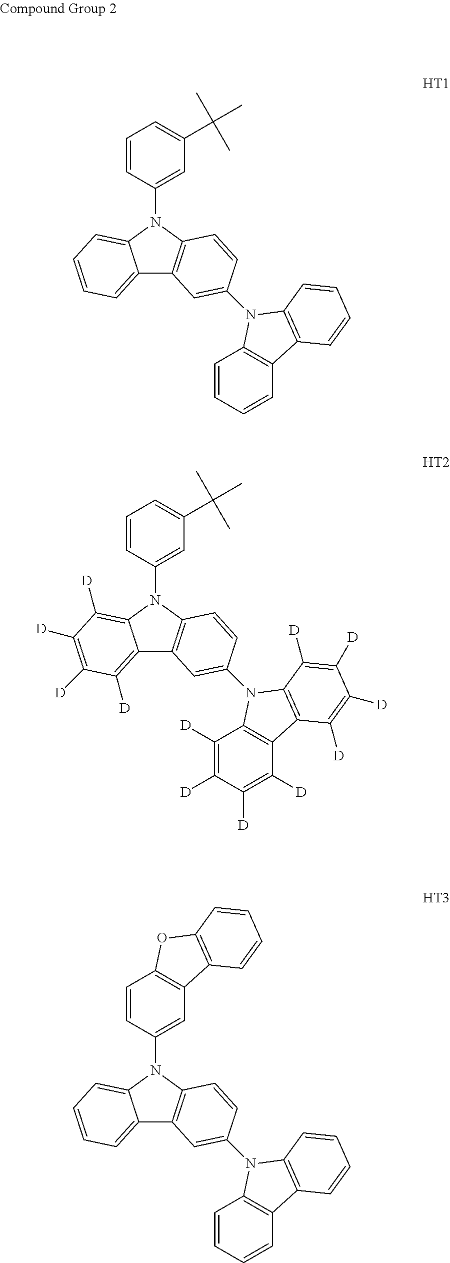 [SMILES: CC(C)(C)c1cccc(-n2c3ccccc3c3cc(-n4c5ccccc5c5ccccc54)ccc32)c1 ; [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12]

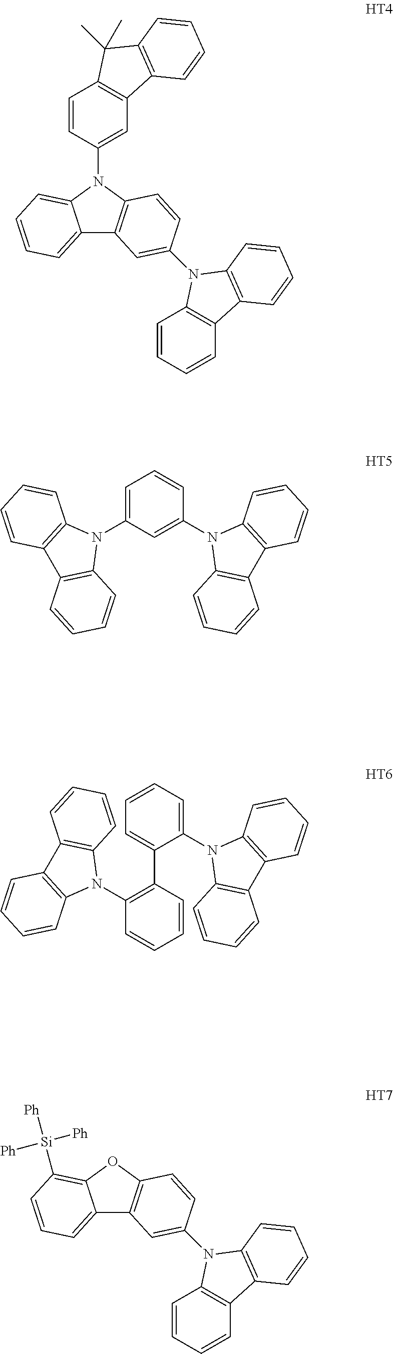 [SMILES: CC1(C)c2ccccc2-c2cc(-n3c4ccccc4c4cc(-n5c6ccccc6c6ccccc65)ccc43)ccc21 ; c1cc(-n2c3ccccc3c3ccccc32)cc(-n2c3ccccc3c3ccccc32)c1 ; c1ccc(-n2c3ccccc3c3ccccc32)c(-c2ccccc2-n2c3ccccc3c3ccccc32)c1 ; c1ccc([Si](c2ccccc2)(c2ccccc2)c2cccc3c2oc2ccc(-n4c5ccccc5c5ccccc54)cc23)cc1]

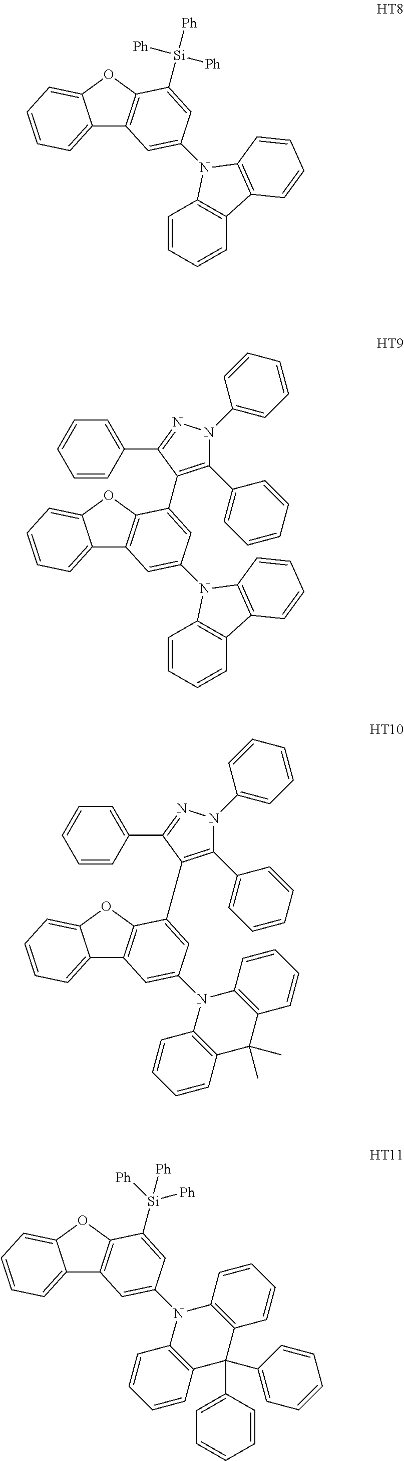 [SMILES: c1ccc([Si](c2ccccc2)(c2ccccc2)c2cc(-n3c4ccccc4c4ccccc43)cc3c2oc2ccccc23)cc1 ; c1ccc(-c2nn(-c3ccccc3)c(-c3ccccc3)c2-c2cc(-n3c4ccccc4c4ccccc43)cc3c2oc2ccccc23)cc1 ; CC1(C)c2ccccc2N(c2cc(-c3c(-c4ccccc4)nn(-c4ccccc4)c3-c3ccccc3)c3oc4ccccc4c3c2)c2ccccc21 ; c1ccc(C2(c3ccccc3)c3ccccc3N(c3cc([Si](c4ccccc4)(c4ccccc4)c4ccccc4)c4oc5ccccc5c4c3)c3ccccc32)cc1]

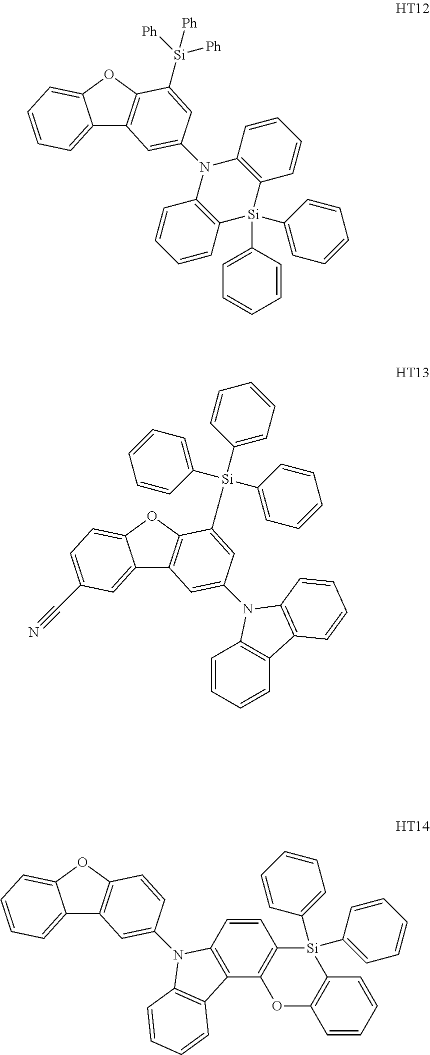 [SMILES: c1ccc([Si]2(c3ccccc3)c3ccccc3N(c3cc([Si](c4ccccc4)(c4ccccc4)c4ccccc4)c4oc5ccccc5c4c3)c3ccccc32)cc1 ; N#Cc1ccc2oc3c([Si](c4ccccc4)(c4ccccc4)c4ccccc4)cc(-n4c5ccccc5c5ccccc54)cc3c2c1 ; c1ccc([Si]2(c3ccccc3)c3ccccc3Oc3c2ccc2c3c3ccccc3n2-c2ccc3oc4ccccc4c3c2)cc1]

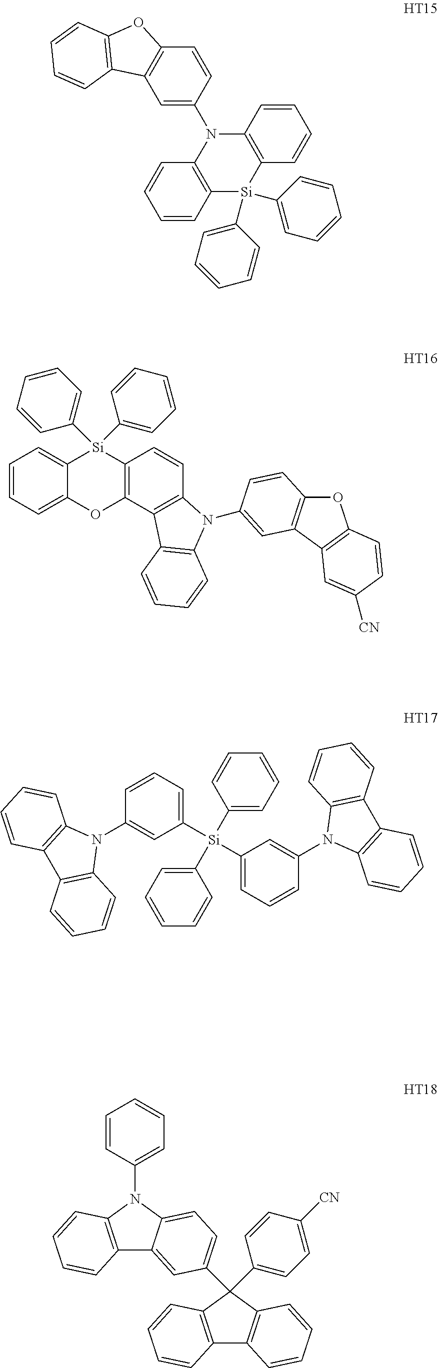 [SMILES: c1ccc([Si]2(c3ccccc3)c3ccccc3N(c3ccc4oc5ccccc5c4c3)c3ccccc32)cc1 ; N#Cc1ccc2oc3ccc(-n4c5ccccc5c5c6c(ccc54)[Si](c4ccccc4)(c4ccccc4)c4ccccc4O6)cc3c2c1 ; c1ccc([Si](c2ccccc2)(c2cccc(-n3c4ccccc4c4ccccc43)c2)c2cccc(-n3c4ccccc4c4ccccc43)c2)cc1 ; N#Cc1ccc(C2(c3ccc4c(c3)c3ccccc3n4-c3ccccc3)c3ccccc3-c3ccccc32)cc1]

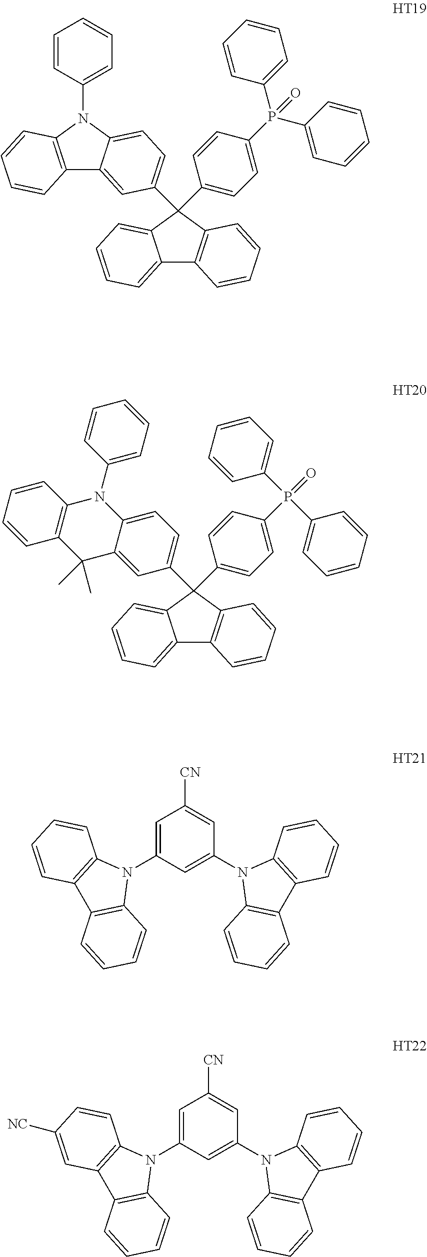 [SMILES: O=P(c1ccccc1)(c1ccccc1)c1ccc(C2(c3ccc4c(c3)c3ccccc3n4-c3ccccc3)c3ccccc3-c3ccccc32)cc1 ; CC1(C)c2ccccc2N(c2ccccc2)c2ccc(C3(c4ccc(P(=O)(c5ccccc5)c5ccccc5)cc4)c4ccccc4-c4ccccc43)cc21 ; N#Cc1cc(-n2c3ccccc3c3ccccc32)cc(-n2c3ccccc3c3ccccc32)c1 ; N#Cc1cc(-n2c3ccccc3c3ccccc32)cc(-n2c3ccccc3c3cc(C#N)ccc32)c1]

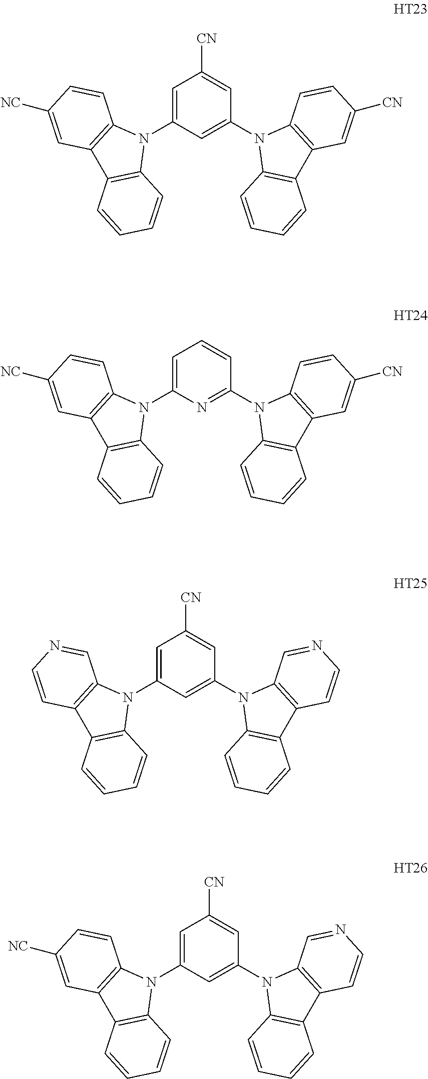 [SMILES: N#Cc1cc(-n2c3ccccc3c3cc(C#N)ccc32)cc(-n2c3ccccc3c3cc(C#N)ccc32)c1 ; N#Cc1ccc2c(c1)c1ccccc1n2-c1cccc(-n2c3ccccc3c3cc(C#N)ccc32)n1 ; N#Cc1cc(-n2c3ccccc3c3ccncc32)cc(-n2c3ccccc3c3ccncc32)c1 ; N#Cc1cc(-n2c3ccccc3c3cc(C#N)ccc32)cc(-n2c3ccccc3c3ccncc32)c1]

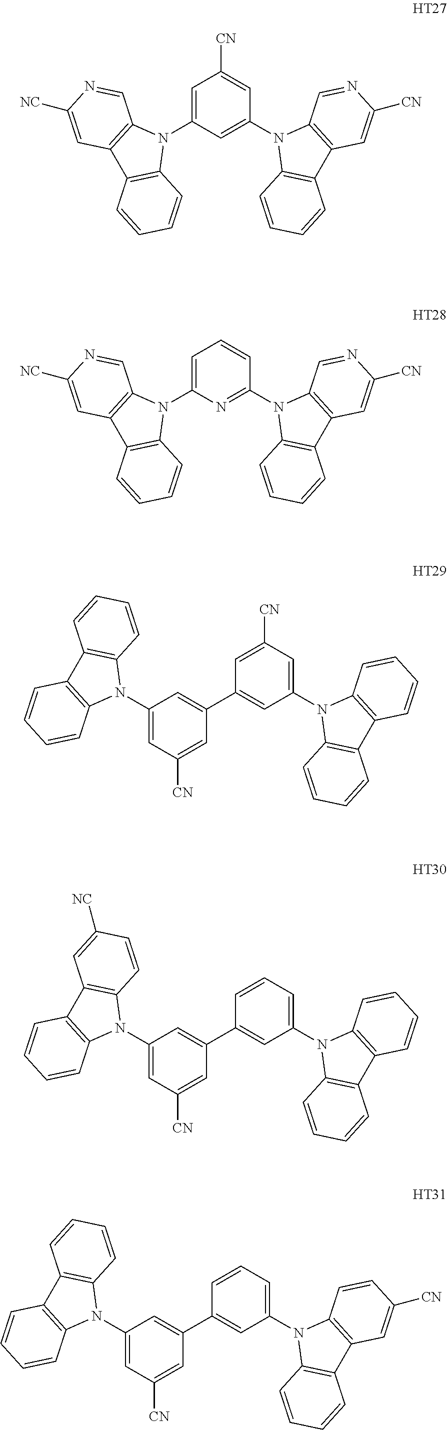 [SMILES: N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)n3)c2cn1 ; N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1]

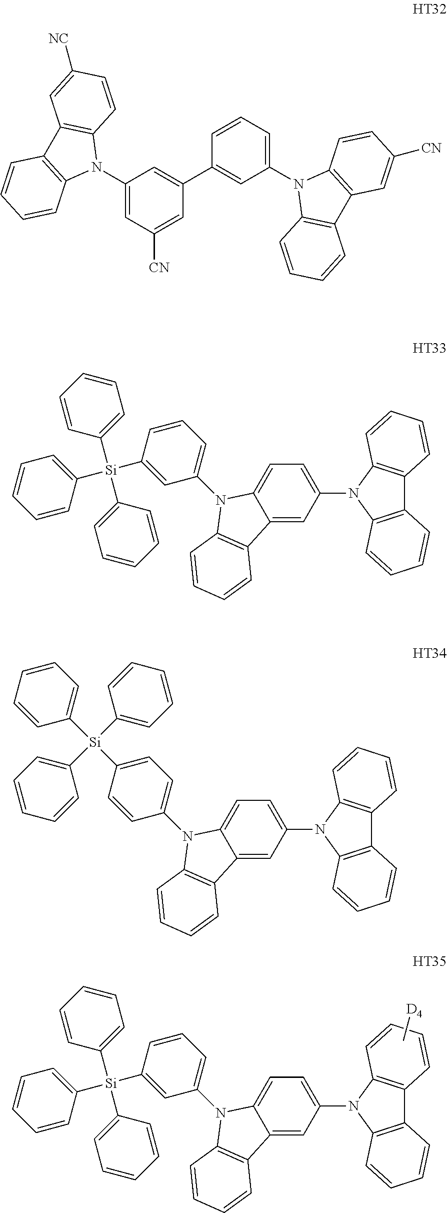

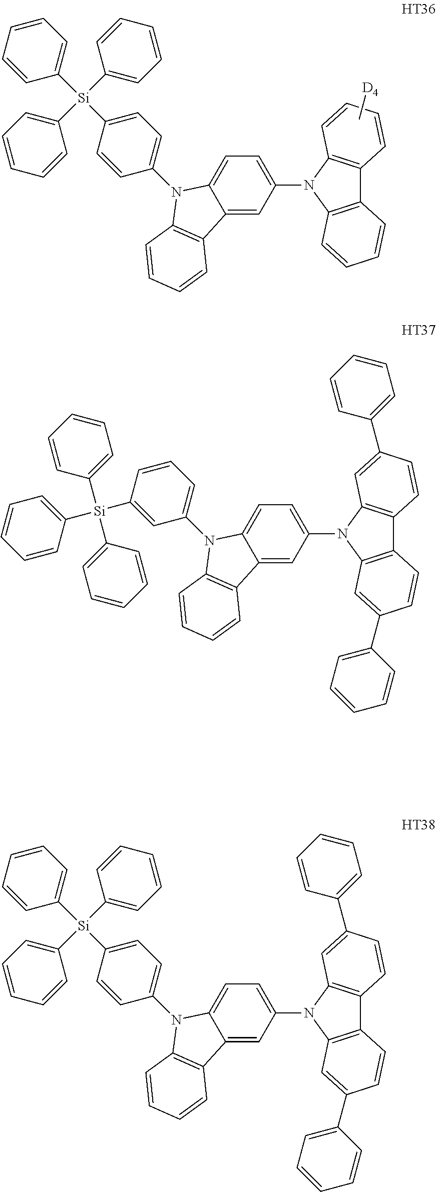

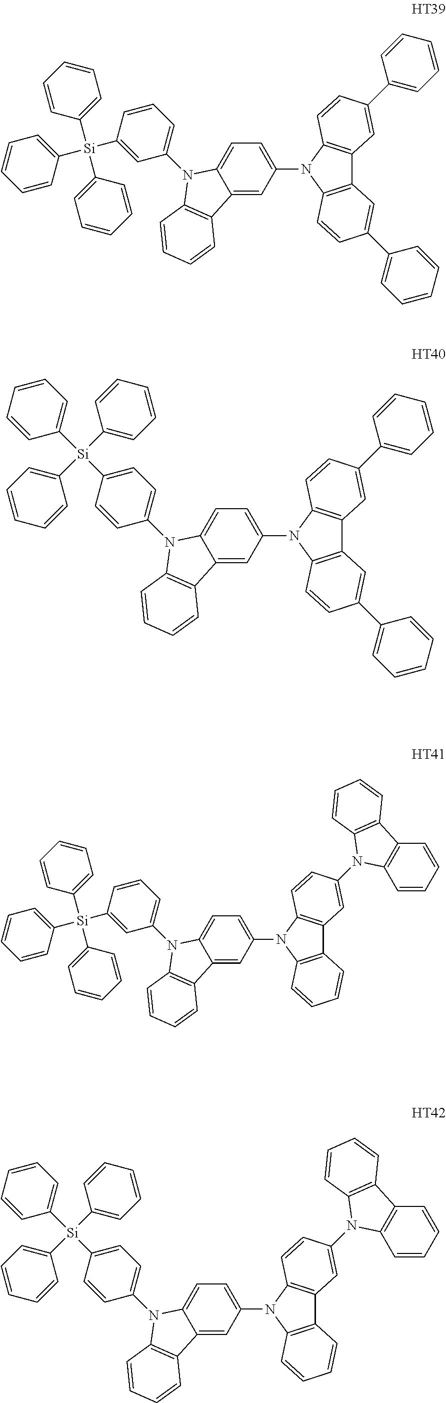 [SMILES: c1ccc(-c2ccc3c(c2)c2cc(-c4ccccc4)ccc2n3-c2ccc3c(c2)c2ccccc2n3-c2cccc([Si](c3ccccc3)(c3ccccc3)c3ccccc3)c2)cc1 ; c1ccc(-c2ccc3c(c2)c2cc(-c4ccccc4)ccc2n3-c2ccc3c(c2)c2ccccc2n3-c2ccc([Si](c3ccccc3)(c3ccccc3)c3ccccc3)cc2)cc1 ; c1ccc([Si](c2ccccc2)(c2ccccc2)c2cccc(-n3c4ccccc4c4cc(-n5c6ccccc6c6cc(-n7c8ccccc8c8ccccc87)ccc65)ccc43)c2)cc1 ; c1ccc([Si](c2ccccc2)(c2ccccc2)c2ccc(-n3c4ccccc4c4cc(-n5c6ccccc6c6cc(-n7c8ccccc8c8ccccc87)ccc65)ccc43)cc2)cc1]

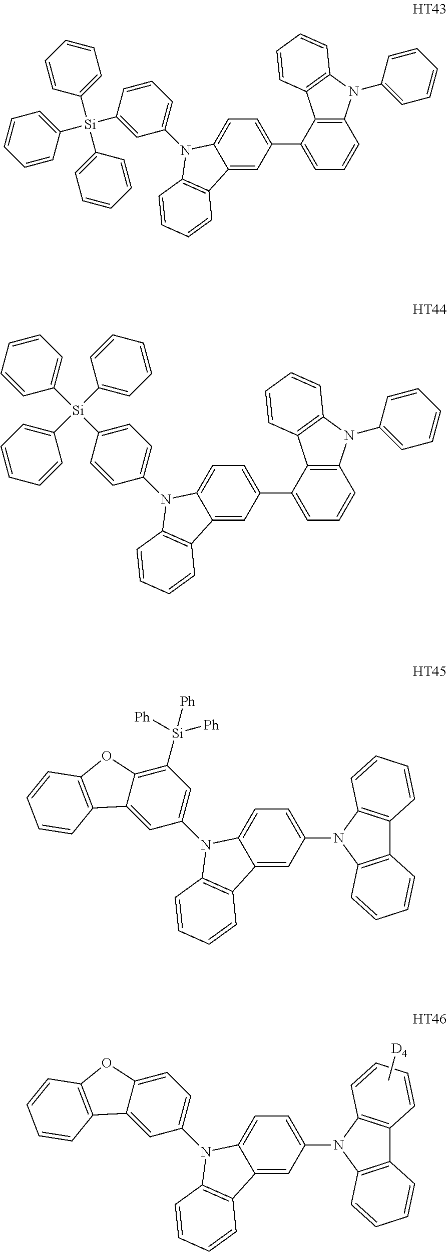

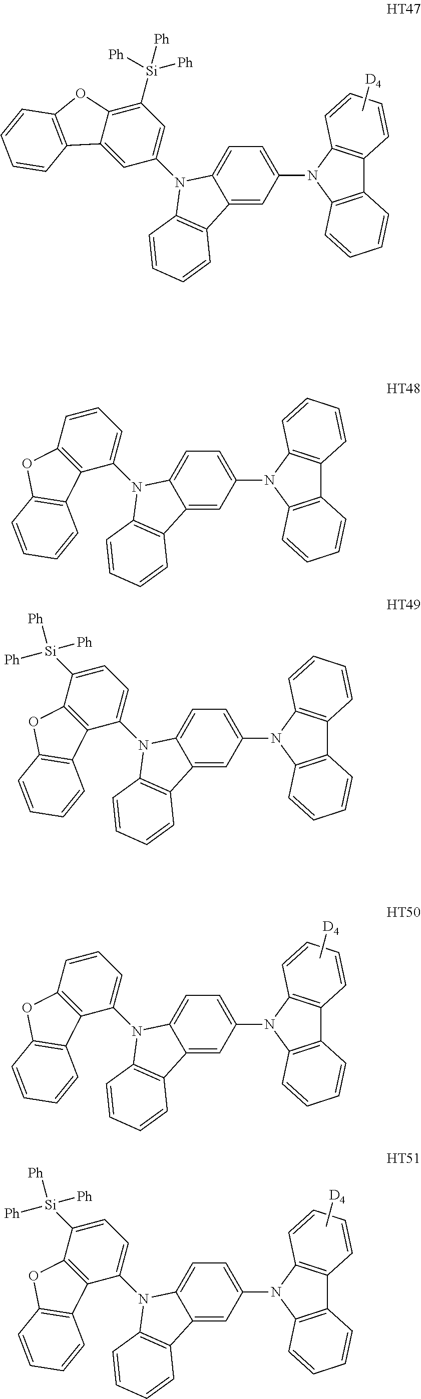

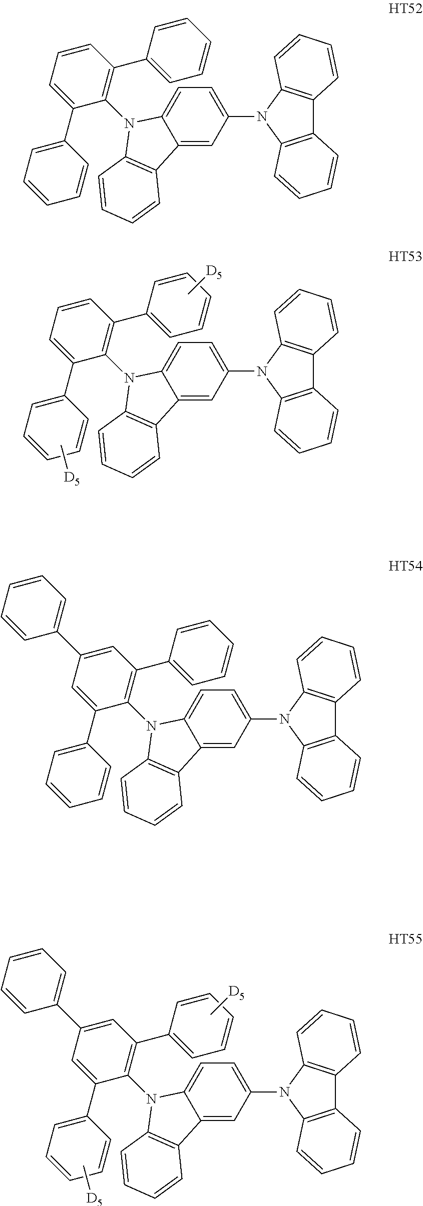

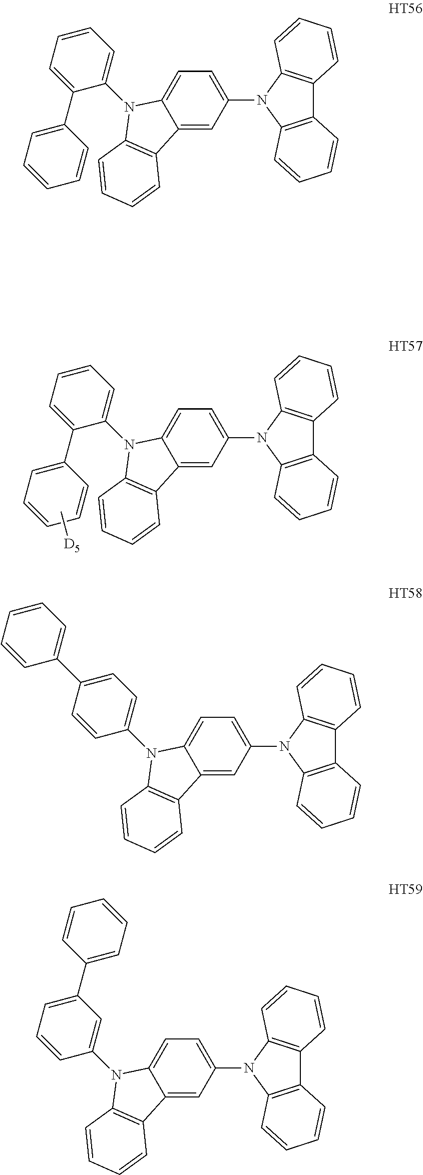

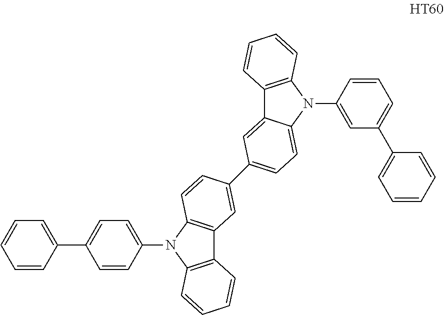 [SMILES: c1ccc(-c2ccc(-n3c4ccccc4c4cc(-c5ccc6c(c5)c5ccccc5n6-c5cccc(-c6ccccc6)c5)ccc43)cc2)cc1]

In the example compounds suggested in Compound Group 2, “D” refers to deuterium, and “Ph” may refer to a substituted or unsubstituted phenyl group. For example, in the example compounds suggested in Compound Group 2, “Ph” may be an unsubstituted phenyl group.

In one or more embodiments, the light-emitting layer EML may contain the third compound represented by Formula ET-1. For example, the third compound may be used as an electron transporting host material of the light-emitting layer EML.

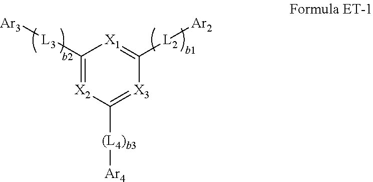

In Formula ET-1, at least one selected from among X₁ to X₃ may be N, and the remainder may be CR₅₆. For example, in one or more embodiments, one selected from among X₁ to X₃ may be N, and the remainder two may each independently be CR₅₆. In these embodiments, the third compound represented by Formula ET-1 may include a pyridine moiety. In one or more embodiments, two selected from among X₁ to X₃ may be N, and the remainder may be CR₅₆. In these embodiments, the third compound represented by Formula ET-1 may include a pyrimidine moiety. In one or more embodiments, X₁ to X₃ may be all N. In these embodiments, the third compound represented by Formula ET-1 may include a triazine moiety.

In Formula ET-1, R₅₆ may be hydrogen, deuterium, a substituted or unsubstituted alkyl group of 1 to 20 carbon atoms, a substituted or unsubstituted aryl group of 6 to 60 ring-forming carbon atoms, or a substituted or unsubstituted heteroaryl group of 2 to 60 ring-forming carbon atoms.

In Formula ET-1, b1 to b3 may each independently be an integer of 0 to 10.

In Formula ET-1, Ar₂ to Ar₄ may each independently be hydrogen, deuterium, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, in one or more embodiments, Ar₂ to Ar₄ may be substituted or unsubstituted phenyl groups or substituted or unsubstituted carbazole groups.

In Formula ET-1, L₂ to L₄ may each independently be a direct linkage, a substituted or unsubstituted arylene group of 6 to 30 ring-forming carbon atoms, or a substituted or unsubstituted heteroarylene group of 2 to 30 ring-forming carbon atoms. In one or more embodiments, if (e.g., when) each of b1 to b3 is an integer of 2 or greater, L₂'s to L₄'s may each independently be a substituted or unsubstituted arylene group of 6 to 30 ring-forming carbon atoms or a substituted or unsubstituted heteroarylene group of 2 to 30 ring-forming carbon atoms.

In one or more embodiments, the third compound may be any one selected from among compounds in Compound Group 3. The light-emitting element ED of one or more embodiments may include any one selected from among the compounds in Compound Group 3.

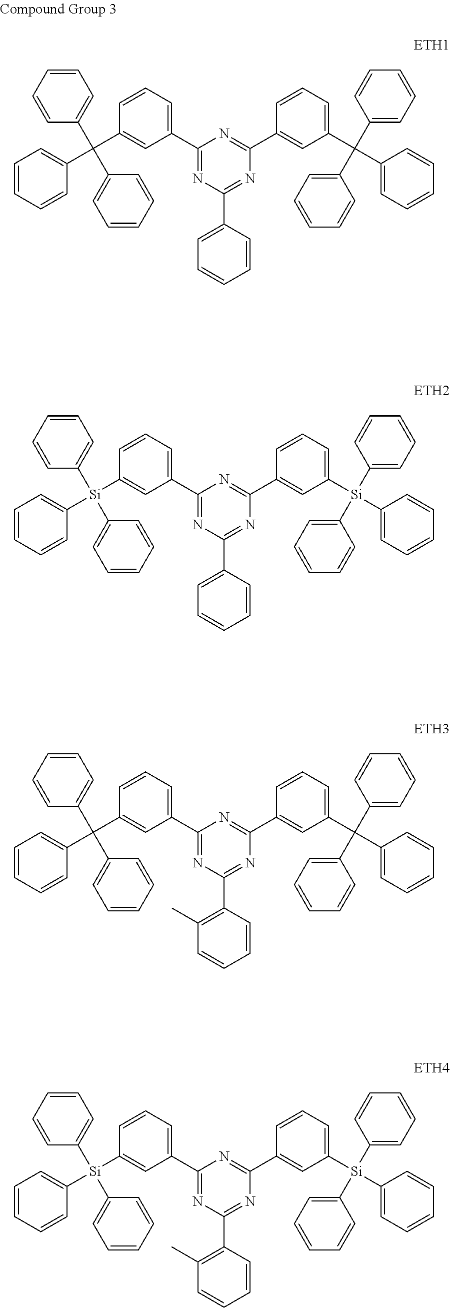 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)nc(-c2cccc([Si](c3ccccc3)(c3ccccc3)c3ccccc3)c2)n1]

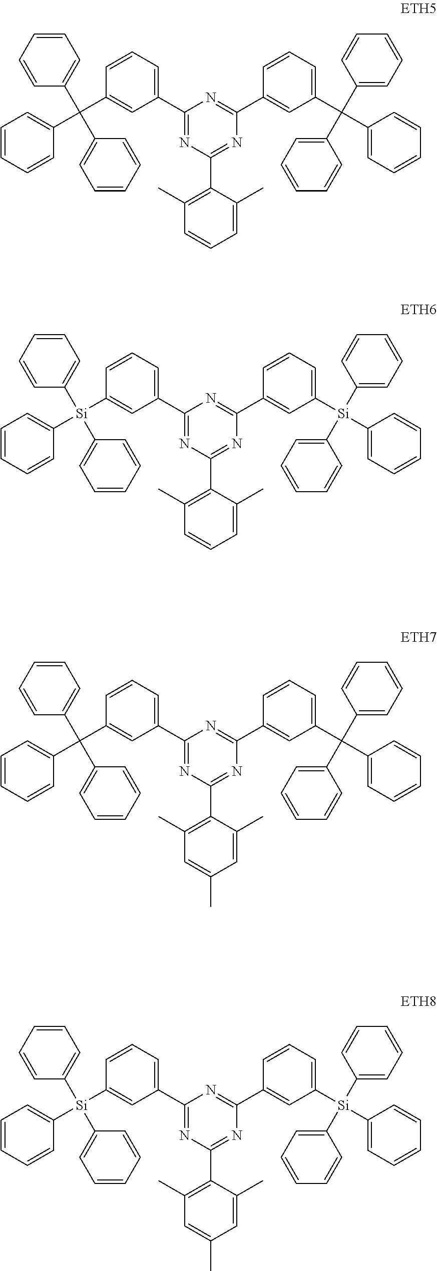 [SMILES: Cc1cccc(C)c1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)nc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)nc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)nc(-c3cccc([Si](c4ccccc4)(c4ccccc4)c4ccccc4)c3)n2)c(C)c1]

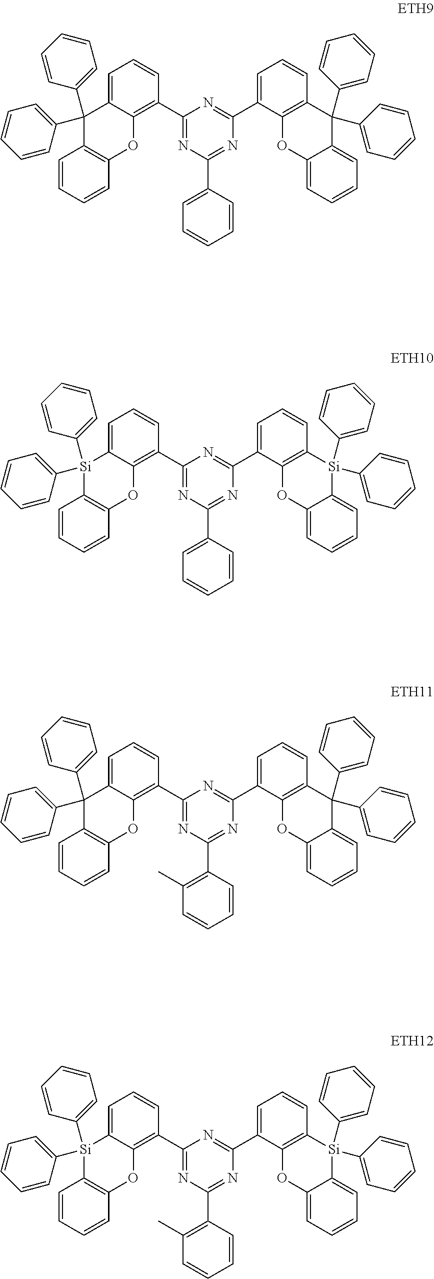 [SMILES: c1ccc(-c2nc(-c3cccc4c3Oc3ccccc3C4(c3ccccc3)c3ccccc3)nc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1]

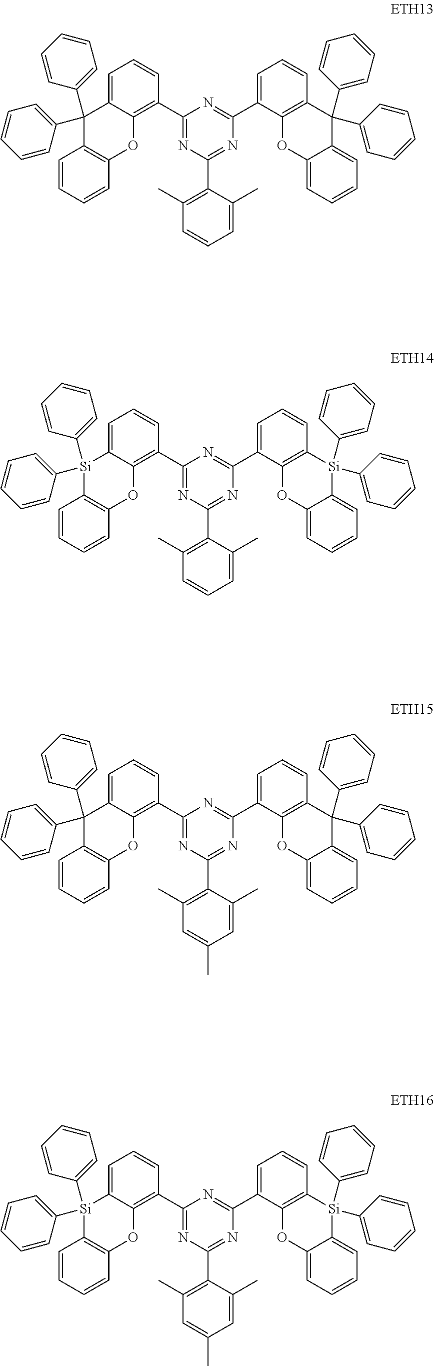 [SMILES: Cc1cccc(C)c1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)nc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)c(C)c1]

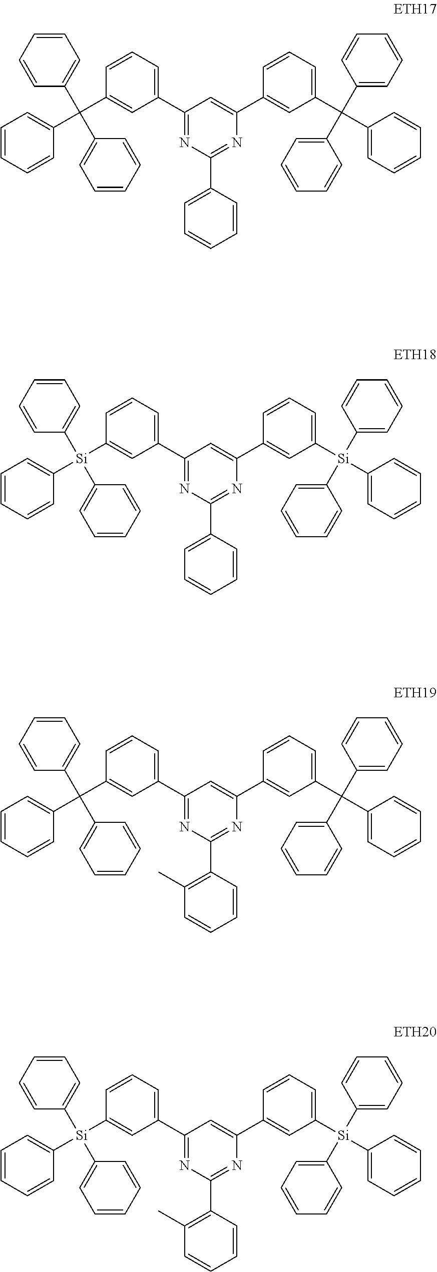 [SMILES: c1ccc(-c2nc(-c3cccc(C(c4ccccc4)(c4ccccc4)c4ccccc4)c3)cc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)cc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)cc(-c2cccc([Si](c3ccccc3)(c3ccccc3)c3ccccc3)c2)n1]

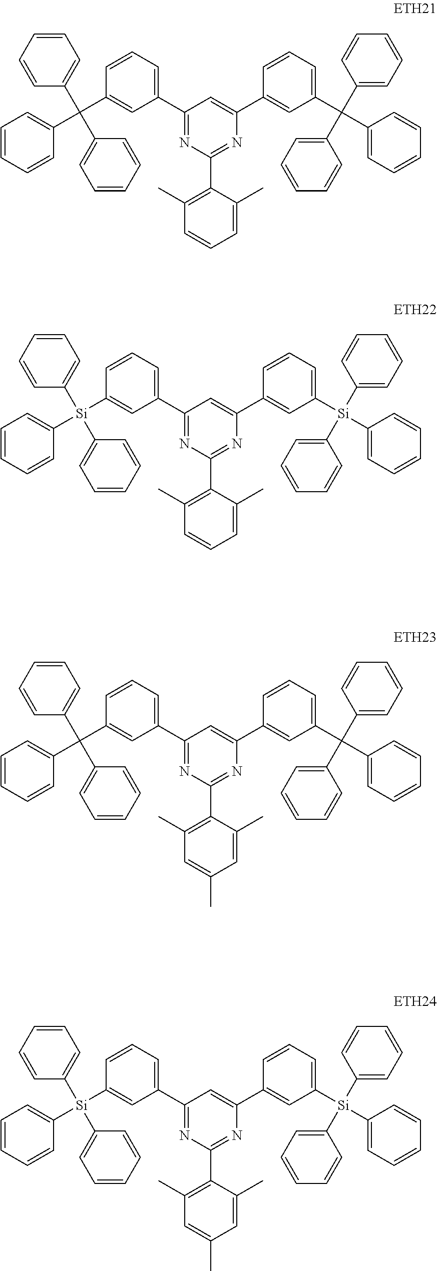 [SMILES: Cc1cccc(C)c1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)cc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)cc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)cc(-c3cccc([Si](c4ccccc4)(c4ccccc4)c4ccccc4)c3)n2)c(C)c1]

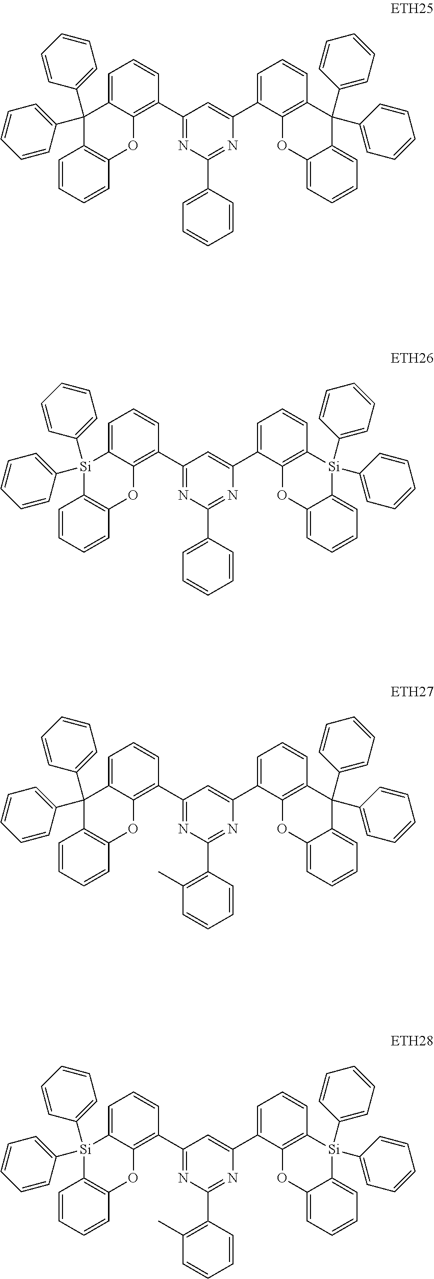 [SMILES: c1ccc(-c2nc(-c3cccc4c3Oc3ccccc3C4(c3ccccc3)c3ccccc3)cc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1]

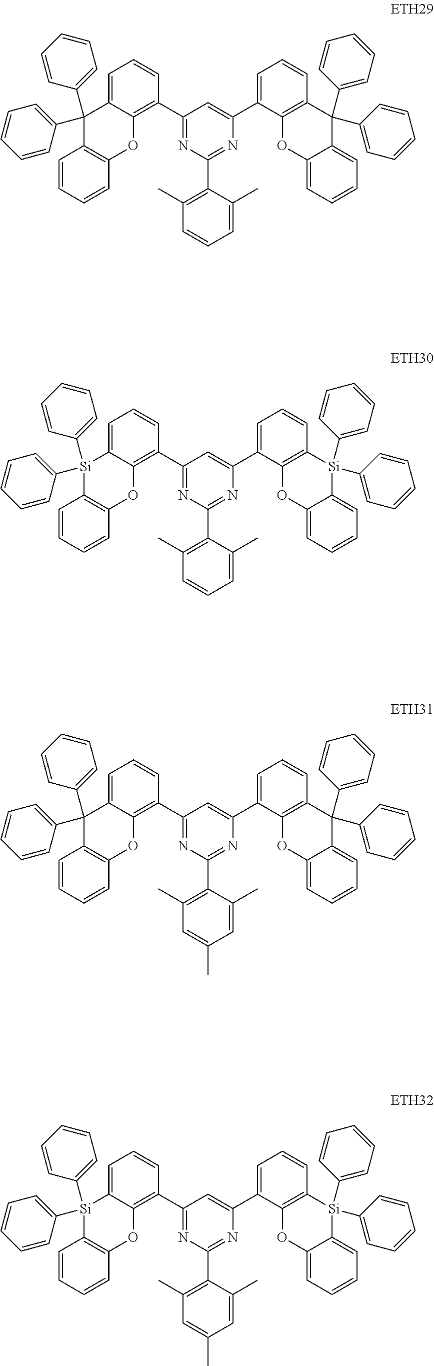 [SMILES: Cc1cccc(C)c1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)cc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)c(C)c1]

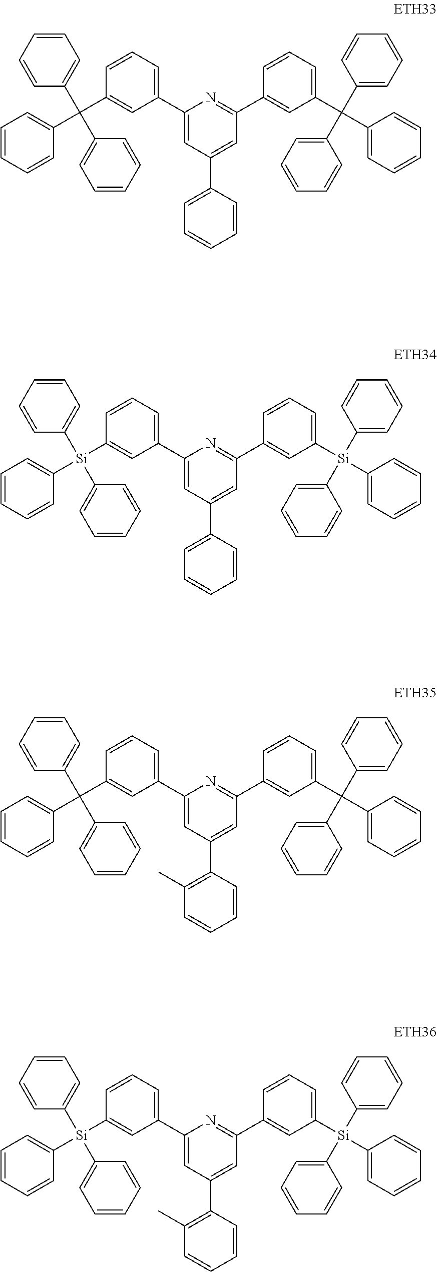 [SMILES: c1ccc(-c2cc(-c3cccc(C(c4ccccc4)(c4ccccc4)c4ccccc4)c3)nc(-c3cccc(C(c4ccccc4)(c4ccccc4)c4ccccc4)c3)c2)cc1 ; c1ccc(-c2cc(-c3cccc([Si](c4ccccc4)(c4ccccc4)c4ccccc4)c3)nc(-c3cccc([Si](c4ccccc4)(c4ccccc4)c4ccccc4)c3)c2)cc1 ; Cc1ccccc1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1ccccc1-c1cc(-c2cccc([Si](c3ccccc3)(c3ccccc3)c3ccccc3)c2)nc(-c2cccc([Si](c3ccccc3)(c3ccccc3)c3ccccc3)c2)c1]

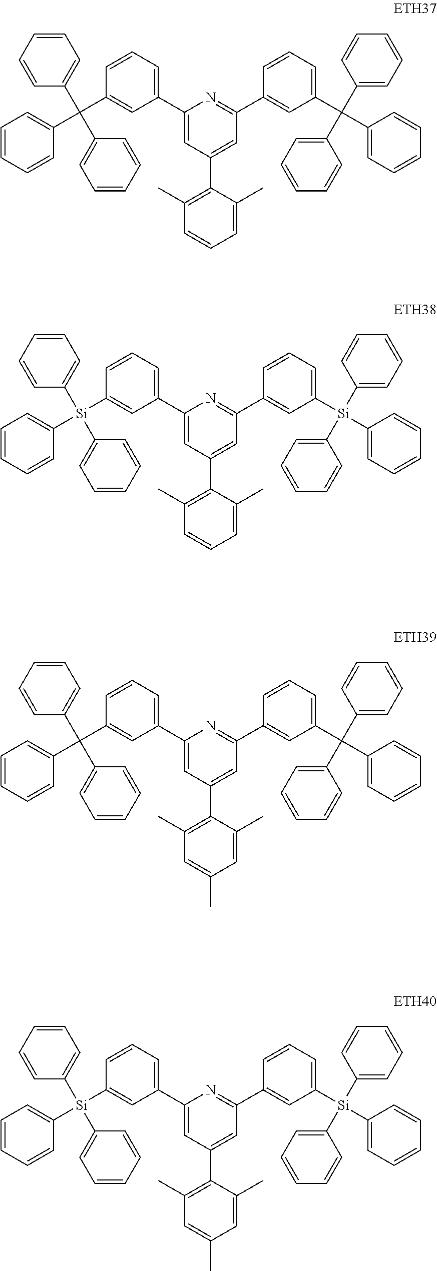 [SMILES: Cc1cccc(C)c1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1cccc(C)c1-c1cc(-c2cccc([Si](c3ccccc3)(c3ccccc3)c3ccccc3)c2)nc(-c2cccc([Si](c3ccccc3)(c3ccccc3)c3ccccc3)c2)c1 ; Cc1cc(C)c(-c2cc(-c3cccc(C(c4ccccc4)(c4ccccc4)c4ccccc4)c3)nc(-c3cccc(C(c4ccccc4)(c4ccccc4)c4ccccc4)c3)c2)c(C)c1 ; Cc1cc(C)c(-c2cc(-c3cccc([Si](c4ccccc4)(c4ccccc4)c4ccccc4)c3)nc(-c3cccc([Si](c4ccccc4)(c4ccccc4)c4ccccc4)c3)c2)c(C)c1]

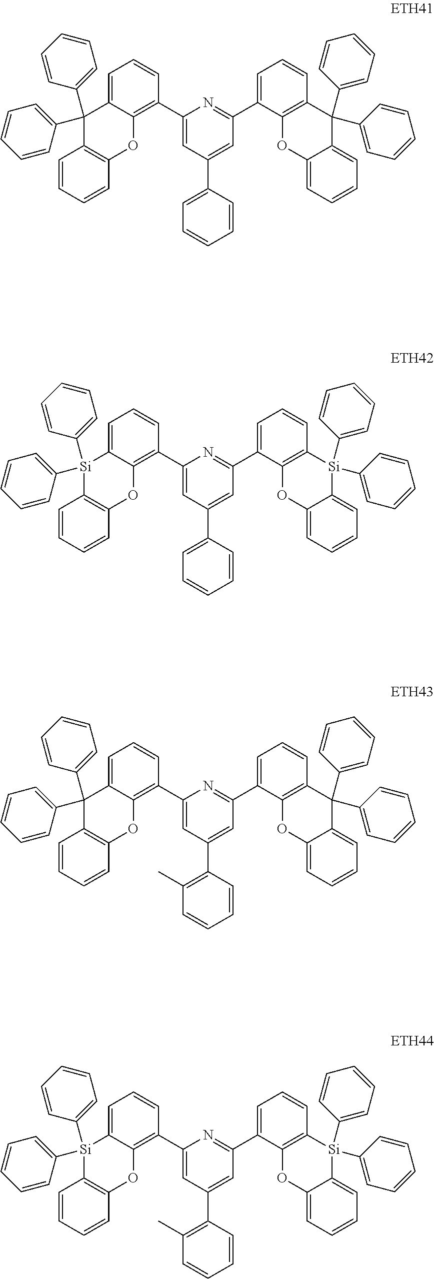 [SMILES: c1ccc(-c2cc(-c3cccc4c3Oc3ccccc3C4(c3ccccc3)c3ccccc3)nc(-c3cccc4c3Oc3ccccc3C4(c3ccccc3)c3ccccc3)c2)cc1 ; c1ccc(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)cc1 ; Cc1ccccc1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1ccccc1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1]

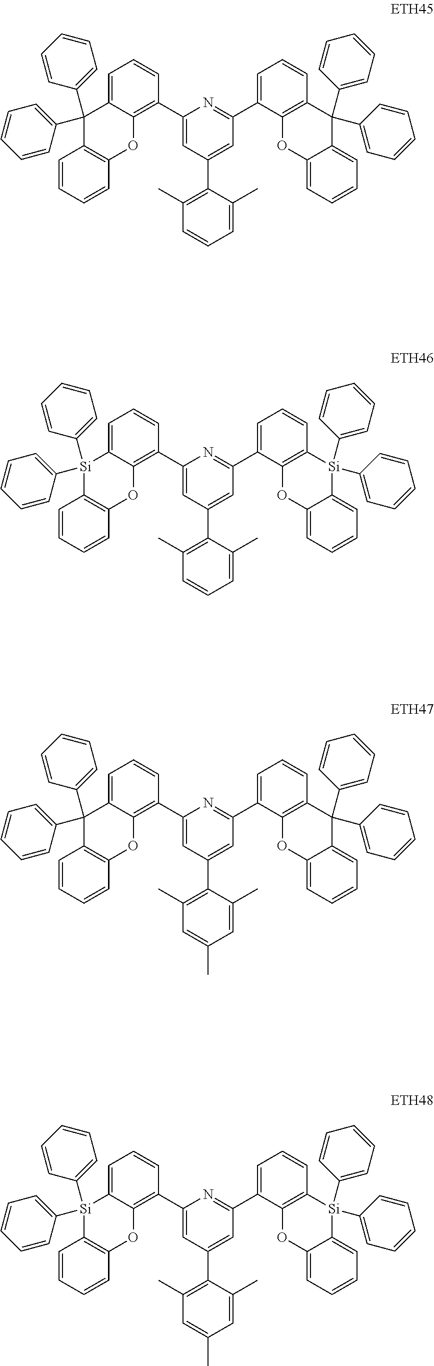 [SMILES: Cc1cccc(C)c1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1cccc(C)c1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1 ; Cc1cc(C)c(-c2cc(-c3cccc4c3Oc3ccccc3C4(c3ccccc3)c3ccccc3)nc(-c3cccc4c3Oc3ccccc3C4(c3ccccc3)c3ccccc3)c2)c(C)c1 ; Cc1cc(C)c(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)c(C)c1]

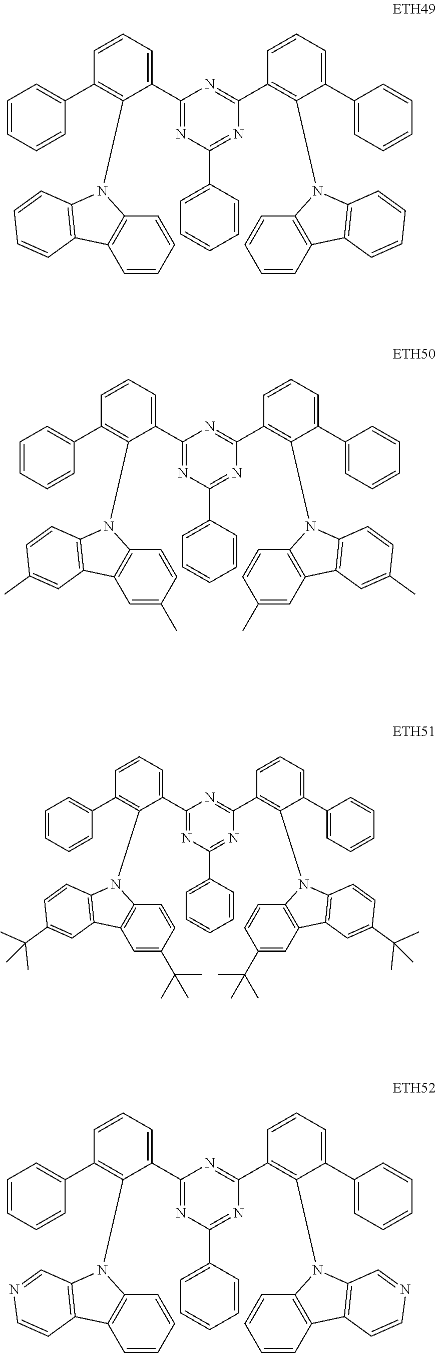

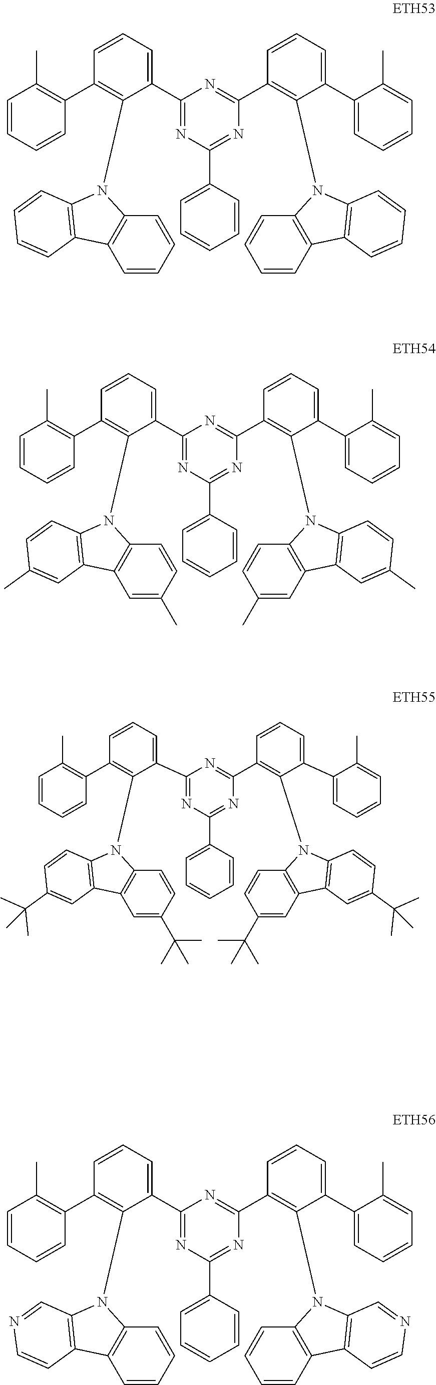

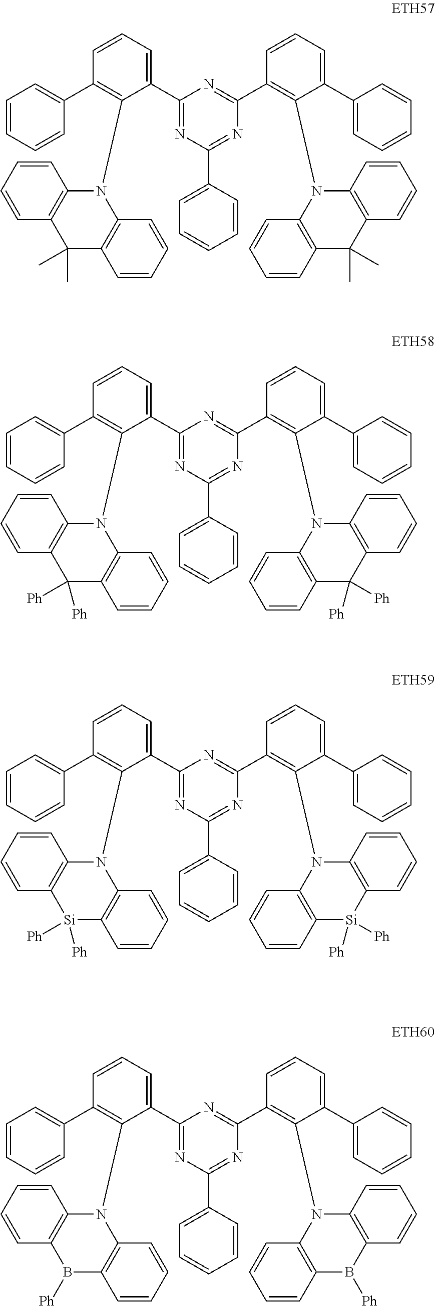

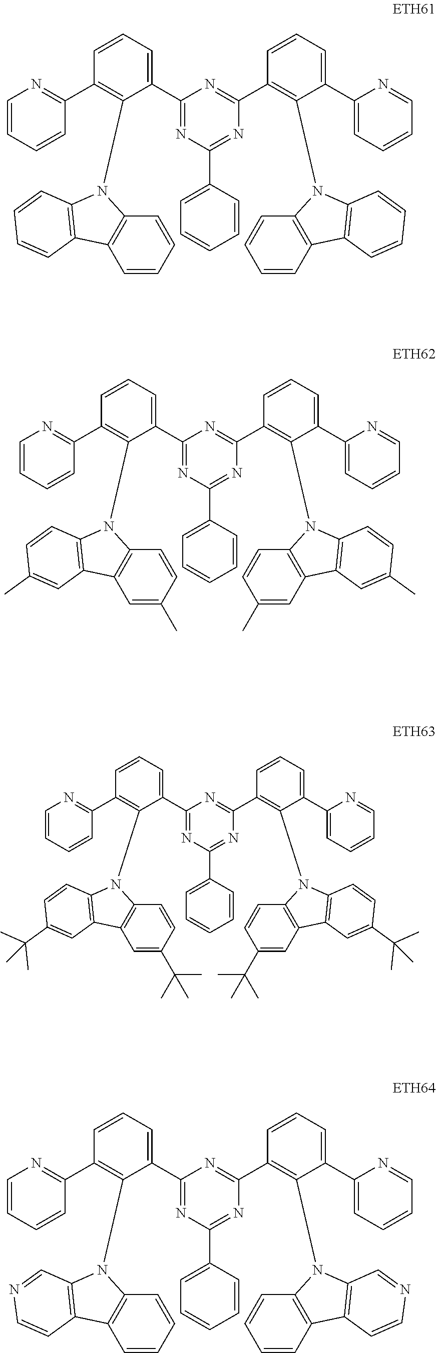

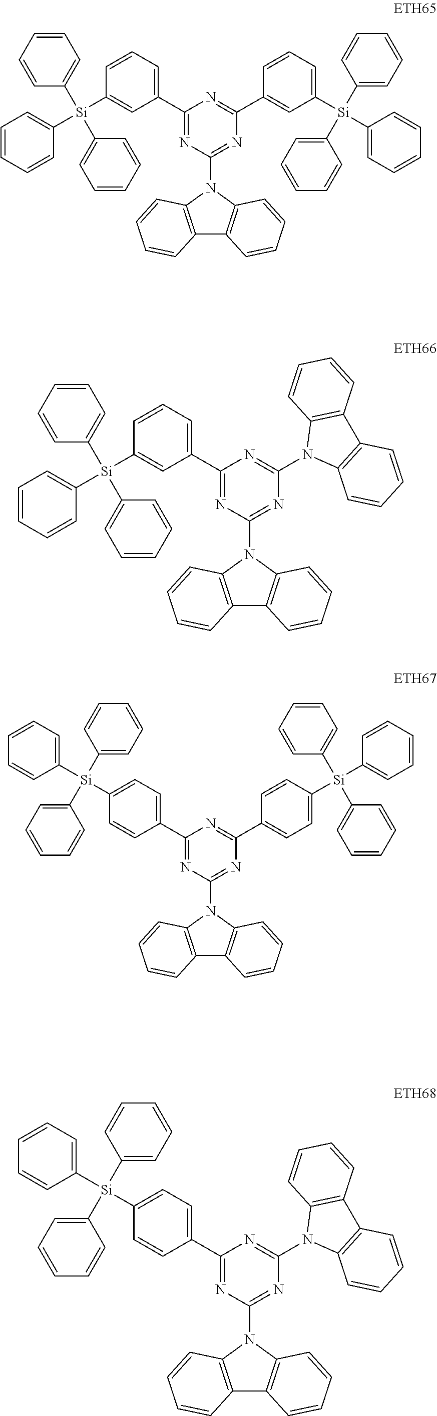 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1]

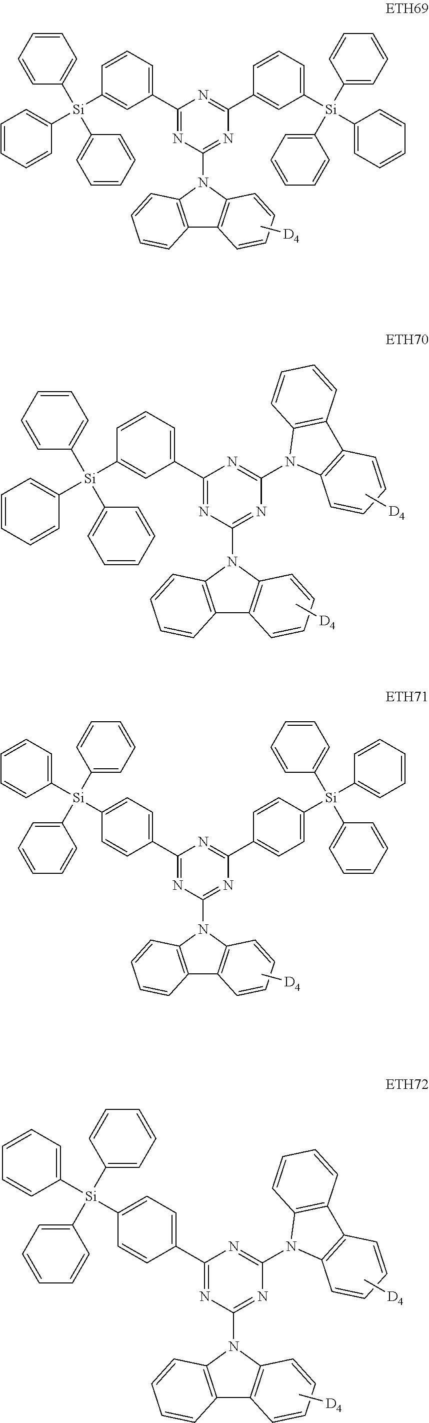

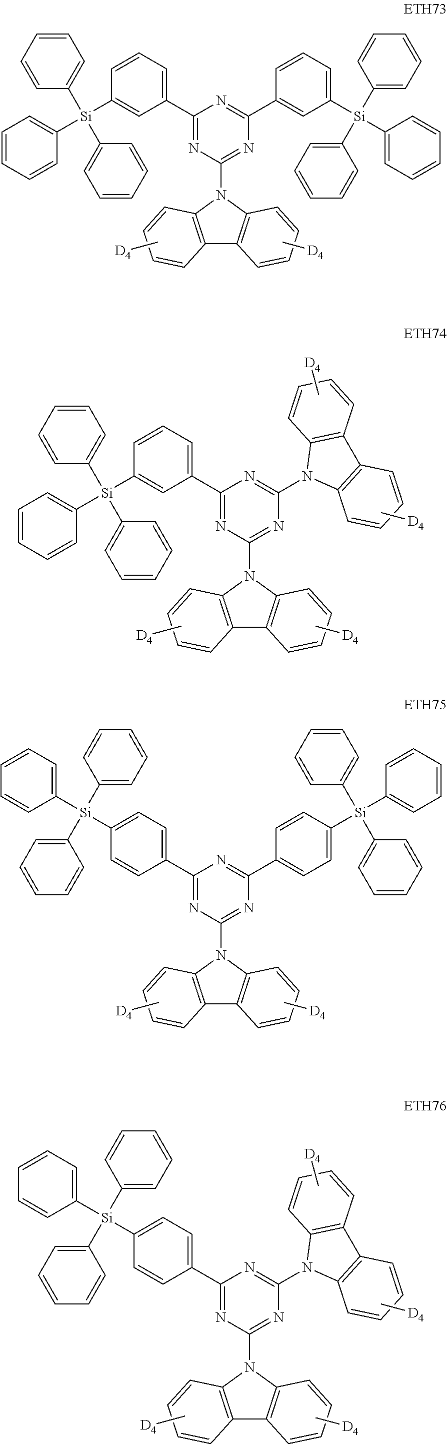

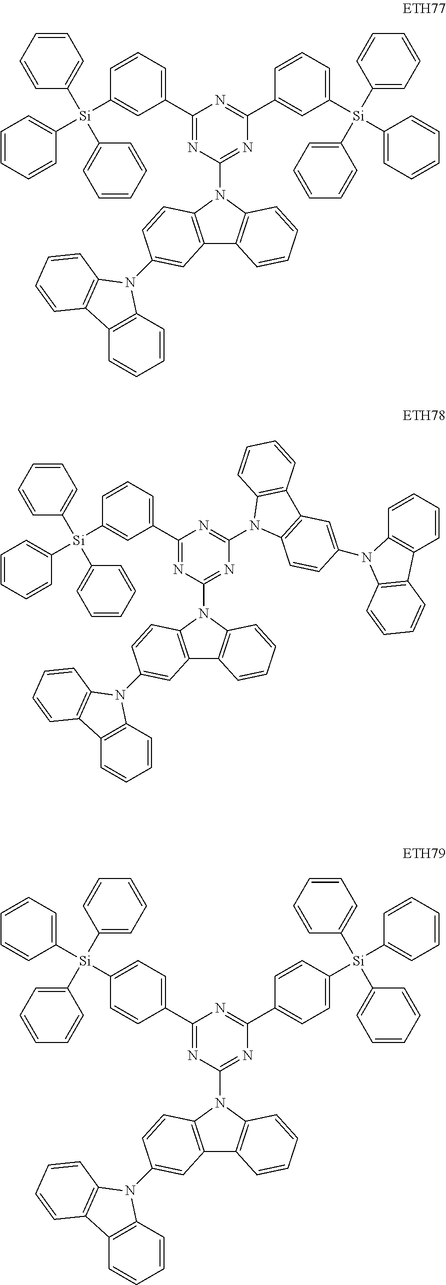 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5cc(-n6c7ccccc7c7ccccc76)ccc54)n3)c2)cc1 ; c1ccc([Si](c2ccccc2)(c2ccccc2)c2cccc(-c3nc(-n4c5ccccc5c5cc(-n6c7ccccc7c7ccccc76)ccc54)nc(-n4c5ccccc5c5cc(-n6c7ccccc7c7ccccc76)ccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5cc(-n6c7ccccc7c7ccccc76)ccc54)n3)cc2)cc1]

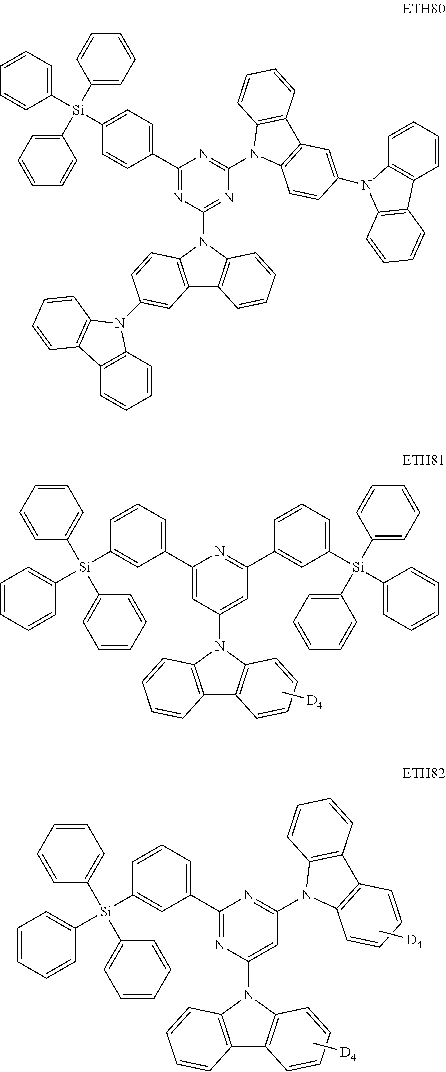

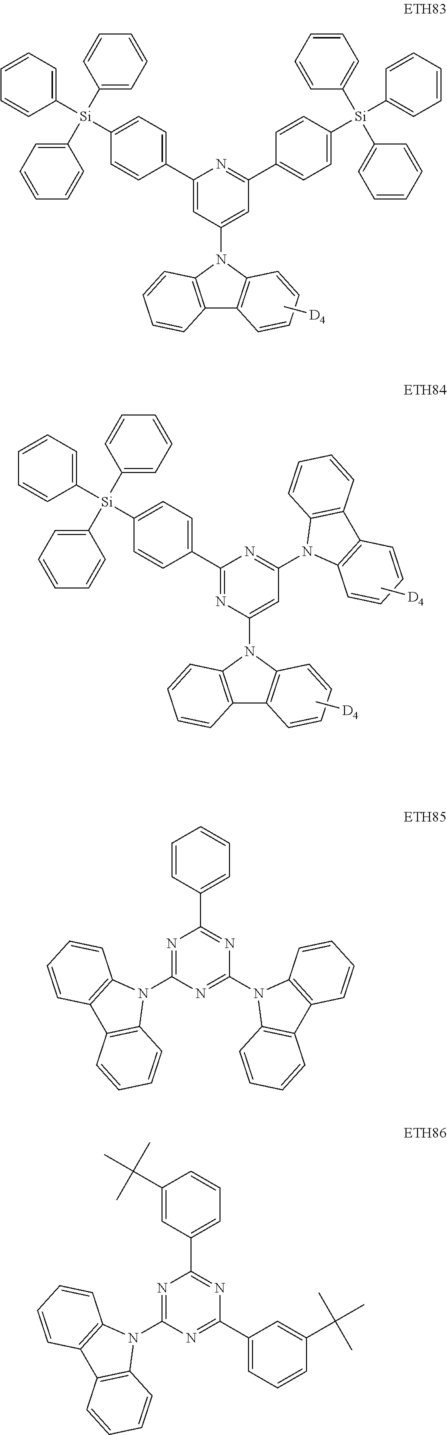

In the example compounds suggested in Compound Group 3, “D” refers to deuterium, and “Ph” refers to an unsubstituted phenyl group.

The light-emitting layer EML may include the second compound and the third compound, and the second compound and the third compound may form an exciplex. In the light-emitting layer EML, the exciplex may be formed by a hole transporting host and an electron transporting host. In this regard, the triplet energy of the exciplex formed by the hole transporting host and the electron transporting host may correspond to a difference between the lowest unoccupied molecular orbital (LUMO) energy level of the electron transporting host and the highest occupied molecular orbital (HOMO) energy level of the hole transporting host.

For example, in one or more embodiments, an absolute value of the triplet energy level (T1) of the exciplex formed by the hole transporting host and the electron transporting host may be about 2.4 eV to about 3.0 eV. In addition, the triplet energy of the exciplex may be a smaller value than the energy gap of each host material. In one or more embodiments, the exciplex may have a triplet energy of about 3.0 eV or less, that is the energy gap between the hole transporting host and the electron transporting host.

In one or more embodiments, the light-emitting layer EML may include a fourth compound in addition to the first compound to the third compound. The fourth compound may be used as a phosphorescence sensitizer of the light-emitting layer EML. Because energy may transfer from the fourth compound to the first compound, light emission may arise.

For example, in one or more embodiments, the light-emitting layer EML may include an organometallic complex which contains platinum (Pt) as a center metal atom and contains ligands bonded to the center metal atom, as the fourth compound. In the light-emitting element ED according to one or more embodiments, the light-emitting layer EML may contain a compound represented by Formula D-1 as the fourth compound.

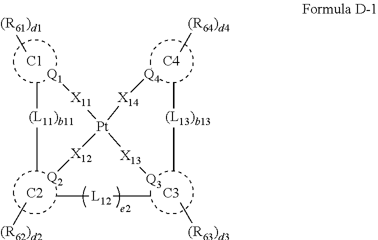

In Formula D-1, Q₁ to Q₄ may each independently be C or N.

In Formula D-1, C1 to C4 may each independently be a substituted or unsubstituted hydrocarbon ring of 5 to 30 ring-forming carbon atoms or a substituted or unsubstituted heterocycle of 2 to 30 ring-forming carbon atoms.

In Formula D-1, X₁₁ to X₁₄ may each independently be a direct linkage or *—O—*. For example, in one or more embodiments, any one selected from X₁₁ to X₁₄ may be *—O—*, and the remainder may be a direct linkage.

In Formula D-1, L₁₁ to L₁₃ may each independently be a direct linkage,

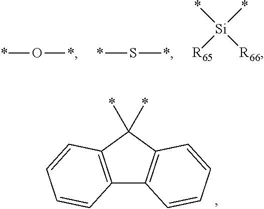

a substituted or unsubstituted alkylene group of 1 to 20 carbon atoms, a substituted or unsubstituted arylene group of 6 to 30 ring-forming carbon atoms, or a substituted or unsubstituted heteroarylene group of 2 to 30 ring-forming carbon atoms. In L₁₁ to L₁₃,

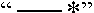

refers to a part coccected with C1 to C4.

In Formula D-1, b11 to b13 may each independently be 0 or 1. If (e.g., when) b11 is 0, C1 and C2 may be unconnected. If (e.g., when) b12 is 0, C2 and C3 may be unconnected. If (e.g., when) b13 is 0, C3 and C4 may be unconnected.

In Formula D-1, R₆₁ to R₆₆ may each independently be hydrogen, deuterium, a halogen, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbon atoms, a substituted or unsubstituted alkenyl group of 2 to 20 carbon atoms, a substituted or unsubstituted aryl group of 6 to 60 ring-forming carbon atoms, or a substituted or unsubstituted heteroaryl group of 2 to 60 ring-forming carbon atoms. In one or more embodiments, one or more selected from among R⁶¹ to R⁶⁶ may be independently combined with an adjacent group to form a ring. In one or more embodiments, R⁶¹ to R⁶⁶ may each independently be a substituted or unsubstituted methyl group or a substituted or unsubstituted t-butyl group.

In Formula D-1, d1 to d4 may each independently be an integer of 0 to 4. In Formula D-1, if (e.g., when) d1 to d4 are 0, the fourth compound may be unsubstituted with R₆₁ to R₆₄, respectively. An embodiment in which d1 to d4 are 4, and R₆₁ to R₆₄ are all hydrogens, may be the same as an embodiment in which d1 to d4 are 0. If (e.g., when) d1 to d4 are integers of 2 or greater, each of multiple R₆₁'s to R₆₄'s may be all the same, or at least one selected from among multiple R₆₁'s to R₆₄'s may be different.

In Formula D-1, C1 to C4 may each independently be a substituted or unsubstituted hydrocarbon ring or a substituted or unsubstituted heterocycle, represented by any one selected from among C-1 to C-5.

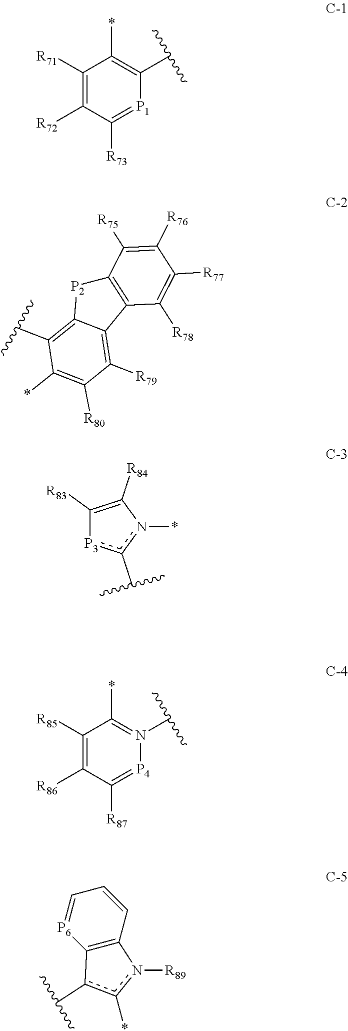

In C-1 to C-5, P₁ may be

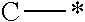 [SMILES: *C]

or CK₇₄, P₂ may be

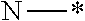 [SMILES: *N]

or NK₈₁, P₃ may be

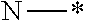 [SMILES: *N]

or NR₈₂, and P₄ may be

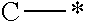 [SMILES: *C]

or CR₈₈, P₆ may be

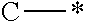 [SMILES: *C]

or CR₉₀. R₇₁ to R₉₀ may each independently be a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring.

In addition, in C-1 to C-5,

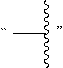

is a part connected with a central metal atom of Pt, and “-*” corresponds to a part connected with an adjacent ring group (C1 to C4) or an adjacent linker (L₁₁ to L₁₃).

The light-emitting layer EML of one or more embodiments may include the first compound that is a fused polycyclic compound, and at least one selected from among the second to fourth compounds. For example, in one or more embodiments, the light-emitting layer EML may include the first compound, the second compound, and the third compound. In the light-emitting layer EML, the second compound and the third compound may form an exciplex, and via the exciplex, energy transfer to the first compound may arise, and light emission may then arise.

In one or more embodiments, the light-emitting layer EML may include the first compound, the second compound, the third compound, and the fourth compound. In the light-emitting layer EML, the second compound and the third compound may form an exciplex, and via the exciplex, energy transfer to the fourth compound and the first compound may arise, and light emission may then arise. In one or more embodiments, the fourth compound may be a sensitizer. In the light-emitting element ED of one or more embodiments, the fourth compound included in the light-emitting layer EML may act as a sensitizer and may play the role of transferring energy from a host to the first compound that is a light-emitting dopant. For example, the fourth compound that plays the role of an auxiliary dopant may accelerate energy transfer to the first compound that is a light emitting dopant and increase the light emitting ratio and efficiency of the first compound. Accordingly, the emission efficiency of the light-emitting layer EML of one or more embodiments may be improved. In addition, if (e.g., when) the energy transfer to the first compound increases, excitons formed in the light-emitting layer EML may not be accumulated but rapidly emit light, and the deterioration of a device may be reduced. Accordingly, the lifetime of the light-emitting element ED of one or more embodiments may increase.

The light-emitting element ED of one or more embodiments includes all of the first compound, the second compound, the third compound, and the fourth compound, and the light-emitting layer EML may include the combination of two host materials and two dopant materials. In the light-emitting element ED of one or more embodiments, the light-emitting layer EML may include the second compound and the third compound, which are two different hosts, the first compound which emits delayed fluorescence, and the fourth compound including an organometallic complex, concurrently (e.g., simultaneously), and may show excellent or suitable emission efficiency properties.

In one or more embodiments, the fourth compound represented by Formula D-1 may be at least one selected from among compounds represented in Compound Group 4. The light-emitting layer EML may include at least one selected from among the compounds represented in Compound Group 4 as a sensitizer material.

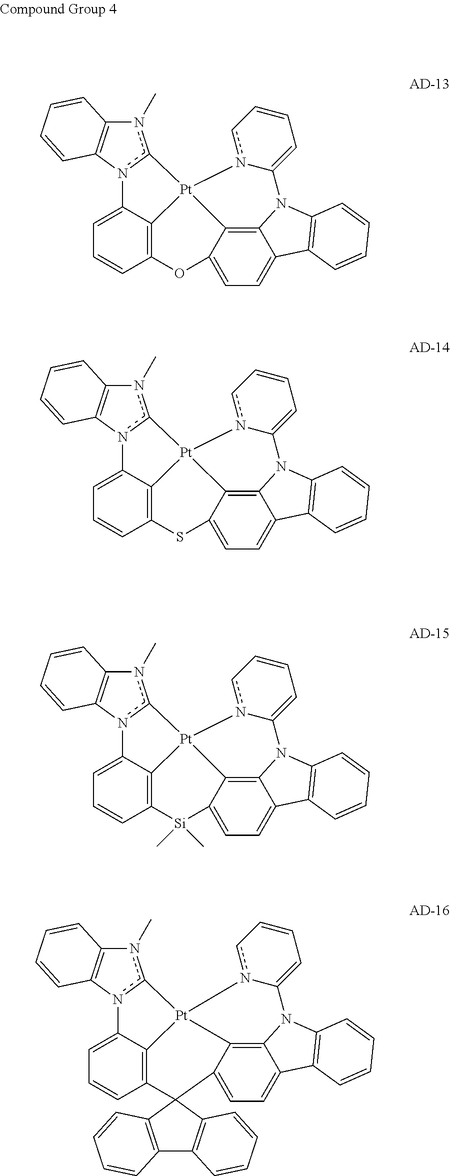

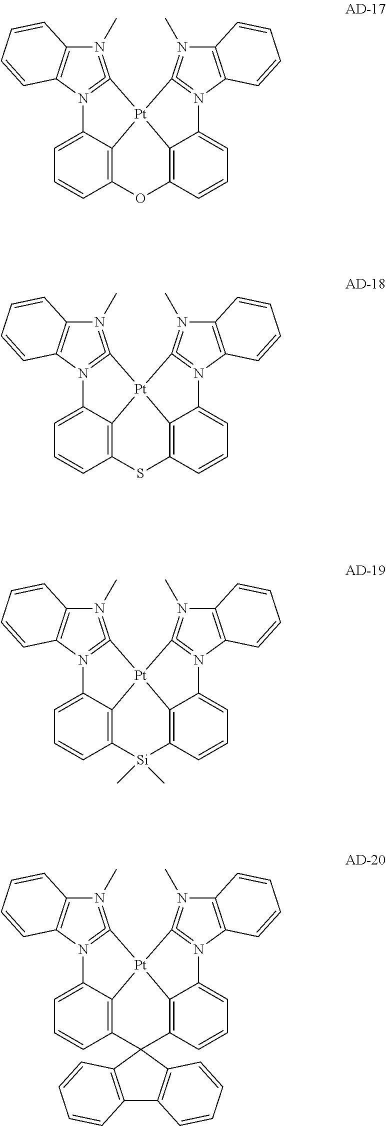

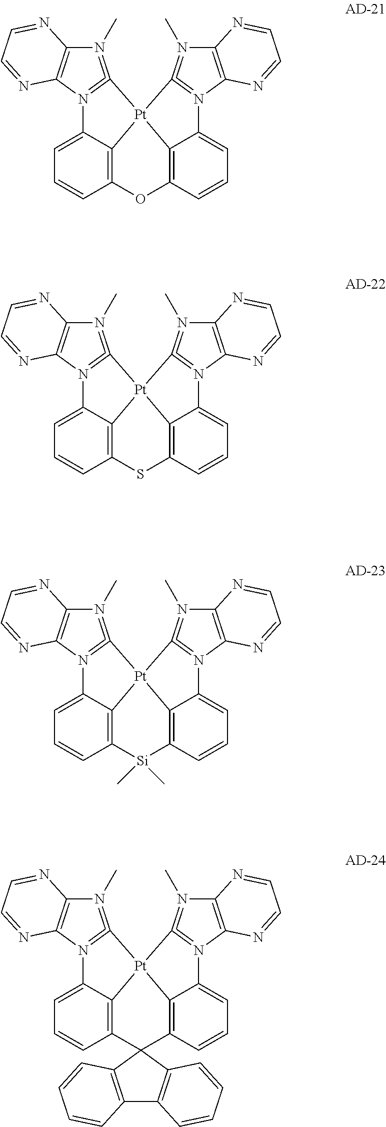

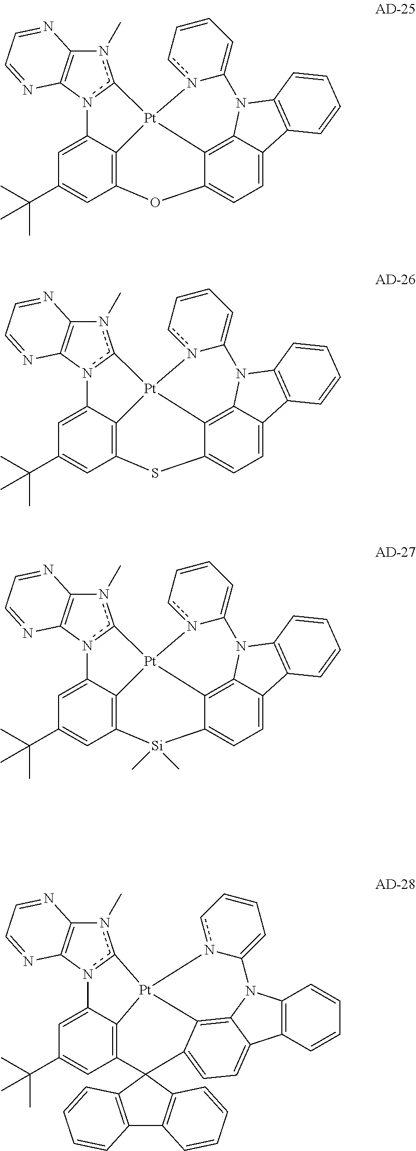

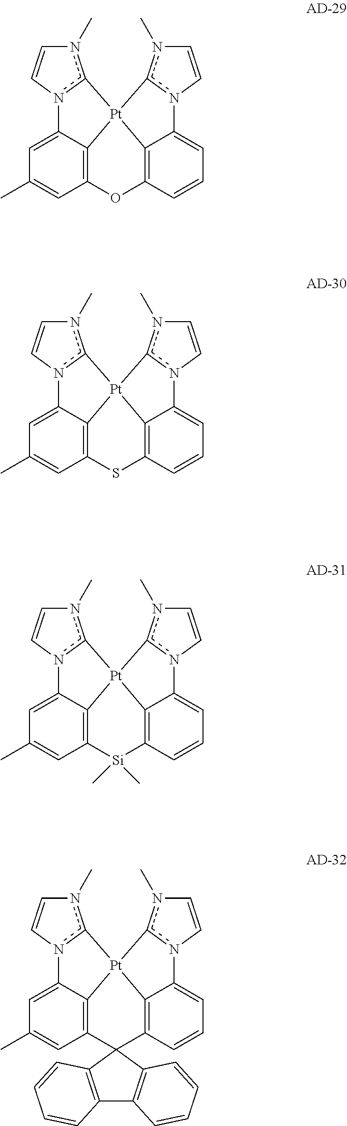

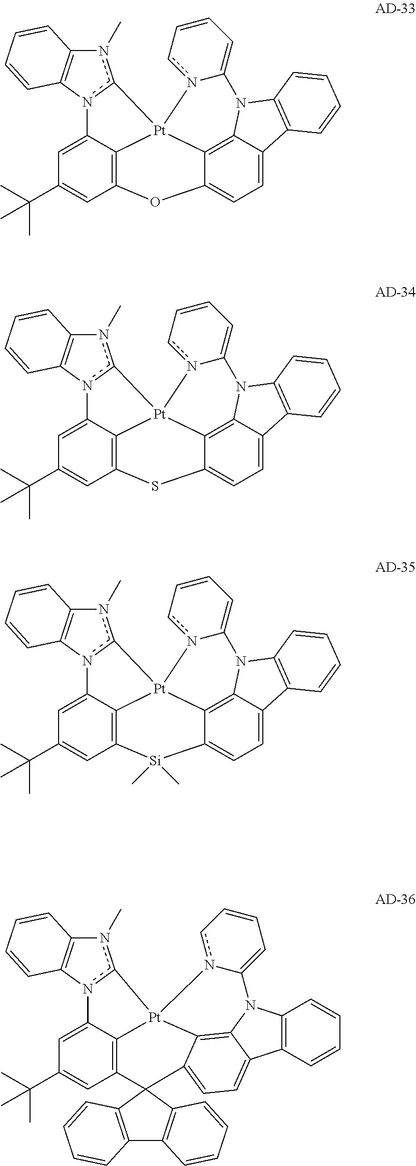

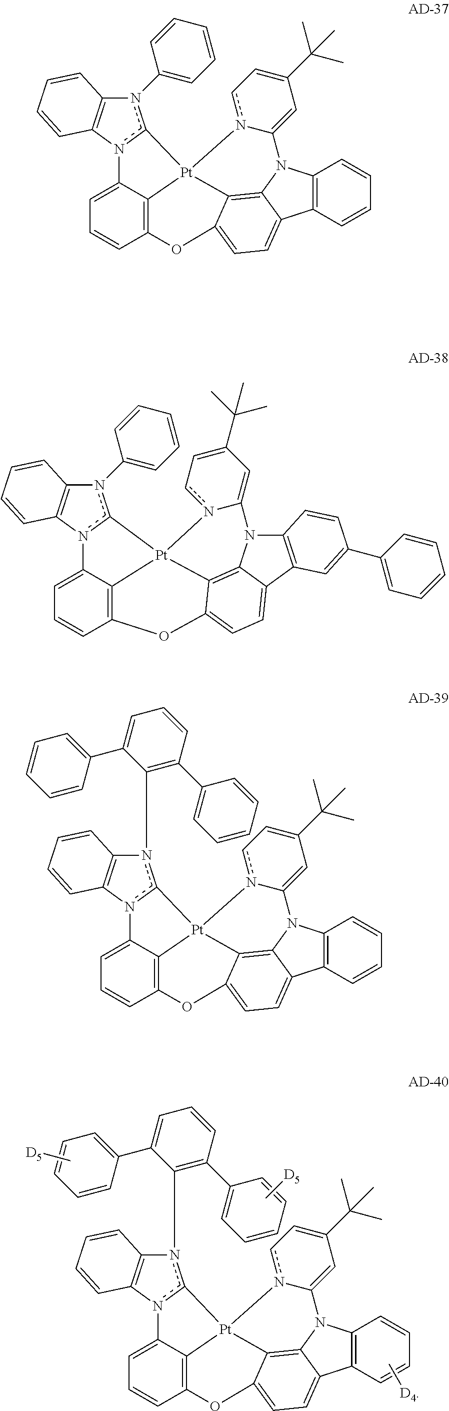

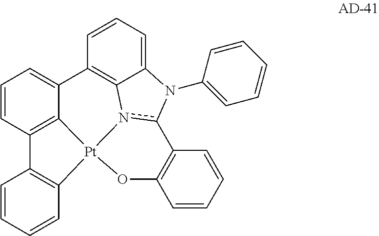

In the example compounds suggested in Compound Group 4, “D” refers to deuterium.

In the light-emitting element ED of one or more embodiments, if (e.g., when) the light-emitting layer EML includes all of the first compound, the second compound, the third compound, and the fourth compound, an amount of the first compound may be about 0.1 wt % to about 5 wt % based on a total weight of 100 wt % of the first compound, the second compound, the third compound, and the fourth compound. However, embodiments of the present disclosure are not limited thereto. If (e.g., when) the amount of the first compound satisfies the above-described ratio, energy transfer from the second compound and the third compound to the first compound may increase, and accordingly, the emission efficiency and device lifetime may increase.

In the light-emitting layer EML, an total amount of the second compound and the third compound may be the remaining amount excluding the amount of the first compound and the fourth compound. For example, in one or more embodiments, the total amount of the second compound and the third compound may be about 65 wt % to about 95 wt % based on the total weight of 100 wt % of the first compound, the second compound, the third compound, and the fourth compound.

In the total amount of the second compound and the third compound, a weight ratio of the second compound to the third compound may be about 3:7 to about 7:3.

If (e.g., when) the total amount of the second compound and the third compound satisfies the above-described ratio, charge balance properties in the light-emitting layer EML may be improved, and emission efficiency and device lifetime may be improved. If (e.g., when) the total amount of the second compound and the third compound deviates from the above-described ratio range, charge balance in the light-emitting layer EML may be broken, emission efficiency may be degraded, and the device may be easily deteriorated.

If (e.g., when) the light-emitting layer EML includes the fourth compound, an amount of the fourth compound may be about 4 wt % to about 30 wt % based on the total weight of 100 wt % of the first compound, the second compound, the third compound, and the fourth compound in the light-emitting layer EML. However, embodiments of the present disclosure are not limited thereto. If (e.g., when) the amount of the fourth compound satisfies the above-described amount, energy transfer from a host to the first compound that is a light emitting dopant may increase, and emission ratio and efficiency may be improved. Accordingly, the emission efficiency of the light-emitting layer EML may be improved. If (e.g., when) the amount ratios of the first compound, the second compound, the third compound, and the fourth compound, included in the light-emitting layer EML satisfies the above-described amount ratio, excellent or suitable emission efficiency and long lifetime may be achieved.

In one or more embodiments, the light-emitting layer EML may include as a suitable dopant material, one or more selected from among styryl derivatives (for 1 example, 1,4-bis[2-(3-N-ethylcarbazolyl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N, N-diphenylamino)phenyl)vinyl]biphenyl (DPAVBi)), perylene and the derivatives thereof (for example, 2,5,8,11-tetra-t-butylperylene (TBP)), pyrene and the derivatives thereof (for example, 1,1-dipyrene, 1,4-dipyrenylbenzene, and 1,4-bis(N,N-diphenylamino)pyrene), and/or the like.

In one or more embodiments, the light-emitting layer EML may include a suitable phosphorescence dopant material. For example, the phosphorescence dopant may use a metal complex including iridium (Ir), platinum (Pt), osmium (Os), gold (Au), titanium (Ti), zirconium (Zr), hafnium (Hf), europium (Eu), terbium (Tb), or thulium (Tm). For example, in one or more embodiments, iridium(III) bis(4,6-difluorophenylpyridinato-N, C2′)picolinate (FIrpic), bis(2,4-difluorophenylpyridinato)-tetrakis (1-pyrazolyl)borate iridium(III) (Fir6), or platinum octaethyl porphyrin (PtOEP) may be used as the phosphorescence dopant. However, embodiments of the present disclosure are not limited thereto.

In one or more embodiments, the light-emitting layer EML may include a hole transporting host and an electron transporting host. In addition, the light-emitting layer EML may include an auxiliary dopant and a light emitting dopant. In one or more embodiments, the auxiliary dopant may include a phosphorescence dopant material or a thermally activated delayed fluorescence dopant. For example, in one or more embodiments, the light-emitting layer EML may include a hole transporting host, an electron transporting host, an auxiliary dopant, and a light emitting dopant.

In one or more embodiments, the light-emitting layer may include a quantum dot.

In the present disclosure, the quantum dot refers to the crystal of a semiconductor compound. The quantum dot may be to emit light in one or more suitable emission wavelengths according to the size of the crystal. The quantum dot may be to emit light in one or more suitable emission wavelengths by controlling an element ratio in the quantum dot compound.

A diameter of the quantum dot may be, for example, about 1 nm to about 10 nm. In the present disclosure, when dot, dots, or dot particles are spherical, “diameter” indicates a particle diameter or an average particle diameter, and when the particles are non-spherical, the “diameter” indicates a major axis length or an average major axis length. The diameter of the particles may be measured utilizing a scanning electron microscope or a particle size analyzer. As the particle size analyzer, for example, HORIBA, LA-950 laser particle size analyzer, may be utilized. When the size of the particles is measured utilizing a particle size analyzer, the average particle diameter is referred to as D50. D50 refers to the average diameter of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size.

The quantum dot may be synthesized by a chemical bath deposition, a metal organic chemical vapor deposition, a molecular beam epitaxy, or a similar process therewith.

The chemical bath deposition is a method of mixing an organic solvent and a precursor material of a quantum dot and then, growing a quantum dot particle crystal. During growing the crystal, the organic solvent may naturally play the role of a dispersant which is coordinated on the surface of the quantum dot crystal and may control the growth of the crystal. Accordingly, the chemical bath deposition is more advantageous and beneficial if (e.g., when) compared to a vapor deposition method including a metal organic chemical vapor deposition (MOCVD) and/or molecular beam epitaxy (MBE), and the growth of the quantum dot particle may be controlled or selected through a low-cost process.

In one or more embodiments, the light-emitting layer EML may include a quantum dot material. The quantum dot may have a core/shell structure. The core of the quantum dot may be selected from among a II-VI group compound, a III-VI group compound, a I-III-VI group compound, a III-V group compound, a III-II-V group compound, a IV-VI group compound, a IV group element, a IV group compound, and/or one or more (e.g., any suitable) combinations thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and a (e.g., any suitable) mixture thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a (e.g., any suitable) mixture thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and a (e.g., any suitable) mixture thereof. In one or more embodiments, the Group II-VI compound may further include a Group I metal and/or a Group IV element. The Group I-II-VI compound may be selected from among CuSnS and CuZnS, and the Group II-IV-VI compound may be selected from among ZnSnS and/or the like. The Group I-II-IV-VI compound may be selected from among quaternary compounds selected from the group consisting of Cu₂ZnSnS₂, Cu₂ZnSnS₄, Cu₂ZnSnSe₄, Ag₂ZnSnS₂, and a (e.g., any suitable) mixture thereof.

The III-VI group compound may include a binary compound such as In₂S₃ and/or In₂Se₃, a ternary compound such as InGaS₃ and/or InGaSe₃, or a (e.g., any suitable) combination thereof.

The I-III-VI group compound may be selected from among a ternary compound selected from the group consisting of AgInS, AgInS₂, CuInS, CuInS₂, AgGaS₂, CuGaS₂, CuGaO₂, AgGaO₂, AgAIO₂ and mixtures thereof, and/or a quaternary compound such as AgInGaS₂ and CuInGaS₂.

The III-V group compound may be selected from the group consisting of a binary compound selected from the group consisting of GaN, GaP, GaAs, GaSb, AlN, AlP, AlAs, AlSb, InN, InP, InAs, InSb, and mixtures thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures thereof. In one or more embodiments, the III-V group compound may further include a II group metal. For example, InZnP, and/or the like may be selected as a III-II-V group compound.

The Group IV-VI compound may be selected from the group consisting of a binary compound selected from the group consisting of SnS, SnSe, SnTe, PbS, PbSe, PbTe, and a (e.g., any suitable) mixture thereof, a ternary compound selected from the group consisting of SnSeS, SnSeTe, SnSTe, PbSeS, PbSeTe, PbSTe, SnPbS, SnPbSe, SnPbTe, and a (e.g., any suitable) mixture thereof, and a quaternary compound selected from the group consisting of SnPbSSe, SnPbSeTe, SnPbSTe, and a (e.g., any suitable) mixture thereof.

The Group II-IV-V compound may be a ternary compound selected from the group consisting of ZnSnP, ZnSnP₂, ZnSnAs₂, ZnGeP₂, ZnGeAs₂, CdSnP₂, and CdGeP₂ and a (e.g., any suitable) mixture thereof.

The Group IV element may be selected from the group consisting of Si, Ge, and a (e.g., any suitable) mixture thereof. The Group IV compound may be a binary compound selected from the group consisting of SiC, SiGe, and a (e.g., any suitable) mixture thereof.

Each element included in a multi-element compound such as the binary compound, ternary compound, or quaternary compound may be present in particles at a substantially uniform concentration or a non-uniform concentration. For example, Formula above indicates the types (kinds) of elements included in a compound, and element ratios in the compound may be different. For example, AgInGaS₂ may indicate AgInₓGa₁₋ₓS₂ (x is a real number between 0 and 1).

In one or more embodiments, the binary compound, the ternary compound, or the quaternary compound may be present at substantially uniform concentration in a particle or may be present at a partially different concentration distribution state in the same particle. In one or more embodiments, a core/shell structure in which one quantum dot wraps another quantum dot may be desired. The interface of the core and the shell may have a concentration gradient in which the concentration of an element present in the shell is decreased toward the center.

In one or more embodiments, the quantum dot may have the above-described core-shell structure including a core including a nanocrystal and a shell wrapping the core. The shell of the quantum dot may play the role of a protection layer for preventing or reducing the chemical deformation of the core to maintain semiconductor properties and/or a charging layer for imparting the quantum dot with electrophoretic properties. The shell may have a single layer or a multilayer. Examples of the shell of the quantum dot may include a metal or non-metal oxide, a semiconductor compound, and/or one or more (e.g., any suitable) combinations thereof.

For example, the metal or non-metal oxide for the shell may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, and/or NiO, or a ternary compound such as MgAl₂O₄, CoFe₂O₄, NiFe₂O₄, and/or CoMn₂O₄, but embodiments of the present disclosure are not limited thereto.

Also, the semiconductor compound suitable as a shell may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, and/or the like, but embodiments of the present disclosure are not limited thereto.

The quantum dot may have a full width of half maximum (FWHM) of emission spectrum of about 45 nm or less, about 40 nm or less, or about 30 nm or less. Within this range, color purity or color reproducibility of the quantum dot may be improved. In addition, light emitted via such quantum dot is emitted in all directions, and light view angle properties may be improved.

In addition, the shape of the quantum dot may be a generally used shape in the art, without specific limitation. For example, spherical nanoparticles, pyramidal nanoparticles, multi-arm nanoparticles, cubic nanoparticles, nanotubes, nanowires, nanofibers, nanoplates, and/or the like, may be used.

As the size of the quantum dot or the ratio of elements in the quantum dot compound is regulated, the energy band gap of the quantum dot may be accordingly controlled or selected to obtain light of one or more suitable wavelengths from a quantum dot emission layer. Therefore, by using the quantum dots as described above (e.g., using quantum dots of different sizes or having different element ratios in the quantum dot compound), a light-emitting element emitting light of one or more suitable wavelengths may be obtained. For example, the size of the quantum dots or the ratio of elements in the quantum dot compound may be regulated to enable the quantum dots to emit red, green, and/or blue light. In one or more embodiments, the quantum dots may be configured to emit white light by combining light of one or more suitable colors.

In each of the light-emitting elements ED of embodiments, as shown in FIG. 3 to FIG. 6, the electron transport region ETR may be provided on the light-emitting layer EML. The electron transport region ETR may include at least one of a hole blocking layer HBL, an electron transport layer ETL, or an electron injection layer EIL. However, embodiments of the present disclosure are not limited thereto.

The electron transport region ETR may have a single layer formed using a single material, a single layer formed using multiple different materials, or a multilayer structure having multiple layers formed using multiple different materials.

For example, in one or more embodiments, the electron transport region ETR may have a single layer structure of an electron injection layer EIL or an electron transport layer ETL, or a single layer structure formed using an electron injection material and/or an electron transport material. Further, in one or more embodiments, the electron transport region ETR may have a single layer structure formed using multiple different materials, or a structure stacked from the light-emitting layer EML of electron transport layer ETL/electron injection layer EIL, or hole blocking layer HBL/electron transport layer ETL/electron injection layer EIL, without limitation. A thickness of the electron transport region ETR may be, for example, from about 1,000 Å to about 1,500 Å.

The electron transport region ETR may be formed using one or more suitable methods such as a vacuum deposition method, a spin coating method, a casting method, a Langmuir-Blodgett (LB) method, an inkjet printing method, a laser printing method, and/or a laser induced thermal imaging (LITI) method.

In one or more embodiments, the electron transport region ETR may include a compound represented by Formula ET-2.

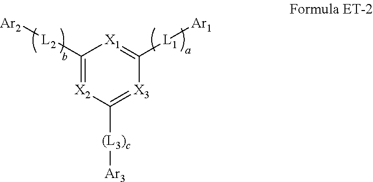

In Formula ET-2, at least one selected from among X₁ to X₃ may be N, and the remainder are CRₐ. Rₐ may be hydrogen, deuterium, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. Ar₁ to Ar₃ may each independently be hydrogen, deuterium, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-2, “a” to “c” may each independently be an integer of 0 to 10. In Formula ET-2, L₁ to L₃ may each independently be a direct linkage, a substituted or unsubstituted arylene group of 6 to 30 ring-forming carbon atoms, or a substituted or unsubstituted heteroarylene group of 2 to 30 ring-forming carbon atoms. In one or more embodiments, if (e.g., when) “a” to “c” are integers of 2 or greater, Li's to Ls's may each independently be a substituted or unsubstituted arylene group of 6 to 30 ring-forming carbon atoms or a substituted or unsubstituted heteroarylene group of 2 to 30 ring-forming carbon atoms.

In one or more embodiments, the electron transport region ETR may include an anthracene-based compound. However, embodiments of the present disclosure are not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzimidazolyl-1-yl)phenyl)-9,10-dinaphthylanthracene, 1,3,5-tri (1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(biphenyl-4-yl)-4-phenyl-5-tert-butylphenyl-1,2,4-triazole (TAZ), 4-(naphthalen-1-yl)-3,5-diphenyl-4H-1,2,4-triazole (NTAZ), 2-(biphenyl-4-yl)-5-(4-tert-butylphenyl)-1,3,4-oxadiazole (tBu-PBD), bis(2-methyl-8-quinolinolato-N1,08)-(1,1′-biphenyl-4-olato)aluminum (BAlq), berylliumbis (benzoquinolin-10-olate (Bebq2), 9,10-di(naphthalen-2-yl) anthracene (ADN), 1,3-bis[3,5-di (pyridin-3-yl)phenyl]benzene (BmPyPhB), CNNPTRZ (4′-(4-(4-(4,6-diphenyl-1,3,5-triazin-2-yl)phenyl)naphthalen-1-yl)-[1,1′-biphenyl]-4-carbonitrile), and/or a (e.g., any suitable) mixture thereof, without limitation.

In one or more embodiments, the electron transport region ETR may include at least one selected from among Compounds ET1 to ET36.

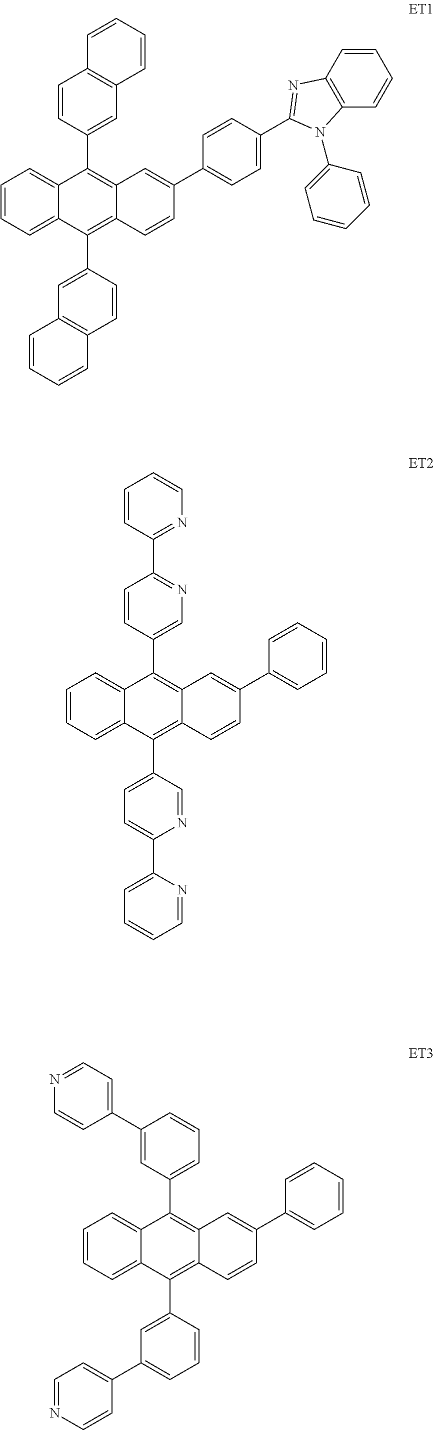 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

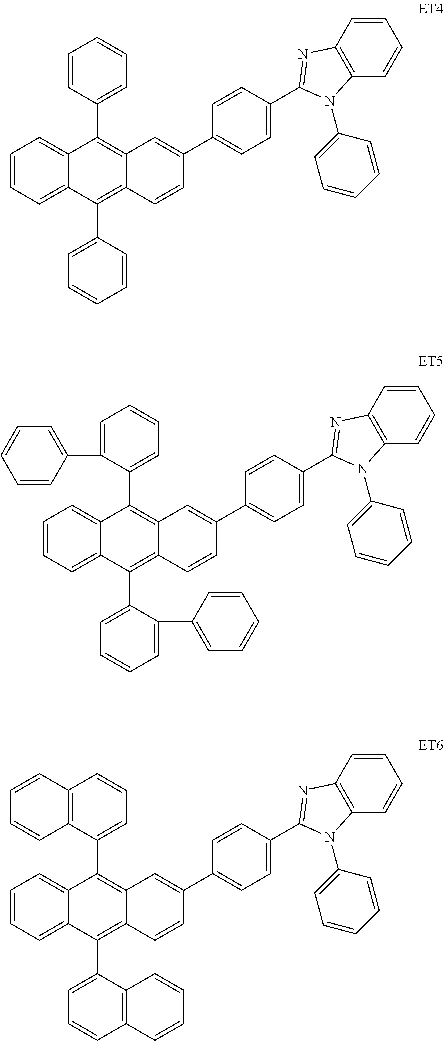 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

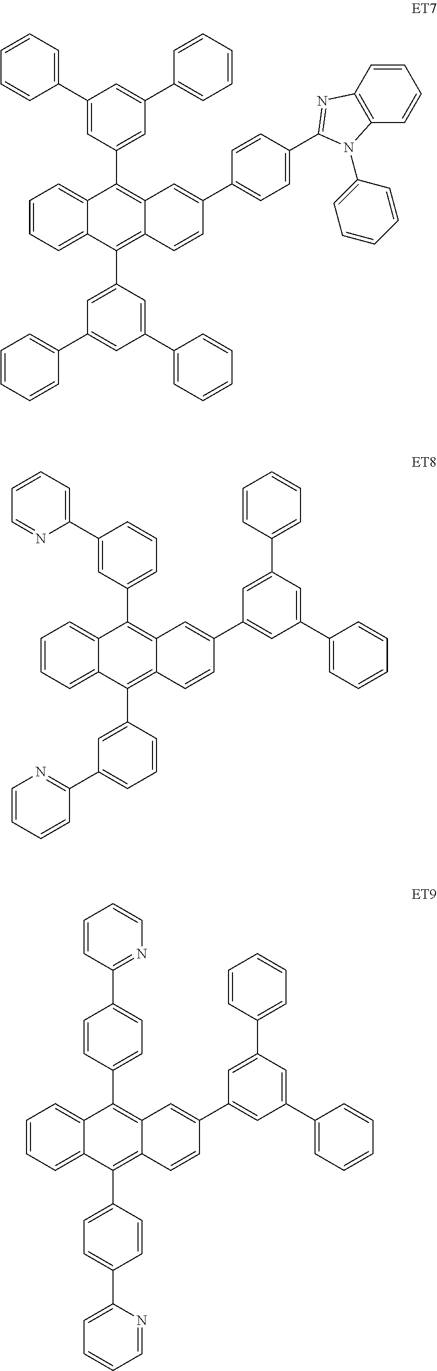 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

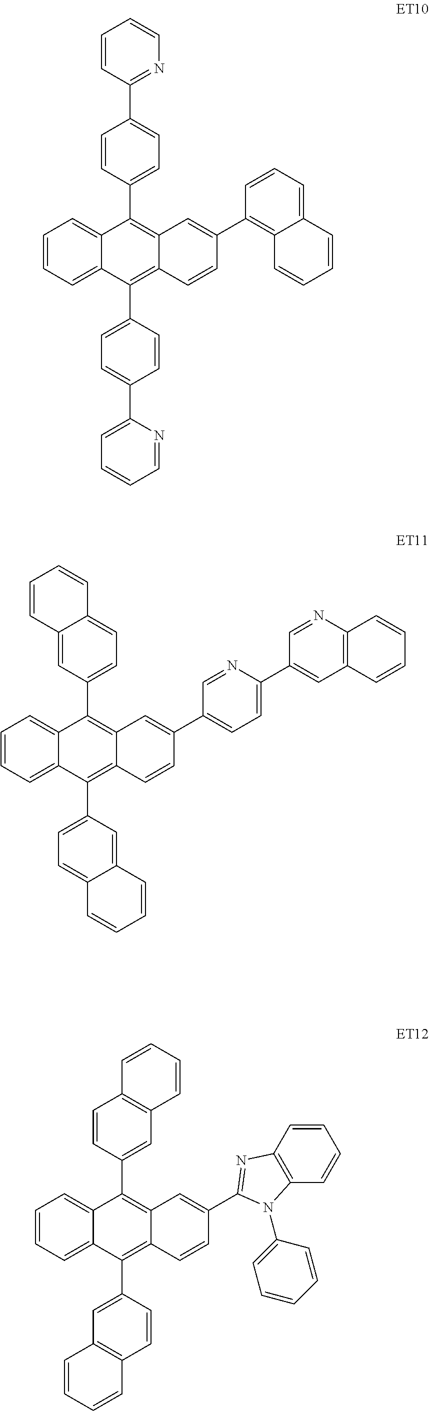 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

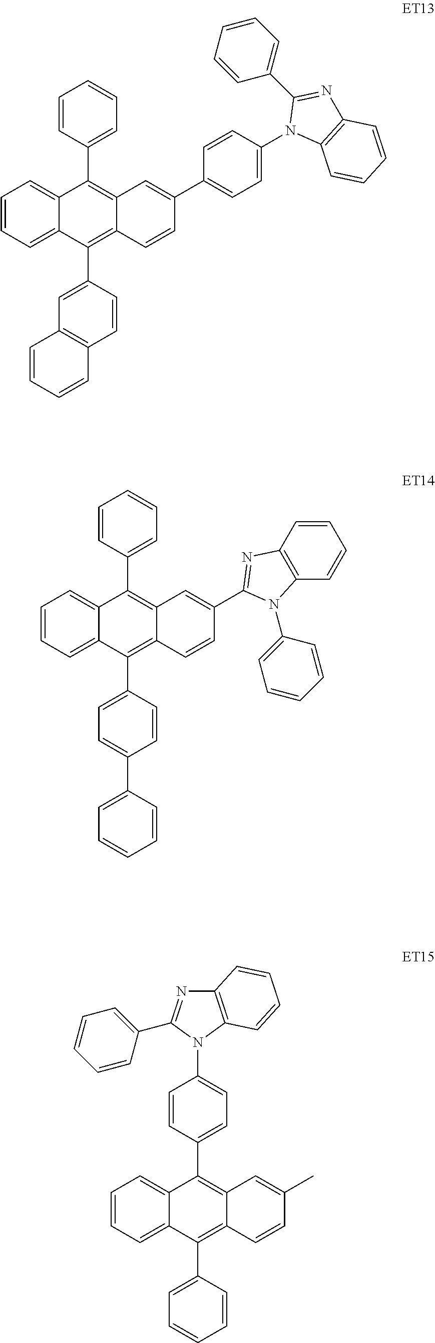 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

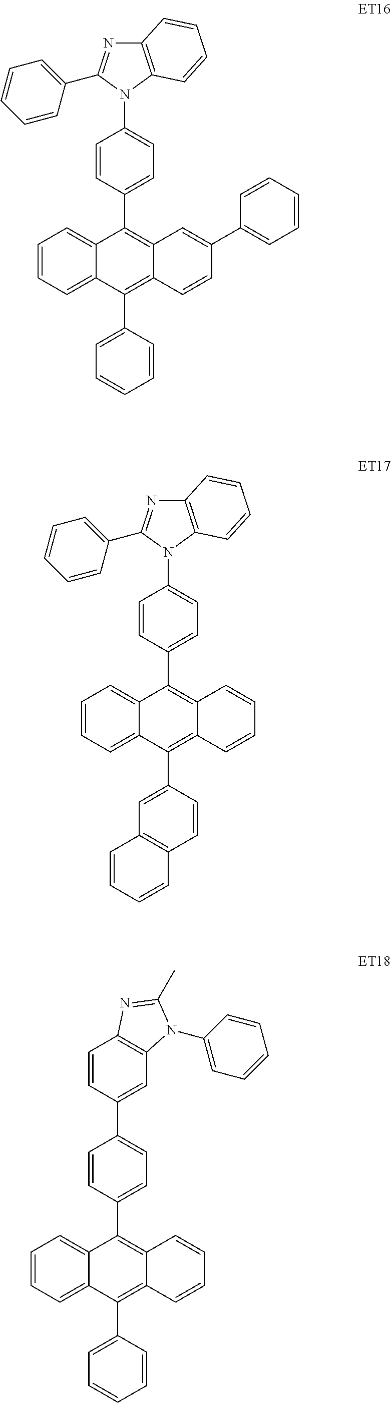 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

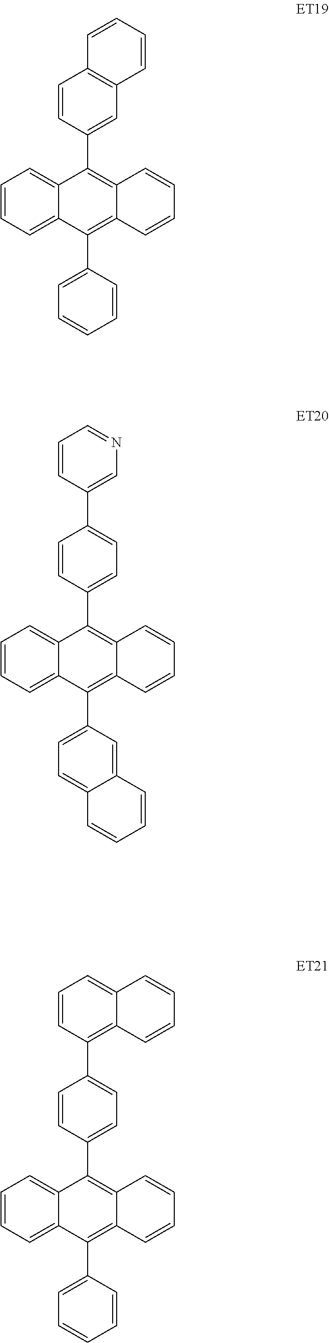

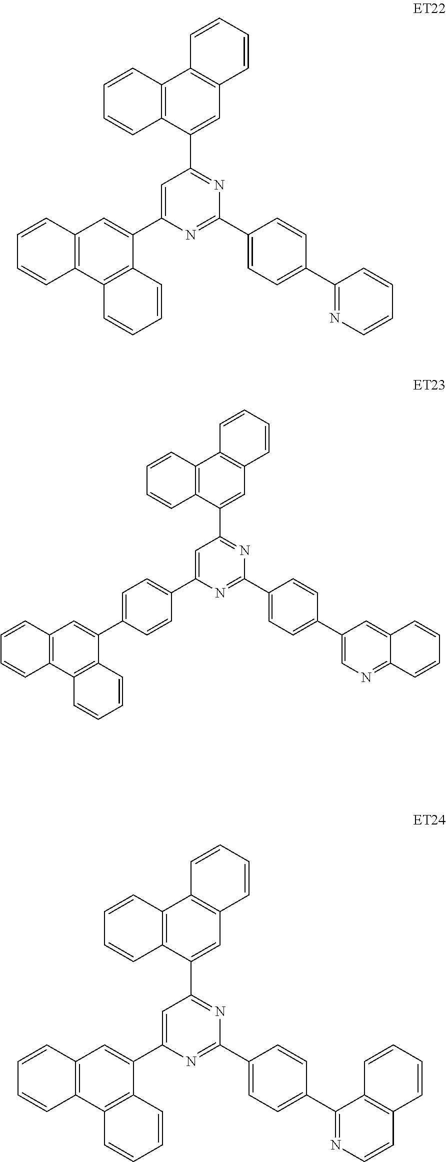 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

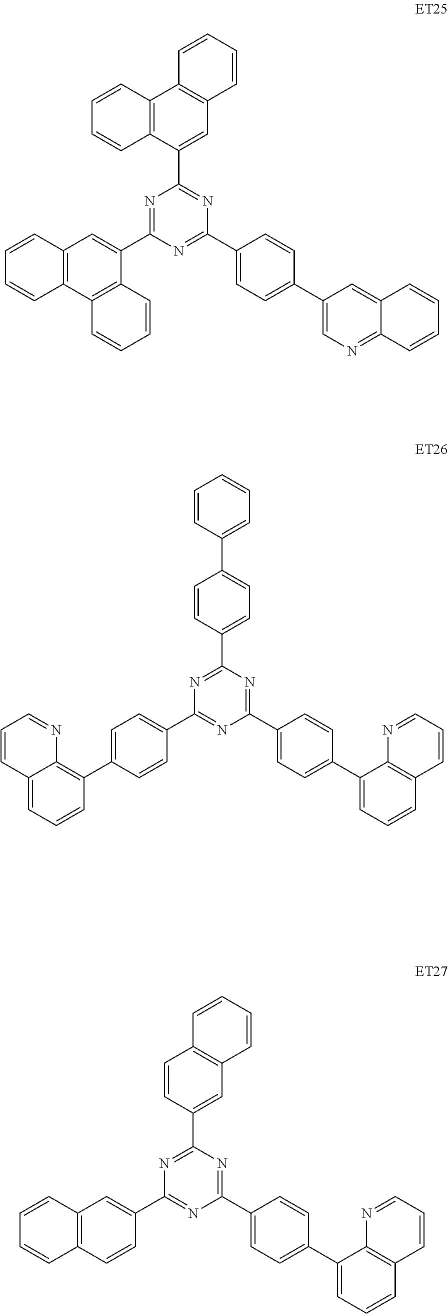 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

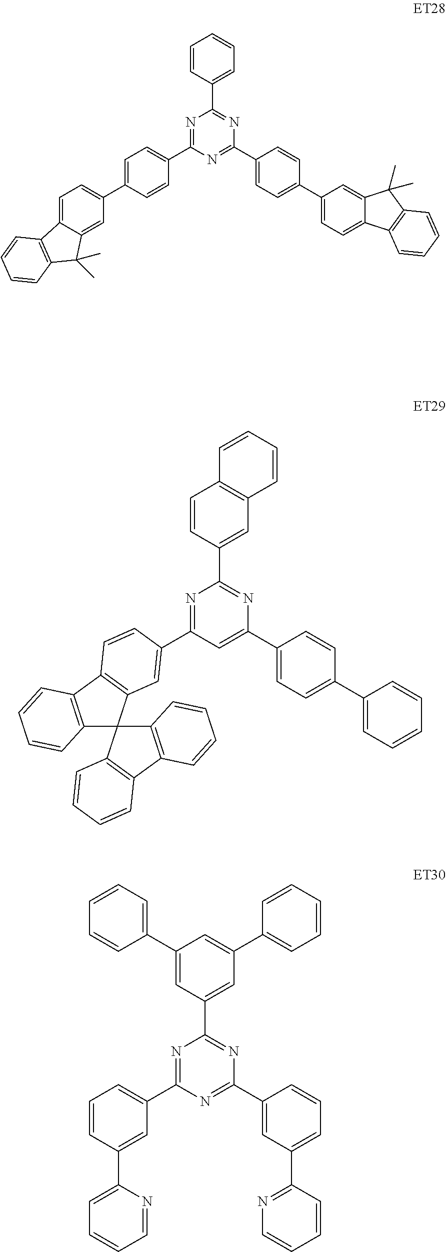 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

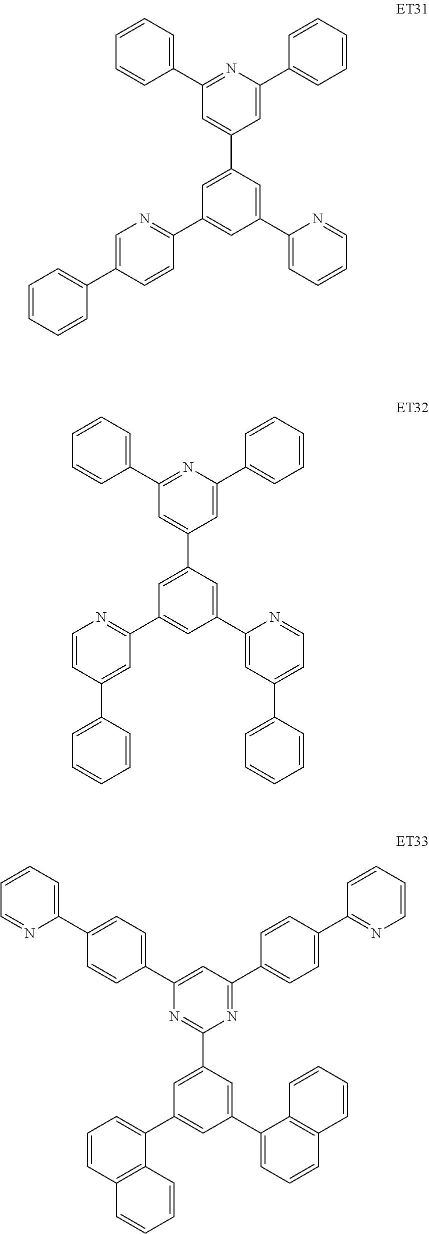 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

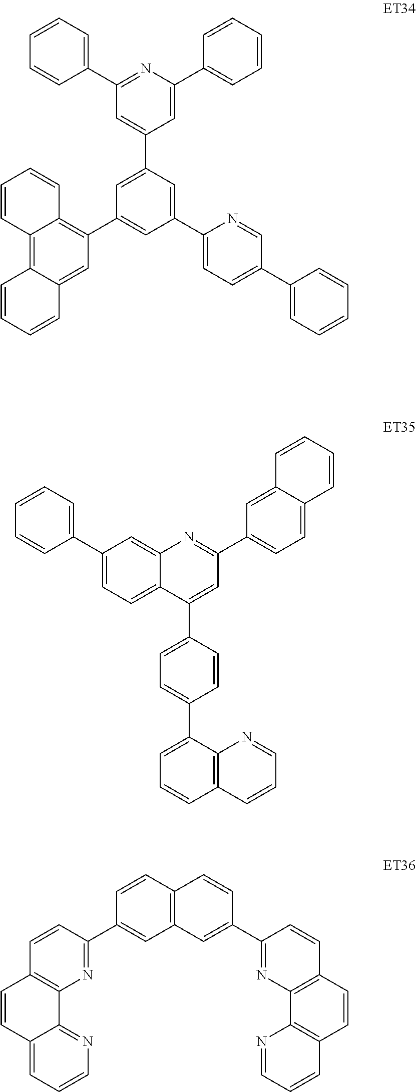 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In one or more embodiments, the electron transport region ETR may include a metal halide such as LiF, NaCl, CsF, RbCl, RbI, CuI, and/or KI, a lanthanide metal such as Yb, or a co-depositing material of the metal halide and the lanthanide metal. For example, in one or more embodiments, the electron transport region ETR may include KI:Yb, RbI:Yb, LiF:Yb, and/or the like, as the co-depositing material. In one or more embodiments, the electron transport region ETR may use a metal oxide such as Li₂O and/or BaO, or 8-hydroxy-lithium quinolate (Liq). However, embodiments of the present disclosure are not limited thereto. The electron transport region ETR also may be formed using a mixture material of an electron transport material and an insulating organo metal salt. The insulating organo metal salt may be a material having an energy band gap of about 4 eV or more. For example, the insulting organo metal salt may include, for example, one or more of metal acetates, metal benzoates, metal acetoacetates, metal acetylacetonates, or metal stearates.

In one or more embodiments, the electron transport region ETR may include at least one of 2,9-dimethyl-4,7-diphenyl-1,10-phenanthroline (BCP), diphenyl (4-(triphenylsilyl)phenyl) phosphine oxide (TSPO1), or 4,7-diphenyl-1,10-phenanthroline (Bphen) in addition to one or more of the aforementioned materials. However, embodiments of the present disclosure are not limited thereto.

The electron transport region ETR may include one or more of the compounds of the electron transport region in at least one selected from among an electron injection layer EIL, an electron transport layer ETL, and a hole blocking layer HBL.

If (e.g., when) the electron transport region ETR includes an electron transport layer ETL, a thickness of the electron transport layer ETL may be from about 100 Å to about 1,000 Å, for example, from about 150 Å to about 500 Å. If (e.g., when) the thickness of the electron transport layer ETL satisfies the above-described range, satisfactory electron transport properties may be obtained without substantial increase of a driving voltage. If (e.g., when) the electron transport region ETR includes an electron injection layer EIL, a thickness of the electron injection layer EIL may be from about 1 Å to about 100 Å, or from about 3 Å to about 90 Å. If (e.g., when) the thickness of the electron injection layer EIL satisfies the above described range, satisfactory electron injection properties may be obtained without inducing a substantial increase in driving voltage.

The second electrode EL2 may be provided on the electron transport region ETR. The second electrode EL2 may be a common electrode. The second electrode EL2 may be a cathode or an anode, but embodiments of the present disclosure are not limited thereto. For example, if (e.g., when) the first electrode EL1 is an anode, the second cathode EL2 may be a cathode, and if (e.g., when) the first electrode EL1 is a cathode, the second electrode EL2 may be an anode. The second electrode EL2 may include at least one selected among Ag, Mg, Cu, Al, Pt, Pd, Au, Ni, Nd, Ir, Cr, Li, Ca, LiF, Mo, Ti, W, In, Sn, and Zn, a (e.g., any suitable) compound of two or more selected therefrom, a (e.g., any suitable) mixture of two or more selected therefrom, or an oxide thereof.

The second electrode EL2 may be a transmissive electrode, a transflective electrode, or a reflective electrode. If (e.g., when) the second electrode EL2 is the transmissive electrode, the second electrode EL2 may include a transparent metal oxide, for example, ITO, IZO, ZnO, ITZO, and/or the like.

If (e.g., when) the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca (stacked structure of LiF and Ca), LiF/Al (stacked structure of LiF and Al), Mo, Ti, Yb, W, a (e.g., any suitable) compound thereof, or a (e.g., any suitable) mixture thereof (for example, AgMg, AgYb, or MgAg). In one or more embodiments, the second electrode EL2 may have a multilayered structure including a reflective layer or a transflective layer formed using one or more of the above-described materials and a transparent conductive layer formed using ITO, IZO, ZnO, ITZO, and/or the like. For example, in one or more embodiments, the second electrode EL2 may include one of the aforementioned metal materials, a (e.g., any suitable) combination of two or more metal materials selected therefrom, or an oxide of the aforementioned metal materials.

In one or more embodiments, the second electrode EL2 may be connected with an auxiliary electrode. If (e.g., when) the second electrode EL2 is connected with the auxiliary electrode, the resistance of the second electrode EL2 may decrease.

In one or more embodiments, on the second electrode EL2 in the light-emitting element ED, a capping layer CPL may be further arranged. The capping layer CPL may include a multilayer or a single layer.

In one or more embodiments, the capping layer CPL may be an organic layer or an inorganic layer. For example, if (e.g., when) the capping layer CPL includes an inorganic material, the inorganic material may include an alkali metal compound such as LiF, an alkaline earth metal compound, SiON, SiNₓ, SiO_{y}, and/or the like.

In one or more embodiments, if (e.g., when) the capping layer CPL includes an organic material, the organic material may include 2,2′-dimethyl-N,N′-di-[(1-naphthyl)-N, N′-diphenyl]-1,1′-biphenyl-4,4′-diamine(α-NPD), NPB, TPD, m-MTDATA, Alq₃, CuPc, N4,N4,N4′,N4′-tetra (biphenyl-4-yl)biphenyl-4,4′-diamine (TPD15), 4,4′,4″-tris(carbazol-9-yl)triphenylamine (TCTA), and/or the like, or may include an epoxy resin or an acrylate such as methacrylate. In one or more embodiments, the capping layer CPL may include at least one selected from among Compounds P1 to P5, but embodiments of the present disclosure are not limited thereto.

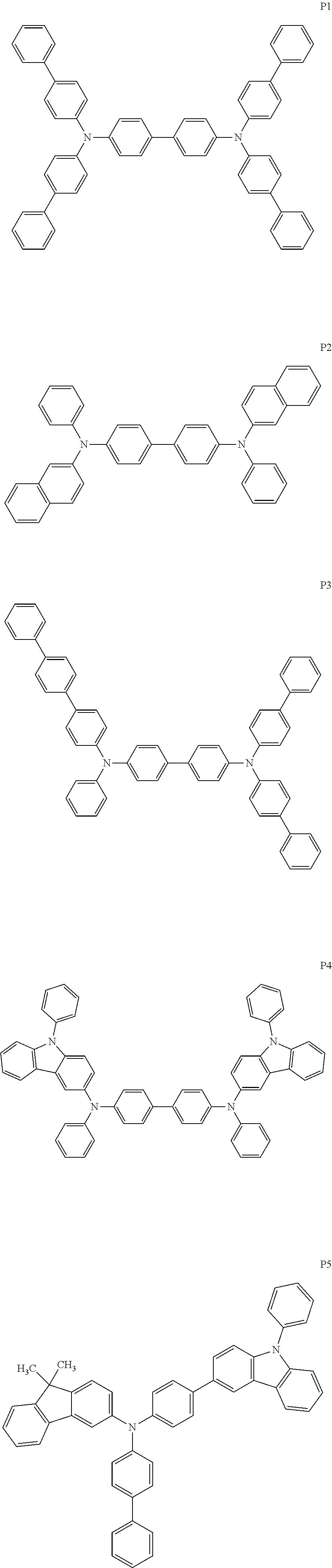 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In one or more embodiments, the refractive index of the capping layer CPL may be about 1.6 or more. For example, in one or more embodiments, the refractive index of the capping layer CPL with respect to light in a wavelength range of about 550 nm to about 660 nm may be about 1.6 or more.

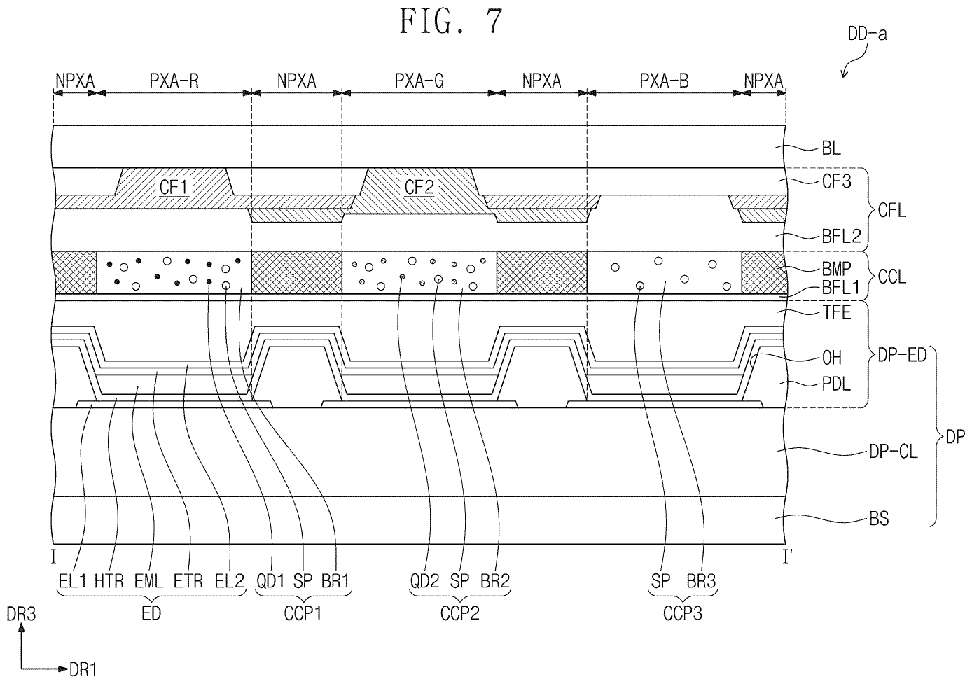

FIG. 7 to FIG. 10 are each a cross-sectional view of a display apparatus according to one or more embodiments of the present disclosure. In the explanation on the display apparatuses of embodiments referring to FIG. 7 to FIG. 10, the overlapping parts with the explanation on FIG. 1 to FIG. 6 will not be explained again, and only different features will be explained chiefly.

Referring to FIG. 7, the display apparatus DD-a according to one or more embodiments may include a display panel DP including a display device layer DP-ED, a light controlling layer CCL arranged on the display panel DP, and a color filter layer CFL.

In one or more embodiments, as shown in FIG. 7, the display panel DP may include a base layer BS, a circuit layer DP-CL provided on the base layer BS, and the display device layer DP-ED, and the display device layer DP-ED may include a light-emitting element ED.

The light-emitting element ED may include a first electrode EL1, a hole transport region HTR arranged on the first electrode EL1, a light-emitting layer EML arranged on the hole transport region HTR, an electron transport region ETR arranged on the light-emitting layer EML, and a second electrode EL2 arranged on the electron transport region ETR. In one or more embodiments, the same structure as any one of the light-emitting elements of FIG. 3 to FIG. 6 may be applied to the structure of the light-emitting element ED shown in FIG. 7.

The hole transport region HTR of the light-emitting element ED included in the display apparatus DD-a according to one or more embodiments may include the amine compound of one or more embodiments described above.

Referring to FIG. 7, the light-emitting layer EML may be arranged in an opening part OH defined in a pixel definition layer PDL. For example, the light-emitting layer EML divided by the pixel definition layer PDL and correspondingly provided to each of luminous areas PXA-R, PXA-G, and PXA-B may be to emit light in substantially the same wavelength region. In the display apparatus DD-a of one or more embodiments, the light-emitting layer EML may be to emit blue light. In one or more embodiments, the light-emitting layer EML may be provided as a common layer across all luminous areas PXA-R, PXA-G, and PXA-B.

The light controlling layer CCL may be arranged on the display panel DP. The light controlling layer CCL may include a light converter. The light converter may be a quantum dot or a phosphor. The light converter may transform the wavelength of light provided and then emit the transformed light. For example, the light controlling layer CCL may be a layer including a quantum dot or a layer including a phosphor.

The light controlling layer CCL may include multiple light controlling parts CCP1, CCP2, and CCP3. The light controlling parts CCP1, CCP2, and CCP3 may be separated from one another.

Referring to FIG. 7, a partition pattern BMP may be arranged between the separated light controlling parts CCP1, CCP2, and CCP3, but embodiments of the present disclosure are not limited thereto. In FIG. 7, the partition pattern BMP is shown not to be overlapped with the light controlling parts CCP1, CCP2, and CCP3, but, in one or more embodiments, at least a portion of the edges of the light controlling parts CCP1, CCP2, and CCP3 may be overlapped with the partition pattern BMP.

The light controlling layer CCL may include a first light controlling part CCP1 including a first quantum dot QD1 converting first color light provided from the light-emitting element ED into second color light, a second light controlling part CCP2 including a second quantum dot QD2 converting the first color light into third color light, and a third light controlling part CCP3 transmitting the first color light.

In one or more embodiments, the first light controlling part CCP1 may provide red light which is the second color light, and the second light controlling part CCP2 may provide green light which is the third color light. The third color controlling part CCP3 may be to transmit and provide blue light which is the first color light provided from the light-emitting element ED. For example, in one or more embodiments, the first quantum dot QD1 may be a red quantum dot to emit red light, and the second quantum dot QD2 may be a green quantum dot to emit green light. On the quantum dots QD1 and QD2, the same contents as those described above on quantum dots may be applied.

In one or more embodiments, the light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include (e.g., may exclude) a quantum dot but include the scatterer SP.

The scatterer SP may be an inorganic particle. For example, the scatterer SP may include at least one selected from among TiO₂, ZnO, Al₂O₃, SiO₂, and hollow silica. In one or more embodiments, the scatterer SP may include at least one selected from among TiO₂, ZnO, Al₂O₃, SiO₂, and hollow silica, or may be a mixture of two or more materials selected among TiO₂, ZnO, Al₂O₃, SiO₂, and hollow silica.

The first light controlling part CCP1, the second light controlling part CCP2, and the third light controlling part CCP3 may respectively include base resins BR1, BR2, and BR3 in which the quantum dots QD1 and QD2 and the scatterer SP are dispersed accordingly. In one or more embodiments, the first light controlling part CCP1 may include the first quantum dot QD1 and the scatterer SP dispersed in a first base resin BR1, the second light controlling part CCP2 may include the second quantum dot QD2 and the scatterer SP dispersed in a second base resin BR2, and the third light controlling part CCP3 may include the scatterer particle SP dispersed in a third base resin BR3.

The base resins BR1, BR2, and BR3 are media in which the quantum dots QD1 and QD2 and the scatterer SP are dispersed accordingly, and may each be composed of one or more suitable resin compositions, which may be generally referred to as a binder. For example, the base resins BR1, BR2, and BR3 may each independently be an acrylic-based resin, a urethane-based resin, a silicone-based resin, an epoxy-based resin, and/or the like. The base resins BR1, BR2, and BR3 may each be transparent resins. In one or more embodiments, the first base resin BR1, the second base resin BR2, and the third base resin BR3 may be the same or different from one another.

In one or more embodiments, the light controlling layer CCL may include a barrier layer BFL1. The barrier layer BFL1 may play the role of blocking the penetration of moisture and/or oxygen (hereinafter, will be referred to as “humidity/oxygen”). The barrier layer BFL1 may block the exposure of the light controlling parts CCP1, CCP2, and CCP3 to humidity/oxygen. In one or more embodiments, the barrier layer BFL1 may cover the light controlling parts CCP1, CCP2, and CCP3. In one or more embodiments, a color filter layer CFL, which will be explained later, may include a barrier layer BFL2 arranged on the light controlling parts CCP1, CCP2, and CCP3.

The barrier layers BFL1 and BFL2 may each include at least one inorganic layer. For example, in one or more embodiments, the barrier layers BFL1 and BFL2 may each be formed by including an inorganic material. For example, the barrier layers BFL1 and BFL2 may be each independently formed by including silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, or a metal thin film securing light transmittance. In one or more embodiments, the barrier layers BFL1 and BFL2 may each independently further include an organic layer. The barrier layers BFL1 and BFL2 may each independently be composed of a single layer of multiple layers.

In the display apparatus DD-a of one or more embodiments, the color filter layer CFL may be arranged on the light controlling layer CCL. For example, in one or more embodiments, the color filter layer CFL may be arranged directly on the light controlling layer CCL. In these embodiments, the barrier layer BFL2 may not be provided.

The color filter layer CFL may include filters CF1, CF2, and CF3. The first to third filters CF1, CF2, and CF3 may be arranged corresponding to a red luminous area PXA-R, a green luminous area PXA-G, and a blue luminous area PXA-B, respectively.

The color filter layer CFL may include a first filter CF1 configured to transmit the second color light, a second filter CF2 configured to transmit the third color light, and a third filter CF3 configured to transmit the first color light. For example, in one or more embodiments, the first filter CF1 may be a red filter, the second filter CF2 may be a green filter, and the third filter CF3 may be a blue filter. Each of the filters CF1, CF2 and CF3 may include a polymer photosensitive resin and a pigment and/or a dye. For example, the first filter CF1 may include a red pigment and/or a red dye, the second filter CF2 may include a green pigment and/or a green dye, and the third filter CF3 may include a blue pigment and/or a blue dye.

Embodiments of the present disclosure are not limited thereto, for example, the third filter CF3 may not include (e.g., may exclude) any pigment or any dye. The third filter CF3 may include a polymer photosensitive resin and not include a pigment or a dye. The third filter CF3 may be transparent. The third filter CF3 may be formed using a transparent photosensitive resin.

In one or more embodiments, the first filter CF1 and the second filter CF2 may each be a yellow filter. The first filter CF1 and the second filter CF2 may be provided in one body. The first to third filters CF1, CF2, and CF3 may be arranged corresponding to the red luminous area PXA-R, the green luminous area PXA-G, and the blue luminous area PXA-B, respectively.

In one or more embodiments, the color filter layer CFL may further include a light blocking part. The color filter layer CFL may include a light blocking part arranged at the boundaries between adjacent filters CF1, CF2, and CF3. The light blocking part may be a black matrix. The light blocking part may be formed by including an organic light blocking material and/or an inorganic light blocking material, including a black pigment and/or a black dye. The light blocking part may divide the boundaries among adjacent filters CF1, CF2, and CF3. In one or more embodiments, the light blocking part may be formed as a blue filter.

On the color filter layer CFL, a base substrate BL may be arranged. The base substrate BL may be a member providing a base surface on which the color filter layer CFL, the light controlling layer CCL, and/or the like are arranged. The base substrate BL may be a glass substrate, a metal substrate, a plastic substrate, and/or the like. However, embodiments of the present disclosure are not limited thereto, and the base substrate BL may be an inorganic layer, an organic layer, or a composite material layer. In one or more embodiments, the base substrate BL may not be provided.

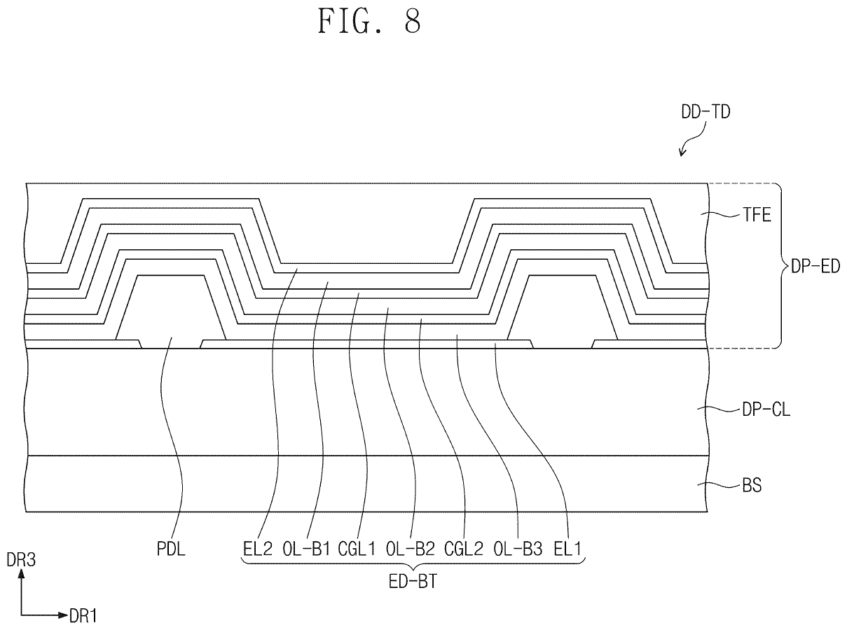

FIG. 8 is a cross-sectional view showing a part of a display apparatus according to one or more embodiments. In a display device DD-TD of one or more embodiments, the light-emitting element ED-BT may include multiple light emitting structures OL-B1, OL-B2, and OL-B3. The light-emitting element ED-BT may include oppositely arranged first electrode EL1 and second electrode EL2, and the multiple light emitting structures OL-B1, OL-B2, and OL-B3 stacked in order in a thickness direction and provided between the first electrode EL1 and the second electrode EL2. Each of the light emitting structures OL-B1, OL-B2, and OL-B3 may include a light-emitting layer EML (FIG. 7), and a hole transport region HTR and an electron transport region ETR, arranged with the light-emitting layer (FIG. 7) therebetween.

For example, the light-emitting element ED-BT included in the display device DD-TD of one or more embodiments may be a light-emitting element of a tandem structure including multiple light-emitting layers.

In one or more embodiments, as shown in FIG. 8, light emitted from the light emitting structures OL-B1, OL-B2, and OL-B3 may be all blue light. However, embodiments of the present disclosure are not limited thereto, for example, in one or more embodiments, the wavelength regions of light emitted from the light emitting structures OL-B1, OL-B2, and OL-B3 may be different from one another. For example, in one or more embodiments, the light-emitting element ED-BT including the multiple light emitting structures OL-B1, OL-B2, and OL-B3 each emitting light in different wavelength regions may be to emit white light (e.g., combined white light).

Between neighboring light emitting structures OL-B1, OL-B2, and OL-B3, charge generating layers CGL1 and CGL2 may be respectively arranged. The charge generating layers CGL1 and CGL2 may independently include a p-type or kind charge (e.g., P-charge) generating layer and/or an n-type or kind charge (e.g., N-charge) generating layer.

At least one selected from among the light-emitting structures OL-B1, OL-B2, and OL-B3 included in the display device DD-TD according to one or more embodiments may contain the above-described amine compound according to one or more embodiments.

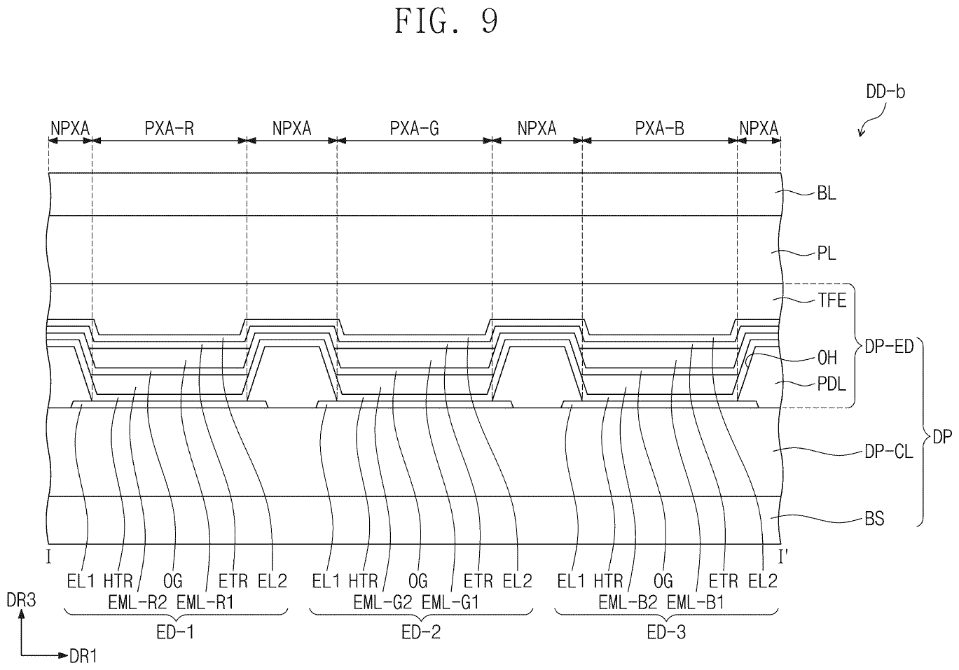

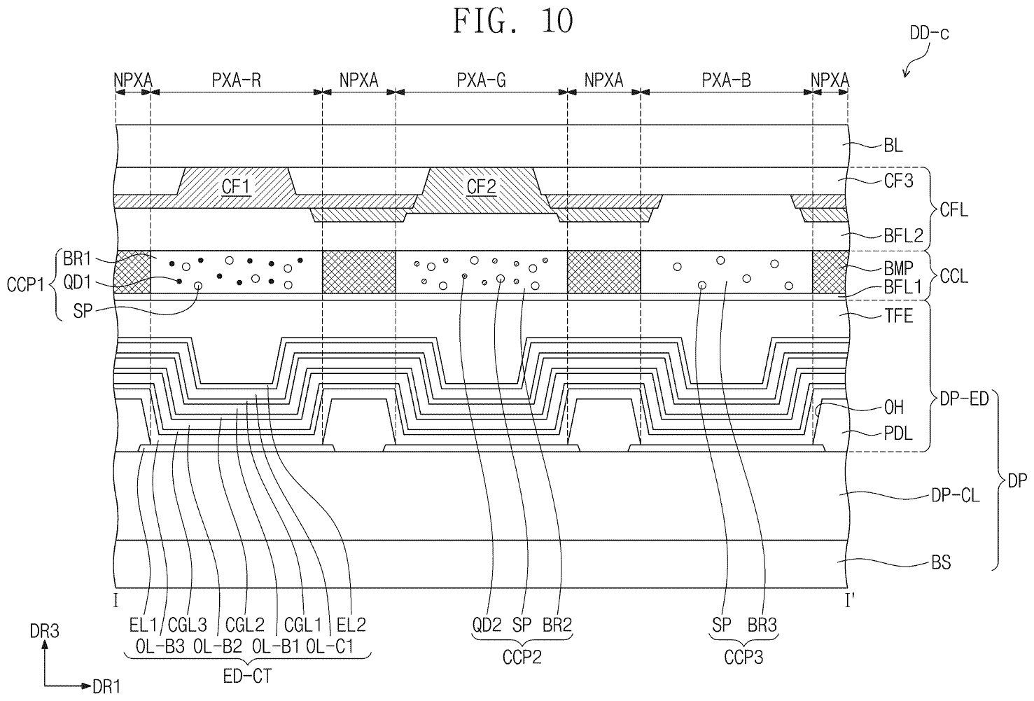

Referring to FIG. 9, a display apparatus DD-b according to one or more embodiments may include light-emitting elements ED-1, ED-2, and ED-3, each formed by stacking two light-emitting layers. Compared to the display device DD shown in FIG. 2, the display apparatus DD-b shown in FIG. 9 is different in that first to third light-emitting elements ED-1, ED-2, and ED-3 each include two light-emitting layers stacked in a thickness direction. In each of the first to third light-emitting elements ED-1, ED-2, and ED-3, two light-emitting layers may be to emit light in substantially the same wavelength region.

In one or more embodiments, the first light-emitting element ED-1 may include a first red light-emitting layer EML-R1 and a second red light-emitting layer EML-R2. The second light-emitting element ED-2 may include a first green light-emitting layer EML-G1 and a second green light-emitting layer EML-G2. In addition, the third light-emitting element ED-3 may include a first blue light-emitting layer EML-B1 and a second blue light-emitting layer EML-B2. Between the first red light-emitting layer EML-R1 and the second red light-emitting layer EML-R2, between the first green light-emitting layer EML-G1 and the second green light-emitting layer EML-G2, and between the first blue light-emitting layer EML-B1 and the second blue light-emitting layer EML-B2, an emission auxiliary part OG may be arranged.

The emission auxiliary part OG may include a single layer or a multilayer. The emission auxiliary part OG may include a charge generating layer. In one or more embodiments, the emission auxiliary part OG may include an electron transport region, a charge generating layer, and a hole transport region stacked in order (e.g., in the stated order). The emission auxiliary part OG may be provided as a common layer across all of the first to third light-emitting elements ED-1, ED-2, and ED-3. However, embodiments of the present disclosure are not limited thereto, and the emission auxiliary part OG may be patterned and provided in an opening part OH defined in a pixel definition layer PDL.

The first red light-emitting layer EML-R1, the first green light-emitting layer EML-G1, and the first blue light-emitting layer EML-B1 may each be arranged between the electron transport region ETR and the emission auxiliary part OG. The second red light-emitting layer EML-R2, the second green light-emitting layer EML-G2, and the second blue light-emitting layer EML-B2 may each be arranged between the emission auxiliary part OG and the hole transport region HTR.

For example, in one or more embodiments, the first light-emitting element ED-1 may include a first electrode EL1, a hole transport region HTR, a second red light-emitting layer EML-R2, an emission auxiliary part OG, a first red light-emitting layer EML-R1, an electron transport region ETR, and a second electrode EL2, stacked in order (e.g., in the stated order). The second light-emitting element ED-2 may include a first electrode EL1, a hole transport region HTR, a second green light-emitting layer EML-G2, an emission auxiliary part OG, a first green light-emitting layer EML-G1, an electron transport region ETR, and a second electrode EL2, stacked in order (e.g., in the stated order). The third light-emitting element ED-3 may include a first electrode EL1, a hole transport region HTR, a second blue light-emitting layer EML-B2, an emission auxiliary part OG, a first blue light-emitting layer EML-B1, an electron transport region ETR, and a second electrode EL2, stacked in order (e.g., in the stated order).

In one or more embodiments, an optical auxiliary layer PL may be arranged on the display device layer DP-ED. The optical auxiliary layer PL may include a polarization layer. The optical auxiliary layer PL may be arranged on the display panel DP and may control reflected light at the display panel DP by external light. In one or more embodiments, the optical auxiliary layer PL may not be provided in the display apparatus.

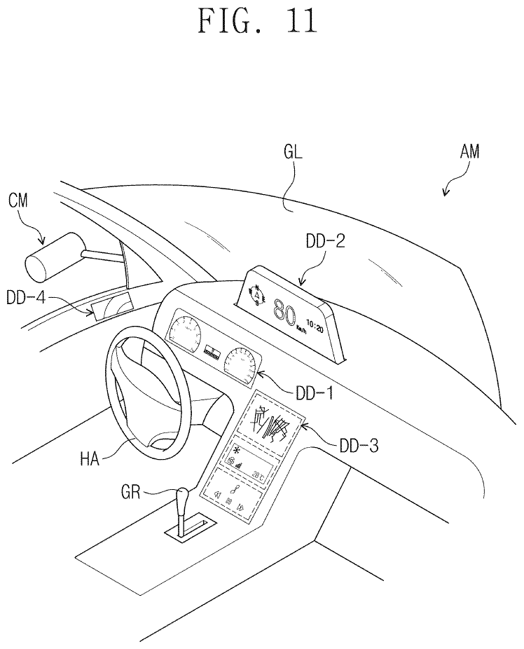

Different from FIG. 8 and FIG. 9, a display apparatus DD-c in FIG. 10 is shown to include four light emitting structures OL-B1, OL-B2, OL-B3, and OL-C1. A light-emitting element ED-CT may include oppositely arranged first electrode EL1 and second electrode EL2, and first to fourth light emitting structures OL-B1, OL-B2, OL-B3, and OL-C1 stacked in a thickness direction between the first electrode EL1 and the second electrode EL2. The third light emitting structures OL-B3, the second light emitting structures OL-B2, the first light emitting structures OL-B1, and the fourth light emitting structures OL-C1 are stacked in order (e.g., in the stated order) in the thickness direction. Between the first to fourth light emitting structures OL-B1, OL-B2, OL-B3, and OL-C1, charge generating layers CGL1, CGL2, and CGL3 may each be separately arranged. For example, A first charge generating layer CGL1 is arranged between the first light emitting structures OL-B1 and the fourth light emitting structures OL-C1. A second charge generating layer CGL2 is arranged between the first light emitting structures OL-B1 and the second light emitting structures OL-B2. A third charge generating layer CGL3 is arranged between the second light emitting structures OL-B2 and the third light emitting structures OL-B3.

In one or more embodiments, among the four light emitting structures, the first to third light emitting structures OL-B1, OL-B2, and OL-B3 may be to emit blue light, and the fourth light emitting structure OL-C1 may be to emit green light. However, embodiments of the present disclosure are not limited thereto, and the first to fourth light emitting structures OL-B1, OL-B2, OL-B3, and OL-C1 may each be to emit different wavelengths of light.

The charge generating layers CGL1, CGL2, and CGL3 arranged among neighboring light emitting structures OL-B1, OL-B2, OL-B3, and OL-C1 may include a p-type or kind charge (e.g., P-charge) generating layer and/or an n-type or kind charge (e.g., N-charge) generating layer.

At least one selected from among the light-emitting structures OL-B1, OL-B2, OL-B3, and OL-C1 included in the display apparatus DD-c according to one or more embodiments may contain the above-described amine compound according to one or more embodiments.

The light-emitting element ED according to one or more embodiments of the disclosure contains the above-described amine compound according to one or more embodiments in at least one functional layer arranged between the first electrode EL1 and the second electrode EL2, and thus may exhibit characteristics of improved luminous efficiency and improved lifespan. The light-emitting element ED according to one or more embodiments may contain the above-described amine compound in at least one selected from among the hole transport region HTR, the light-emitting layer EML, and the electron transport region ETR, arranged between the first electrode EL1 and the second electrode EL2, or in a capping layer CPL. For example, the amine compound according to one or more embodiments may be contained in the hole transport region HTR of the light-emitting element ED according to one or more embodiments, and the light-emitting element according to one or more embodiments may exhibit characteristics of high efficiency and long lifespan.

Because the above-described amine compound according to one or more embodiments includes an amine group, the first substituent, the second substituent, and the third substituent, stability of the amine compound may increase, and hole transportability thereof may be improved. Therefore, the light-emitting element containing the amine compound according to one or more embodiments may have increased lifespan and efficiency. In addition, the light-emitting element according to one or more embodiments contains the amine compound according to one or more embodiments in the hole transport layer, and thus may exhibit characteristics of increased efficiency and lifespan.

FIG. 11 is a diagram showing an automobile AM in which first to fourth display apparatuses DD-1, DD-2, DD-3, and DD-4 are arranged. At least one selected from among the first to fourth display apparatuses DD-1, DD-2, DD-3, and DD-4 may include the same configurations as one of the display apparatuses DD, DD-TD, DD-a, DD-b, and DD-c, explained referring to FIGS. 1, 2, and 7 to 10.

In FIG. 11, a vehicle is shown as an automobile AM, but this is a mere example, for example, the first to fourth display apparatuses DD-1, DD-2, DD-3, and DD-4 may be arranged on other transport apparatuses such as bicycles, motorcycles, trains, ships, and/or airplanes. In addition, at least one selected from among the first to fourth display apparatuses DD-1, DD-2, DD-3, and DD-4 including the same configuration as one of the display apparatuses DD, DD-TD, DD-a, DD-b, and DD-c may be introduced in personal computers, laptop computers, personal digital terminals, game consoles, portable electronic devices, televisions, monitors, external billboards, and/or the like. These are suggested as mere examples, and the display apparatus may be introduced in other electronic devices as long as not deviated from the disclosure.

In one or more embodiments, at least one selected from among the first to fourth display apparatuses DD-1, DD-2, DD-3, and DD-4 may include the light-emitting element ED of one or more embodiments as described with reference to FIGS. 3 to 6. The light-emitting element ED of one or more embodiments may include the amine compound of one or more embodiments. At least one selected from among the first to fourth display apparatuses DD-1, DD-2, DD-3, and DD-4 may include the light-emitting element ED including the amine compound of one or more embodiments, thereby improving display service life.

Referring to FIG. 11, an automobile AM may include a steering wheel HA for the operation of the automobile AM and a gear GR. In addition, the automobile AM may include a front window GL arranged to face a driver.

A first display apparatus DD-1 may be arranged in a first region overlapping with the steering wheel HA. For example, the first display apparatus DD-1 may be a digital cluster displaying first information of the automobile AM. The first information may include a first graduation showing a driving speed of the automobile AM, a second graduation showing the number of revolution of an engine (i.e., revolutions per minute (RPM)), and an image showing a fuel state. The first graduation and the second graduation may each be represented by a digital image.

A second display apparatus DD-2 may be arranged in a second region opposite to (e.g., facing) a driver seat and overlapping with the front window GL. The driver seat may be a seat where the steering wheel HA faces. For example, the second display apparatus DD-2 may be a head up display (HUD) showing second information of the automobile AM. The second display apparatus DD-2 may be optically clear. The second information may include digital numbers showing the driving speed of the automobile AM and may further include information including the current time. In one or more embodiments, the second information of the second display apparatus DD-2 may be projected and displayed on the front window GL.

A third display apparatus DD-3 may be arranged in a third region adjacent to the gear GR. For example, the third display apparatus DD-3 may be a center information display (CID) for the automobile, arranged between the driver seat and a passenger seat and showing third information. The passenger seat may be a seat separated from the driver seat with the gear GR therebetween. The third information may include information on road conditions (for example, navigation information), on playing music or radio, on playing a dynamic image (or image), on the temperature in the automobile AM, and/or the like.

A fourth display apparatus DD-4 may be arranged in a fourth region separated from the steering wheel HA and the gear GR and adjacent to a side of the automobile AM. For example, the fourth display apparatus DD-4 may be a digital wing mirror displaying fourth information. The fourth display apparatus DD-4 may display an external image of the automobile AM, taken by a camera module CM arranged at the outside of the automobile AM. The fourth information may include the external image of the automobile AM.

The above-described first to fourth information is for illustration, and the first to fourth display apparatuses DD-1, DD-2, DD-3, and DD-4 may further display information on the inside and outside of the automobile. The first to fourth information may include different information from one another. However, embodiments of the present disclosure are not limited thereto, and a portion of the first to fourth information may include the same information as one another.

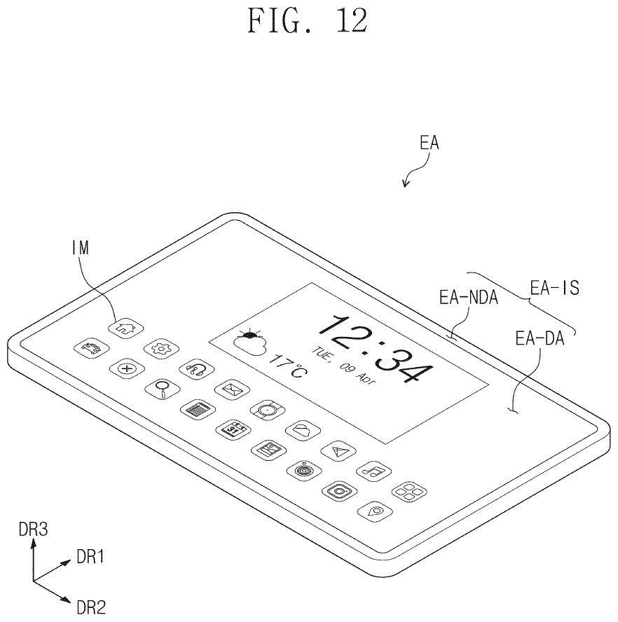

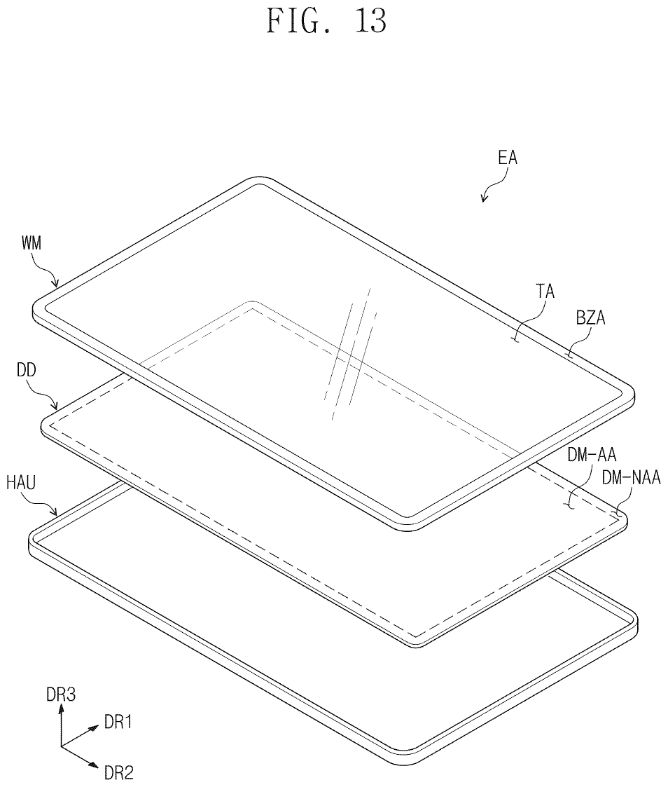

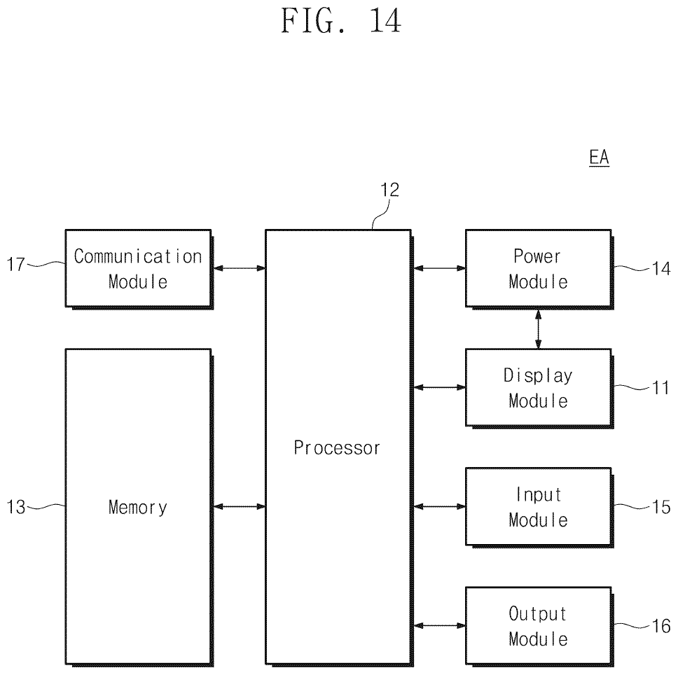

FIG. 12 is a perspective view showing an electronic apparatus of one or more embodiments. FIG. 13 is an exploded perspective view showing an electronic apparatus of one or more embodiments.

An electronic apparatus EA may display an image IM through a display surface EA-IS. The image IM may include a dynamic image as well as a static image. The display surface EA-IS may be parallel to a plane defined by a first direction axis DR1 and a second direction axis DR2. FIG. 12 illustrates the electronic apparatus EA having a flat display surface EA-IS, but embodiments of the present disclosure are not limited thereto. For example, in one or more embodiments, the electronic apparatus EA may include a curved display surface or a three-dimensional display surface. The three-dimensional display surface may include a plurality of display areas indicating different directions from each other.

The display surface EA-IS may include a display area EA-DA and a non-display area EA-NDA. The electronic apparatus EA may display an image IM through the display area EA-DA.

The non-display area EA-NDA may have a predetermined color. The non-display area EA-NDA may be adjacent to the display area EA-DA. The non-display area EA-NDA may surround the display area EA-DA. Accordingly, a shape of the display area EA-DA may be substantially defined by the non-display area EA-NDA. However, FIG. 12 is an example, and the non-display area EA-NDA may be arranged adjacent to only one side of the display area EA-DA or may not be provided.

Referring to FIG. 13, the electronic apparatus EA may include a display device DD. In addition, the electronic apparatus EA may further include a window member WM and a housing HAU.

The window member WM may cover the entire outer side of the electronic apparatus EA. The window member WM may include a transparent area TA and a bezel area BZA. A front surface of the window member WM including the transparent area TA and the bezel area BZA may correspond to a front surface of the electronic apparatus EA. The transparent area TA may correspond to the display area EA-DA of the electronic apparatus EA illustrated in FIG. 12, and the bezel area BZA may correspond to the non-display area EA-NDA of the electronic apparatus EA illustrated in FIG. 12.

The transparent area TA may be an optically transparent area. The bezel area BZA may be an area having a relatively low light transmittance compared to the transparent area TA. The bezel area BZA may have a predetermined color. The bezel area BZA may be adjacent to the transparent area TA and may surround the transparent area TA. The bezel area BZA may define the shape of the transparent area TA. However, embodiments of the present disclosure are not limited thereto, for example, in one or more embodiments, the bezel area BZA may be arranged adjacent to only one side of the transparent area TA, or a portion thereof may not be provided.

The housing HAU may include a material having relatively high rigidity. For example, the housing HAU may include a frame and/or a plate made of glass, plastic, or metal. The frames and/or plates may be provided in multiple pieces. The housing HAU may provide a predetermined receiving space. The display device DD may be accommodated in the receiving space and protected from external impact.

The display device DD may include substantially the same configurations as those of at least one of the display devices DD, DD-TD, DD-a, DD-b, or DD-c of the embodiments described with reference to FIGS. 1, 2, and 7 to 10. The display device DD may include the light emitting element ED described with reference to FIGS. 3 to 6. Accordingly, the electronic apparatus EA including the display device DD according to one or more embodiments may exhibit excellent reliability.

An active area DM-AA and a peripheral area DM-NAA may be defined in the display device DD. The active area DM-AA may overlap the display area EA-DA illustrated in FIG. 12, and the peripheral area DM-NAA may overlap the non-display area EA-NDA illustrated in FIG. 12.

The active area DM-AA may be an area activated according to an electrical signal. The peripheral area DM-NAA may be an area positioned adjacent to at least one side of the active area DM-AA. The active area DM-AA may include the non-light emitting area NPXA and light emitting areas PXA-R, PXA-G and PXA-B, illustrated in FIG. 1. The peripheral area DM-NAA may be arranged to surround the active area DM-AA. However, embodiments of the present disclosure are not limited thereto, for example, in one or more embodiments, some of the peripheral areas DM-NAA may not be provided. A driving circuit or driving wirings for driving the active area DM-AA may be arranged in the peripheral area DM-NAA.

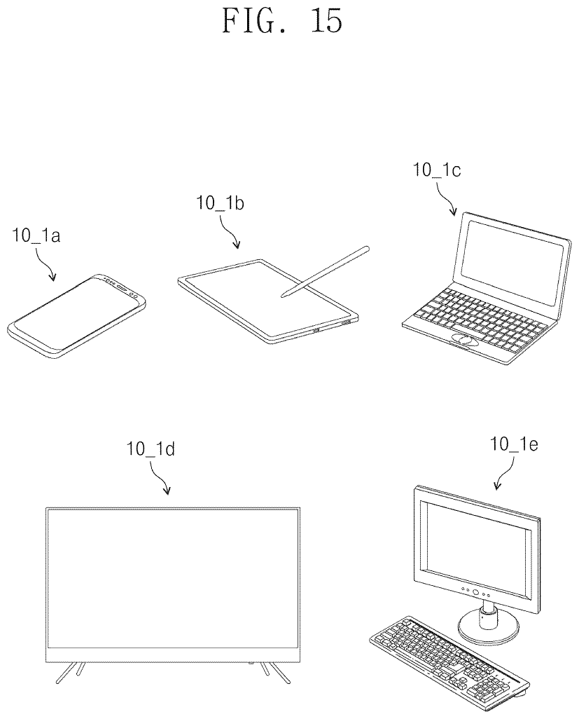

The electronic apparatus EA according to one or more embodiments includes the display device described above, and may further include a module or a device having an additional function, in addition to the display device. FIG. 14 is a block diagram of an electronic apparatus according to one or more embodiments of the present disclosure. Referring to FIG. 14, an electronic apparatus EA according to one or more embodiments may include a display module 11, a processor 12, a memory 13, and a power module 14.

The processor 12 may include at least one of a central processing unit (CPU), an application processor (AP), a graphic processing unit (GPU), a communication processor (CP), an image signal processor (ISP), or a controller. Data information necessary for the operation of the processor 12 or the display module 11 may be stored in the memory 13. If (e.g., when) the processor 12 executes an application stored in the memory 13, an image data signal and/or an input control signal are transmitted to the display module 11, and the display module 11 may process the received signal and output image information through a display screen.

The power module 14 may include a power supply module such as a power adapter or a battery device, and a power conversion module that converts the power supplied by the power supply module to generate power necessary for the operation of the electronic apparatus EA.

The display module 11 may include at least some configurations of the display devices DD, DD-TD, DD-a, DD-b, and/or DD-c, described with reference to FIGS. 1, 2, and 7 to 10. For example, the display module 11 may include a base layer BS, a circuit layer DP-CL, and a display element layer DP-ED among the configurations of the display devices DD, DD-TD, DD-a, DD-b, and/or DD-c, described with reference to FIGS. 1, 2, and 7 to 10. In addition, the display module 11 may further include at least one of an optical layer PP (FIG. 2), a light controlling layer CCL (FIGS. 7 and 10), a color filter layer CFL (FIGS. 7 and 10), and an optical auxiliary layer PL (FIG. 9).

The electronic apparatus EA may further include an input module 15, a non-image output module 16, and/or a communication module 17.

The input module 15 may provide input information to the processor 12 and/or the display module 11. The input module 15 may include various sensor modules as well as physical buttons, a keyboard, and a microphone. Non-limiting examples of sensor module include touch sensors, pressure sensors, distance sensors, position sensors, digitizers, motion recognition sensors, camera sensors, photodetector, photoelectric conversion sensors, temperature sensors, and biosensors such as blood pressure sensors, blood sugar sensors, electrocardiogram sensors, and heart rate sensors.

The non-image output module 16 may receive information other than images transmitted from the processor 12 and provide the information to a user. Non-limiting examples of the non-image output module 16 include an audio module, a haptic module, a light emitting module, and the like, and may include other electronic device-specific functional modules (e.g., a cooling module of a refrigerator, and the like).

The communication module 17 is a module responsible for transmitting and receiving information between the electronic apparatus EA and an external device, and may include a receiving part and a transmitting part. The communication module 17 may include various wireless communication modules such as a mobile communication module, a Wi-Fi module, and a Bluetooth module, or various wired communication modules.

At least one of the configurations of the electronic apparatus EA described above may be included in the above-described display device (at least one of DD, DD-TD, DD-a, DD-b, and DD-c, FIGS. 1, 2, and 7 to 10) according to one or more embodiments. In addition, some of the individual modules functionally included in one module may be included in the display device, and other modules may be provided separately from the display device. For example, the display device may include the display module 11, and the processor 12, the memory 13, and the power module 14 may be provided in the form of other devices within the electronic apparatus EA other than the display device.

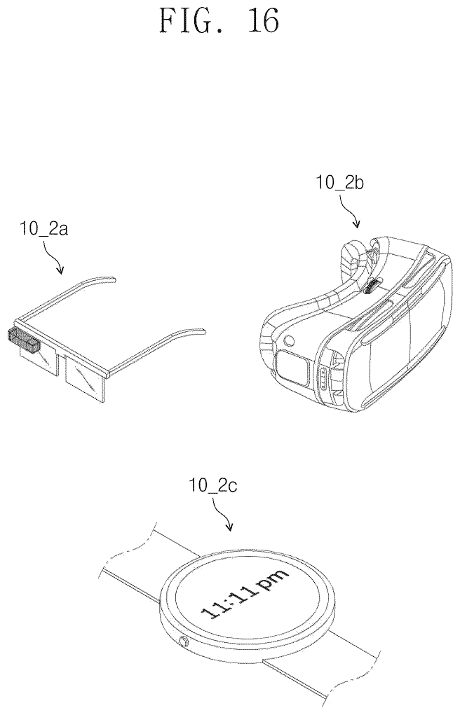

FIG. 15 and FIG. 16 are each a schematic diagram showing electronic apparatuses according to one or more embodiments. Referring to FIG. 15 and FIG. 16, various electronic apparatuses to which a display device (at least one of DD, DD-TD, DD-a, DD-b, or DD-c, FIGS. 1, 2, and 7 to 10) according to one or more embodiments is applied may include not only image display electronic apparatuses such as a smart phone 10_1a, a tablet PC 10_1b, a laptop 10_1c, a TV 10_1d, and a monitor for a desk 10_1e, but also wearable electronic apparatuses including display modules such as smart glasses 10_2a, a head-mounted display 10_2b, and a smart watch 10_2c. However, these are example embodiments, and the electronic apparatus according to one or more embodiments is not limited thereto.

Hereinafter, referring to example embodiments and comparative embodiments, the amine compound according to one or more embodiments of the present disclosure and the light-emitting element according to one or more embodiments will be explained in more detail. In addition, the example embodiments described herein are illustrations to assist the understanding of the disclosure, but the scope of the disclosure is not limited thereto.

### Examples

### 1. Synthesis of Amine Compound

First, the synthetic methods of the amine compounds according to one or more embodiments will be explained by exemplifying the synthetic methods of Compounds 32, 33, 35, 38, 40, 84, 159, 172, 185, 409, and 201. However, the synthetic methods of the amine compounds which will be explained hereinafter are example embodiments, and the synthetic method of the amine compound according to one or more embodiments of the disclosure is not limited to these example embodiments described herein.

The amine compound according to one or more embodiments may be synthesized according to Reaction Scheme 1.

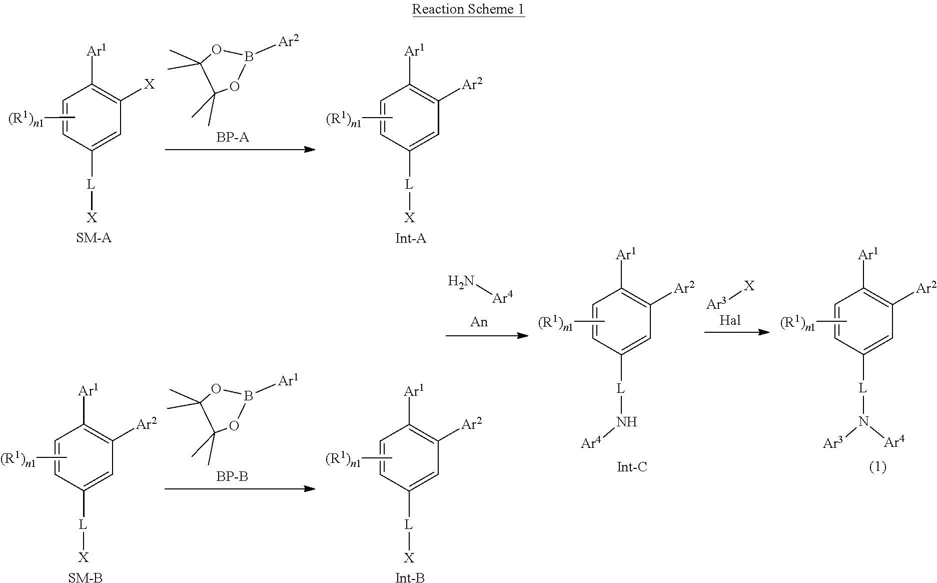

In Reaction Scheme 1, Compound BP-A in which Ar² is a phenanthrenyl group may react with SM-A, which is a start material to obtain Intermediate Int-A. Primary Amine An may react with the Intermediate Int-A to obtain Secondary Amine Intermediate Int-C, and aryl halide Hal may react with the Secondary Amine Intermediate Int-C to obtain an amine compound in which Ar² is a phenanthrenyl group. In addition, in Reaction Scheme 1, Compound BP-B in which Ar¹ is a phenanthrenyl group may react with SM-B, which is a start material to obtain Intermediate Int-B. Primary Amine An may react with the Intermediate Int-B to obtain Secondary Amine Intermediate Int-C, and aryl halide Hal may react with the Secondary Amine Intermediate Int-C to obtain an amine compound in which Ar¹ is a phenanthrenyl group.

In other words, Reaction Scheme 1 describes a process where Compound BP-A, with a phenanthrenyl group Ar², reacts with starting material SM-A to form Intermediate Int-A. This intermediate then reacts with Primary Amine An to produce Secondary Amine Intermediate Int-C, which further reacts with aryl halide Hal to yield an amine compound with a phenanthrenyl group Ar². Similarly, Compound BP-B, with a phenanthrenyl group Ar¹, reacts with starting material SM-B to form Intermediate Int-B. This intermediate also reacts with Primary Amine An to produce Secondary Amine Intermediate Int-C, which then reacts with aryl halide Hal to yield an amine compound with a phenanthrenyl group Ar¹.

### 1-1. Synthesis of Compounds 32, 33, 35, 84, 159, 172, 185, 409, and 201

Example Compounds 32, 33, 35, 84, 159, 172, 185, 409, and 201 may be synthesized with reference to the steps of Reaction Scheme 2 by suitably adjusting reactants.

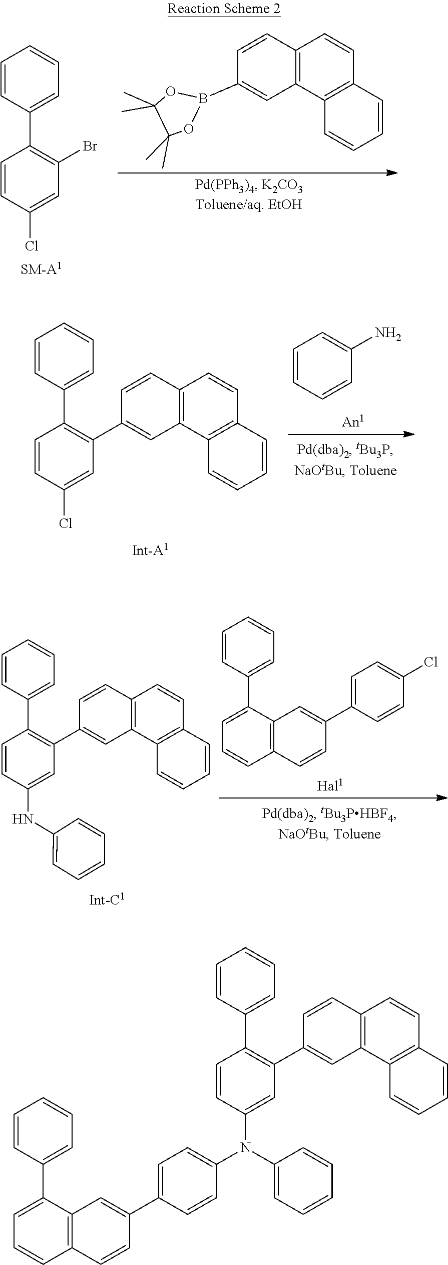

Structures of BP-A², BP-A³, Int-A², Int-A³, Int-C², Int-C³, Hal², Hal³, Hal⁴, Hal⁵, Hal⁶, and Hal⁷, which were used in the following synthetic examples, are described herein in more detail.

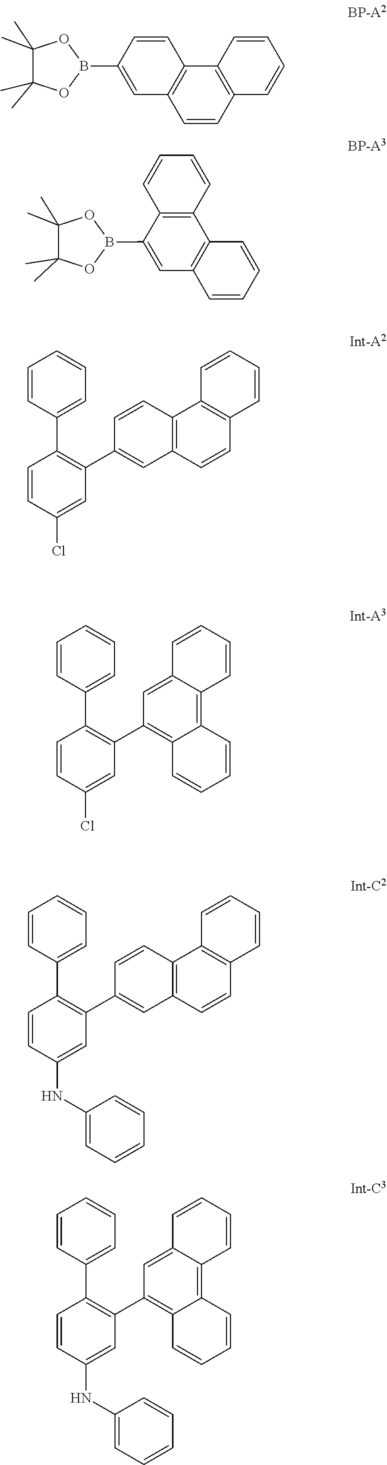

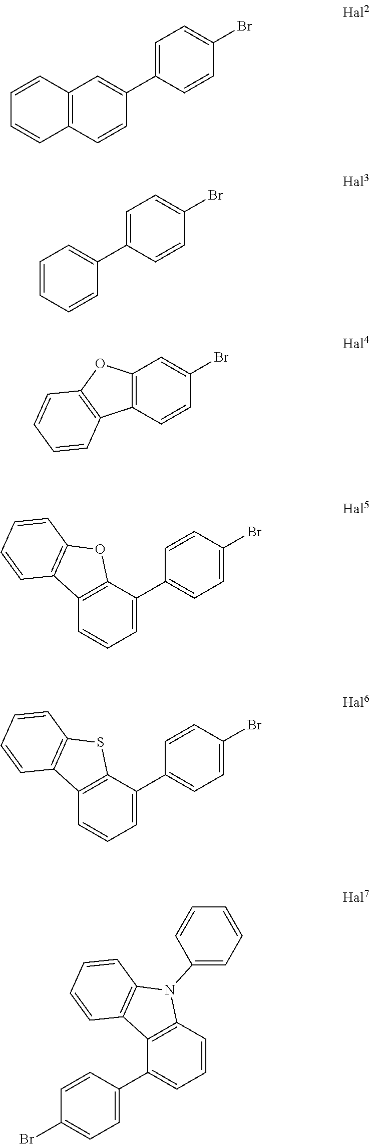 [SMILES: Brc1ccc(-c2ccc3ccccc3c2)cc1 ; Brc1ccc(-c2ccccc2)cc1 ; Brc1ccc2c(c1)oc1ccccc12 ; Brc1ccc(-c2cccc3c2oc2ccccc23)cc1 ; Brc1ccc(-c2cccc3c2sc2ccccc23)cc1 ; Brc1ccc(-c2cccc3c2c2ccccc2n3-c2ccccc2)cc1]

### Synthesis of Intermediate Int-A¹

A mixture of SM-A¹ (16.7 g, 62.5 mmol), BP-A¹ (19.0 g, 62.5 mmol), Tetrakis (triphenylphosphine) palladium (0) (9.39 g, 8.13 mmol), potassium carbonate (25.9 g, 187 mmol), toluene (200 mL), and aq. ethanol (ethanol: water=1:2, 100 mL) was degassed, and then was stirred for about 10 hours under an argon atmosphere while heating at about 80° C. The reaction mixture was cooled, then was extracted with water and toluene (e.g., mixed with water and then extracted with toluene), an organic layer was washed with saturated saline solution, was dried over anhydrous magnesium sulfate, and then was concentrated. The obtained residue was purified by column chromatography (stationary phase: SiO₂, eluent: toluene-hexane) to obtain Int-A¹ (16.5 g, yield of 72%)

### Synthesis of Intermediate Int-A²

Int-A² was obtained in substantially the same manner as Int-A¹, except that BP-A² was used in place of BP-A¹. (yield of 75%)

### Synthesis of Intermediate Int-A³

Int-A³ was obtained in substantially the same manner as Int-A¹, except that BP-A³ was used in place of BP-A¹. (yield of 66%)

### Synthesis of Intermediate Int-C¹

A mixture of Int-A¹ (16.5 g, 45.3 mmol), An¹ (11.8 g, 127 mmol), Bis(dibenzylideneacetone)palladium(0) (2.35 g, 4.08 mmol), tri-(tert-butyl)phosphine (2 M in toluene, 3.63 mL, 7.25 mmol), and sodium tert-butoxide (8.72 g, 90.7 mmol) in toluene was degassed, and then was stirred for about 4 hours under an argon atmosphere while heating at about 90° C. The reaction mixture was cooled, then was filtered with Florisil, and was concentrated. The obtained residue was purified by column chromatography (stationary phase: SiO₂, eluent: toluene-hexane) and crystallized to obtain Int-C¹ (9.95 g, yield of 52%).

### Synthesis of Intermediate Int-C²

Int-C² was obtained in substantially the same manner as Int-C¹, except that Int-A² was used in place of Int-A¹. (yield of 55%)

### Synthesis of Intermediate Int-C³

Int-C³ was obtained in substantially the same manner as Int-C¹, except that Int-A³ was used in place of Int-A¹. (yield of 43%)

### Synthesis of Compound 409

A mixture of Int-C¹ (9.95 g, 23.6 mmol), Hal¹ (7.34 g, 23.6 mmol), Bis (dibenzylideneacetone) palladium (0) (1.76 g, 3.07 mmol), tri-tert-butylphosphonium tetrafluoroborate (3.15 g, 10.9 mmol), sodium tert-butoxide (12.5 g, 130 mmol), and toluene (150 mL) was degassed, and then was stirred for about 9 hours under an argon atmosphere while heating at about 110° C. The reaction mixture was cooled, then was filtered with Florisil, and was concentrated. The obtained residue was purified by column chromatography (stationary phase: SiO₂, eluent: toluene-hexane) to obtain Compound 409 (16.2 g, yield of 98%, Product identified by Fast atom bombardment-Mass spectroscopy (FAB-MS) measurement with an m/z value of 699.3).

In other words, a mixture of Int-C¹ (9.95 g, 23.6 mmol), Hal¹ (7.34 g, 23.6 mmol), Bis(dibenzylideneacetone)palladium(0) (1.76 g, 3.07 mmol), tri-tert-butylphosphonium tetrafluoroborate (3.15 g, 10.9 mmol), sodium tert-butoxide (12.5 g, 130 mmol), and toluene (150 mL) was degassed and stirred under an argon atmosphere at 110° C. for about 9 hours. After cooling, the reaction mixture was filtered using Florisil and concentrated. The resulting residue was purified by column chromatography (SiO₂, toluene-hexane) to obtain Compound 409 (16.2 g, 98% yield).

### Synthesis of Compound 32

Compound 32 was obtained in substantially the same manner as Compound 409, except that Hal² was used in place of Hal¹. (yield of 97%, Product identified by FAB-MS measurement with an m/z value of 623.3)

### Synthesis of Compound 84

Compound 84 was obtained in substantially the same manner as Compound 409, except that Hal³ was used in place of Hal¹. (yield of 99%, Product identified by FAB-MS measurement with an m/z value of 573.3)

### Synthesis of Compound 33

Compound 33 was obtained in substantially the same manner as Compound 409, except that Hal² was used in place of Hal¹ and Int-C² was used in place of Int-C¹. (yield of 97%, Product identified by FAB-MS measurement with an m/z value of 623.3)

### Synthesis of Compound 35

Compound 35 was obtained in substantially the same manner as Compound 409, except that Hal² was used in place of Hal¹ and Int-C³ was used in place of Int-C¹. (yield of 90%, Product identified by FAB-MS measurement with an m/z value of 623.3)

### Synthesis of Compound 201

Compound 201 was obtained in substantially the same manner as Compound 409, except that Hal⁴ was used in place of Hal¹ and Int-C³ was used in place of Int-C¹. (yield of 88%, Product identified by FAB-MS measurement with an m/z value of 587.2)

### Synthesis of Compound 159

Compound 159 was obtained in substantially the same manner as Compound 409, except that Hal⁵ was used in place of Hal¹. (yield of 93%, Product identified by FAB-MS measurement with an m/z value of 663.3)

### Synthesis of Compound 172

Compound 172 was obtained in substantially the same manner as Compound 409, except that Hal⁶ was used in place of Hal¹. (yield of 90%, Product identified by FAB-MS measurement with an m/z value of 679.2)

### Synthesis of Compound 185

Compound 185 was obtained in substantially the same manner as Compound 409, except that Hal⁷ was used in place of Hal¹. (yield of 95%, Product identified by FAB-MS measurement with an m/z value of 738.3)

### 1-2. Synthesis of Compounds 38 and 40

Example Compounds 38 and 40 were synthesized with reference to the steps of Reaction Scheme 3 by suitably adjusting reactants.

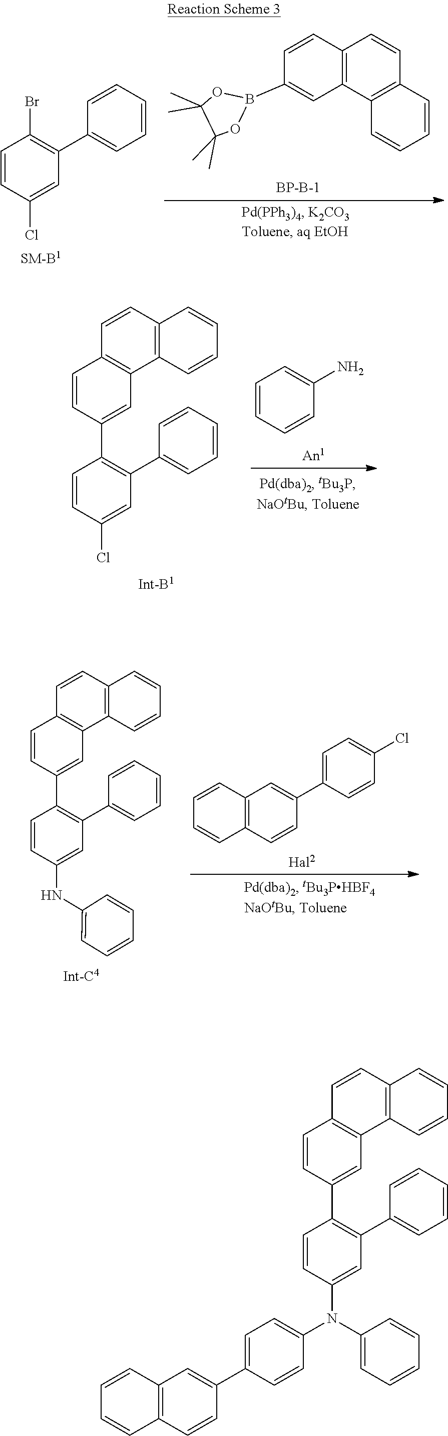

Structures of BP-B², Int-B², and Int-C⁵ used in the following Synthesis Examples are disclosed herein.

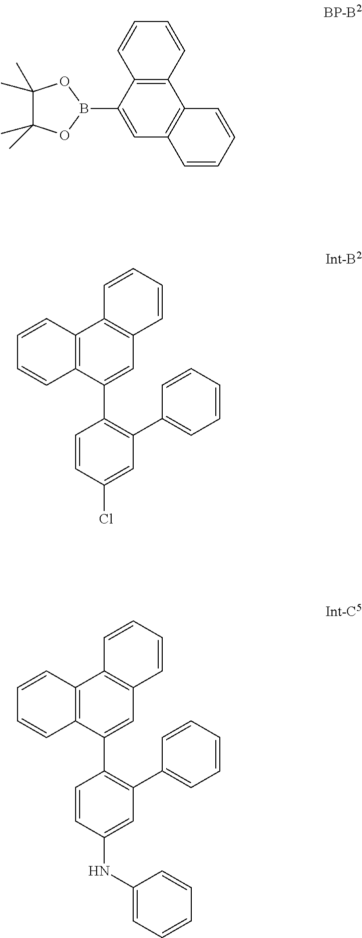 [SMILES: CC1(C)OB(c2cc3ccccc3c3ccccc23)OC1(C)C ; Clc1ccc(-c2cc3ccccc3c3ccccc23)c(-c2ccccc2)c1 ; c1ccc(Nc2ccc(-c3cc4ccccc4c4ccccc34)c(-c3ccccc3)c2)cc1]

### Synthesis of Intermediate Int-B¹

Int-B¹ was obtained in substantially the same manner as Int-A¹, except that SM-B¹ was used in place of SM-A¹ and BP-B¹ was used in place of BP-A¹ in Reaction Scheme 2. (yield of 70%)

### Synthesis of Intermediate Int-B²

Int-B² was obtained in substantially the same manner as Int-B¹, except that BP-B² was used in place of BP-B¹. (yield of 65%)

### Synthesis of Intermediate Int-C⁴

Int-C⁴ was obtained in substantially the same manner as Int-C¹, except that Int-B¹ was used in place of Int-A¹ in Reaction Scheme 2. (yield of 50%)

### Synthesis of Intermediate Int-C⁵

Int-C⁵ was obtained in substantially the same manner as Int-C⁴, except that Int-B² was used in place of Int-B¹. (yield of 42%)

### Synthesis of Compound 38

Compound 38 was obtained in substantially the same manner as Compound 409, except that Hal² was used in place of Hal¹ and Int-C⁴ was used in place of Int-C¹. (yield of 98%, Product identified by FAB-MS measurement with an m/z value of 623.3)

### Synthesis of Compound 40

Compound 40 was obtained in substantially the same manner as Compound 409, except that Hal² was used in place of Hal¹ and Int-C⁵ was used in place of Int-C¹. (yield of 96%, Product identified by FAB-MS measurement with an m/z value of 623.3)

### 2. Manufacture and Evaluation of Light-Emitting Element

A light-emitting element according to one or more embodiments, including the amine compound according to one or more embodiments in a hole transport layer was manufactured by a method described herein. Light-emitting elements according to Example 1 to Example 11 were each manufactured by respectively using the amine compounds of Compounds 32, 84, 33, 38, 40, 35, 201, 159, 172, 409, and 185, which are the above-explained Example Compounds, as a material for the hole transport layer. Light-emitting elements according to Comparative Example 1 to Comparative Example 8 correspond to light-emitting elements manufactured by respectively using Comparative Example Compound C1 to Comparative Example Compound C8 as a material for the hole transport layer.

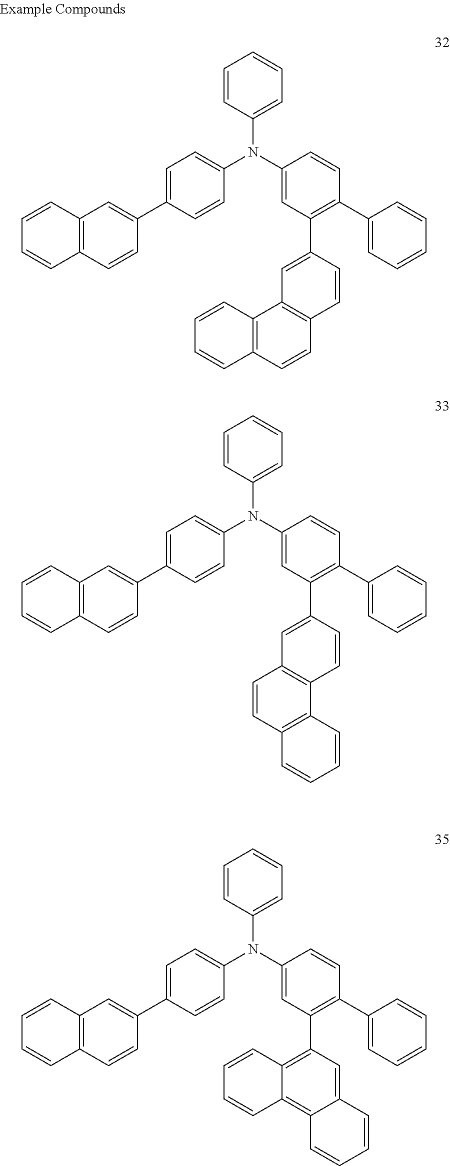 [SMILES: c1ccc(-c2ccc(N(c3ccccc3)c3ccc(-c4ccc5ccccc5c4)cc3)cc2-c2ccc3ccc4ccccc4c3c2)cc1 ; c1ccc(-c2ccc(N(c3ccccc3)c3ccc(-c4ccc5ccccc5c4)cc3)cc2-c2ccc3c(ccc4ccccc43)c2)cc1 ; c1ccc(-c2ccc(N(c3ccccc3)c3ccc(-c4ccc5ccccc5c4)cc3)cc2-c2cc3ccccc3c3ccccc23)cc1]

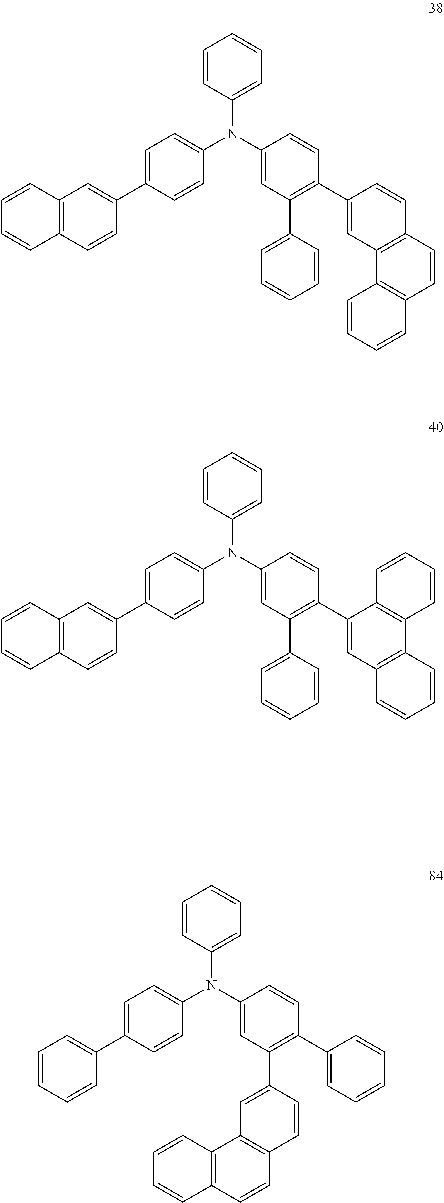 [SMILES: c1ccc(-c2cc(N(c3ccccc3)c3ccc(-c4ccc5ccccc5c4)cc3)ccc2-c2ccc3ccc4ccccc4c3c2)cc1 ; c1ccc(-c2cc(N(c3ccccc3)c3ccc(-c4ccc5ccccc5c4)cc3)ccc2-c2cc3ccccc3c3ccccc23)cc1 ; c1ccc(-c2ccc(N(c3ccccc3)c3ccc(-c4ccccc4)c(-c4ccc5ccc6ccccc6c5c4)c3)cc2)cc1]

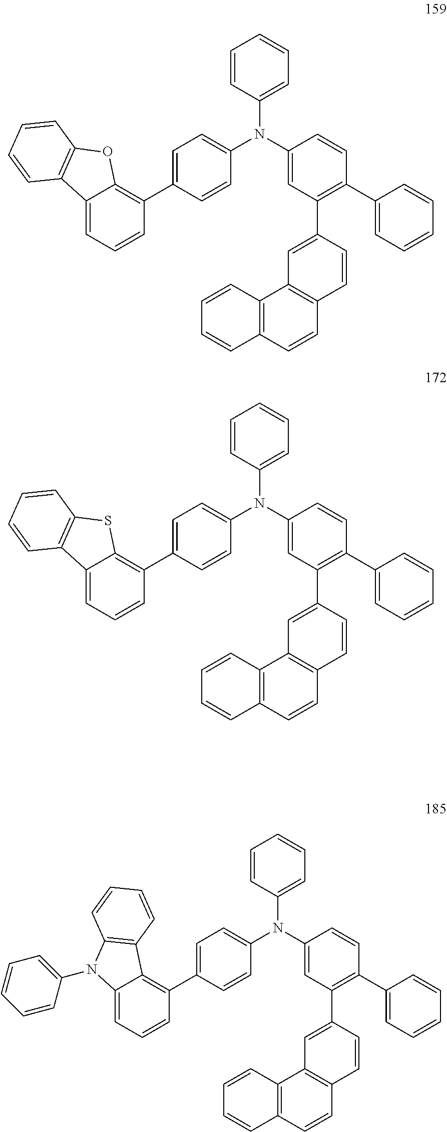 [SMILES: c1ccc(-c2ccc(N(c3ccccc3)c3ccc(-c4cccc5c4oc4ccccc45)cc3)cc2-c2ccc3ccc4ccccc4c3c2)cc1 ; c1ccc(-c2ccc(N(c3ccccc3)c3ccc(-c4cccc5c4sc4ccccc45)cc3)cc2-c2ccc3ccc4ccccc4c3c2)cc1 ; c1ccc(-c2ccc(N(c3ccccc3)c3ccc(-c4cccc5c4c4ccccc4n5-c4ccccc4)cc3)cc2-c2ccc3ccc4ccccc4c3c2)cc1]

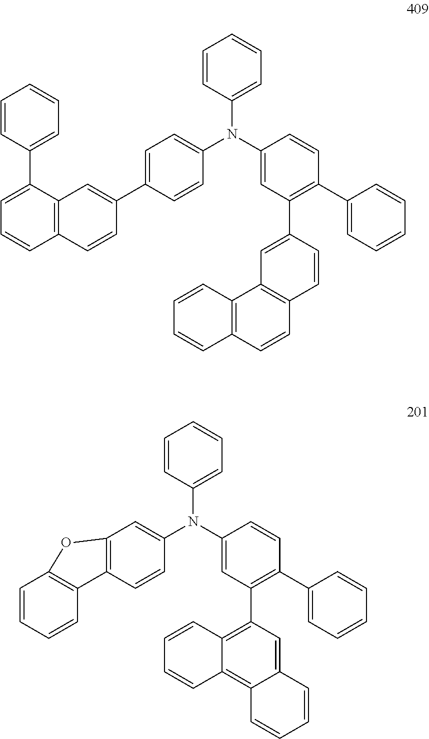 [SMILES: c1ccc(-c2ccc(N(c3ccccc3)c3ccc(-c4ccc5cccc(-c6ccccc6)c5c4)cc3)cc2-c2ccc3ccc4ccccc4c3c2)cc1 ; c1ccc(-c2ccc(N(c3ccccc3)c3ccc4c(c3)oc3ccccc34)cc2-c2cc3ccccc3c3ccccc23)cc1]

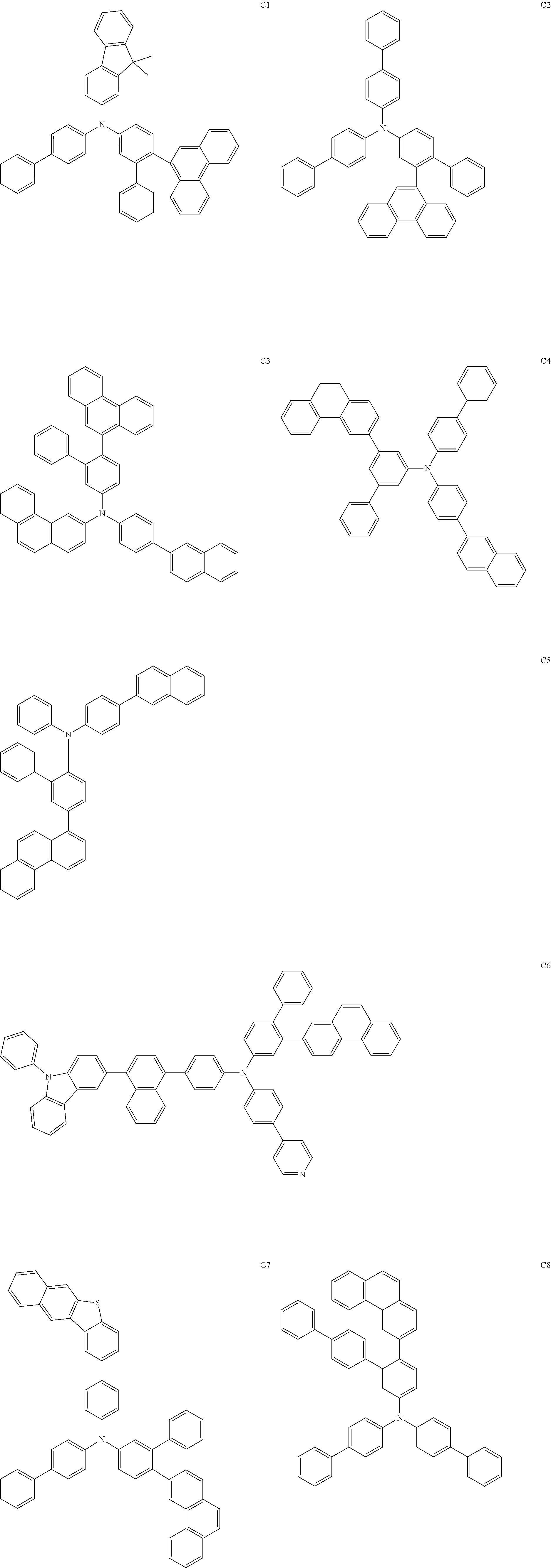

An ITO glass substrate with about 15 Ω/cm² (about 1500 Å) of Corning Co. was cut into a size of 50 mm×50 mm×0.7 mm, washed with isopropyl alcohol and then ultrapure water, cleansed using ultrasonic waves for about 5 minutes each, exposed to UV for about 30 minutes and then treated with ozone. Then, 4,4′,4″-tris{N,-(2-naphthyl)-N-phenylamino}-triphenylamine (2-TNATA) was vacuum deposited to a thickness of about 600 Å to form a hole injection layer. After that, the Example Compound or Comparative Example Compound was vacuum deposited to a thickness of about 300 Å to form a hole transport layer.

On the hole transport layer, a blue fluorescence host of 9,10-di(naphthalen-2-yl) anthracene (ADN) and a fluorescence dopant of 2,5,8,11-tetra-t-butylperylene (TBP) were co-deposited in a ratio (e.g., a weight ratio) of about 97:3 to form an light-emitting layer with a thickness of about 250 Å.

On the light-emitting layer, an electron transport layer was formed to a thickness of about 250 Å using tris(8-hydroxyquinolinato)aluminum (Alq₃), and then, an electron injection layer was formed to a thickness of about 10 Å by depositing LiF. On the electron injection layer, a second electrode was formed to a thickness of about 1000 Å using aluminum (Al).

The compounds in each of functional layers used for the manufacture of the light-emitting element are as follows.

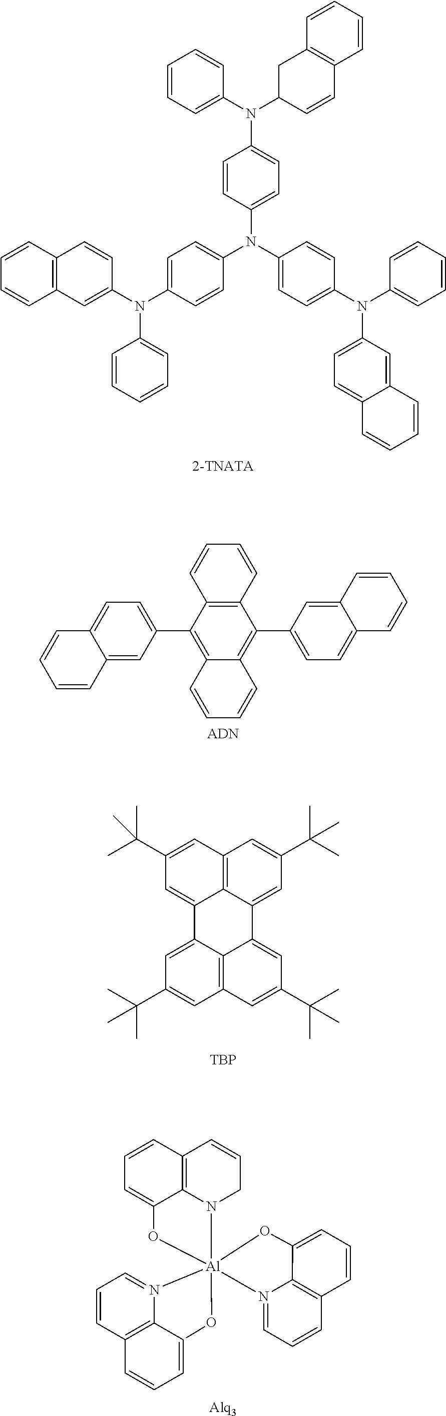

### Evaluation of Light-Emitting Element

The properties of each of the light-emitting elements according to Examples and Comparative Examples were evaluated and the evaluation results are listed in Table 1. In Table 1, luminous efficiency and lifespan of each of the manufactured light-emitting elements are shown. Luminous efficiency shows efficiency values at a current density of about 10 mA/cm², the element lifespan shows luminance half-lifespan values at a current density of about 10 mA/cm². Decrease in purity is expressed as a percentage (%) of the difference in purity of materials, i.e., amine compound, before and after deposition.

The evaluation of the current density and luminous efficiency of each of the light-emitting elements was conducted using a Source Meter of 2400 series, which is a product of Keithley Instruments Co., a luminance and color meter of CS-200, which is a product of Konica Minolta Co., Ltd., and PC Program LabVIEW 8.2 for measurement, which is a product of National Instruments Co., Ltd., in a dark room.

In addition, relative luminous efficiency and element lifespan values of each of the light-emitting elements were listed with respect to 100% of luminous efficiency and element lifespan values of the light-emitting element according to Comparative Example 2.

TABLE 1

| | | Luminous | | Decrease |
|---|---|---|---|---|
| | Hole transport | efficiency | Lifespan | in purity |
| | materials | [%] | [%] | [%] |
| Example 1 | Compound 32 | 120 | 120 | 0.0 |
| Example 2 | Compound 84 | 115 | 115 | 0.0 |
| Example 3 | Compound 33 | 120 | 120 | 0.0 |
| Example 4 | Compound 38 | 120 | 120 | 0.0 |
| Example 5 | Compound 40 | 120 | 115 | 0.0 |
| Example 6 | Compound 35 | 120 | 115 | 0.0 |
| Example 7 | Compound 201 | 120 | 115 | 0.0 |
| Example 8 | Compound 159 | 120 | 120 | 0.0 |
| Example 9 | Compound 172 | 120 | 120 | 0.0 |
| Example 10 | Compound 409 | 120 | 125 | 0.0 |
| Example 11 | Compound 185 | 120 | 115 | 0.0 |
| Comparative | Comparative Example | 100 | 90 | 0.2 |
| Example 1 | Compound C1 | | | |
| Comparative | Comparative Example | 100 | 100 | 0.0 |
| Example 2 | Compound C2 | | | |
| Comparative | Comparative Example | 105 | 80 | 0.2 |
| Example 3 | Compound C3 | | | |
| Comparative | Comparative Example | 100 | 80 | 0.2 |
| Example 4 | Compound C4 | | | |
| Comparative | Comparative Example | 95 | 60 | 0.4 |
| Example 5 | Compound C5 | | | |
| Comparative | Comparative Example | 90 | 60 | 0.2 |
| Example 6 | Compound C6 | | | |
| Comparative | Comparative Example | 95 | 60 | 0.4 |
| Example 7 | Compound C7 | | | |
| Comparative | Comparative Example | 95 | 90 | 0.2 |
| Example 8 | Compound C8 | | | |

Referring to the results in Table 1, it can be seen that each of the light-emitting elements according to Examples, which uses an amine compounds according to one or more embodiments of the disclosure as a material for a hole transport layer, exhibits higher luminous efficiency and longer lifespan than the light-emitting elements according to comparative examples. It can be seen that, compared to Example Compounds, when Comparative Example Compounds are applied to a light-emitting element, the light-emitting element has decreased luminous efficiency and lifespan. For example, referring to Table 1, it can be seen that the light-emitting element using the amine compound according to one or more embodiments of the disclosure has improved element characteristics of luminous efficiency and element lifespan compared to the light-emitting element according to comparative example embodiment.

The amine compound according to one or more embodiments includes a first substituent, a second substituent, and a third substituent of the disclosure, which are connected to a core nitrogen atom of the amine group of the amine compound, and thus may achieve improvements in high efficiency and long lifespan of the light-emitting element.

In the amine compound according to one or more embodiments, represented by Formula 1, the first substituent may include a benzene moiety and a substituted or unsubstituted phenanthrenyl group and an unsubstituted phenyl group, which are each connected to the benzene moiety. In this regard, the substituted or unsubstituted phenanthrenyl group and the unsubstituted phenyl group may be connected to the benzene moiety to be in an ortho relationship with each other. In addition, in the first substituent, any one selected from among the substituted or unsubstituted phenanthrenyl group and the unsubstituted phenyl group may be connected to the benzene moiety at a para position with respect to the nitrogen atom of the amine group, and the other may be connected to the benzene moiety at an ortho position with respect to the one. The amine compound according to one or more embodiments may have excellent or suitable thermal stability and high charge transportability due to introduction of such substituents and specification of a substitution position. Therefore, the amine compound according to one or more embodiments may have improved efficiency and lifespan. Accordingly, the light-emitting element according to one or more embodiments including the amine compound according to one or more embodiments may have improved luminous efficiency and lifespan.

Comparative Example Compound C1 contained in the light-emitting element according to Comparative Example 1 corresponds to a case where one among Ar³ and Ar⁴ is a 9,9-dimethylfluorenyl group in Formula 1 of the disclosure. Like Comparative Example Compound C1, if (e.g., when) any one selected from among Ar³ and Ar⁴ includes a 9,9-dimethylfluorenyl group, hole transport characteristics and thermal stability of molecules may be reduced. Therefore, it can be confirmed that the light-emitting element according to Comparative Example 1 including Comparative Example Compound C1 has reduced luminous efficiency and element lifespan compared to the light-emitting elements according to Examples.

Comparative Example Compound C2 contained in the light-emitting element according to Comparative Example 2 corresponds to a case where Ar² in Formula 1 of the disclosure is a 9-phenanthrenyl group. The 9-phenanthrenyl group, unlike phenanthrenyl groups with other substitution positions, may induce increased steric distortion at the binding site. Therefore, in a compound in which Ar¹ or Ar² is a 9-phenanthrenyl group, a substituent (e.g. a naphthylphenyl group, a dibenzofuranyl group, a dibenzothiophenyl group, a 9-phenylcarbazolyl group, or a 9,9-diphenylfluorenyl group) that may compensate for stability deterioration caused by the introduction of 9-phenanthrenyl substituent, is required and necessary to be introduced, but Comparative Example Compound C2 does not include such a substituent, thereby causing a decrease in thermal stability. Therefore, it is observed that the light-emitting element according to Comparative Example 2 containing Comparative Example Compound C2 has reduced luminous efficiency and element lifespan compared to the light-emitting elements according to Examples.

Compared to this, although containing a 9-phenanthrenyl group, Example Compounds 40, 35, and 201 contains a substituent such as naphthylphenyl, or a dibenzofuranyl group, and thus thermal stability thereof may be improved. Therefore, it can be seen that the light-emitting elements according to Examples 5, 6, and 7 containing Example Compounds 40, 35 and 201, respectively, have improved efficiency and lifespan, compared to the light-emitting element according to Comparative Example 2.

Comparative Example Compound C3 contained in the light-emitting element according to Comparative Example 3 corresponds to a case where one among Ar³ and Ar⁴ in Formula 1 of the disclosure is a phenanthrenyl group. As Comparative Example Compound C3, if (e.g., when), in addition to Ar¹ or Ar², an additional phenanthrenyl group is directly bonded to the nitrogen atom of the amine group, there are effects in that intermolecular interactions increase, and thus a deposition temperature may excessively (or substantially) raise. Therefore, Comparative Example Compound C3 shows a tendency for materials to be easily decomposed during deposition due to the increased deposition temperature, and the light-emitting element according to Comparative Example 3 containing Comparative Example Compound C3 shows reduced luminous efficiency and element lifespan compared to the light-emitting elements according to Examples.

Comparative Example Compounds C4 and C5 contained in the light-emitting elements according to Comparative Examples 4 and 5, respectively, each corresponds to a compound in which a bond relationship between phenanthrene and an unsubstituted phenyl group is different from those of Example Compounds. In Comparative Example Compounds C4 and C5, the phenanthrenyl group and the unsubstituted phenyl group contained in the amine compound are connected in a meta relationship rather than an ortho relationship, the beneficial steric effect induced in Example Compounds may not be obtained with such a bond relationship, resulting in reduced characteristics of charge balance adjusting and charge transport. In addition, if (e.g., when) any one selected from among the phenanthrenyl group and the phenyl group is not in a para relationship, as Comparative Example Compound C4, with respect to the core nitrogen atom of the amine group, conjugation effect with the nitrogen atom of the amine group may not be obtained, resulting in reduced molecular stability. In addition, Comparative Example Compound C5 includes a phenyl group in which phenylene arranged between nitrogen-phenanthrenyl group is positioned at an ortho position with respect to the nitrogen atom of the amine group, but the corresponding phenyl group causes a significant steric distortion near the nitrogen of the amine group, thereby reducing molecular stability, Therefore, the light-emitting elements according to Comparative Examples 4 and 5 containing Comparative Example Compounds C4 and C5, respectively, exhibit reduced luminous efficiency and element lifespan compared to the light-emitting elements according to Examples.

Compared to Comparative Examples 4 and 5, the amine compounds according to Examples each contain a substituted or unsubstituted phenanthreny group and an unsubstituted phenyl group, bonded with a specific position relationship, and thus result in excellent or suitable charge transportability and material stability. It can be seen that, as a result, the light-emitting elements according to Examples each have improved efficiency and lifespan compared to the light-emitting elements according to Comparative Examples 4 and 5.

Comparative Example Compound C6 contained in the light-emitting element according to Comparative Example 6 corresponds to a case where one among Ar³ and Ar⁴ in Formula 1 of the disclosure contains a pyridyl group. The pyridyl group is a substituent unsuitable for hole transporting, and thus if (e.g., when) a compound containing such a substituent is applied in a light-emitting element, a charge balance in the light-emitting element may collapse. Therefore, it can be confirmed that the light-emitting element according to Comparative Example 6 containing Comparative Example Compound C6 exhibits reduced luminous efficiency and element lifespan compared to the light-emitting elements according to Examples.

Comparative Example Compound C7 contained in the light-emitting element according to Comparative Example 7 is a compound having a structure in which a heterocyclic substituent (benzonaphthothiophene) with high planarity is linked to the amine group via a phenylene linker. Like this, the heterocyclic substituent with high planarity is linked to the amine group via the linker, intermolecular interactions excessively (or substantially) increase, thereby increasing deposition temperature, which may cause the decomposition of molecules to increase during deposition. Therefore, it can be confirmed that the light-emitting element according to Example 7 has reduced luminous efficiency and element lifespan.

Comparative Example Compound C8 contained in the light-emitting element according to Comparative Example 8 corresponds to, compared with Example Compounds, a compound where a biphenylyl group rather than a phenyl group is substituted at an ortho position with respect to the phenanthrene group. When substituted with the biphenylyl group having a larger volume than a phenyl group, steric distortion near nitrogen of the amine group may increase. Therefore, Comparative Example Compound C8 has reduced molecular stability due to the steric distortion by the introduction of the biphenylyl group, and thus the light-emitting element according to Comparative Example 8 containing Comparative Example Compound C8 shows decreases in luminous efficiency and element lifespan, compared to the light-emitting elements according to Examples.

The light-emitting element according to one or more embodiments contains the amine compound according to one or more embodiments, and thus may exhibit characteristics of high efficiency and long lifespan.

When the amine compound according to one or more embodiments is applied to a light-emitting element, the light-emitting element may exhibit characteristics of high efficiency and long lifespan.

The display device according to one or more embodiments may exhibit excellent or suitable display quality by including the light-emitting element of one or more embodiments of the disclosure.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, or 5% of the stated value.

In the context of the present application and unless otherwise defined, the terms “use,” “using,” and “used” may be considered synonymous with the terms “utilize,” “utilizing,” and “utilized,” respectively.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The light-emitting element, the display device, the display apparatus, or any other relevant apparatuses/devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

In the present disclosure, each suitable feature of the various embodiments of the disclosure may be combined or combined with each other, partially or entirely, and may be technically interlocked and operated in various suitable ways, and each embodiment may be implemented independently of each other or in conjunction with each other in any suitable manner unless otherwise stated or implied.

Hitherto, although one or more embodiments of disclosure have been described with reference to example embodiments, it is understood that one or more suitable changes and modifications can be made by one ordinary skilled in the art or having ordinary knowledge in the art without departing from the spirit and technical scope of the present disclosure as hereinafter claimed. Accordingly, the technical scope of the disclosure is not limited to the contents set forth in the detailed description of the disclosure, but is defined by the appended claims and equivalents thereof.

What is claimed is:

1. A light-emitting element comprising:
a first electrode;
a second electrode on the first electrode; and
at least one functional layer between the first electrode and the second electrode, and comprising an amine compound represented by Formula 1: 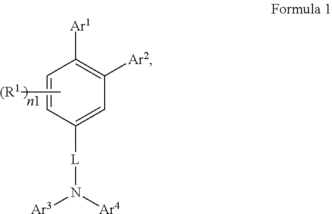
wherein, in Formula 1,
any one selected from among Ar¹ and Ar² is represented by Formula 2 or Formula 3, and the other one selected from among Ar¹ and Ar² is an unsubstituted phenyl group,
Ar³ and Ar⁴ are each independently a substituted or unsubstituted aryl group having 6 to 30 ring-forming carbons or a substituted or unsubstituted heteroaryl group having 2 to 30 ring-forming carbons,
L is a direct linkage, a substituted or unsubstituted arylene group having 6 to 30 ring-forming carbons, a substituted or unsubstituted heteroarylene group having 2 to 30 ring-forming carbons,
R¹ is hydrogen, deuterium, or a substituted or unsubstituted alkyl group having 1 to 10 carbons, and
n1 is an integer of 0 to 3: 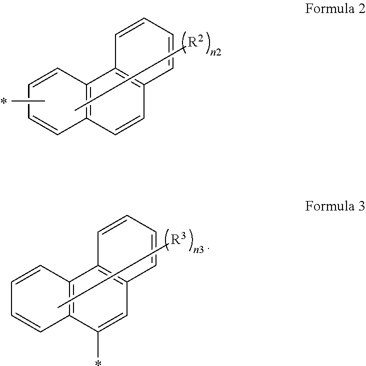
wherein, in Formula 2 and Formula 3,
R² and R³ are each independently hydrogen, deuterium, a substituted or unsubstituted alkyl group having 1 to 10 carbons, a substituted or unsubstituted aryl group having 6 to 30 ring-forming carbons, or a substituted or unsubstituted heteroaryl group having 2 to 30 ring-forming carbons,
n2 and n3 are each independently an integer of 0 to 9, and
-* is a position at which the rest portion of Formula 1 that is not represented by Formula 2 or Formula 3 is connected, and
wherein, in Formula 1,
when any one selected from among Ar¹ and Ar² is represented by Formula 3, then at least one of Ar³ or Ar⁴ comprises a substituted or unsubstituted naphthylphenyl group, a substituted or unsubstituted dibenzofuranyl group, a substituted or unsubstituted dibenzothiophenyl group, a substituted or unsubstituted 9-phenylcarbazolyl group, or a substituted or unsubstituted 9,9-diphenylfluorenyl group,
Ar³ and Ar⁴ do not comprise a substituted or unsubstituted nitrogen-containing six-membered hetero ring, a substituted or unsubstituted 9,9-dimethylfluorenyl group, and a boron-containing moiety,
when the amine compound comprises a four-membered fused hetero ring, then the four-membered fused hetero ring is directly bonded to the nitrogen atom in Formula 1,
a case where at least one selected from among Ar³ and Ar⁴ is a substituted or unsubstituted phenanthrenyl group is excluded, and
at least one hydrogen of the amine compound represented by Formula 1 is optionally substituted with deuterium.

2. The light-emitting element of claim 1, wherein the at least one functional layer comprises:
a light-emitting layer;
a hole transport region between the first electrode and the light-emitting layer; and
an electron transport region between the light-emitting layer and the second electrode, and
wherein the hole transport region comprises the amine compound represented by formula 1.

3. The light-emitting element of claim 2, wherein the hole transport region comprises:
a hole injection layer on the first electrode; and
a hole transport layer on the hole injection layer, and
wherein the hole transport layer comprises the amine compound represented by Formula 1.

4. The light-emitting element of claim 1, wherein the amine compound represented by Formula 1 is represented by Formula 2-1 or Formula 2-2: 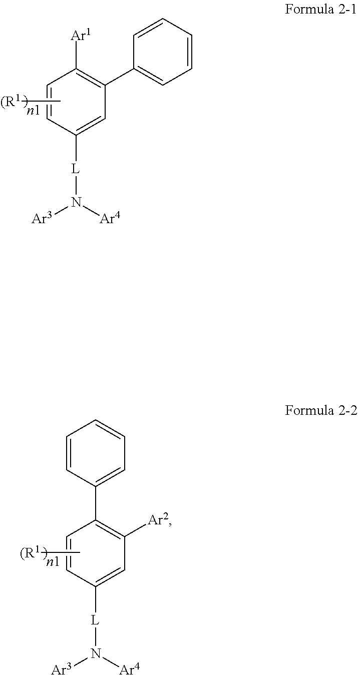
in Formula 2-1 and Formula 2-2,
R¹, n1, L, Ar¹, Ar², Ar³, and Ar⁴ each being the same as defined in Formula 1.

5. The light-emitting element of claim 1, wherein Ar³ and Ar⁴ are each independently a substituted or unsubstituted phenyl group, a substituted or unsubstituted biphenylyl group, a substituted or unsubstituted naphthyl group, a substituted or unsubstituted naphthylphenyl group, a substituted or unsubstituted dibenzofuranyl group, a substituted or unsubstituted dibenzothiophenyl group, a substituted or unsubstituted carbazolyl group, or a substituted or unsubstituted fluorenyl group.

6. The light-emitting element of claim 5, wherein, when Ar³ and Ar⁴ are each substituted, then a substituent thereof is deuterium, a substituted or unsubstituted phenyl group, a substituted or unsubstituted naphthyl group, a substituted or unsubstituted naphthylphenyl group, a substituted or unsubstituted fluorenyl group, a substituted or unsubstituted carbazolyl group, a substituted or unsubstituted dibenzofuranyl group, a substituted or unsubstituted dibenzothiophenyl group, or a substituted or unsubstituted phenanthrenyl group.

7. The light-emitting element of claim 1, wherein the amine compound represented by Formula 1 is represented by Formula 3: 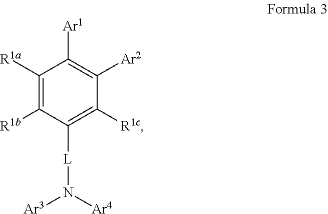
in Formula 3,
R^{1a}, R^{1b}, and R^{1c} being each independently hydrogen or deuterium, and
L, Ar¹, Ar², Ar³, and Ar⁴ being each the same as defined in Formula 1.

8. The light-emitting element of claim 1, wherein the amine compound represented by Formula 1 is represented by any one selected from among Formula 4-1 to Formula 4-4: 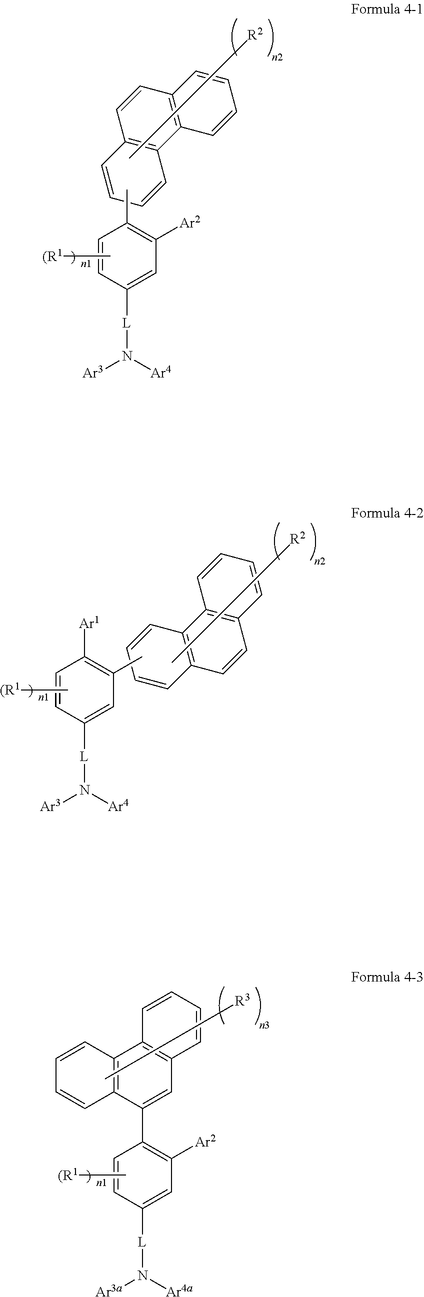 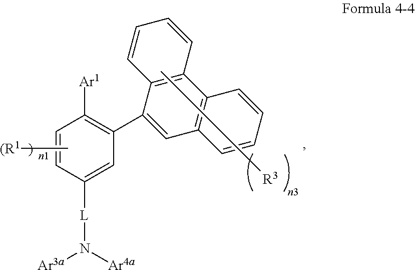
in Formula 4-1 to Formula 4-4,
Ar^{3a} and Ar^{4a} being each independently a substituted or unsubstituted aryl group having 6 to 30 ring-forming carbons or a substituted or unsubstituted heteroaryl group having 2 to 30 ring-forming carbons,
at least one selected from among Ar^{3a} and Ar^{4a} comprising a substituted or unsubstituted naphthylphenyl group, a substituted or unsubstituted dibenzofuranyl group, a substituted or unsubstituted dibenzothiophenyl group, a substituted or unsubstituted 9-phenylcarbazolyl group, or a substituted or unsubstituted 9,9-diphenylfluorenyl group,
R¹, n1, L, Ar¹, Ar², Ar³, and Ar⁴ each being the same as defined in Formula 1, and
R², R³, n2, and n3 each being the same as defined in Formula 2 and Formula 3.

9. The light-emitting element of claim 1, wherein the amine compound represented by Formula 1 is represented by any one selected from among Formula 5-1 to Formula 5-3: 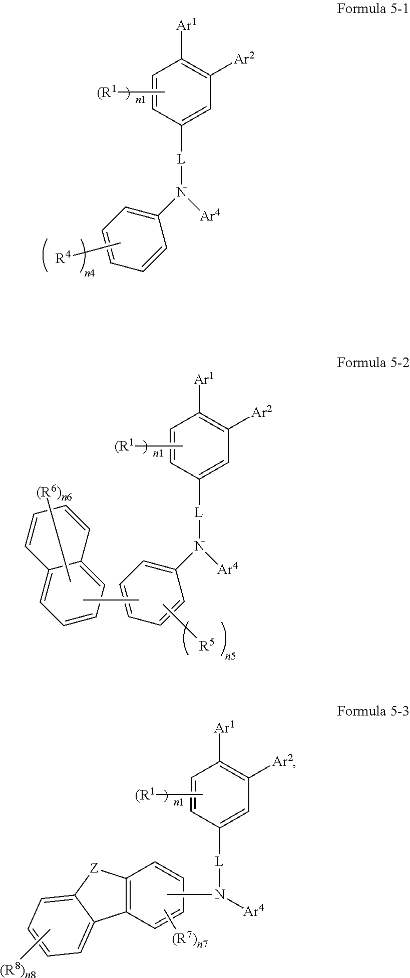
and
wherein, in Formula 5-1 to Formula 5-3,
R⁴ to R⁸ are each independently hydrogen, deuterium, a substituted or unsubstituted alkyl group having 1 to 20 carbons, a substituted or unsubstituted aryl group having 6 to 30 ring-forming carbons, or a substituted or unsubstituted heteroaryl group having 2 to 30 ring-forming carbons,
Z is O, S, NR⁹, or CR¹⁰R¹¹,
R⁹ to R¹¹ are each independently a substituted or unsubstituted aryl group having 6 to 30 ring-forming carbons,
n4 is an integer of 0 to 5,
n5 and n8 are each independently an integer of 0 to 4,
n6 is an integer of 0 to 7,
n7 is an integer of 0 to 3,
in Formula 5-1, when any one selected from among Ar¹ and Ar² is represented by Formula 3, then R⁴ is a substituted or unsubstituted dibenzofuranyl group, a substituted or unsubstituted dibenzothiophenyl group, a substituted or unsubstituted 9-phenylcarbazolyl group, or a substituted or unsubstituted 9,9-diphenylfluorenyl group, and
R¹, n1, L, Ar¹, Ar², and Ar⁴ are each the same as defined in Formula 1.

10. The light-emitting element of claim 1,
wherein the amine compound represented by Formula 1 is represented by Formula 6, and
the amine compound satisfies any one selected from among combinations present in Compound Combination Table 1: 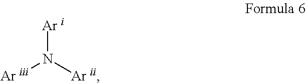
in Formula 6,
Arⁱ being any one selected from Substituent Group A,
Arⁱⁱ being any one selected from Substituent Group B, and
Arⁱⁱⁱ being any one selected from Substituent Group C, 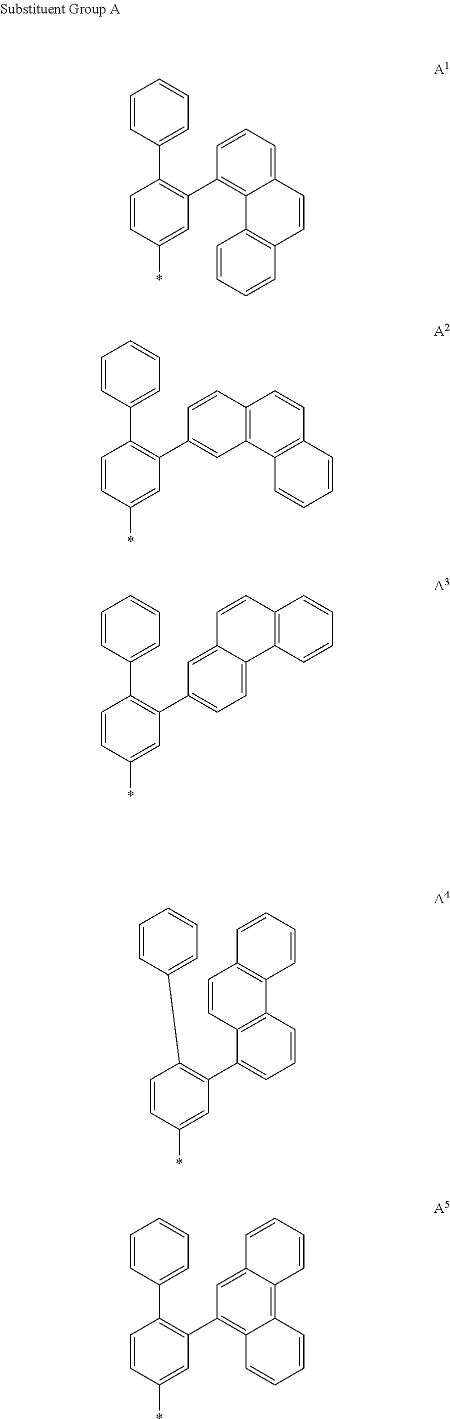 [SMILES: *c1ccc(-c2ccccc2)c(-c2cccc3ccc4ccccc4c23)c1 ; *c1ccc(-c2ccccc2)c(-c2ccc3ccc4ccccc4c3c2)c1 ; *c1ccc(-c2ccccc2)c(-c2ccc3c(ccc4ccccc43)c2)c1 ; *c1ccc(-c2ccccc2)c(-c2cccc3c2ccc2ccccc23)c1 ; *c1ccc(-c2ccccc2)c(-c2cc3ccccc3c3ccccc23)c1] 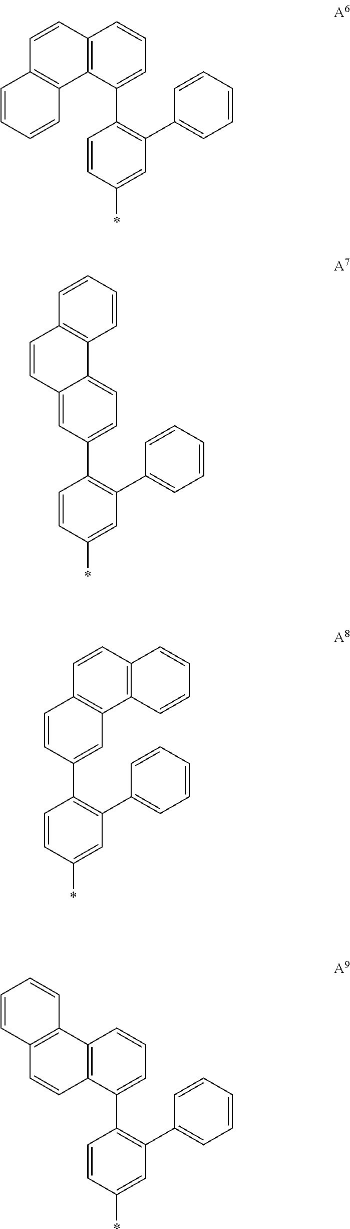 [SMILES: *c1ccc(-c2cccc3ccc4ccccc4c23)c(-c2ccccc2)c1 ; *c1ccc(-c2ccc3c(ccc4ccccc43)c2)c(-c2ccccc2)c1 ; *c1ccc(-c2ccc3ccc4ccccc4c3c2)c(-c2ccccc2)c1 ; *c1ccc(-c2cccc3c2ccc2ccccc23)c(-c2ccccc2)c1] 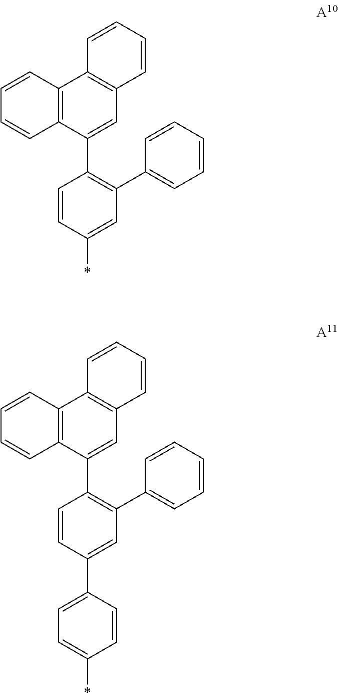 [SMILES: *c1ccc(-c2cc3ccccc3c3ccccc23)c(-c2ccccc2)c1 ; *c1ccc(-c2ccc(-c3cc4ccccc4c4ccccc34)c(-c3ccccc3)c2)cc1] 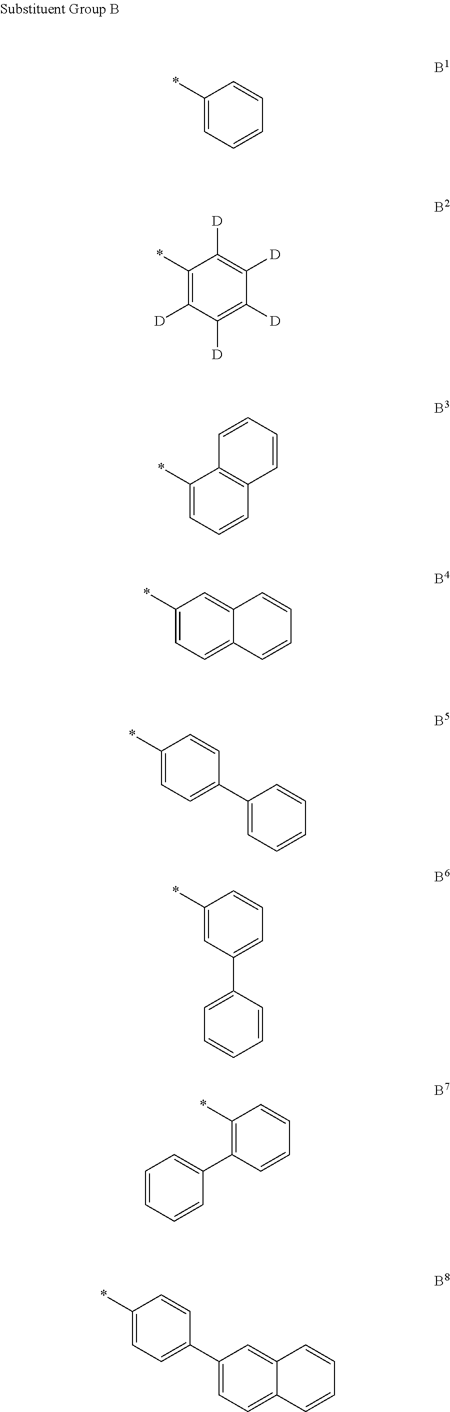 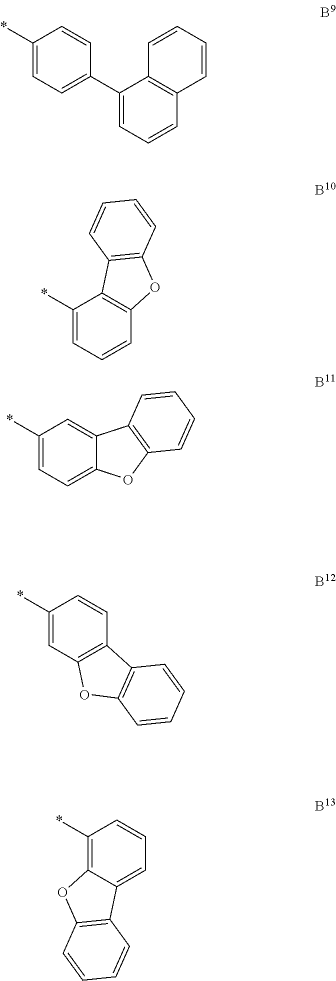 [SMILES: *c1ccc(-c2cccc3ccccc23)cc1 ; *c1cccc2oc3ccccc3c12 ; *c1ccc2oc3ccccc3c2c1 ; *c1ccc2c(c1)oc1ccccc12 ; *c1cccc2c1oc1ccccc12] 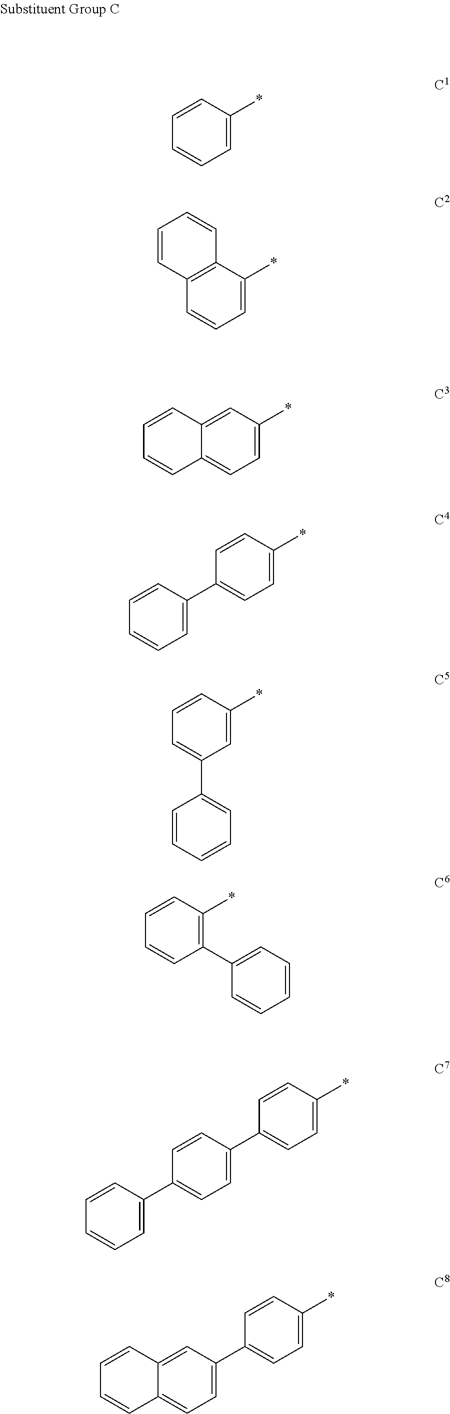 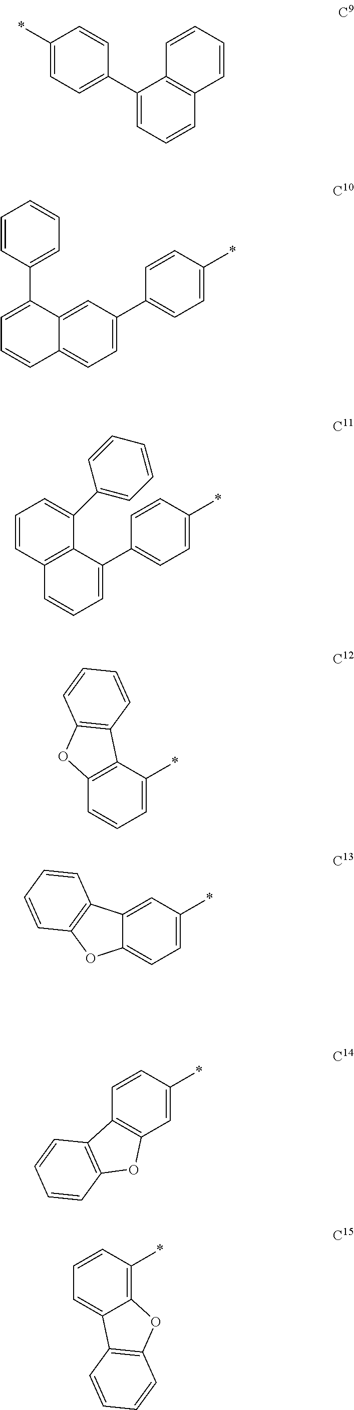 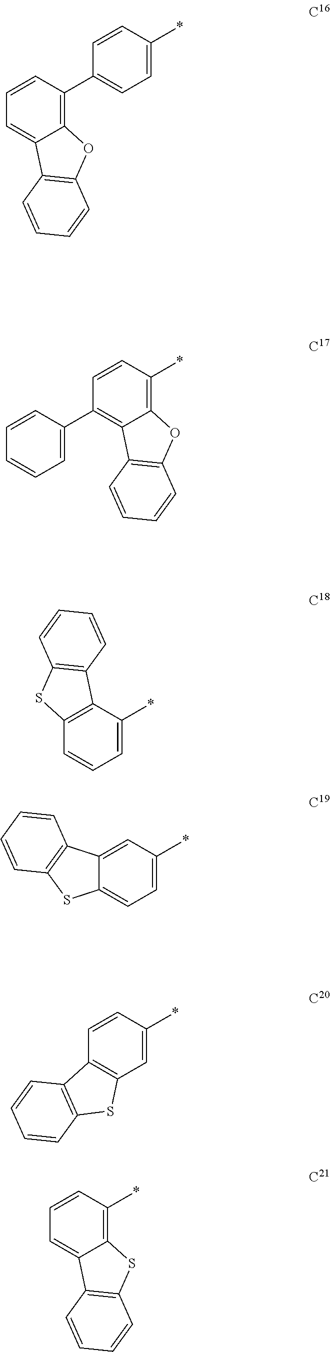 [SMILES: *c1ccc(-c2cccc3c2oc2ccccc23)cc1 ; *c1ccc(-c2ccccc2)c2c1oc1ccccc12 ; *c1cccc2sc3ccccc3c12 ; *c1ccc2sc3ccccc3c2c1 ; *c1ccc2c(c1)sc1ccccc12 ; *c1cccc2c1sc1ccccc12] 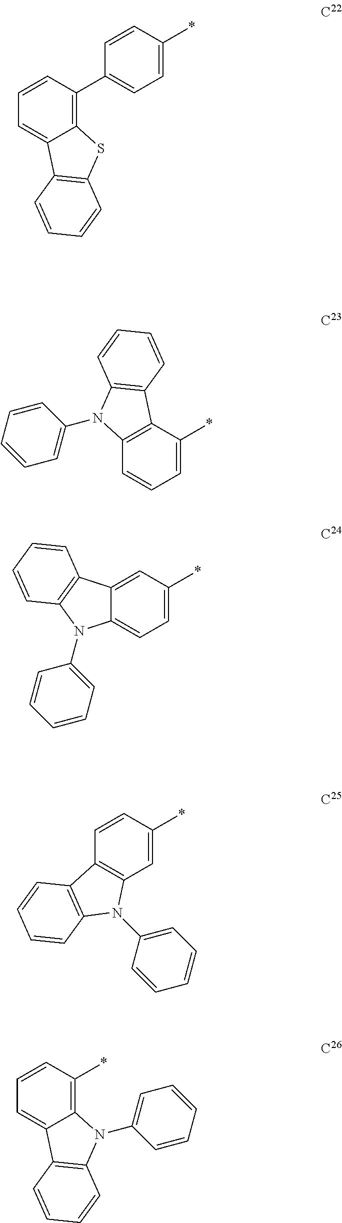 [SMILES: *c1ccc(-c2cccc3c2sc2ccccc23)cc1 ; *c1cccc2c1c1ccccc1n2-c1ccccc1 ; *c1ccc2c(c1)c1ccccc1n2-c1ccccc1 ; *c1ccc2c3ccccc3n(-c3ccccc3)c2c1 ; *c1cccc2c3ccccc3n(-c3ccccc3)c12] 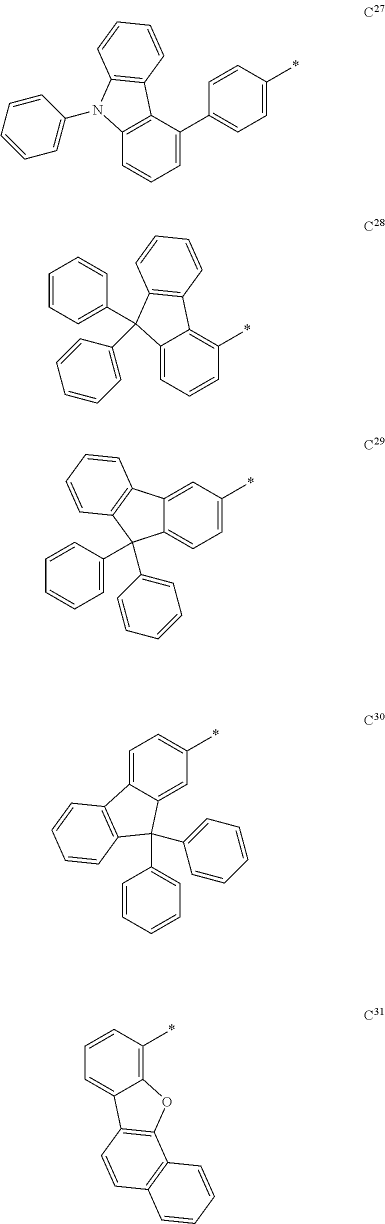 [SMILES: *c1ccc(-c2cccc3c2c2ccccc2n3-c2ccccc2)cc1 ; *c1cccc2c1-c1ccccc1C2(c1ccccc1)c1ccccc1 ; *c1ccc2c(c1)-c1ccccc1C2(c1ccccc1)c1ccccc1 ; *c1ccc2c(c1)C(c1ccccc1)(c1ccccc1)c1ccccc1-2 ; *c1cccc2c1oc1c3ccccc3ccc21] 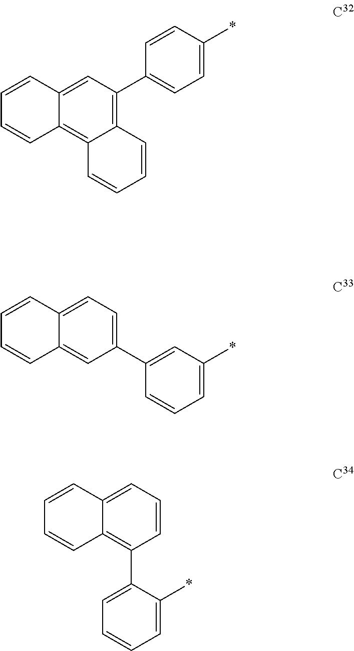 [SMILES: *c1ccc(-c2cc3ccccc3c3ccccc23)cc1 ; *c1cccc(-c2ccc3ccccc3c2)c1 ; *c1ccccc1-c1cccc2ccccc12]
Compound Combination Table 1

| No. | A | B | C |
|---|---|---|---|
| 1 | 1 | 1 | 1 |
| 2 | 2 | 1 | 1 |
| 3 | 3 | 1 | 1 |
| 4 | 4 | 1 | 1 |
| 5 | 6 | 1 | 1 |
| 6 | 7 | 1 | 1 |
| 7 | 8 | 1 | 1 |
| 8 | 9 | 1 | 1 |
| 9 | 1 | 1 | 2 |
| 10 | 2 | 1 | 2 |
| 11 | 3 | 1 | 2 |
| 12 | 4 | 1 | 2 |
| 13 | 5 | 1 | 33 |
| 14 | 6 | 1 | 2 |
| 15 | 7 | 1 | 2 |
| 16 | 8 | 1 | 2 |
| 17 | 9 | 1 | 2 |
| 18 | 10 | 1 | 33 |
| 19 | 11 | 1 | 33 |
| 20 | 1 | 1 | 3 |
| 21 | 2 | 1 | 3 |
| 22 | 3 | 1 | 3 |
| 23 | 4 | 1 | 3 |
| 24 | 5 | 1 | 34 |
| 25 | 6 | 1 | 3 |
| 26 | 7 | 1 | 3 |
| 27 | 8 | 1 | 3 |
| 28 | 9 | 1 | 3 |
| 29 | 10 | 1 | 34 |
| 30 | 11 | 1 | 34 |
| 31 | 1 | 1 | 8 |
| 32 | 2 | 1 | 8 |
| 33 | 3 | 1 | 8 |
| 34 | 4 | 1 | 8 |
| 35 | 5 | 1 | 8 |
| 36 | 6 | 1 | 8 |
| 37 | 7 | 1 | 8 |
| 38 | 8 | 1 | 88 |
| 39 | 9 | 1 | 88 |
| 40 | 10 | 1 | 8 |
| 41 | 11 | 1 | 8 |
| 42 | 1 | 2 | 1 |
| 43 | 2 | 2 | 1 |
| 44 | 3 | 2 | 1 |
| 45 | 4 | 2 | 1 |
| 46 | 6 | 2 | 1 |
| 47 | 7 | 2 | 1 |
| 48 | 8 | 2 | 1 |
| 49 | 9 | 2 | 1 |
| 50 | 1 | 2 | 2 |
| 51 | 2 | 2 | 2 |
| 52 | 3 | 2 | 2 |
| 53 | 4 | 2 | 2 |
| 54 | 5 | 2 | 33 |
| 55 | 6 | 2 | 2 |
| 56 | 7 | 2 | 2 |
| 57 | 8 | 2 | 2 |
| 58 | 9 | 2 | 2 |
| 59 | 10 | 2 | 33 |
| 60 | 11 | 2 | 33 |
| 61 | 1 | 2 | 3 |
| 62 | 2 | 2 | 3 |
| 63 | 3 | 2 | 3 |
| 64 | 4 | 2 | 3 |
| 65 | 5 | 2 | 34 |
| 66 | 6 | 2 | 3 |
| 67 | 7 | 2 | 3 |
| 68 | 8 | 2 | 3 |
| 69 | 9 | 2 | 3 |
| 70 | 10 | 2 | 34 |
| 71 | 11 | 2 | 34 |
| 72 | 1 | 2 | 8 |
| 73 | 2 | 2 | 8 |
| 74 | 3 | 2 | 8 |
| 75 | 4 | 2 | 8 |
| 76 | 5 | 2 | 8 |
| 77 | 6 | 2 | 8 |
| 78 | 7 | 2 | 8 |
| 79 | 8 | 2 | 8 |
| 80 | 9 | 2 | 8 |
| 81 | 10 | 2 | 8 |
| 82 | 11 | 2 | 8 |
| 83 | 1 | 5 | 1 |
| 84 | 2 | 5 | 1 |
| 85 | 3 | 5 | 1 |
| 86 | 4 | 5 | 1 |
| 87 | 6 | 5 | 1 |
| 88 | 7 | 5 | 1 |
| 89 | 8 | 5 | 1 |
| 90 | 9 | 5 | 1 |
| 91 | 1 | 5 | 2 |
| 92 | 2 | 5 | 2 |
| 93 | 3 | 5 | 2 |
| 94 | 4 | 5 | 2 |
| 95 | 5 | 5 | 33 |
| 96 | 6 | 5 | 2 |
| 97 | 7 | 5 | 2 |
| 98 | 8 | 5 | 2 |
| 99 | 9 | 5 | 2 |
| 100 | 10 | 5 | 33 |
| 101 | 11 | 5 | 33 |
| 102 | 1 | 5 | 3 |
| 103 | 2 | 5 | 3 |
| 104 | 3 | 5 | 3 |
| 105 | 4 | 5 | 3 |
| 106 | 5 | 5 | 34 |
| 107 | 6 | 5 | 3 |
| 108 | 7 | 5 | 3 |
| 109 | 8 | 5 | 3 |
| 110 | 9 | 5 | 3 |
| 111 | 10 | 5 | 34 |
| 112 | 11 | 5 | 34 |
| 113 | 1 | 5 | 8 |
| 114 | 2 | 5 | 8 |
| 115 | 3 | 5 | 8 |
| 116 | 4 | 5 | 8 |
| 117 | 5 | 5 | 8 |
| 118 | 6 | 5 | 8 |
| 119 | 7 | 5 | 8 |
| 120 | 8 | 5 | 8 |
| 121 | 9 | 5 | 8 |
| 122 | 10 | 5 | 8 |
| 123 | 11 | 5 | 8 |
| 124 | 2 | 6 | 1 |
| 125 | 2 | 7 | 1 |
| 126 | 2 | 9 | 1 |
| 127 | 2 | 10 | 1 |
| 128 | 2 | 11 | 1 |
| 129 | 2 | 12 | 1 |
| 130 | 2 | 13 | 1 |
| 131 | 2 | 3 | 2 |
| 132 | 2 | 4 | 2 |
| 133 | 2 | 6 | 2 |
| 134 | 2 | 7 | 2 |
| 135 | 2 | 8 | 2 |
| 136 | 2 | 9 | 2 |
| 137 | 2 | 10 | 2 |
| 138 | 2 | 11 | 2 |
| 139 | 2 | 12 | 2 |
| 140 | 2 | 13 | 2 |
| 141 | 2 | 2 | 2 |
| 142 | 2 | 5 | 4 |
| 143 | 2 | 6 | 4 |
| 144 | 2 | 7 | 4 |
| 145 | 2 | 9 | 4 |
| 146 | 2 | 10 | 4 |
| 147 | 2 | 11 | 4 |
| 148 | 2 | 12 | 4 |
| 149 | 2 | 13 | 4 |
| 150 | 2 | 4 | 8 |
| 151 | 2 | 6 | 8 |
| 152 | 2 | 7 | 8 |
| 153 | 2 | 8 | 8 |
| 154 | 2 | 9 | 8 |
| 155 | 2 | 10 | 8 |
| 156 | 2 | 11 | 8 |
| 157 | 2 | 12 | 8 |
| 158 | 2 | 13 | 8 |
| 159 | 2 | 1 | 16 |
| 160 | 2 | 2 | 16 |
| 161 | 2 | 3 | 16 |
| 162 | 2 | 4 | 16 |
| 163 | 2 | 5 | 16 |
| 164 | 2 | 6 | 16 |
| 165 | 2 | 7 | 16 |
| 166 | 2 | 8 | 16 |
| 167 | 2 | 9 | 16 |
| 168 | 2 | 10 | 16 |
| 169 | 2 | 11 | 16 |
| 170 | 2 | 12 | 16 |
| 171 | 2 | 13 | 16 |
| 172 | 2 | 1 | 22 |
| 173 | 2 | 2 | 22 |
| 174 | 2 | 3 | 22 |
| 175 | 2 | 4 | 22 |
| 176 | 2 | 5 | 22 |
| 177 | 2 | 6 | 22 |
| 178 | 2 | 7 | 22 |
| 179 | 2 | 8 | 22 |
| 180 | 2 | 9 | 22 |
| 181 | 2 | 10 | 22 |
| 182 | 2 | 11 | 22 |
| 183 | 2 | 12 | 22 |
| 184 | 2 | 13 | 22 |
| 185 | 2 | 1 | 27 |
| 186 | 2 | 2 | 27 |
| 187 | 2 | 3 | 27 |
| 188 | 2 | 4 | 27 |
| 189 | 2 | 5 | 27 |
| 190 | 2 | 6 | 27 |
| 191 | 2 | 7 | 27 |
| 192 | 2 | 8 | 27 |
| 193 | 2 | 9 | 27 |
| 194 | 2 | 10 | 27 |
| 195 | 2 | 11 | 27 |
| 196 | 2 | 12 | 27 |
| 197 | 2 | 13 | 27 |
| 198 | 5 | 9 | 1 |
| 199 | 5 | 10 | 1 |
| 200 | 5 | 11 | 1 |
| No. | A | B | C |
|---|---|---|---|
| 201 | 5 | 12 | 1 |
| 202 | 5 | 13 | 1 |
| 203 | 5 | 3 | 33 |
| 204 | 5 | 4 | 33 |
| 205 | 5 | 6 | 33 |
| 206 | 5 | 7 | 33 |
| 207 | 5 | 8 | 2 |
| 208 | 5 | 9 | 2 |
| 209 | 5 | 10 | 2 |
| 210 | 5 | 11 | 2 |
| 211 | 5 | 12 | 2 |
| 212 | 5 | 13 | 2 |
| 213 | 5 | 9 | 4 |
| 214 | 5 | 10 | 4 |
| 215 | 5 | 11 | 4 |
| 216 | 5 | 12 | 4 |
| 217 | 5 | 13 | 4 |
| 218 | 5 | 4 | 8 |
| 219 | 5 | 6 | 8 |
| 220 | 5 | 7 | 8 |
| 221 | 5 | 8 | 8 |
| 222 | 5 | 9 | 8 |
| 223 | 5 | 10 | 8 |
| 224 | 5 | 11 | 8 |
| 225 | 5 | 12 | 8 |
| 226 | 5 | 13 | 8 |
| 227 | 5 | 1 | 16 |
| 228 | 5 | 2 | 16 |
| 229 | 5 | 3 | 16 |
| 230 | 5 | 4 | 16 |
| 231 | 5 | 5 | 16 |
| 232 | 5 | 6 | 16 |
| 233 | 5 | 7 | 16 |
| 234 | 5 | 8 | 16 |
| 235 | 5 | 9 | 16 |
| 236 | 5 | 10 | 16 |
| 237 | 5 | 11 | 16 |
| 238 | 5 | 12 | 16 |
| 239 | 5 | 13 | 16 |
| 240 | 5 | 1 | 22 |
| No. | A | B | C |
|---|---|---|---|
| 241 | 5 | 2 | 22 |
| 242 | 5 | 3 | 22 |
| 243 | 5 | 4 | 22 |
| 244 | 5 | 5 | 22 |
| 245 | 5 | 6 | 22 |
| 246 | 5 | 7 | 22 |
| 247 | 5 | 8 | 22 |
| 248 | 5 | 9 | 22 |
| 249 | 5 | 10 | 22 |
| 250 | 5 | 11 | 22 |
| 251 | 5 | 12 | 22 |
| 252 | 5 | 13 | 22 |
| 253 | 5 | 1 | 27 |
| 254 | 5 | 2 | 27 |
| 255 | 5 | 3 | 27 |
| 256 | 5 | 4 | 27 |
| 257 | 5 | 5 | 27 |
| 258 | 5 | 6 | 27 |
| 259 | 5 | 7 | 27 |
| 260 | 5 | 8 | 27 |
| 261 | 5 | 9 | 27 |
| 262 | 5 | 10 | 27 |
| 263 | 5 | 11 | 27 |
| 264 | 5 | 12 | 27 |
| 265 | 5 | 13 | 27 |
| 266 | 8 | 6 | 1 |
| 267 | 8 | 7 | 1 |
| 268 | 8 | 9 | 1 |
| 269 | 8 | 10 | 1 |
| 270 | 8 | 11 | 1 |
| 271 | 8 | 12 | 1 |
| 272 | 8 | 13 | 1 |
| 273 | 8 | 3 | 2 |
| 274 | 8 | 4 | 2 |
| 275 | 8 | 6 | 2 |
| 276 | 8 | 7 | 2 |
| 277 | 8 | 8 | 2 |
| 278 | 8 | 9 | 2 |
| 279 | 8 | 10 | 2 |
| 280 | 8 | 11 | 2 |
| No. | A | B | C |
|---|---|---|---|
| 281 | 8 | 12 | 2 |
| 282 | 8 | 13 | 2 |
| 283 | 8 | 2 | 4 |
| 284 | 8 | 5 | 4 |
| 285 | 8 | 6 | 4 |
| 286 | 8 | 7 | 4 |
| 287 | 8 | 9 | 4 |
| 288 | 8 | 10 | 4 |
| 289 | 8 | 11 | 4 |
| 290 | 8 | 12 | 4 |
| 291 | 8 | 13 | 4 |
| 292 | 8 | 4 | 8 |
| 293 | 8 | 6 | 8 |
| 294 | 8 | 7 | 8 |
| 295 | 8 | 8 | 8 |
| 296 | 8 | 9 | 8 |
| 297 | 8 | 10 | 8 |
| 298 | 8 | 11 | 8 |
| 299 | 8 | 12 | 8 |
| 300 | 8 | 13 | 8 |
| 301 | 8 | 1 | 16 |
| 302 | 8 | 2 | 16 |
| 303 | 8 | 3 | 16 |
| 304 | 8 | 4 | 16 |
| 305 | 8 | 5 | 16 |
| 306 | 8 | 6 | 16 |
| 307 | 8 | 7 | 16 |
| 308 | 8 | 8 | 16 |
| 309 | 8 | 9 | 16 |
| 310 | 8 | 10 | 16 |
| 311 | 8 | 11 | 16 |
| 312 | 8 | 12 | 16 |
| 313 | 8 | 13 | 16 |
| 314 | 8 | 1 | 22 |
| 315 | 8 | 2 | 22 |
| 316 | 8 | 3 | 22 |
| 317 | 8 | 4 | 22 |
| 318 | 8 | 5 | 22 |
| 319 | 8 | 6 | 22 |
| 320 | 8 | 7 | 22 |
| No. | A | B | C |
|---|---|---|---|
| 321 | 8 | 8 | 22 |
| 322 | 8 | 9 | 22 |
| 323 | 8 | 10 | 22 |
| 324 | 8 | 11 | 22 |
| 325 | 8 | 12 | 22 |
| 326 | 8 | 13 | 22 |
| 327 | 8 | 1 | 27 |
| 328 | 8 | 2 | 27 |
| 329 | 8 | 3 | 27 |
| 330 | 8 | 4 | 27 |
| 331 | 8 | 5 | 27 |
| 332 | 8 | 6 | 27 |
| 333 | 8 | 7 | 27 |
| 334 | 8 | 8 | 27 |
| 335 | 8 | 9 | 27 |
| 336 | 8 | 10 | 27 |
| 337 | 8 | 11 | 27 |
| 338 | 8 | 12 | 27 |
| 339 | 8 | 13 | 27 |
| 340 | 10 | 9 | 1 |
| 341 | 10 | 10 | 1 |
| 342 | 10 | 11 | 1 |
| 343 | 10 | 12 | 1 |
| 344 | 10 | 13 | 1 |
| 345 | 10 | 3 | 33 |
| 346 | 10 | 4 | 33 |
| 347 | 10 | 6 | 33 |
| 348 | 10 | 7 | 33 |
| 349 | 10 | 8 | 2 |
| 350 | 10 | 9 | 2 |
| 351 | 10 | 10 | 2 |
| 352 | 10 | 11 | 2 |
| 353 | 10 | 12 | 2 |
| 354 | 10 | 13 | 2 |
| 355 | 10 | 9 | 4 |
| 356 | 10 | 10 | 4 |
| 357 | 10 | 11 | 4 |
| 358 | 10 | 12 | 4 |
| 359 | 10 | 13 | 4 |
| 360 | 10 | 4 | 8 |
| No. | A | B | C |
|---|---|---|---|
| 361 | 10 | 6 | 8 |
| 362 | 10 | 7 | 8 |
| 363 | 10 | 8 | 8 |
| 364 | 10 | 9 | 8 |
| 365 | 10 | 10 | 8 |
| 366 | 10 | 11 | 8 |
| 367 | 10 | 12 | 8 |
| 368 | 10 | 13 | 8 |
| 369 | 10 | 1 | 16 |
| 370 | 10 | 2 | 16 |
| 371 | 10 | 3 | 16 |
| 372 | 10 | 4 | 16 |
| 373 | 10 | 5 | 16 |
| 374 | 10 | 6 | 16 |
| 375 | 10 | 7 | 16 |
| 376 | 10 | 8 | 16 |
| 377 | 10 | 9 | 16 |
| 378 | 10 | 10 | 16 |
| 379 | 10 | 11 | 16 |
| 380 | 10 | 12 | 16 |
| 381 | 10 | 13 | 16 |
| 382 | 10 | 1 | 22 |
| 383 | 10 | 2 | 22 |
| 384 | 10 | 3 | 22 |
| 385 | 10 | 4 | 22 |
| 386 | 10 | 5 | 22 |
| 387 | 10 | 6 | 22 |
| 388 | 10 | 7 | 22 |
| 389 | 10 | 8 | 22 |
| 390 | 10 | 9 | 22 |
| 391 | 10 | 10 | 22 |
| 392 | 10 | 11 | 22 |
| 393 | 10 | 12 | 22 |
| 394 | 10 | 13 | 22 |
| 395 | 10 | 1 | 27 |
| 396 | 10 | 2 | 27 |
| 397 | 10 | 3 | 27 |
| 398 | 10 | 4 | 27 |
| 399 | 10 | 5 | 27 |
| 400 | 10 | 6 | 27 |
| No. | A | B | C |
|---|---|---|---|
| 401 | 10 | 7 | 27 |
| 402 | 10 | 8 | 27 |
| 403 | 10 | 9 | 27 |
| 404 | 10 | 10 | 27 |
| 405 | 10 | 11 | 27 |
| 406 | 10 | 12 | 27 |
| 407 | 10 | 13 | 27 |
| 408 | 2 | 1 | 7 |
| 409 | 2 | 1 | 10 |
| 410 | 2 | 1 | 11 |
| 411 | 2 | 1 | 17 |
| 412 | 2 | 1 | 18 |
| 413 | 2 | 1 | 19 |
| 414 | 2 | 1 | 20 |
| 415 | 2 | 1 | 21 |
| 416 | 2 | 1 | 23 |
| 417 | 2 | 1 | 24 |
| 418 | 2 | 1 | 25 |
| 419 | 2 | 1 | 26 |
| 420 | 2 | 1 | 28 |
| 421 | 2 | 1 | 2 |
| 422 | 2 | 1 | 30 |
| 423 | 2 | 1 | 31 |
| 424 | 2 | 1 | 32 |
| 425 | 2 | 2 | 5 |
| 426 | 2 | 2 | 6 |
| 427 | 2 | 2 | 7 |
| 428 | 2 | 2 | 9 |
| 429 | 2 | 2 | 10 |
| 430 | 2 | 2 | 11 |
| 431 | 2 | 2 | 12 |
| 432 | 2 | 2 | 13 |
| 433 | 2 | 2 | 14 |
| 434 | 2 | 2 | 15 |
| 435 | 2 | 2 | 17 |
| 436 | 2 | 2 | 18 |
| 437 | 2 | 2 | 19 |
| 438 | 2 | 2 | 20 |
| 439 | 2 | 2 | 21 |
| 440 | 2 | 2 | 23 |
| No. | A | B | C |
|---|---|---|---|
| 441 | 2 | 2 | 24 |
| 442 | 2 | 2 | 25 |
| 443 | 2 | 2 | 26 |
| 444 | 2 | 2 | 28 |
| 445 | 2 | 2 | 29 |
| 446 | 2 | 2 | 30 |
| 447 | 2 | 2 | 31 |
| 448 | 2 | 2 | 32 |
| 449 | 2 | 12 | 3 |
| 450 | 2 | 12 | 5 |
| 451 | 2 | 12 | 6 |
| 452 | 2 | 12 | 7 |
| 453 | 2 | 12 | 9 |
| 454 | 2 | 12 | 10 |
| 455 | 2 | 12 | 11 |
| 456 | 2 | 12 | 12 |
| 457 | 2 | 12 | 13 |
| 458 | 2 | 12 | 14 |
| 459 | 2 | 12 | 15 |
| 460 | 2 | 12 | 17 |
| 461 | 2 | 12 | 18 |
| 462 | 2 | 12 | 19 |
| 463 | 2 | 12 | 20 |
| 464 | 2 | 12 | 21 |
| 465 | 2 | 12 | 23 |
| 466 | 2 | 12 | 24 |
| 467 | 2 | 12 | 25 |
| 468 | 2 | 12 | 26 |
| 469 | 2 | 12 | 28 |
| 470 | 2 | 12 | 29 |
| 471 | 2 | 12 | 30 |
| 472 | 2 | 3 | 31 |
| 473 | 2 | 3 | 32 |
| 474 | 5 | 1 | 10 |
| 475 | 5 | 1 | 11 |
| 476 | 5 | 1 | 17 |
| 477 | 5 | 1 | 18 |
| 478 | 5 | 1 | 19 |
| 479 | 5 | 1 | 20 |
| 480 | 5 | 1 | 21 |
| No. | A | B | |
|---|---|---|---|
| 481 | 5 | 1 | 23 |
| 482 | 5 | 1 | 24 |
| 483 | 5 | 1 | 25 |
| 484 | 5 | 1 | 26 |
| 485 | 5 | 1 | 28 |
| 486 | 5 | 1 | 29 |
| 487 | 5 | 1 | 30 |
| 488 | 5 | 1 | 31 |
| 489 | 5 | 1 | 32 |
| 490 | 5 | 2 | 9 |
| 491 | 5 | 2 | 10 |
| 492 | 5 | 2 | 11 |
| 493 | 5 | 2 | 12 |
| 494 | 5 | 2 | 13 |
| 495 | 5 | 2 | 14 |
| 496 | 5 | 2 | 15 |
| 497 | 5 | 2 | 17 |
| 498 | 5 | 2 | 18 |
| 499 | 5 | 2 | 19 |
| 500 | 5 | 2 | 20 |
| 501 | 5 | 2 | 21 |
| 502 | 5 | 2 | 23 |
| 503 | 5 | 2 | 24 |
| 504 | 5 | 2 | 25 |
| 505 | 5 | 2 | 26 |
| 506 | 5 | 2 | 28 |
| 507 | 5 | 2 | 29 |
| 508 | 5 | 2 | 30 |
| 509 | 5 | 2 | 31 |
| 510 | 5 | 2 | 32 |
| 511 | 5 | 12 | 3 |
| 512 | 5 | 12 | 5 |
| 513 | 5 | 12 | 6 |
| 514 | 5 | 12 | 7 |
| 515 | 5 | 12 | 9 |
| 516 | 5 | 12 | 10 |
| 517 | 5 | 12 | 11 |
| 518 | 5 | 12 | 12 |
| 519 | 5 | 12 | 13 |
| 520 | 5 | 12 | 14 |
| No. | A | B | C |
|---|---|---|---|
| 521 | 5 | 12 | 15 |
| 522 | 5 | 12 | 17 |
| 523 | 5 | 12 | 18 |
| 524 | 5 | 12 | 19 |
| 525 | 5 | 12 | 20 |
| 526 | 5 | 12 | 21 |
| 527 | 5 | 12 | 23 |
| 528 | 5 | 12 | 24 |
| 529 | 5 | 12 | 25 |
| 530 | 5 | 12 | 26 |
| 531 | 5 | 12 | 28 |
| 532 | 5 | 12 | 29 |
| 533 | 5 | 12 | 30 |
| 534 | 5 | 3 | 31 |
| 535 | 5 | 3 | 32 |
| 536 | 8 | 1 | 7 |
| 537 | 8 | 1 | 10 |
| 538 | 8 | 1 | 11 |
| 539 | 8 | 1 | 17 |
| 540 | 8 | 1 | 18 |
| 541 | 8 | 1 | 19 |
| 542 | 8 | 1 | 20 |
| 543 | 8 | 1 | 21 |
| 544 | 8 | 1 | 23 |
| 545 | 8 | 1 | 24 |
| 546 | 8 | 1 | 25 |
| 547 | 8 | 1 | 26 |
| 548 | 8 | 1 | 28 |
| 549 | 8 | 1 | 29 |
| 550 | 8 | 1 | 30 |
| 551 | 8 | 1 | 31 |
| 552 | 8 | 1 | 32 |
| 553 | 8 | 2 | 5 |
| 554 | 8 | 2 | 6 |
| 555 | 8 | 2 | 7 |
| 556 | 8 | 2 | 9 |
| 557 | 8 | 2 | 10 |
| 558 | 8 | 2 | 11 |
| 559 | 8 | 2 | 12 |
| 560 | 8 | 2 | 13 |
| No. | A | B | C |
|---|---|---|---|
| 561 | 8 | 2 | 14 |
| 562 | 8 | 2 | 15 |
| 563 | 8 | 2 | 17 |
| 564 | 8 | 2 | 18 |
| 565 | 8 | 2 | 19 |
| 566 | 8 | 2 | 20 |
| 567 | 8 | 2 | 21 |
| 568 | 8 | 2 | 23 |
| 569 | 8 | 2 | 24 |
| 570 | 8 | 2 | 25 |
| 571 | 8 | 2 | 26 |
| 572 | 8 | 2 | 28 |
| 573 | 8 | 2 | 29 |
| 574 | 8 | 2 | 30 |
| 575 | 8 | 2 | 31 |
| 576 | 8 | 2 | 32 |
| 577 | 8 | 12 | 3 |
| 578 | 8 | 12 | 5 |
| 579 | 8 | 12 | 6 |
| 580 | 8 | 12 | 7 |
| 581 | 8 | 12 | 9 |
| 582 | 8 | 12 | 10 |
| 583 | 8 | 12 | 11 |
| 584 | 8 | 12 | 12 |
| 585 | 8 | 12 | 13 |
| 586 | 8 | 12 | 14 |
| 587 | 8 | 12 | 15 |
| 588 | 8 | 12 | 17 |
| 589 | 8 | 12 | 18 |
| 590 | 8 | 12 | 19 |
| 591 | 8 | 12 | 20 |
| 592 | 8 | 12 | 21 |
| 593 | 8 | 12 | 23 |
| 594 | 8 | 12 | 24 |
| 595 | 8 | 12 | 25 |
| 596 | 8 | 12 | 26 |
| 597 | 8 | 12 | 28 |
| 598 | 8 | 12 | 29 |
| 599 | 8 | 12 | 30 |
| 600 | 8 | 12 | 31 |
| No. | A | B | C |
|---|---|---|---|
| 601 | 8 | 12 | 32 |
| 602 | 10 | 1 | 10 |
| 603 | 10 | 1 | 11 |
| 604 | 10 | 1 | 17 |
| 605 | 10 | 1 | 18 |
| 606 | 10 | 1 | 19 |
| 607 | 10 | 1 | 20 |
| 608 | 10 | 1 | 21 |
| 609 | 10 | 1 | 23 |
| 610 | 10 | 1 | 24 |
| 611 | 10 | 1 | 25 |
| 612 | 10 | 1 | 26 |
| 613 | 10 | 1 | 28 |
| 614 | 10 | 1 | 29 |
| 615 | 10 | 1 | 30 |
| 616 | 10 | 1 | 31 |
| 617 | 10 | 1 | 32 |
| 618 | 10 | 2 | 9 |
| 619 | 10 | 2 | 10 |
| 620 | 10 | 2 | 11 |
| 621 | 10 | 2 | 12 |
| 622 | 10 | 2 | 13 |
| 623 | 10 | 2 | 14 |
| 624 | 10 | 2 | 15 |
| 625 | 10 | 2 | 17 |
| 626 | 10 | 2 | 18 |
| 627 | 10 | 2 | 19 |
| 628 | 10 | 2 | 20 |
| 629 | 10 | 2 | 21 |
| 630 | 10 | 2 | 23 |
| 631 | 10 | 2 | 24 |
| 632 | 10 | 2 | 25 |
| 683 | 10 | 2 | 26 |
| 634 | 10 | 2 | 28 |
| 635 | 10 | 2 | 29 |
| 636 | 10 | 2 | 30 |
| 637 | 10 | 2 | 31 |
| 638 | 10 | 2 | 32 |
| 639 | 10 | 12 | 3 |
| 640 | 10 | 12 | 5 |
| No. | A | B | C |
|---|---|---|---|
| 641 | 10 | 12 | 6 |
| 642 | 10 | 12 | 7 |
| 643 | 10 | 12 | 9 |
| 644 | 10 | 12 | 10 |
| 645 | 10 | 12 | 11 |
| 646 | 10 | 12 | 12 |
| 647 | 10 | 12 | 13 |
| 648 | 10 | 12 | 14 |
| 649 | 10 | 12 | 15 |
| 650 | 10 | 12 | 17 |
| 651 | 10 | 12 | 18 |
| 652 | 10 | 12 | 19 |
| 653 | 10 | 12 | 20 |
| 654 | 10 | 12 | 21 |
| 655 | 10 | 12 | 23 |
| 656 | 10 | 12 | 24 |
| 657 | 10 | 12 | 25 |
| 658 | 10 | 12 | 26 |
| 659 | 10 | 12 | 28 |
| 660 | 10 | 12 | 29 |
| 661 | 10 | 12 | 30 |
| 662 | 10 | 3 | 31 |
| 663 | 10 | 3 | 32 |

11. An electronic apparatus comprising:
a base layer;
a circuit layer on the base layer; and
a display element layer on the circuit layer and comprising a light-emitting element,
wherein, the light-emitting element comprises a first electrode, a second electrode on the first electrode, and at least one functional layer between the first and the second electrode and comprising an amine compound represented by Formula 1: 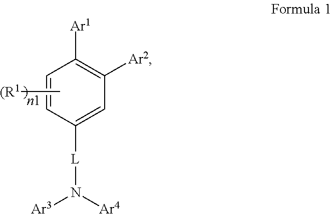
wherein, in Formula 1,
any one selected from among Ar¹ and Ar² is represented by Formula 2 or Formula 3, and the other one selected from among Ar¹ and Ar² is an unsubstituted phenyl group,
Ar³ and Ar⁴ are each independently a substituted or unsubstituted aryl group having 6 to 30 ring-forming carbons or a substituted or unsubstituted heteroaryl group having 2 to 30 ring-forming carbons,
L is a direct linkage, a substituted or unsubstituted arylene group having 6 to 30 ring-forming carbons, or a substituted or unsubstituted heteroarylene group having 2 to 30 ring-forming carbons,
R¹ is hydrogen, deuterium, or a substituted or unsubstituted alkyl group having 1 to 10 carbons, and
n1 is an integer of 0 to 3: 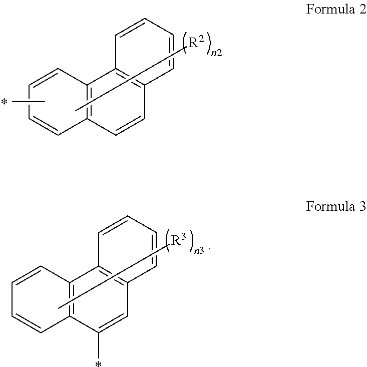
wherein, in Formula 2 and Formula 3,
R² and R³ are each independently hydrogen, deuterium, a substituted or unsubstituted alkyl group having 1 to 10 carbons, a substituted or unsubstituted aryl group having 6 to 30 ring-forming carbons, or a substituted or unsubstituted heteroaryl group having 2 to 30 ring-forming carbons,
n2 and n3 are each independently an integer of 0 to 9, and 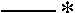 [SMILES: *C]
is a position at which the rest portion of Formula 1 that is not represented by Formula 2 or Formula 3 is connected, and
wherein, in Formula 1,
when any one selected from among Ar¹ and Ar² is represented by Formula 3, then at least one selected from among Ar³ and Ar⁴ comprises a substituted or unsubstituted naphthylphenyl group, a substituted or unsubstituted dibenzofuranyl group, a substituted or unsubstituted dibenzothiophenyl group, a substituted or unsubstituted 9-phenylcarbazolyl group, or a substituted or unsubstituted 9,9-diphenylfluorenyl group,
Ar³ and Ar⁴ do not comprise a substituted or unsubstituted nitrogen-containing six-membered hetero ring, a substituted or unsubstituted 9,9-dimethylfluorenyl group, and a boron-containing moiety,
when the amine compound represented by Formula 1 comprises a four-membered fused hetero ring, then the four-membered fused hetero ring is directly bonded to the nitrogen atom in Formula 1,
a case where at least one selected from among Ar³ and Ar⁴ is a substituted or unsubstituted phenanthrenyl group is excluded, and
at least one hydrogen of the amine compound represented by Formula 1 is optionally substituted with deuterium.

12. The electronic apparatus of claim 11, wherein:
the light-emitting element further comprises a capping layer on the second electrode, and
the capping layer has a refractive index of at least about 1.6 for light in a wavelength range of about 550 nm to about 660 nm.

13. The electronic apparatus of claim 11, further comprising a light control layer on the display element layer and comprising a quantum dot,
wherein the light-emitting element is to emit first color light, and
the light control layer comprises:
a first light control part comprising a first quantum dot that converts the first color light to second color light in a longer wavelength region than the first color light;
a second light control part comprising a second quantum dot that converts the first color light to third color light in a longer wavelength region than the first color light and the second color light; and
a third light control part that transmits the first color light.

14. The electronic apparatus of claim 13, further comprising a color filter layer on the light control layer,
wherein the color filter layer comprises:
a first filter configured to transmit the second color light;
a second filter configured to transmit the third color light; and
a third filter configured to transmit the first color light.

15. An amine compound represented by Formula 1: 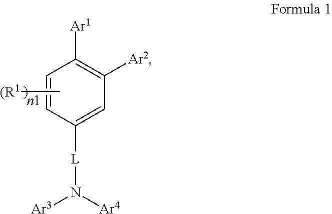
wherein, in Formula 1,
any one selected from among Ar¹ and Ar² is represented by Formula 2 or Formula 3, and the other is an unsubstituted phenyl group,
Ar³ and Ar⁴ are each independently a substituted or unsubstituted aryl group having 6 to 30 ring-forming carbons or a substituted or unsubstituted heteroaryl group having 2 to 30 ring-forming carbons,
L is a direct linkage, a substituted or unsubstituted arylene group having 6 to 30 ring-forming carbons, or a substituted or unsubstituted heteroarylene group having 2 to 30 ring-forming carbons,
R¹ is hydrogen, deuterium, or a substituted or unsubstituted alkyl group having 1 to 10 carbons, and
n1 is an integer of 0 to 3, 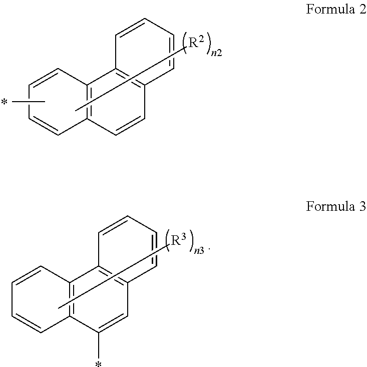
wherein, in Formula 2 and Formula 3,
R² and R³ are each independently hydrogen, deuterium, a substituted or unsubstituted alkyl group having 1 to 10 carbons, a substituted or unsubstituted aryl group having 6 to 30 ring-forming carbons, or a substituted or unsubstituted heteroaryl group having 2 to 30 ring-forming carbons,
n2 and n3 are each independently an integer 0 to 9, and
-* is a position at which the rest portion of Formula 1 that is not represented by Formula 2 or Formula 3 is connected, and
wherein, in Formula 1,
when any one selected from among Ar¹ and Ar² is represented by Formula 3, then at least one selected from among Ar³ and Ar⁴ comprises a substituted or unsubstituted naphthylphenyl group, a substituted or unsubstituted dibenzofuranyl group, a substituted or unsubstituted dibenzothiophenyl group, a substituted or unsubstituted 9-phenylcarbazolyl group, or a substituted or unsubstituted 9,9-diphenylfluorenyl group,
Ar³ and Ar⁴ do not comprise a substituted or unsubstituted nitrogen-containing six-membered hetero ring, a substituted or unsubstituted 9,9-dimethylfluorenyl group, and a boron-containing moiety,
when the amine compound represented by Formula 1 comprises a four-membered fused hetero ring, then the four-membered fused hetero ring is directly bonded to the nitrogen atom in Formula 1,
a case where at least one selected from among Ar³ and Ar⁴ is a substituted or unsubstituted phenanthrenyl group is excluded, and
at least one hydrogen of the amine compound represented by Formula 1 is optionally substituted with deuterium.

16. The amine compound of claim 15, wherein the amine compound represented by Formula 1 is represented by Formula 2-1 or Formula 2-2: 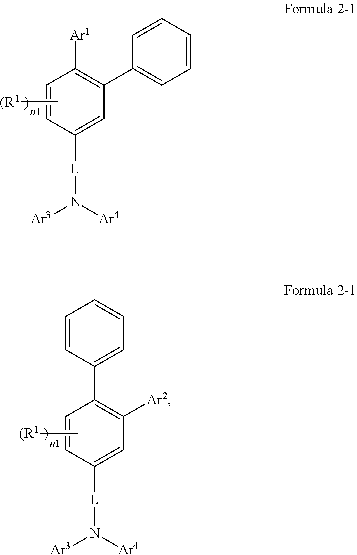
in Formula 2-1 and Formula 2-2,
R¹, n1, L, Ar¹, Ar², Ar³, and Ar⁴ each being the same as defined in Formula 1.

17. The amine compound of claim 15, wherein the amine compound represented by Formula 1 is represented by Formula 3: 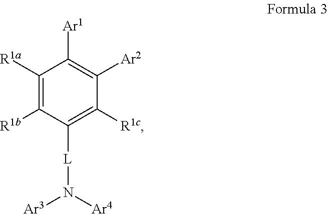
in Formula 3,
R^{1a}, R^{1b}, and R^{1c} being each independently hydrogen or deuterium, and
L, Ar¹, Ar², Ar³, and Ar⁴ each being the same as defined in Formula 1.

18. The amine compound of claim 15, wherein the amine compound represented by Formula 1 is represented by any one selected from among Formula 4-1 to Formula 4-4: 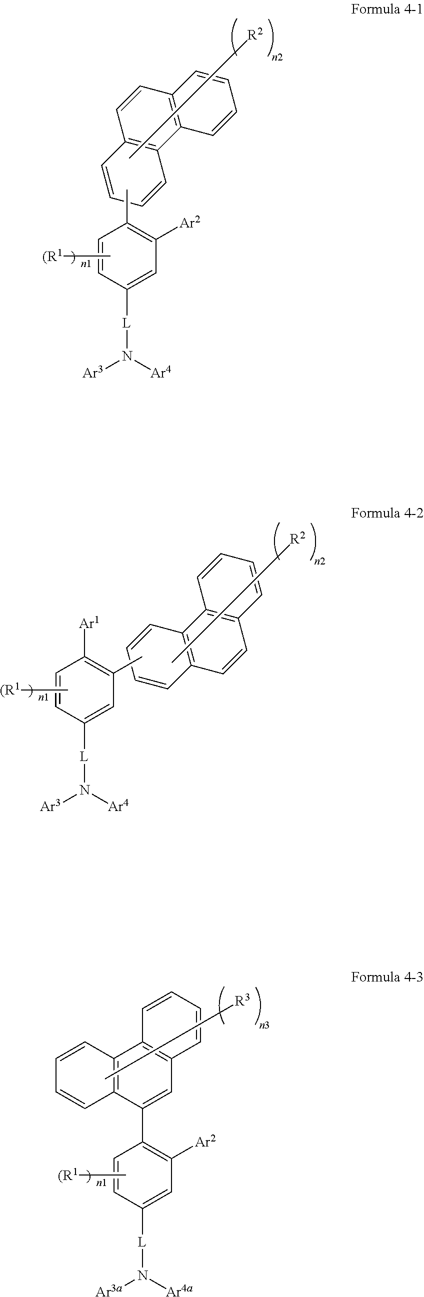 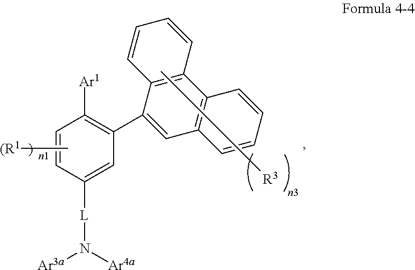
in Formula 4-1 to Formula 4-4,
Ar^{3a} and Ar^{4a} being each independently a substituted or unsubstituted aryl group having 6 to 30 ring-forming carbons or a substituted or unsubstituted heteroaryl group having 2 to 30 ring-forming carbons,
at least one selected from among Ar^{3a} and Ar^{4a} comprising a substituted or unsubstituted naphthylphenyl group, a substituted or unsubstituted dibenzofuranyl group, a substituted or unsubstituted dibenzothiophenyl group, a substituted or unsubstituted 9-phenylcarbazolyl group, or a substituted or unsubstituted 9,9-diphenylfluorenyl group, and
R¹, n1, L, Ar¹, Ar², Ar³, and Ar⁴ each being the same as defined in Formula 1.

19. The amine compound of claim 15, wherein the amine compound represented by Formula 1 is represented by any one among Formula 5-1 to Formula 5-3: 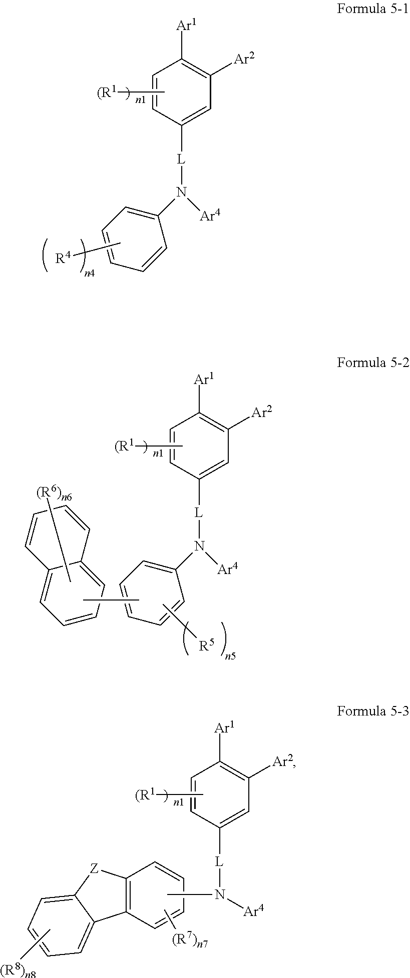
and
wherein, in Formula 5-1 to Formula 5-3, R⁴ to R⁸ are each independently hydrogen, deuterium, a substituted or unsubstituted alkyl group having 1 to 20 carbons, a substituted or unsubstituted aryl group having 6 to 30 ring-forming carbons, or a substituted or unsubstituted heteroaryl group having 2 to 30 ring-forming carbons,
Z is O, S, NR⁹, or CR¹⁰R¹¹
R⁹ to R¹¹ are each independently a substituted or unsubstituted aryl group having 6 to 30 ring-forming carbons,
n4 is an integer of 0 to 5,
n5 and n8 are each independently an integer of 0 to 4,
n6 is an integer of 0 to 7,
n7 is an integer of 0 to 3,
in Formula 5-1, when any one selected from among Ar¹ and Ar² is represented by Formula 3, then R⁴ is a substituted or unsubstituted dibenzofuranyl group, a substituted or unsubstituted dibenzothiophenyl group, a substituted or unsubstituted 9-phenylcarbazolyl group, or a substituted or unsubstituted 9,9-diphenylfluorenyl group, and
R¹, n1, L, Ar¹, Ar², and Ar⁴ are each the same as defined in Formula 1.

20. The amine compound of claim 15,
wherein the amine compound represented by Formula 1 is represented by Formula 6, and
the amine compound satisfies any one selected from among combinations present in Compound Combination Table 1: 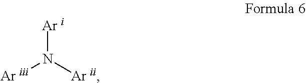
in Formula 6,
Arⁱ being any one selected from Substituent Group A,
Arⁱⁱ being any one selected from Substituent Group B, and
Arⁱⁱⁱ being any one selected from Substituent Group C, 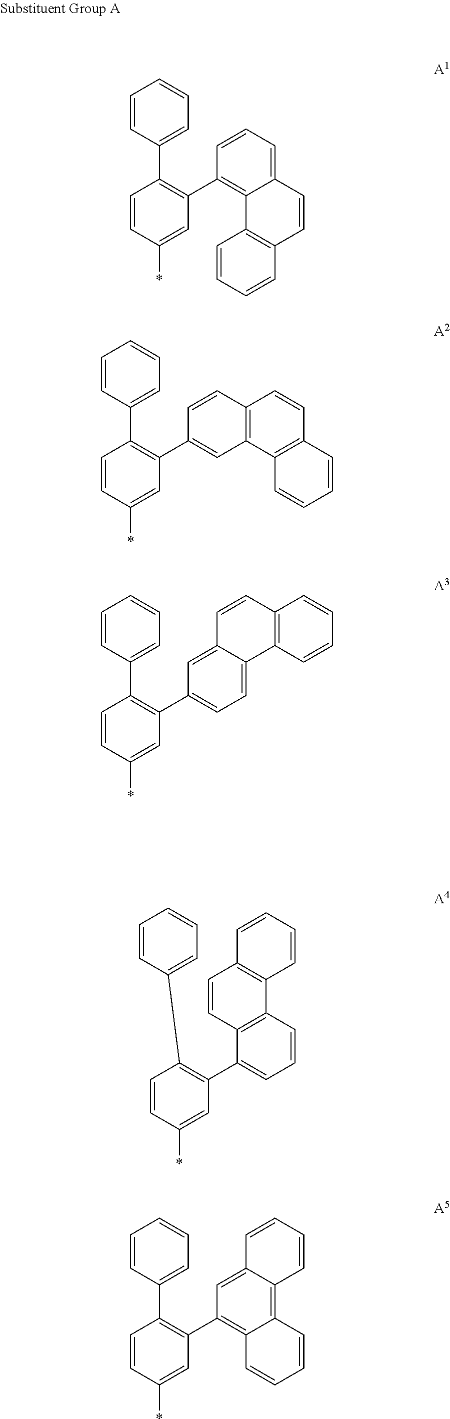 [SMILES: *c1ccc(-c2ccccc2)c(-c2cccc3ccc4ccccc4c23)c1 ; *c1ccc(-c2ccccc2)c(-c2ccc3ccc4ccccc4c3c2)c1 ; *c1ccc(-c2ccccc2)c(-c2ccc3c(ccc4ccccc43)c2)c1 ; *c1ccc(-c2ccccc2)c(-c2cccc3c2ccc2ccccc23)c1 ; *c1ccc(-c2ccccc2)c(-c2cc3ccccc3c3ccccc23)c1] 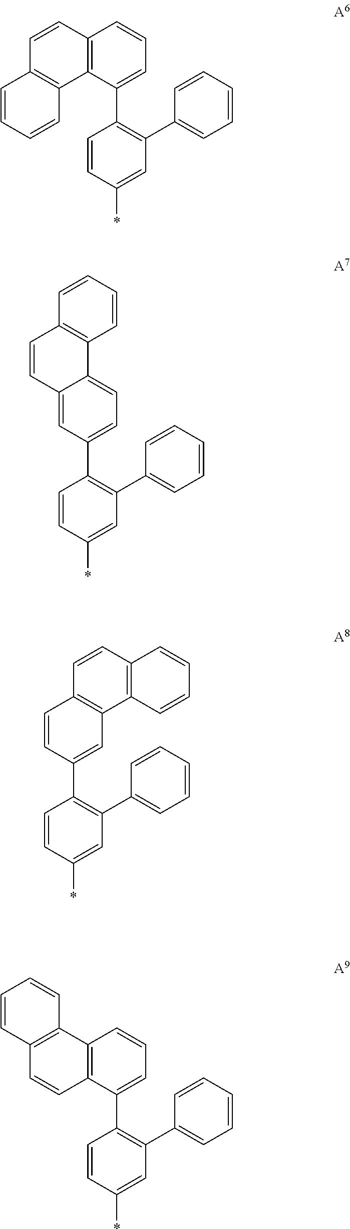 [SMILES: *c1ccc(-c2cccc3ccc4ccccc4c23)c(-c2ccccc2)c1 ; *c1ccc(-c2ccc3c(ccc4ccccc43)c2)c(-c2ccccc2)c1 ; *c1ccc(-c2ccc3ccc4ccccc4c3c2)c(-c2ccccc2)c1 ; *c1ccc(-c2cccc3c2ccc2ccccc23)c(-c2ccccc2)c1] 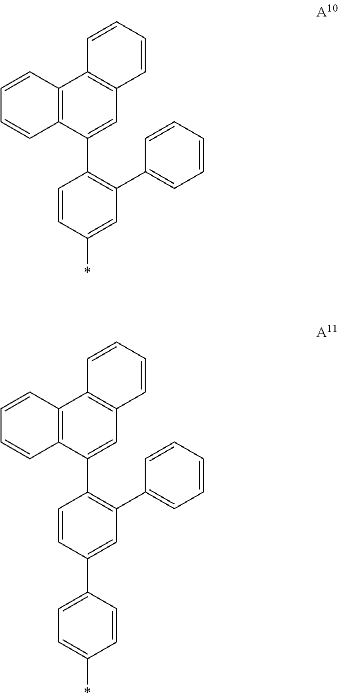 [SMILES: *c1ccc(-c2cc3ccccc3c3ccccc23)c(-c2ccccc2)c1 ; *c1ccc(-c2ccc(-c3cc4ccccc4c4ccccc34)c(-c3ccccc3)c2)cc1] 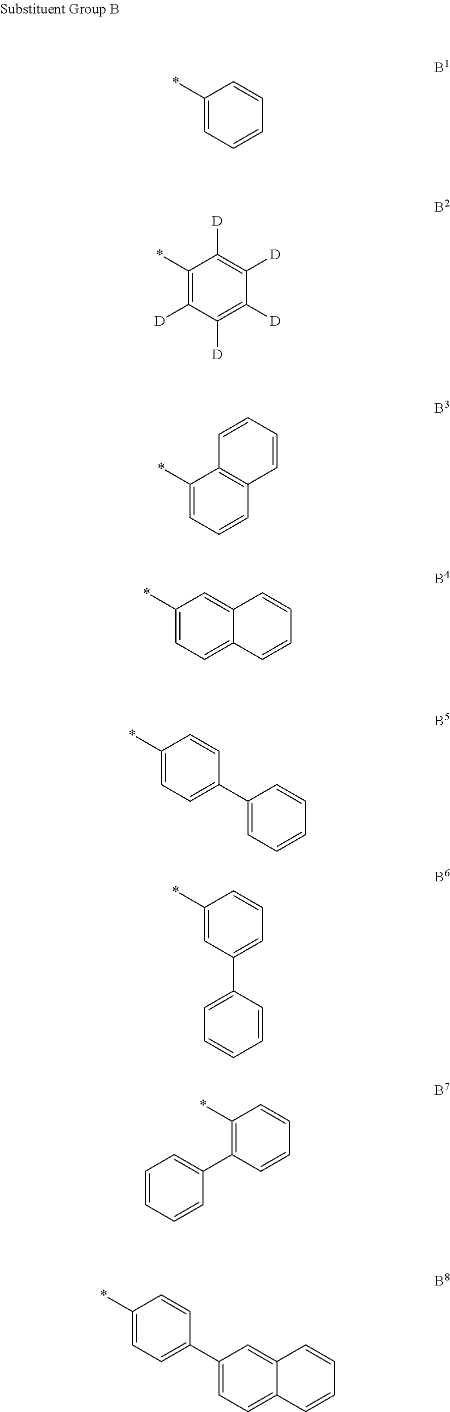 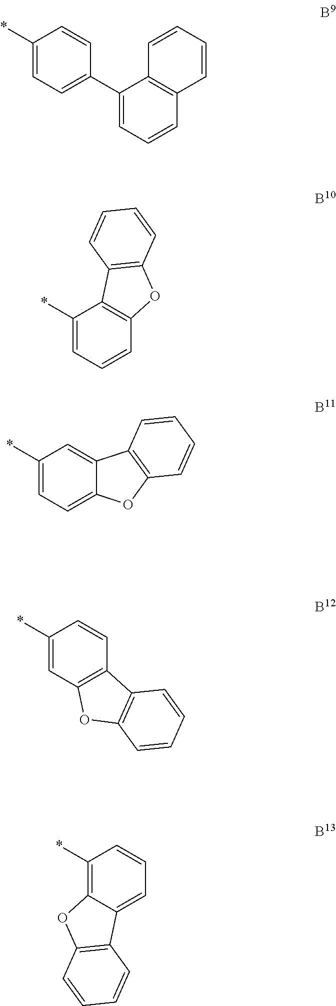 [SMILES: *c1ccc(-c2cccc3ccccc23)cc1 ; *c1cccc2oc3ccccc3c12 ; *c1ccc2oc3ccccc3c2c1 ; *c1ccc2c(c1)oc1ccccc12 ; *c1cccc2c1oc1ccccc12] 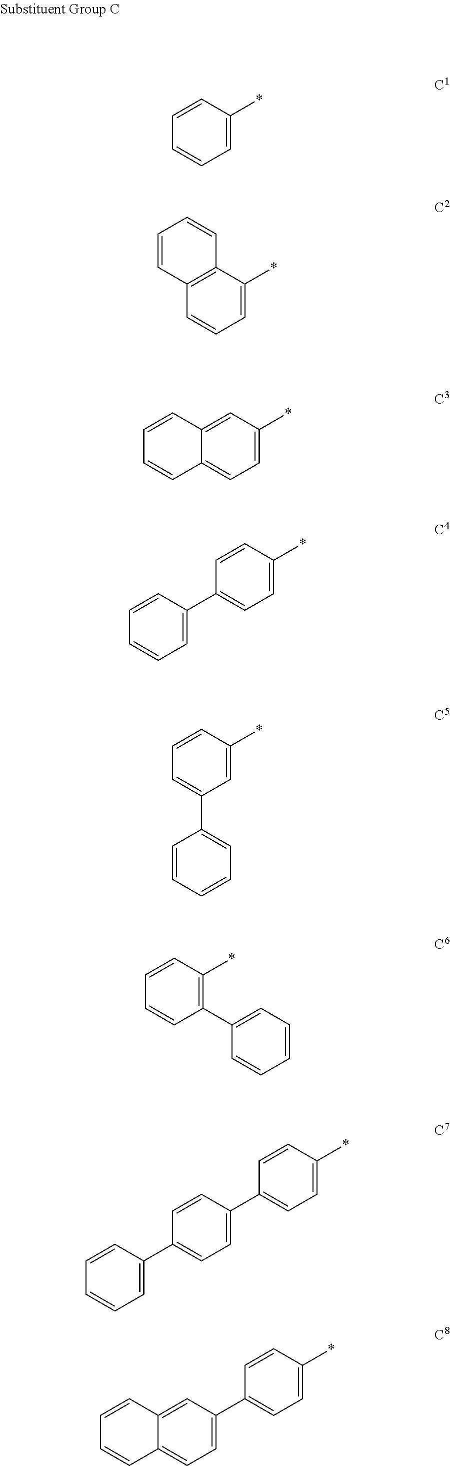 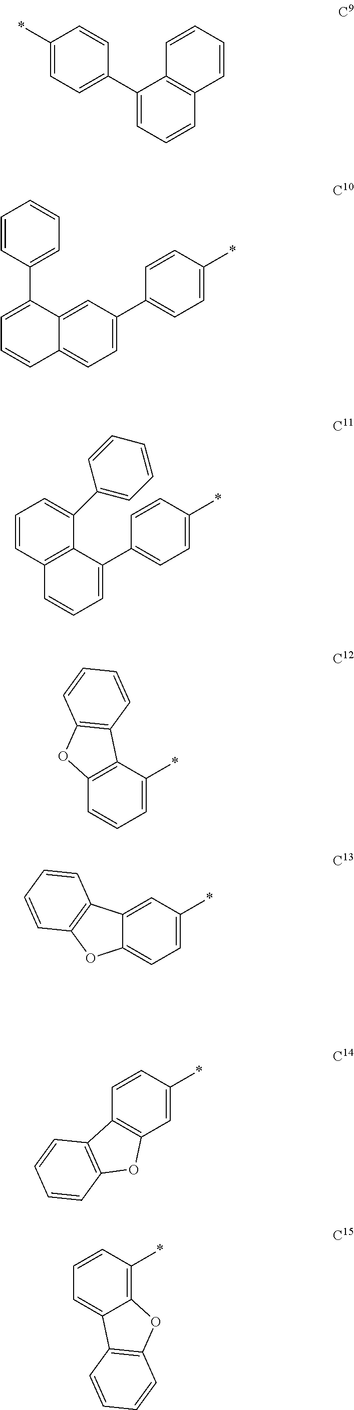 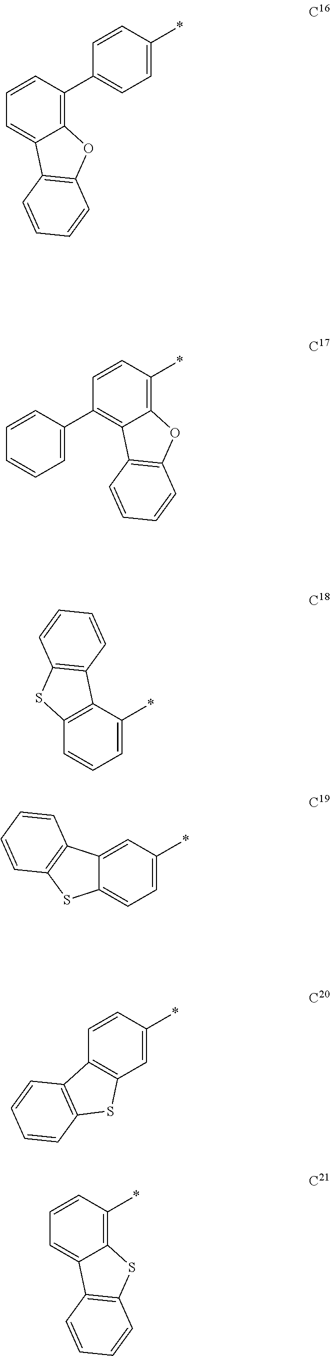 [SMILES: *c1ccc(-c2cccc3c2oc2ccccc23)cc1 ; *c1ccc(-c2ccccc2)c2c1oc1ccccc12 ; *c1cccc2sc3ccccc3c12 ; *c1ccc2sc3ccccc3c2c1 ; *c1ccc2c(c1)sc1ccccc12 ; *c1cccc2c1sc1ccccc12] 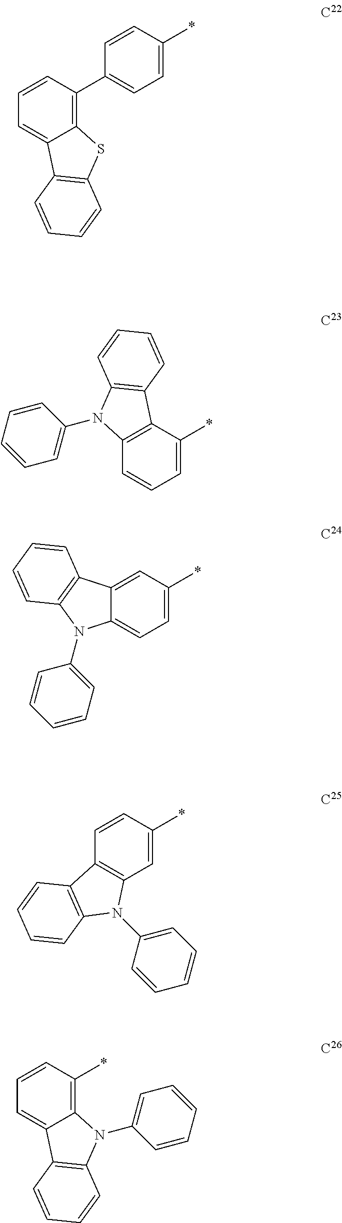 [SMILES: *c1ccc(-c2cccc3c2sc2ccccc23)cc1 ; *c1cccc2c1c1ccccc1n2-c1ccccc1 ; *c1ccc2c(c1)c1ccccc1n2-c1ccccc1 ; *c1ccc2c3ccccc3n(-c3ccccc3)c2c1 ; *c1cccc2c3ccccc3n(-c3ccccc3)c12] 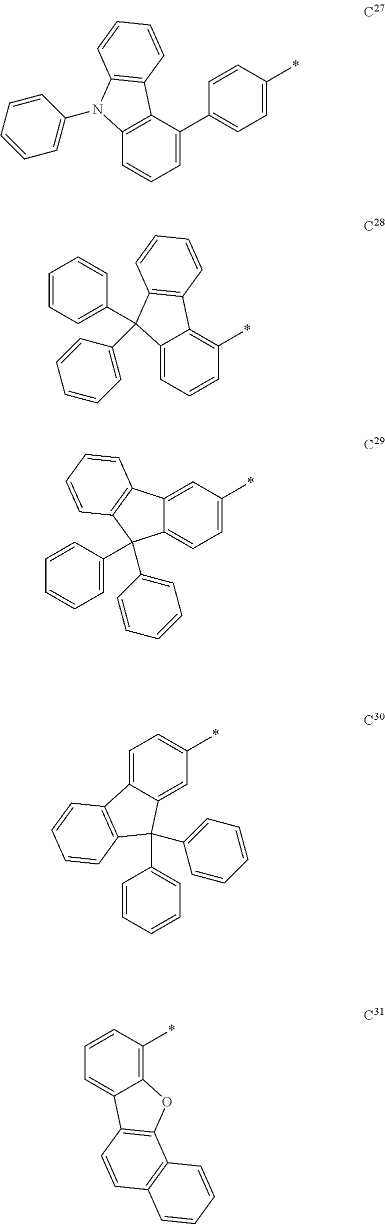 [SMILES: *c1ccc(-c2cccc3c2c2ccccc2n3-c2ccccc2)cc1 ; *c1cccc2c1-c1ccccc1C2(c1ccccc1)c1ccccc1 ; *c1ccc2c(c1)-c1ccccc1C2(c1ccccc1)c1ccccc1 ; *c1ccc2c(c1)C(c1ccccc1)(c1ccccc1)c1ccccc1-2 ; *c1cccc2c1oc1c3ccccc3ccc21] 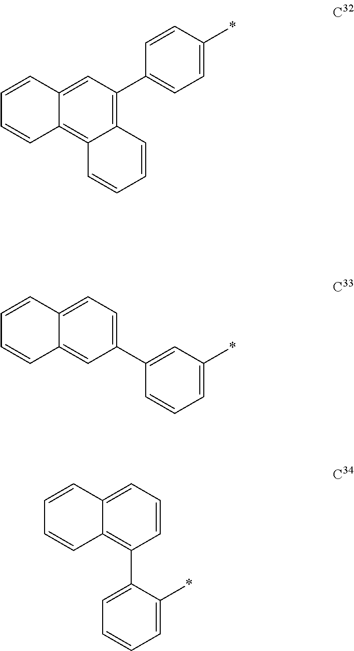 [SMILES: *c1ccc(-c2cc3ccccc3c3ccccc23)cc1 ; *c1cccc(-c2ccc3ccccc3c2)c1 ; *c1ccccc1-c1cccc2ccccc12]
COMPOUND COMBINATION TABLE 1

| No. | A | B | C |
|---|---|---|---|
| 1 | 1 | 1 | 1 |
| 2 | 2 | 1 | 1 |
| 3 | 3 | 1 | 1 |
| 4 | 4 | 1 | 1 |
| 5 | 6 | 1 | 1 |
| 6 | 7 | 1 | 1 |
| 7 | 8 | 1 | 1 |
| 8 | 9 | 1 | 1 |
| 9 | 1 | 1 | 2 |
| 10 | 2 | 1 | 2 |
| 11 | 3 | 1 | 2 |
| 12 | 4 | 1 | 2 |
| 13 | 5 | 1 | 33 |
| 14 | 6 | 1 | 2 |
| 15 | 7 | 1 | 2 |
| 16 | 8 | 1 | 2 |
| 17 | 9 | 1 | 2 |
| 18 | 10 | 1 | 33 |
| 19 | 11 | 1 | 33 |
| 20 | 1 | 1 | 3 |
| 21 | 2 | 1 | 3 |
| 22 | 3 | 1 | 3 |
| 23 | 4 | 1 | 3 |
| 24 | 5 | 1 | 34 |
| 25 | 6 | 1 | 3 |
| 26 | 7 | 1 | 3 |
| 27 | 8 | 1 | 3 |
| 28 | 9 | 1 | 3 |
| 29 | 10 | 1 | 34 |
| 30 | 11 | 1 | 34 |
| 31 | 1 | 1 | 8 |
| 32 | 2 | 1 | 8 |
| 33 | 3 | 1 | 8 |
| 34 | 4 | 1 | 8 |
| 35 | 5 | 1 | 8 |
| 36 | 6 | 1 | 8 |
| 37 | 7 | 1 | 8 |
| 38 | 8 | 1 | 8 |
| 39 | 9 | 1 | 8 |
| 40 | 10 | 1 | 8 |
| 41 | 11 | 1 | 8 |
| 42 | 1 | 2 | 1 |
| 43 | 2 | 2 | 1 |
| 44 | 3 | 2 | 1 |
| 45 | 4 | 2 | 1 |
| 46 | 6 | 2 | 1 |
| 47 | 7 | 2 | 1 |
| 48 | 8 | 2 | 1 |
| 49 | 9 | 2 | 1 |
| 50 | 1 | 2 | 2 |
| 51 | 2 | 2 | 2 |
| 52 | 3 | 2 | 2 |
| 53 | 4 | 2 | 2 |
| 54 | 5 | 2 | 33 |
| 55 | 6 | 2 | 2 |
| 56 | 7 | 2 | 2 |
| 57 | 8 | 2 | 2 |
| 58 | 9 | 2 | 2 |
| 59 | 10 | 2 | 33 |
| 60 | 11 | 2 | 33 |
| 61 | 1 | 2 | 3 |
| 62 | 2 | 2 | 3 |
| 63 | 3 | 2 | 3 |
| 64 | 4 | 2 | 3 |
| 65 | 5 | 2 | 34 |
| 66 | 6 | 2 | 3 |
| 67 | 7 | 2 | 3 |
| 68 | 8 | 2 | 3 |
| 69 | 9 | 2 | 3 |
| 70 | 10 | 2 | 34 |
| 71 | 11 | 2 | 34 |
| 72 | 1 | 2 | 8 |
| 73 | 2 | 2 | 8 |
| 74 | 3 | 2 | 8 |
| 75 | 4 | 2 | 8 |
| 76 | 5 | 2 | 8 |
| 77 | 6 | 2 | 8 |
| 78 | 7 | 2 | 8 |
| 79 | 8 | 2 | 8 |
| 80 | 9 | 2 | 8 |
| 81 | 10 | 2 | 8 |
| 82 | 11 | 2 | 8 |
| 83 | 1 | 5 | 1 |
| 84 | 2 | 5 | 1 |
| 85 | 3 | 5 | 1 |
| 86 | 4 | 5 | 1 |
| 87 | 6 | 5 | 1 |
| 88 | 7 | 5 | 1 |
| 89 | 8 | 5 | 1 |
| 90 | 9 | 5 | 1 |
| 91 | 1 | 5 | 2 |
| 92 | 2 | 5 | 2 |
| 93 | 3 | 5 | 2 |
| 94 | 4 | 5 | 2 |
| 95 | 5 | 5 | 33 |
| 96 | 6 | 5 | 2 |
| 97 | 7 | 5 | 2 |
| 98 | 8 | 5 | 2 |
| 99 | 9 | 5 | 2 |
| 100 | 10 | 5 | 33 |
| 101 | 11 | 5 | 33 |
| 102 | 1 | 5 | 3 |
| 103 | 2 | 5 | 3 |
| 104 | 3 | 5 | 3 |
| 105 | 4 | 5 | 3 |
| 106 | 5 | 5 | 34 |
| 107 | 6 | 5 | 3 |
| 108 | 7 | 5 | 3 |
| 109 | 8 | 5 | 3 |
| 110 | 9 | 5 | 3 |
| 111 | 10 | 5 | 34 |
| 112 | 11 | 5 | 34 |
| 113 | 1 | 5 | 8 |
| 114 | 2 | 5 | 8 |
| 115 | 3 | 5 | 8 |
| 116 | 4 | 5 | 8 |
| 117 | 5 | 5 | 8 |
| 118 | 6 | 5 | 8 |
| 119 | 7 | 5 | 8 |
| 120 | 8 | 5 | 8 |
| 121 | 9 | 5 | 8 |
| 122 | 10 | 5 | 8 |
| 123 | 11 | 5 | 8 |
| 124 | 2 | 6 | 1 |
| 125 | 2 | 7 | 1 |
| 126 | 2 | 9 | 1 |
| 127 | 2 | 10 | 1 |
| 128 | 2 | 11 | 1 |
| 129 | 2 | 12 | 1 |
| 130 | 2 | 13 | 1 |
| 131 | 2 | 3 | 2 |
| 132 | 2 | 4 | 2 |
| 133 | 2 | 6 | 2 |
| 134 | 2 | 7 | 2 |
| 135 | 2 | 8 | 2 |
| 136 | 2 | 9 | 2 |
| 137 | 2 | 10 | 2 |
| 138 | 2 | 11 | 2 |
| 139 | 2 | 12 | 2 |
| 140 | 2 | 13 | 2 |
| 141 | 2 | 2 | 4 |
| 142 | 2 | 5 | 4 |
| 143 | 2 | 6 | 4 |
| 144 | 2 | 7 | 4 |
| 145 | 2 | 9 | 4 |
| 146 | 2 | 10 | 4 |
| 147 | 2 | 11 | 4 |
| 148 | 2 | 12 | 4 |
| 149 | 2 | 13 | 4 |
| 150 | 2 | 4 | 8 |
| 151 | 2 | 6 | 8 |
| 152 | 2 | 7 | 8 |
| 153 | 2 | 8 | 8 |
| 154 | 2 | 9 | 8 |
| 155 | 2 | 10 | 8 |
| 156 | 2 | 11 | 8 |
| 157 | 2 | 12 | 8 |
| 158 | 2 | 13 | 8 |
| 159 | 2 | 1 | 16 |
| 160 | 2 | 2 | 16 |
| 161 | 2 | 3 | 16 |
| 162 | 2 | 4 | 16 |
| 163 | 2 | 5 | 16 |
| 164 | 2 | 6 | 16 |
| 165 | 2 | 7 | 16 |
| 166 | 2 | 8 | 16 |
| 167 | 2 | 9 | 16 |
| 168 | 2 | 10 | 16 |
| 169 | 2 | 11 | 16 |
| 170 | 2 | 12 | 16 |
| 171 | 2 | 13 | 16 |
| 172 | 2 | 1 | 22 |
| 173 | 2 | 2 | 22 |
| 174 | 2 | 3 | 22 |
| 175 | 2 | 4 | 22 |
| 176 | 2 | 5 | 22 |
| 177 | 2 | 6 | 22 |
| 178 | 2 | 7 | 22 |
| 179 | 2 | 8 | 22 |
| 180 | 2 | 9 | 22 |
| 181 | 2 | 10 | 22 |
| 182 | 2 | 11 | 22 |
| 183 | 2 | 12 | 22 |
| 184 | 2 | 13 | 22 |
| 185 | 2 | 1 | 27 |
| 186 | 2 | 2 | 27 |
| 187 | 2 | 3 | 27 |
| 188 | 2 | 4 | 27 |
| 189 | 2 | 5 | 27 |
| 190 | 2 | 6 | 27 |
| 191 | 2 | 7 | 27 |
| 192 | 2 | 8 | 27 |
| 193 | 2 | 9 | 27 |
| 194 | 2 | 10 | 27 |
| 195 | 2 | 11 | 27 |
| 196 | 2 | 12 | 27 |
| 197 | 2 | 13 | 27 |
| 198 | 5 | 9 | 1 |
| 199 | 5 | 10 | 1 |
| 200 | 5 | 11 | 1 |
| 201 | 5 | 12 | 4 |
| 202 | 5 | 13 | 1 |
| 203 | 5 | 3 | 33 |
| 204 | 5 | 4 | 33 |
| 205 | 5 | 6 | 33 |
| 206 | 5 | 7 | 33 |
| 207 | 5 | 8 | 2 |
| 208 | 5 | 9 | 2 |
| 209 | 5 | 10 | 2 |
| 210 | 5 | 11 | 2 |
| 211 | 5 | 12 | 2 |
| 212 | 5 | 13 | 2 |
| 213 | 5 | 9 | 4 |
| 214 | 5 | 10 | 4 |
| 215 | 5 | 11 | 4 |
| 216 | 5 | 12 | 4 |
| 217 | 5 | 13 | 4 |
| 218 | 5 | 4 | 8 |
| 219 | 5 | 6 | 8 |
| 220 | 5 | 7 | 8 |
| 221 | 5 | 8 | 8 |
| 222 | 5 | 9 | 8 |
| 223 | 5 | 10 | 8 |
| 224 | 5 | 11 | 8 |
| 225 | 5 | 12 | 8 |
| 226 | 5 | 13 | 8 |
| 227 | 5 | 1 | 16 |
| 228 | 5 | 2 | 16 |
| 229 | 5 | 3 | 16 |
| 230 | 5 | 4 | 16 |
| 231 | 5 | 5 | 16 |
| 232 | 5 | 6 | 16 |
| 233 | 5 | 7 | 16 |
| 234 | 5 | 8 | 16 |
| 235 | 5 | 9 | 16 |
| 236 | 5 | 10 | 16 |
| 237 | 5 | 11 | 16 |
| 238 | 5 | 12 | 16 |
| 239 | 5 | 13 | 16 |
| 240 | 5 | 1 | 22 |
| 241 | 5 | 2 | 22 |
| 242 | 5 | 3 | 22 |
| 243 | 5 | 4 | 22 |
| 244 | 5 | 5 | 22 |
| 245 | 5 | 6 | 22 |
| 246 | 5 | 7 | 22 |
| 247 | 5 | 8 | 22 |
| 248 | 5 | 9 | 22 |
| 249 | 5 | 10 | 22 |
| 250 | 5 | 11 | 22 |
| 251 | 5 | 12 | 22 |
| 252 | 5 | 13 | 22 |
| 253 | 5 | 1 | 27 |
| 254 | 5 | 2 | 27 |
| 255 | 5 | 3 | 27 |
| 256 | 5 | 4 | 27 |
| 257 | 5 | 5 | 27 |
| 258 | 5 | 6 | 27 |
| 259 | 5 | 7 | 27 |
| 260 | 5 | 8 | 27 |
| 261 | 5 | 9 | 27 |
| 262 | 5 | 10 | 27 |
| 263 | 5 | 11 | 27 |
| 264 | 5 | 12 | 27 |
| 265 | 5 | 13 | 27 |
| 266 | 8 | 6 | 1 |
| 267 | 8 | 7 | 1 |
| 268 | 8 | 9 | 1 |
| 269 | 8 | 10 | 1 |
| 270 | 8 | 11 | 1 |
| 271 | 8 | 12 | 1 |
| 272 | 8 | 13 | 1 |
| 273 | 8 | 3 | 2 |
| 274 | 8 | 4 | 2 |
| 275 | 8 | 6 | 2 |
| 276 | 8 | 7 | 2 |
| 277 | 8 | 8 | 2 |
| 278 | 8 | 9 | 2 |
| 279 | 8 | 10 | 2 |
| 280 | 8 | 11 | 2 |
| 281 | 8 | 12 | 2 |
| 282 | 8 | 13 | 2 |
| 283 | 8 | 2 | 4 |
| 284 | 8 | 5 | 4 |
| 285 | 8 | 6 | 4 |
| 286 | 8 | 7 | 4 |
| 287 | 8 | 9 | 4 |
| 288 | 8 | 10 | 4 |
| 289 | 8 | 11 | 4 |
| 290 | 8 | 12 | 4 |
| 291 | 8 | 13 | 4 |
| 292 | 8 | 4 | 8 |
| 293 | 8 | 6 | 8 |
| 294 | 8 | 7 | 8 |
| 295 | 8 | 8 | 8 |
| 296 | 8 | 9 | 8 |
| 297 | 8 | 10 | 8 |
| 298 | 8 | 11 | 8 |
| 299 | 8 | 12 | 8 |
| 300 | 8 | 13 | 8 |
| 301 | 8 | 1 | 16 |
| 302 | 8 | 2 | 16 |
| 303 | 8 | 3 | 16 |
| 304 | 8 | 4 | 16 |
| 305 | 8 | 5 | 16 |
| 306 | 8 | 6 | 16 |
| 307 | 8 | 7 | 16 |
| 308 | 8 | 8 | 16 |
| 309 | 8 | 9 | 16 |
| 310 | 8 | 10 | 16 |
| 311 | 8 | 11 | 16 |
| 312 | 8 | 12 | 16 |
| 313 | 8 | 13 | 16 |
| 314 | 8 | 1 | 22 |
| 315 | 8 | 2 | 22 |
| 316 | 8 | 3 | 22 |
| 317 | 8 | 4 | 22 |
| 318 | 8 | 5 | 22 |
| 319 | 8 | 6 | 22 |
| 320 | 8 | 7 | 22 |
| 321 | 8 | 8 | 22 |
| 322 | 8 | 9 | 22 |
| 323 | 8 | 10 | 22 |
| 324 | 8 | 11 | 22 |
| 325 | 8 | 12 | 22 |
| 326 | 8 | 13 | 22 |
| 327 | 8 | 1 | 27 |
| 328 | 8 | 2 | 27 |
| 329 | 8 | 3 | 27 |
| 330 | 8 | 4 | 27 |
| 331 | 8 | 5 | 27 |
| 332 | 8 | 6 | 27 |
| 333 | 8 | 7 | 27 |
| 334 | 8 | 8 | 27 |
| 335 | 8 | 9 | 27 |
| 336 | 8 | 10 | 27 |
| 337 | 8 | 11 | 27 |
| 338 | 8 | 12 | 27 |
| 339 | 8 | 13 | 27 |
| 340 | 10 | 9 | 1 |
| 341 | 10 | 10 | 1 |
| 342 | 10 | 11 | 1 |
| 343 | 10 | 12 | 1 |
| 344 | 10 | 13 | 1 |
| 345 | 10 | 3 | 33 |
| 346 | 10 | 4 | 33 |
| 347 | 10 | 6 | 33 |
| 348 | 10 | 7 | 33 |
| 349 | 10 | 8 | 2 |
| 350 | 10 | 9 | 2 |
| 351 | 10 | 10 | 2 |
| 352 | 10 | 11 | 2 |
| 353 | 10 | 12 | 2 |
| 354 | 10 | 13 | 2 |
| 355 | 10 | 9 | 4 |
| 356 | 10 | 10 | 4 |
| 357 | 10 | 11 | 4 |
| 358 | 10 | 12 | 4 |
| 359 | 10 | 13 | 4 |
| 360 | 10 | 4 | 8 |
| 361 | 10 | 6 | 8 |
| 362 | 10 | 7 | 8 |
| 363 | 10 | 8 | 8 |
| 364 | 10 | 9 | 8 |
| 365 | 10 | 10 | 8 |
| 366 | 10 | 11 | 8 |
| 367 | 10 | 12 | 8 |
| 368 | 10 | 13 | 8 |
| 369 | 10 | 1 | 16 |
| 370 | 10 | 2 | 16 |
| 371 | 10 | 3 | 16 |
| 372 | 10 | 4 | 16 |
| 373 | 10 | 5 | 16 |
| 374 | 10 | 6 | 16 |
| 375 | 10 | 7 | 16 |
| 376 | 10 | 8 | 16 |
| 377 | 10 | 9 | 16 |
| 378 | 10 | 10 | 16 |
| 379 | 10 | 11 | 16 |
| 380 | 10 | 12 | 16 |
| 381 | 10 | 13 | 16 |
| 382 | 10 | 1 | 22 |
| 383 | 10 | 2 | 22 |
| 384 | 10 | 3 | 22 |
| 385 | 10 | 4 | 22 |
| 386 | 10 | 5 | 22 |
| 387 | 10 | 6 | 22 |
| 388 | 10 | 7 | 22 |
| 389 | 10 | 8 | 22 |
| 390 | 10 | 9 | 22 |
| 391 | 10 | 10 | 22 |
| 392 | 10 | 11 | 22 |
| 393 | 10 | 12 | 22 |
| 394 | 10 | 13 | 22 |
| 395 | 10 | 1 | 27 |
| 396 | 10 | 2 | 27 |
| 397 | 10 | 3 | 27 |
| 398 | 10 | 4 | 27 |
| 399 | 10 | 5 | 27 |
| 400 | 10 | 6 | 27 |
| 401 | 10 | 7 | 27 |
| 402 | 10 | 8 | 27 |
| 403 | 10 | 9 | 27 |
| 404 | 10 | 10 | 27 |
| 405 | 10 | 11 | 27 |
| 406 | 10 | 12 | 27 |
| 407 | 10 | 13 | 27 |
| 408 | 2 | 1 | 7 |
| 409 | 2 | 1 | 10 |
| 410 | 2 | 1 | 11 |
| 411 | 2 | 1 | 17 |
| 412 | 2 | 1 | 18 |
| 413 | 2 | 1 | 19 |
| 414 | 2 | 1 | 20 |
| 415 | 2 | 1 | 21 |
| 416 | 2 | 1 | 23 |
| 417 | 2 | 1 | 24 |
| 418 | 2 | 1 | 25 |
| 419 | 2 | 1 | 26 |
| 420 | 2 | 1 | 28 |
| 421 | 2 | 1 | 29 |
| 422 | 2 | 1 | 30 |
| 423 | 2 | 1 | 31 |
| 424 | 2 | 1 | 32 |
| 425 | 2 | 2 | 5 |
| 426 | 2 | 2 | 6 |
| 427 | 2 | 2 | 7 |
| 428 | 2 | 2 | 9 |
| 429 | 2 | 2 | 10 |
| 430 | 2 | 2 | 11 |
| 431 | 2 | 2 | 12 |
| 432 | 2 | 2 | 13 |
| 433 | 2 | 2 | 14 |
| 434 | 2 | 2 | 15 |
| 435 | 2 | 2 | 17 |
| 436 | 2 | 2 | 18 |
| 437 | 2 | 2 | 19 |
| 438 | 2 | 2 | 20 |
| 439 | 2 | 2 | 21 |
| 440 | 2 | 2 | 23 |
| 441 | 2 | 2 | 24 |
| 442 | 2 | 2 | 25 |
| 443 | 2 | 2 | 26 |
| 444 | 2 | 2 | 28 |
| 445 | 2 | 2 | 29 |
| 446 | 2 | 2 | 30 |
| 447 | 2 | 2 | 31 |
| 448 | 2 | 2 | 32 |
| 449 | 2 | 12 | 3 |
| 450 | 2 | 12 | 5 |
| 451 | 2 | 12 | 6 |
| 452 | 2 | 12 | 7 |
| 453 | 2 | 12 | 9 |
| 454 | 2 | 12 | 10 |
| 455 | 2 | 12 | 11 |
| 456 | 2 | 12 | 12 |
| 457 | 2 | 12 | 13 |
| 458 | 2 | 12 | 14 |
| 459 | 2 | 12 | 15 |
| 460 | 2 | 12 | 17 |
| 461 | 2 | 12 | 18 |
| 462 | 2 | 12 | 19 |
| 463 | 2 | 12 | 20 |
| 464 | 2 | 12 | 21 |
| 465 | 2 | 12 | 23 |
| 466 | 2 | 12 | 24 |
| 467 | 2 | 12 | 25 |
| 468 | 2 | 12 | 26 |
| 469 | 2 | 12 | 28 |
| 470 | 2 | 12 | 29 |
| 471 | 2 | 12 | 30 |
| 472 | 2 | 3 | 31 |
| 473 | 2 | 3 | 32 |
| 474 | 5 | 1 | 10 |
| 475 | 5 | 1 | 11 |
| 476 | 5 | 1 | 17 |
| 477 | 5 | 1 | 18 |
| 478 | 5 | 1 | 19 |
| 479 | 5 | 1 | 20 |
| 480 | 5 | 1 | 21 |
| 481 | 5 | 1 | 23 |
| 482 | 5 | 1 | 24 |
| 483 | 5 | 1 | 25 |
| 484 | 5 | 1 | 26 |
| 485 | 5 | 1 | 28 |
| 486 | 5 | 1 | 29 |
| 487 | 5 | 1 | 30 |
| 488 | 5 | 1 | 31 |
| 489 | 5 | 1 | 32 |
| 490 | 5 | 2 | 9 |
| 491 | 5 | 2 | 10 |
| 492 | 5 | 2 | 11 |
| 493 | 5 | 2 | 12 |
| 494 | 5 | 2 | 13 |
| 495 | 5 | 2 | 14 |
| 496 | 5 | 2 | 15 |
| 497 | 5 | 2 | 17 |
| 498 | 5 | 2 | 18 |
| 499 | 5 | 2 | 19 |
| 500 | 5 | 2 | 20 |
| 501 | 5 | 2 | 21 |
| 502 | 5 | 2 | 23 |
| 503 | 5 | 2 | 24 |
| 504 | 5 | 2 | 25 |
| 505 | 5 | 2 | 26 |
| 506 | 5 | 2 | 28 |
| 507 | 5 | 2 | 29 |
| 508 | 5 | 2 | 30 |
| 509 | 5 | 2 | 31 |
| 510 | 5 | 2 | 32 |
| 511 | 5 | 12 | 3 |
| 512 | 5 | 12 | 5 |
| 513 | 5 | 12 | 6 |
| 514 | 5 | 12 | 7 |
| 515 | 5 | 12 | 9 |
| 516 | 5 | 12 | 10 |
| 517 | 5 | 12 | 11 |
| 518 | 5 | 12 | 12 |
| 519 | 5 | 12 | 13 |
| 520 | 5 | 12 | 14 |
| 521 | 5 | 12 | 15 |
| 522 | 5 | 12 | 17 |
| 523 | 5 | 12 | 18 |
| 524 | 5 | 12 | 19 |
| 525 | 5 | 12 | 20 |
| 526 | 5 | 12 | 21 |
| 527 | 5 | 12 | 23 |
| 528 | 5 | 12 | 24 |
| 529 | 5 | 12 | 25 |
| 530 | 5 | 12 | 26 |
| 531 | 5 | 12 | 28 |
| 532 | 5 | 12 | 29 |
| 533 | 5 | 12 | 30 |
| 534 | 5 | 3 | 31 |
| 535 | 5 | 3 | 32 |
| 536 | 8 | 1 | 7 |
| 537 | 8 | 1 | 10 |
| 538 | 8 | 1 | 11 |
| 539 | 8 | 1 | 17 |
| 540 | 8 | 1 | 18 |
| 541 | 8 | 1 | 19 |
| 542 | 8 | 1 | 20 |
| 543 | 8 | 1 | 21 |
| 544 | 8 | 1 | 23 |
| 545 | 8 | 1 | 24 |
| 546 | 8 | 1 | 25 |
| 547 | 8 | 1 | 26 |
| 548 | 8 | 1 | 28 |
| 549 | 8 | 1 | 29 |
| 550 | 8 | 1 | 30 |
| 551 | 8 | 1 | 31 |
| 552 | 8 | 1 | 32 |
| 553 | 8 | 2 | 5 |
| 554 | 8 | 2 | 6 |
| 555 | 8 | 2 | 7 |
| 556 | 8 | 2 | 9 |
| 557 | 8 | 2 | 10 |
| 558 | 8 | 2 | 11 |
| 559 | 8 | 2 | 12 |
| 560 | 8 | 2 | 13 |
| 561 | 8 | 2 | 14 |
| 562 | 8 | 2 | 15 |
| 563 | 8 | 2 | 17 |
| 564 | 8 | 2 | 18 |
| 565 | 8 | 2 | 19 |
| 566 | 8 | 2 | 20 |
| 567 | 8 | 2 | 21 |
| 568 | 8 | 2 | 23 |
| 569 | 8 | 2 | 24 |
| 570 | 8 | 2 | 25 |
| 571 | 8 | 2 | 26 |
| 572 | 8 | 2 | 28 |
| 573 | 8 | 2 | 29 |
| 574 | 8 | 2 | 30 |
| 575 | 8 | 2 | 31 |
| 576 | 8 | 2 | 32 |
| 577 | 8 | 12 | 3 |
| 578 | 8 | 12 | 5 |
| 579 | 8 | 12 | 6 |
| 580 | 8 | 12 | 7 |
| 581 | 8 | 12 | 9 |
| 582 | 8 | 12 | 10 |
| 583 | 8 | 12 | 11 |
| 584 | 8 | 12 | 12 |
| 585 | 8 | 12 | 13 |
| 586 | 8 | 12 | 14 |
| 587 | 8 | 12 | 15 |
| 588 | 8 | 12 | 17 |
| 589 | 8 | 12 | 18 |
| 590 | 8 | 12 | 19 |
| 591 | 8 | 12 | 20 |
| 592 | 8 | 12 | 21 |
| 593 | 8 | 12 | 23 |
| 594 | 8 | 12 | 24 |
| 595 | 8 | 12 | 25 |
| 596 | 8 | 12 | 26 |
| 597 | 8 | 12 | 28 |
| 598 | 8 | 12 | 29 |
| 599 | 8 | 12 | 30 |
| 600 | 8 | 12 | 31 |
| 601 | 8 | 12 | 32 |
| 602 | 10 | 1 | 10 |
| 603 | 10 | 1 | 11 |
| 604 | 10 | 1 | 17 |
| 605 | 10 | 1 | 18 |
| 606 | 10 | 1 | 19 |
| 607 | 10 | 1 | 20 |
| 608 | 10 | 1 | 21 |
| 609 | 10 | 1 | 23 |
| 610 | 10 | 1 | 24 |
| 611 | 10 | 1 | 25 |
| 612 | 10 | 1 | 26 |
| 613 | 10 | 1 | 28 |
| 614 | 10 | 1 | 29 |
| 615 | 10 | 1 | 30 |
| 616 | 10 | 1 | 31 |
| 617 | 10 | 1 | 32 |
| 618 | 10 | 2 | 9 |
| 619 | 10 | 2 | 10 |
| 620 | 10 | 2 | 11 |
| 621 | 10 | 2 | 12 |
| 622 | 10 | 2 | 13 |
| 623 | 10 | 2 | 14 |
| 624 | 10 | 2 | 15 |
| 625 | 10 | 2 | 17 |
| 626 | 10 | 2 | 18 |
| 627 | 10 | 2 | 19 |
| 628 | 10 | 2 | 20 |
| 629 | 10 | 2 | 21 |
| 630 | 10 | 2 | 23 |
| 631 | 10 | 2 | 24 |
| 632 | 10 | 2 | 25 |
| 633 | 10 | 2 | 26 |
| 634 | 10 | 2 | 28 |
| 635 | 10 | 2 | 29 |
| 636 | 10 | 2 | 30 |
| 637 | 10 | 2 | 31 |
| 638 | 10 | 2 | 32 |
| 639 | 10 | 12 | 3 |
| 640 | 10 | 12 | 5 |
| 641 | 10 | 12 | 6 |
| 642 | 10 | 12 | 7 |
| 643 | 10 | 12 | 9 |
| 644 | 10 | 12 | 10 |
| 645 | 10 | 12 | 11 |
| 646 | 10 | 12 | 12 |
| 647 | 10 | 12 | 13 |
| 648 | 10 | 12 | 14 |
| 649 | 10 | 12 | 15 |
| 650 | 10 | 12 | 17 |
| 651 | 10 | 12 | 18 |
| 652 | 10 | 12 | 19 |
| 663 | 10 | 12 | 20 |
| 654 | 10 | 12 | 21 |
| 655 | 10 | 12 | 23 |
| 656 | 10 | 12 | 24 |
| 657 | 10 | 12 | 25 |
| 658 | 10 | 12 | 26 |
| 659 | 10 | 12 | 28 |
| 660 | 10 | 12 | 29 |
| 661 | 10 | 12 | 30 |
| 662 | 10 | 3 | 31 |
| 663 | 10 | 3 | 32 |
